(19) United States
(12) Patent Application Publication
(10) Pub. No.: US 2019/0057186 A1
(43) Pub. Date: Feb. 21, 2019

(54) METHODS AND COMPOSITIONS FOR THE DETECTION, CLASSIFICATION, AND DIAGNOSIS OF SCHIZOPHRENIA

(71) Applicant: Washington University, St. Louis, MO (US)

(72) Inventors: Jorge Sergio Zwir, St. Louis, MO (US); Claude Robert Cloninger, St. Louis, MO (US); Francisco Javier Arnedo Fernandez, Granada (ES); Dragan Svrakic, St. Louis, MO (US); Maria Coral del Val Munoz, Granada (ES); Gabriel Alejandro de Erausquin, Tampa, FL (US)

(21) Appl. No.: 16/168,525

(22) Filed: Oct. 23, 2018

Related U.S. Application Data
(63) Continuation of application No. 14/840,806, filed on Aug. 31, 2015.
(60) Provisional application No. 62/043,871, filed on Aug. 29, 2014.

(51) Int. Cl. G06F 19/18; G06F 19/22; C12Q 1/6883

(52) U.S. Cl. CPC G06F 19/18; C12Q 2600/112; C12Q 2600/16; C12Q 2600/158; C12Q 1/6883; G06F 19/22; C12Q 2600/156

(57) ABSTRACT

Disclosed are compositions and methods for the diagnosis and classification of schizophrenia.

### CROSS REFERENCE TO RELATED APPLICATIONS

This application is a continuation of U.S. application Ser. No. 14/840,806, filed on Aug. 31, 2015 which claims the benefit of U.S. Provisional Application No. 62/043,871, filed on Aug. 29, 2014, each of which is incorporated herein by reference in its entirety.

### I. BACKGROUND

Patients with metal disorders may receive the same diagnosis, and yet share few symptoms in common, vary widely in severity, and respond differently to treatments. Genetic association studies of mental disorders were plagued by weak and inconsistent findings, largely as a result of the clinical and etiologic heterogeneity of the cases when people were described only as having the disorder or not (cases vs controls). Classifications based on clinical features without regard for measured genotypic differences also failed to predict response to treatment.

A disorder is “complex” when it is influenced by the combined effects of interacting genes. Individual genes do not consistently cause a mental disorder; rather, it takes many genes operating in concert, possibly interacting with specific environmental factors, in order for a person to develop mental illness. Complex diseases, such as schizophrenia, may be influenced by hundreds or thousands of genetic variants that interact with one another in complex ways, and consequently display a multifaceted genetic architecture. The genetic architecture of heritable diseases refers to the number, frequency, and effect sizes of genetic risk alleles and the way they are organized into genotypic networks. In complex disorders, the same genotypic networks may lead to different clinical outcomes (a concept known as multifinality, which is called pleiotropy in genetics), and different genotypic networks may lead to the same clinical outcome (equifinality, which is also described as heterogeneity). In general, geneticists must expect the likelihood that many genes affect each trait and each gene affects many traits. Consequently, research on complex heritable disorders like schizophrenia is likely to yield weak and inconsistent results unless the complexity of their genetic and phenotypic architecture is taken into account.

For example, twin and family studies of schizophrenia consistently indicate that the variability in risk of disease is highly heritable (81%), but only 25% of the variability has been explained by specific genetic variants identified in genome-wide association studies (GWAS). This is not surprising for complex disorders like schizophrenia because current GWAS methods have been unable to characterize the gene-gene interactions (FIG. 1A) that influence the developing clinical profiles (FIG. 1B) in complex ways. The frequent failure to account for most of the heritability of complex disorders has been called the “missing” or “hidden” heritability problem.

In past studies of schizophrenia, the missing heritability problem has been approached by analyzing the explained variance in large individual samples or by using meta-analysis to combine data sets. Efforts have also been made to consider the impact of variation related to ethnicity, sex, chromosomes, functional observations, or allele frequency. Nevertheless, most of the heritability of schizophrenia remains unexplained. What is needed are new diagnostic methods that look at both the genetic and phenotypic characteristic of schizophrenia and tools for the performance and analysis of such methods.

### II. SUMMARY

Disclosed are methods and compositions related to diagnosing, assessing the risk, and classifying a subject with schizophrenia.

In one aspect, disclosed herein are diagnostic systems for diagnosing schizophrenia, wherein the diagnostic system comprises one or more expression panels, wherein the one or more expression panels each comprise one or more of the single nucleotide polymorphism (SNP) sets comprising 19_2, 88_64, 81_13, 87_76, 58_29, 83_41, 9_9, 10_4, 14_6, 56_30, 42_37, 65_25, 71_55, 12_11, 90_78, 77_5, 88_8, 51_28, 59_48, 41_12, 22_11, 13_12, 31_22, 85_84, 87_84, 16_10, 56_19, 75_31, 81_73, 85_23, 21_8, 76_74, 61_39, 75_67, 76_63, 81_3, 87_26, 88_43, 25_10, 12_2, 52_42, and/or 54_51.

Also disclosed is the diagnostic system of any preceding aspect, wherein the system selects for “severe process, with positive and negative symptom schizophrenia”, and wherein the one or more SNP sets comprise 56_30, 75_67, and/or 76_74.

Also disclosed is the diagnostic system of any preceding aspect, wherein the system selects for “positive and negative symptom Schizophrenia”, and wherein the one or more SNP sets comprise 59_48, 71_55, 21_8, 54_51, 31_22, 65_25, and/or 87_84.

Also disclosed is the diagnostic system of any preceding aspect, wherein the system selects for “negative Schizophrenia”, and wherein the one or more SNP sets comprise 58_29, 9_9, 22_11, 81_3, 13_12, 61_39, 10_4, 81_73, 75_31, 56_19, 88_8, and/or 12_2.

Also disclosed is the diagnostic system of any preceding aspect, wherein the system selects for “Positive Schizophrenia”, and wherein the one or more SNP sets comprise 88_64, 85_84, and/or 41_12.

Also disclosed is the diagnostic system of any preceding aspect, wherein the system selects for “severe process, positive schizophrenia”, and wherein the one or more SNP sets comprise 77_5, 81_13, and/or 25_10.

Also disclosed is the diagnostic system of any preceding aspect, wherein the system selects for “moderate process, disorganized negative schizophrenia”, and wherein the one or more SNP sets comprise 19_2, 52_42, 90_78, 12_11, 87_76, and/or 14_6.

Also disclosed is the diagnostic system of any preceding aspect, wherein the system selects for “moderate process, positive and negative schizophrenia”, and wherein the one or more SNP sets comprise 42_37, 88_43, and/or 51_28.

Also disclosed is the diagnostic system of any preceding aspect, wherein the system selects for “moderate process, continuous positive schizophrenia”, and wherein the one or more SNP sets comprise 16_10, 83_41, and/or 87_26.

Also disclosed herein are diagnostic systems of the invention, further comprising one or more phenotype panels, wherein each phenotype panel comprises one or more phenotypic sets selected from the group comprising 15_13, 12_11, 21_1, 50_46, 9_6, 46_23, 54_11, 30_17, 18_13, 27_6, 61_18, 64_11, 65_64, 12_4, 42_9, 52_28, 7_3, 48_41, 26_8, 69_41, 10_5, 17_2, 63_24, 69_66, 22_13, 53_6, 59_41, 20_19, 55_7, 34_17, 27_7, 4_1, 66_54, 8_4, 51_38, 42_7, 18_3, 46_29, 5_2, 57_39, 11_5, 24_4, 48_7, 28_23, and/or 25_20.

Also disclosed is the diagnostic system of any preceding aspect, wherein the system selects for “severe process, with positive and negative symptom schizophrenia”, and wherein the one or more phenotypic sets comprise 15_13, 12_11, 21_1, 50_46, 9_6, 46_23, 54_11, 30_17, 18_13, 27_6, 61_18, 64_11, and/or 65_64.

Also disclosed is the diagnostic system of any preceding aspect, wherein the system selects for “positive and negative schizophrenia”, and wherein the one or more phenotypic sets comprise 12_4 and/or 42_9.

Also disclosed is the diagnostic system of any preceding aspect, wherein the system selects for “negative schizophrenia”, and wherein the one or more phenotypic sets comprise 52_28, 7_3, 48_41, 26_8, 69_41, 10_5, and/or 17_2.

Also disclosed is the diagnostic system of any preceding aspect, wherein the system selects for “positive schizophrenia”, and wherein the one or more phenotypic sets comprise 63_24 and/or 69_66.

Also disclosed is the diagnostic system of any preceding aspect, wherein the system selects for “severe process, positive schizophrenia”, and wherein the one or more phenotypic sets comprise 22_13, 18_13, 53_6, 59_41, 20_19, 55_7, 34_17, 69_66, 27_7, 18_13, 4_1, 66_54, and/or 8_4.

Also disclosed is the diagnostic system of any preceding aspect, wherein the system selects for “moderate process, disorganized negative schizophrenia”, and wherein the one or more phenotypic sets comprise 51_38, 42_7, 18_3, and/or 46_29.

Also disclosed is the diagnostic system of any preceding aspect, wherein the system selects for “moderate process, positive and negative schizophrenia”, and wherein the one or more phenotypic sets comprise 5_2, 57_39, 11_5, and/or 24_4.

Also disclosed is the diagnostic system of any preceding aspect, wherein the system selects for “moderate process, continuous positive schizophrenia”, and wherein the one or more phenotypic sets comprise 48_7, 28_23, and/or 25_20.

Also disclosed is the diagnostic system of any preceding aspect, further comprising a means for reading the one or more expression panels, a computer operationally linked to the means for reading the one or more expression panels, and a display for visualizing the diagnostic risk; wherein the computer identifies the expression profile of an expression panel, compares the expression profile to a control, and catalogs that data, wherein the computer provides an input source for inputting phenotypic into a phenomic database; wherein the computer compares the expression and phenomic data and calculates relationships between the genomic and phenotypic data; wherein the computer compares the genomic and phenotypic relationship data to a reference standard; and wherein the computer outputs the relationship data and the standard on the display.

In one aspect, disclosed herein are methods of diagnosing a subject with schizophrenia comprising obtaining a biological sample from the subject, obtaining clinical data from the subject, and applying the biological sample and clinical data to the diagnostic system of any preceding aspect.

In one aspect, disclosed herein are methods of diagnosing a subject with schizophrenia and determining the schizophrenia class comprising: obtaining a biological sample from the subject; obtaining clinical data from the subject; applying the biological sample and clinical data to a diagnostic system for diagnosing schizophrenia, wherein the diagnostic system comprises one or more expression panels and one or more phenotypic panels; comparing the genomic and phenotypic panels results to a reference standard; wherein the presence of one or more SNP sets and phenotypic sets in the subjects sample indicates the presence of schizophrenia, and wherein the genomic and phenotypic profile of the reference standard most closely correlating with the subjects genomic and phenotypic profile indicates schizophrenia class of the subject.

### III. BRIEF DESCRIPTION OF THE DRAWINGS

The patent or application file contains at least one drawing executed in color. Copies of this patent or patent application with color drawing(s) will be provided by the Office by request and payment of the necessary fee.

FIG. 1 shows the perception and visualization of a Genome-Wide Association Study (GWAS). Panel A is a matrix corresponding to the genome-wide association data set utilized in this work: Genetic Association Information Network (GAIN) and non-GAIN schizophrenia samples of the Molecular Genetics of Schizophrenia study. Allele values are indicated as BB (dark blue), AB (intermediate blue), AA (light blue), and missing (black). Panel B is a matrix corresponding to the distinct phenotypic consequences using data at the symptom level from the Diagnostic Interview for Genetic Studies corresponding to the GWAS in panel A (see FIG. 2). Values are indicated as present (garnet), absent (salmon), and missing (black). Panel C presents schematics of the “divide and conquer” approach, in which natural partitions of GWAS data (identified as sets of interacting single-nucleotide polymorphisms [SNPs] or SNP sets) were cross-matched with decomposed schizophrenia phenotype (identified as clusters of naturally occurring schizophrenia symptoms or phenotypic sets), revealing a specific and distributed genotypic-phenotypic architecture (networks of SNPs associated with sets of schizophrenia symptoms). This complex architecture is “invisible” or “hidden” to traditional GWAS.

FIG. 2 shows the methodology workflow of the divide & conquer strategy. Processes involving SNP and phenotypic sets are indicated in blue and red, respectively, whereas procedures concerning phenotypic-genotypic relations are shown in violet. Statistical analysis was performed by the SNP-Set Kernel Association Test (SKAT), which is also accessible via the web server cited above.

FIG. 3 shows examples of Identified Single-Nucleotide Polymorphism (SNP) Sets Represented as Heat Map Submatrices and their Corresponding Risk. Allele values are indicated as BB (dark blue), AB (intermediate blue), AA (light blue), and missing (black). Subject status (i.e., cases and controls) was superimposed after SNP set identification: cases in red and controls in green. Genotypic SNP sets are labeled by a pair of numbers representing the maximum number of clusters and the order in which they were selected by the method. All SNP sets are calculated with the generalized factorization method based on the non-negative matrix factorization method. Dendrograms were artificially superimposed for visualization purposes. (See FIG. 4 for all SNP sets at more than 70% of risk.) Panels A-F illustrate SNP sets, representing submatrices of the original genome-wide association study matrix and composed of shared SNPs and/or subjects. Panel A presents a SNP set exhibiting a homogeneous configuration in which all subjects in that group share the same interaction among a specific set of homozygotic alleles (i.e., SNP.times . . . times.SNP interactions). Panel B presents a SNP set encoding subjects exhibiting a particular heterozygotic genotype with respect to the A allele in a subset of SNPs and another heterozygote genotype with respect to the B allele in a different subset of SNPs (i.e., AND-type of interactions). Panel C presents a SNP set composed of subjects who share a particular genotype value for a subset of SNPs, and another subset of subjects sharing a different genotype value for the same subset of SNPs (i.e., OR-type of interactions). Inclusion-type relations are exemplified by a SNP set (panel A) subsumed under a more general SNP set (panel C), and both sets provide different descriptions of target subjects. Panels D-F present SNP sets that combine all previous interactions into more complex structures. Panel G presents a surface representing the risk function of the uncovered SNP sets. The risk (z-axis; red=high, blue=low) was calculated based on the distribution subject status (i.e., cases and controls) within each SNP set, and the surface was plotted interpolating the relation domains. Dendrograms reflect the order adopted for plotting SNP sets. SNP sets were clustered by shared SNP (x-axis) and by shared subjects (y-axis) using hypergeometric statistics. (Close-located SNP sets in an edge share more SNPs and/or subjects than those located far away.)

FIG. 4 shows SNP Sets represented as submatrices composed of SNPs (y-axis) shared by distinct subsets of subjects (x-axis). Allele values are indicated as AA (light blue), AB (intermediate blue), BB (dark blue), and missing (black). SNP and subject names/codes are not shown. Subject status was superimposed after SNP set identification: cases (red) and controls (green). SNP sets are labeled by a pair of numbers representing the maximum number of sub-matrices and the order in which they were selected by the method, as described in FIG. 3. Row and column dendograms were superimposed a posteriori into each sub-matrix for visualization purposes.

FIGS. 5A and 5B show dissection of a Genome-Wide Association Study (GWAS) and Identification of the Genotypic and Phenotypic Architecture of Schizophrenia. FIG. 5A presents a genotypic network, in which nodes indicate SNP sets linked by shared SNPs (blue lines) and/or subjects (red lines). The risk value, which was incorporated after the SNP set identification, was color-coded. The 42 SNP sets harboring.gtoreq.70% of risk were topologically organized into 17 disjoint subnetworks. Subsets of implicated genes are indicated. Highly connected SNP sets based on shared SNPs (blue lines) and subjects (red lines) might share a phenotypic profile (e.g., 81_13 and 88_64; see Table 7). Yet a super-SNP set, such as 81_13, may have unique—in addition to common—descriptive phenotypic features (see Table 7). Disconnected SNP sets, such as 71_55 and 14_6, belong to disjoint networks that may include the same gene (i.e., NTKR3; see Table 2 and FIG. 6B but carry SNPs that are located in different regions of that gene, such as the promoter and coding regions, respectively. Both SNPs may produce distinct molecular consequences (see Table 4 and FIG. 6B) and phenotypic profiles (see Table 7). FIG. 5B shows the classes of schizophrenia mapped to the disease architecture (see Table 7). Eight classes of schizophrenia were identified by independently characterizing each phenotypic feature included in a genotypic-phenotypic relationship; classifying each item based on the symptoms as purely positive, purely negative, primarily positive, or primarily negative symptoms; and clustering these relationships based on their recoded phenotypic domain using non-negative matrix factorization. SNP sets harboring only positive symptoms are indicated in green, whereas those displaying negative symptoms are in red. Intermediate combinations including severe and/or moderate processes combined with positive and/or negative and/or disorganized symptoms were also color-coded. Dashed lines indicate nonsignificant matching.

FIG. 6 shows the bioinformatics analysis of SNPs derived from SNP Sets targeting genomic regions. (A) Multiple SNPs within a SNP set can affect a single gene in many ways. 5 SNPs from the SNP set 19_2 (100% of risk) can affect GOLGA1: SNPs rs10986471 and rs640052 may produce downstream variations; SNP rs634710 can generate missense variations; SNP rs7031479 may introduce intron variants; and SNP rs687434 may create non-coding exon variants (Tables 2 and 4). Two SNP variants of the SNP set 19_2 affect the regulatory region of ncRNAs genes: miRNA AL354928.1 and small nuclear RNA (U4 snRNA) (Table 2). The rs640052 SNP lies between regulatory regions downstream and upstream of U4 and the GOLGA1 gene, which may be functionally related. The U4 snRNAs conform the splicesome, which is involved in the splicing process that generates diverse mRNA species from a single pre-mRNA. Consistently, the GOLGA1 gene has substantial variation in alternative splice isoform expression and alternative polyadenylation in cerebellar cortex between normal individuals and SZ patients. (B) All SNPs from SNP set 7_55 are located in the intergenic region upstream of the NTRK3 gene, in the location of a predicted enhancer (Table 2). Nevertheless, those SNPs of the 14_6 SNP set are located within NTRK3, principally in intronic regions and within the upstream region of pseudogene RP11-356B18.1 (Table 2). The latter pseudogene is harbored in an intron of NTRK3 that is processed in the NTRK-005 transcript variant, which does not code neurotrophin receptor-3 protein. This suggests that a mutation in the first SNP set may inhibit the transcription of the corresponding gene, whereas mutations in the second SNP set may block or decrease production of the corresponding protein (Table 4). The protein coding genes include the 5′ and 3′ untranslated region (3′UTR, 5° UTR), exons that code for the coding sequence (CDS) and introns. The ncRNA genes are defined only in terms of exons and introns. The promoter upstream and downstream region for both types of genes have been defined as the segment of 5000 bp before the beginning of the 5′ UTR, and 5000 bp after the 3′UTR end. The remaining space between the upstream and downstream region of a gene is here defined as the intergenic region.

FIG. 7 shows a pathway analysis. Distinct pathways identified by the SNP sets are well known, relevant and interconnected signaling pathways for neural development, neurotrophin function, neurotransmission, and neurodegenerative disorders (see Tables 2 and 6). Other genes uncovered are also overwhelmingly expressed in the brain, and participate in regulation of intracellular signaling, oxidative stress, apoptosis, neuroimmune regulation, protein synthesis, and epigenetic gene expression.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

### B. COMPOSITIONS

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

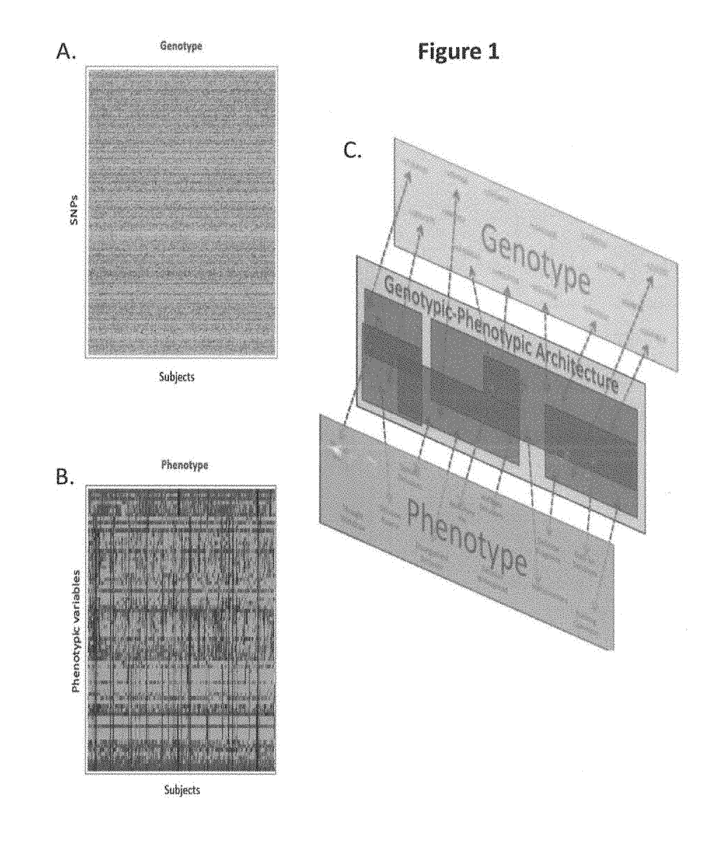

We have chosen to measure and characterize the complexity of both the genotypic and the phenotypic architecture of schizophrenia (FIG. 1C). Past studies have generally ignored variation in clinical features, categorizing people as either having or not having schizophrenia, and they have looked only at the average effects of genetic variants, ignoring their organization into interactive genotypic networks. We show herein that schizophrenia heritability is not missing but is distributed into different networks of interacting genes that influence different people. Unlike previous studies that neglected clinical heterogeneity among subjects with schizophrenia, we characterized the clinical phenotype in detail. We also allowed for possible developmental complexity, including equifinality (or heterogeneity) and multifinality (or pleiotropy).

We investigated the architecture of schizophrenia in the Molecular Genetics of Schizophrenia (MGS) study, in which all subjects had consistent and detailed genotypic and phenotypic assessments. We then replicated the results in two other independent samples in which comparable genotypic and phenotypic features were available: the Clinical Antipsychotic Trial of Intervention Effectiveness (CATIE) and the Portuguese Island studies from the Psychiatric Genomics Consortium (PGC).

The result of this work is a diagnostic system that is able to diagnose a subject as having schizophrenia, but more importantly classify the category of schizophrenia with which the subject is suffering. To accomplish this, the diagnostic system can comprise an expression panel that can be used to detect nucleic acid or protein expression. Thus, in one aspect, disclosed herein are diagnostic systems for diagnosing schizophrenia, wherein the diagnostic system comprises one or more expression panels, wherein the one or more expression panels can comprise one or more one or more expression sets (such as, for example, one or more SNP sets).

The expression panels disclosed herein can be assayed by any means to measure differential expression of a gene or protein known in the art. Specifically contemplated herein are methods of assessing the risk, diagnosing, or classifying schizophrenia comprising performing an assay that measures differential expression of a nucleic acid, gene, peptide, or protein. Specifically contemplated are methods of assessing the risk, diagnosing, or classifying schizophrenia comprising performing an assay that measures differential gene or protein expression, wherein the assay is selected from the group of assays comprising Northern analysis, RNAse protection assay, PCR, QPCR, genome microarray, DNA microarray, MMCHipslow density PCR array, oligo array, protein array, peptide array, phenotype microarray, SAGE, and/or high throughput sequencing. Therefore, it is understood that the microarray panel can measure differential expression of a phenotypes, proteins, peptides, RNAs, microRNAs, DNAs, Single Nucleotide Polymorphisms (SNPs), or genes or sets of said phenotypes, proteins, peptides, RNAs, microRNAs, DNAs, Single Nucleotide Polymorphisms (SNPs), or genes. For example, in one aspect, the disclosed panel can be a microarray such as a those developed and sold by Affymetrix, Agilent, Applied Microarrays, Arrayit, and IIlumina

In one aspect, the panel can comprise Single Nucleotide Polymorphism (SNP) sets. The SNP set can be any SNP set that has a greater than 70% association with risk for schizophrenia, including but not limited to 19_2, 88_64, 81_13, 87_76, 58_29, 83_41, 9_9, 10_4, 14_6, 56_30, 42_37, 65_25, 71_55, 12_11, 90_78, 77_5, 88_8, 51_28, 59_48, 41_12, 22_11, 13_12, 31_22, 85_84, 87_84, 16_10, 56_19, 75_31, 81_73, 85_23, 21_8, 76_74, 61_39, 75_67, 76_63, 81_3, 87_26, 88_43, 25_10, 12_2, 52_42, and 54_51, which are specifically listed in Table 1.

TABLE 1
Single-Nucleotide Polymorphism (SNP) Sets Reported With ≥70% Risk of Schizophrenia,
Statistical Comparison With Individual SNPs and Compositions ^{a}

| | SKAT p Values | | | | | | |
|---|---|---|---|---|---|---|---|
| SNP set | Group | Average SNP | Best SNP | Worst SNP | Subjects (N) | SNPs (N) | Risk (%) |
| 19_2 | 2.88E−05 | 3.43E−02 | 4.60E−04 | 1.38E−02 | 9 | 9 | 100 |
| 88_64 | 1.43E−11 | 2.06E−03 | 2.15E−07 | 1.79E−02 | 176 | 6 | 96 |
| 81_13 | 1.46E−10 | 5.44E−03 | 2.15E−07 | 3.70E−02 | 234 | 10 | 95 |
| 87_76 | 7.11E−07 | 1.05E−02 | 1.37E−05 | 3.13E−02 | 74 | 3 | 95 |
| 58_29 | 5.41E−04 | 6.52E−03 | 2.07E−04 | 2.83E−02 | 125 | 6 | 94 |
| 83_41 | 3.87E−05 | 1.56E−04 | 1.01E−04 | 2.68E−04 | 61 | 4 | 93 |
| 9_9 | 1.51E−06 | 2.52E−03 | 1.23E−04 | 1.18E−02 | 144 | 19 | 92 |
| 10_4 | 3.83E−05 | 1.72E−02 | 2.11E−04 | 1.05E−02 | 58 | 11 | 91 |
| 14_6 | 2.38E−06 | 1.85E−03 | 1.23E−04 | 5.87E−03 | 22 | 11 | 90 |
| 56_30 | 1.91E−10 | 4.33E−03 | 2.15E−07 | 2.10E−02 | 382 | 11 | 88 |
| 42_37 | 4.15E−06 | 2.35E−02 | 6.59E−05 | 1.38E−02 | 70 | 24 | 86 |
| 65_25 | 3.95E−05 | 1.99E−02 | 2.53E−04 | 8.83E−02 | 62 | 5 | 86 |
| 71_55 | 1.90E−05 | 3.99E−04 | 2.63E−05 | 1.08E−03 | 63 | 6 | 86 |
| 12_11 | 6.53E−04 | 2.28E−02 | 7.34E−03 | 1.05E−01 | 94 | 11 | 84 |
| 90_78 | 7.87E−04 | 2.99E−02 | 3.58E−02 | 9.53E−02 | 200 | 4 | 83 |
| 77_5 | 4.86E−05 | 5.01E−04 | 2.08E−05 | 1.49E−03 | 297 | 5 | 82 |
| 88_8 | 2.88E−04 | 2.95E−02 | 3.58E−02 | 8.36E−02 | 32 | 10 | 82 |
| 51_28 | 2.07E−04 | 2.25E−02 | 1.75E−02 | 3.13E−02 | 258 | 3 | 81 |
| 59_48 | 2.32E−09 | 9.48E−03 | 2.38E−05 | 2.96E−02 | 174 | 7 | 80 |
| 41_12 | 1.36E−03 | 1.62E−02 | 1.12E−01 | 2.17E−02 | 78 | 3 | 76 |
| 22_11 | 6.24E−05 | 4.29E−04 | 1.33E−04 | 1.08E−03 | 97 | 12 | 75 |
| 13_12 | 4.52E−05 | 3.61E−04 | 5.88E−05 | 1.45E−03 | 148 | 10 | 75 |
| 31_22 | 1.01E−04 | 2.37E−04 | 1.11E−04 | 4.03E−04 | 92 | 7 | 74 |
| 85_84 | 1.53E−05 | 1.01E−04 | 1.37E−05 | 1.81E−04 | 39 | 4 | 74 |
| 87_84 | 1.19E−04 | 1.40E−02 | 1.37E−05 | 1.30E−02 | 22 | 13 | 74 |
| 16_10 | 1.81E−03 | 1.59E−02 | 2.92E−03 | 5.92E−02 | 141 | 12 | 73 |
| 56_19 | 2.02E−04 | 6.69E−04 | 1.02E−04 | 1.76E−03 | 90 | 5 | 73 |
| 75_31 | 2.61E−05 | 1.37E−02 | 1.02E−04 | 9.53E−02 | 197 | 8 | 73 |
| 81_73 | 1.13E−05 | 2.99E−02 | 2.57E−04 | 1.29E−02 | 213 | 10 | 73 |
| 85_23 | 6.20E−03 | 9.46E−03 | 5.58E−03 | 1.16E−02 | 53 | 4 | 73 |
| 21_8 | 6.24E−05 | 4.29E−04 | l.33E−04 | 1.08E−03 | 188 | 12 | 71 |
| 76_74 | 1.58E−17 | 1.33E−02 | 1.12E−05 | 1.17E−02 | 284 | 14 | 71 |
| 61_39 | 1.04E−03 | 2.43E−02 | 1.90E−03 | 5.45E−02 | 51 | 3 | 71 |
| 75_67 | 3.76E−18 | 7.16E−02 | 2.15E−07 | 1.00E−03 | 877 | 32 | 71 |
| 76_63 | 2.07E−02 | 2.25E−02 | 1.75E−02 | 3.13E−02 | 34 | 3 | 71 |
| 81_3 | 6.24E−05 | 4.29E−04 | 1.33E−04 | 1.08E−03 | 107 | 12 | 71 |
| 87_26 | 2.49E−03 | 6.03E−03 | 4.14E−03 | 1.12E−02 | 28 | 5 | 71 |
| 88_43 | 1.37E−04 | 1.85E−03 | 6.03E−04 | 4.82E−03 | 70 | 7 | 71 |
| 25_10 | 3.49E−06 | 1.67E−03 | 1.11E−04 | 1.53E−02 | 124 | 9 | 70 |
| 12_2 | 1.81E−03 | 1.59E−02 | 2.92E−04 | 5.92E−02 | 194 | 12 | 70 |
| 52_42 | 5.70E−05 | 5.06E−03 | 6.59E−05 | 3.60E−02 | 87 | 16 | 70 |
| 54_51 | 1.49E−05 | 5.01E−04 | 2.08E−04 | 1.49E−03 | 132 | 5 | 70 |

^{a} SKAT = SNP-Set Kernel Association Test.

Accordingly, in one aspect, disclosed herein are diagnostic systems for diagnosing schizophrenia, wherein the diagnostic system comprises one or more expression panels, wherein the one or more expression panels each comprise one or more of the single nucleotide polymorphism (SNP) sets selected from the group comprising, but not limited to 19_2, 88_64, 81_13, 87_76, 58_29, 83_41, 9_9, 10_4, 14_6, 56_30, 42_37, 65_25, 71_55, 12_11, 90_78, 77_5, 88_8, 51_28, 59_48, 41_12, 22_11, 13_12, 31_22, 85_84, 87_84, 16_10, 56_19, 75_31, 81_73, 85_23, 21_8, 76_74, 61_39, 75_67, 76_63, 81_3, 87_26, 88_43, 25_10, 12_2, 52_42, and/or 54_51. It is understood and herein contemplated that each of the SNP sets disclosed herein maps to one or more nucleic acid molecules. Therefore, a single SNP set will not necessarily be comprised solely of primers or probes for detection of a single SNP, but can be comprised of multiple primers and probes for the detection of SNPs mapping to at least one, two, three, four, five, six, seven, eight, nine, ten, eleven, twelve, thirteen, fourteen, fifteen, sixteen, seventeen, eighteen, nineteen, or twenty nucleic acid locations. As disclosed in Table 2, each of the SNP sets disclosed herein maps to particular locations on a gene, including protein coding and non-coding regulatory variants.

TABLE 2
Mapping SNP sets into genomic information. (Information obtained from HaploReg v2, dbSNP and NCBI databases)

| | | | dbSNP func- | | NCBI GWAS | NCBI association to | |
|---|---|---|---|---|---|---|---|
| Group | Chr | Gene | tion annotation | Neuronal Function | association to SZ | other CNS disorders | Summary |
| 9_9 | 15 | NTRK3 | intronic | neurotrophic tyrosine kinase, receptor, | Yes | | This gene encodes a member of the neurotrophic |
| | | | | type 3 | | | tyrosine receptor kinase (NTRK) family. This |
| | | | | | | | kinase is a membrane-bound receptor that, upon |
| | | | | | | | neurotrophin binding, phosphorylates itself and |
| | | | | | | | members of the MAPK pathway. Signalling |
| | | | | | | | through this kinase leads to cell differentiation and |
| | | | | | | | may play a role in the development of |
| | | | | | | | proprioceptive neurons that sense body position. |
| | | | | | | | Mutations in this gene have been associated with |
| | | | | | | | medulloblastomas, secretory breast carcinomas and |
| | | | | | | | other cancers. Several transcript variants encoding |
| | | | | | | | different isoforms have been found for this gene |
| 9_9 | 7 | SEMA3A | intronic | regulation of axonal growth | Yes | | This gene is a member of the semaphorin family |
| | | | | | | | and encodes a protein with an Ig-like C2-type |
| | | | | | | | (immunoglobulin-like) domain, a PSI domain and a |
| | | | | | | | Sema domain. This secreted protein can function as |
| | | | | | | | either a chemorepulsive agent, inhibiting axonal |
| | | | | | | | outgrowth, or as a chemoattractive agent, |
| | | | | | | | stimulating the growth of apical dendrites. In both |
| | | | | | | | cases, the protein is vital for normal neuronal |
| | | | | | | | pattern development. Increased expression of this |
| | | | | | | | protein is associated with schizophrenia and is seen |
| | | | | | | | in a variety of human tumor cell lines. Also, |
| | | | | | | | aberrant release of this protein is associated with |
| | | | | | | | the progression of Alzheimer's disease. |
| 10_4 | 14 | C14orf102 | intronic | mRNA suppression | | yes | NRDE-2, necessary for RNA interference, domain |
| | | | | | | (autism and ADHD) | containing |
| 10_4 | 14 | C14orf102(5′) | | mRNA suppression | | yes | NRDE-2, necessary for RNA interference, domain |
| | | | | | | (autism and ADHD) | containing |
| 10_4 | 14 | PSMC1 | intronic | Ubiquitin dependent ATPase, | | yes | The 26S proteasome is a multicatalytic proteinase |
| | | | | NFkB pathway | | (Spinocerebellar atrophy 7) | complex with a highly ordered structure composed |
| | | | | | | | of 2 complexes, a 20S core and a 19S regulator. |
| | | | | | | | The 20S core is composed of 4 rings of 28 non- |
| | | | | | | | identical subunits; 2 rings are composed of 7 alpha |
| | | | | | | | subunits and 2 rings are composed of 7 beta |
| | | | | | | | subunits. The 19S regulator is composed of a base, |
| | | | | | | | which contains 6 ATPase subunits and 2 non- |
| | | | | | | | ATPase subunits, and a lid, which contains up to 10 |
| | | | | | | | non-ATPase subunits. Proteasomes are distributed |
| | | | | | | | throughout eukaryotic cells at a high concentration |
| | | | | | | | and cleave peptides in an ATP/ubiquitin-dependent |
| | | | | | | | process in a non-lysosomal pathway. An essential |
| | | | | | | | function of a modified proteasome, the |
| | | | | | | | immunoproteasome, is the processing of class I |
| | | | | | | | MHC peptides. This gene encodes one of the |
| | | | | | | | ATPase subunits, a member of the triple-A family |
| | | | | | | | of ATPases which have a chaperone-like activity. |
| | | | | | | | This subunit and a 20S core alpha subunit interact |
| | | | | | | | specifically with the hepatitis B virus X protein, a |
| | | | | | | | protein critical to viral replication. This subunit also |
| | | | | | | | interacts with the adenovirus E1A protein and this |
| | | | | | | | interaction alters the activity of the proteasome. |
| | | | | | | | Finally, this subunit interacts with ataxin-7, |
| | | | | | | | suggesting a role for the proteasome in the |
| | | | | | | | development of Spinocerebellar ataxia type 7, a |
| | | | | | | | progressive neurodegenerative disorder. |
| 10_4 | 14 | PSMC1(3′) | | Ubiquitin dependent ATPase, | | yes | The 26S proteasome is a multicatalytic proteinase |
| | | | | NFkB pathway | | (Spinocerebellar atrophy 7) | complex with a highly ordered structure composed |
| | | | | | | | of 2 complexes, a 20S core and a 19S regulator. |
| | | | | | | | The 20S core is composed of 4 rings of 28 non- |
| | | | | | | | identical subunits; 2 rings are composed of 7 alpha |
| | | | | | | | subunits and 2 rings are composed of 7 beta |
| | | | | | | | subunits. The 19S regulator is composed of a base, |
| | | | | | | | which contains 6 ATPase subunits and 2 non- |
| | | | | | | | ATPase subunits, and a lid, which contains up to 10 |
| | | | | | | | non-ATPase subunits. Proteasomes are distributed |
| | | | | | | | throughout eukaryotic cells at a high concentration |
| | | | | | | | and cleave peptides in an ATP/ubiquitin-dependent |
| | | | | | | | process in a non-lysosomal pathway. An essential |
| | | | | | | | function of a modified proteasome, the |
| | | | | | | | immunoproteasome, is the processing of class I |
| | | | | | | | MHC peptides. This gene encodes one of the |
| | | | | | | | ATPase subunits, a member of the triple-A family |
| | | | | | | | of ATPases which have a chaperone-like activity. |
| | | | | | | | This subunit and a 20S core alpha subunit interact |
| | | | | | | | specifically with the hepatitis B virus X protein, a |
| | | | | | | | protein critical to viral replication. This subunit also |
| | | | | | | | interacts with the adenovirus E1A protein and this |
| | | | | | | | interaction alters the activity of the proteasome. |
| | | | | | | | Finally, this subunit interacts with ataxin-7, |
| | | | | | | | suggesting a role for the proteasome in the |
| | | | | | | | development of Spinocerebellar ataxia type 7, a |
| | | | | | | | progressive neurodegenerative disorder. |
| 10_4 | 14 | PSMC1(5′) | | Ubiquitin dependent ATPase, | | yes | The 26S proteasome is a multicatalytic proteinase |
| | | | | NFkB pathway | | (Spinocerebellar atrophy 7) | complex with a highly ordered structure composed |
| | | | | | | | of 2 complexes, a 20S core and a 19S regulator. The |
| | | | | | | | 20S core is composed of 4 rings of 28 non-identical |
| | | | | | | | subunits; 2 rings are composed of 7 alpha subunits |
| | | | | | | | and 2 rings are composed of 7 beta subunits. The |
| | | | | | | | 19S regulator is composed of a base, which contains |
| | | | | | | | 6 ATPase subunits and 2 non-ATPase subunits, and |
| | | | | | | | a lid, which contains up to 10 non-ATPase subunits. |
| | | | | | | | Proteasomes are distributed throughout eukaryotic |
| | | | | | | | cells at a high concentration and cleave peptides in |
| | | | | | | | an ATP/ubiquitin-dependent process in a non- |
| | | | | | | | lysosomal pathway. An essential function of a |
| | | | | | | | modified proteasome, the immunoproteasome, is |
| | | | | | | | the processing of class I MHC peptides. This gene |
| | | | | | | | encodes one of the ATPase subunits, a member of |
| | | | | | | | the triple-A family of ATPases which have a |
| | | | | | | | chaperone-like activity. This subunit and a 20S core |
| | | | | | | | alpha subunit interact specifically with the hepatitis |
| | | | | | | | B virus X protein, a protein critical to viral |
| | | | | | | | replication. This subunit also interacts with the |
| | | | | | | | adenovirus E1A protein and this interaction alters |
| | | | | | | | the activity of the proteasome. Finally, this subunit |
| | | | | | | | interacts with ataxin-7, suggesting a role for the |
| | | | | | | | proteasome in the development of spinocerebellar |
| | | | | | | | ataxia type 7, a progressive neurodegenerative |
| | | | | | | | disorder. |
| 12_11 | 14 | C14orf102 | intronic | mRNA suppression | | yes | NRDE-2, necessary for RNA interference, domain |
| | | | | | | (autism and ADHD) | containing |
| 12_11 | 14 | C14orf102(5′) | | mRNA suppression | | yes | NRDE-2, necessary for RNA interference, domain |
| | | | | | | (autism and ADHD) | containing |
| 12_11 | 14 | PSMC1 | intronic | Ubiquitin dependent ATPase, | | yes | The 26S proteasome is a multicatalytic proteinase |
| | | | | NFkB pathway | | (Spinocerebellar atrophy 7) | complex with a highly ordered structure composed |
| | | | | | | | of 2 complexes, a 20S core and a 19S regulator. The |
| | | | | | | | 20S core is composed of 4 rings of 28 non-identical |
| | | | | | | | subunits; 2 rings are composed of 7 alpha subunits |
| | | | | | | | and 2 rings are composed of 7 beta subunits. The |
| | | | | | | | 19S regulator is composed of a base, which contains |
| | | | | | | | 6 ATPase subunits and 2 non-ATPase subunits, and |
| | | | | | | | a lid, which contains up to 10 non-ATPase subunits. |
| | | | | | | | Proteasomes are distributed throughout eukaryotic |
| | | | | | | | cells at a high concentration and cleave peptides in |
| | | | | | | | an ATP/ubiquitin-dependent process in a non- |
| | | | | | | | lysosomal pathway. An essential function of a |
| | | | | | | | modified proteasome, the immunoproteasome, is |
| | | | | | | | the processing of class I MHC peptides. This gene |
| | | | | | | | encodes one of the ATPase subunits, a member of |
| | | | | | | | the triple-A family of ATPases which have a |
| | | | | | | | chaperone-like activity. This subunit and a 20S core |
| | | | | | | | alpha subunit interact specifically with the hepatitis |
| | | | | | | | B virus X protein, a protein critical to viral |
| | | | | | | | replication. This subunit also interacts with the |
| | | | | | | | adenovirus E1A protein and this interaction alters |
| | | | | | | | the activity of the proteasome. Finally, this subunit |
| | | | | | | | interacts with ataxin-7, suggesting a role for the |
| | | | | | | | proteasome in the development of spinocerebellar |
| | | | | | | | ataxia type 7, a progressive neurodegenerative |
| | | | | | | | disorder. |
| 12_11 | 14 | PSMC1(3′) | | Ubiquitin dependent ATPase, | | yes | The 26S proteasome is a multicatalytic proteinase |
| | | | | NFkB pathway | | (Spinocerebellar atrophy 7) | complex with a highly ordered structure composed |
| | | | | | | | of 2 complexes, a 20S core and a 19S regulator. The |
| | | | | | | | 20S core is composed of 4 rings of 28 non-identical |
| | | | | | | | subunits; 2 rings are composed of 7 alpha subunits |
| | | | | | | | and 2 rings are composed of 7 beta subunits. The |
| | | | | | | | 19S regulator is composed of a base, which contains |
| | | | | | | | 6 ATPase subunits and 2 non-ATPase subunits, and |
| | | | | | | | a lid, which contains up to 10 non-ATPase subunits. |
| | | | | | | | Proteasomes are distributed throughout eukaryotic |
| | | | | | | | cells at a high concentration and cleave peptides in |
| | | | | | | | an ATP/ubiquitin-dependent process in a non- |
| | | | | | | | lysosomal pathway. An essential function of a |
| | | | | | | | modified proteasome, the immunoproteasome, is |
| | | | | | | | the processing of class I MHC peptides. This gene |
| | | | | | | | encodes one of the ATPase subunits, a member of |
| | | | | | | | the triple-A family of ATPases which have a |
| | | | | | | | chaperone-like activity. This subunit and a 20S core |
| | | | | | | | alpha subunit interact specifically with the hepatitis |
| | | | | | | | B virus X protein, a protein critical to viral |
| | | | | | | | replication. This subunit also interacts with the |
| | | | | | | | adenovirus E1A protein and this interaction alters |
| | | | | | | | the activity of the proteasome. Finally, this subunit |
| | | | | | | | interacts with ataxin-7, suggesting a role for the |
| | | | | | | | proteasome in the development of spinocerebellar |
| | | | | | | | ataxia type 7, a progressive neurodegenerative |
| | | | | | | | disorder. |
| 12_11 | 14 | PSMC1(5′) | | Ubiquitin dependent ATPase, | | yes | The 26S proteasome is a multicatalytic proteinase |
| | | | | NFkB pathway | | (Spinocerebellar atrophy 7) | complex with a highly ordered structure composed |
| | | | | | | | of 2 complexes, a 20S core and a 19S regulator. The |
| | | | | | | | 20S core is composed of 4 rings of 28 non-identical |
| | | | | | | | subunits; 2 rings are composed of 7 alpha subunits |
| | | | | | | | and 2 rings are composed of 7 beta subunits. The |
| | | | | | | | 19S regulator is composed of a base, which contains |
| | | | | | | | 6 ATPase subunits and 2 non-ATPase subunits, and |
| | | | | | | | a lid, which contains up to 10 non-ATPase subunits. |
| | | | | | | | Proteasomes are distributed throughout eukaryotic |
| | | | | | | | cells at a high concentration and cleave peptides in |
| | | | | | | | an ATP/ubiquitin-dependent process in a non- |
| | | | | | | | lysosomal pathway. An essential function of a |
| | | | | | | | modified proteasome, the immunoproteasome, is |
| | | | | | | | the processing of class I MHC peptides. This gene |
| | | | | | | | encodes one of the ATPase subunits, a member of |
| | | | | | | | the triple-A family of ATPases which have a |
| | | | | | | | chaperone-like activity. This subunit and a 20S core |
| | | | | | | | alpha subunit interact specifically with the hepatitis |
| | | | | | | | B virus X protein, a protein critical to viral |
| | | | | | | | replication. This subunit also interacts with the |
| | | | | | | | adenovirus E1A protein and this interaction alters |
| | | | | | | | the activity of the proteasome. Finally, this subunit |
| | | | | | | | interacts with ataxin-7, suggesting a role for the |
| | | | | | | | proteasome in the development of spinocerebellar |
| | | | | | | | ataxia type 7, a progressive neurodegenerative |
| | | | | | | | disorder. |
| 12_2 | 4 | HPGDS | 3′-UTR | prostaglandin D synthase | Yes | | Prostaglandin-D synthase is a sigma class |
| | | | | | | | glutathione-S-transferase family member. The |
| | | | | | | | enzyme catalyzes the conversion of PGH2 to PGD2 |
| | | | | | | | and plays a role in the production of prostanoids in |
| | | | | | | | the immune system and mast cells. The presence of |
| | | | | | | | this enzyme can be used to identify the |
| | | | | | | | differentiation stage of human megakaryocytes. |
| | | | | | | | [provided by RefSeq, July 2008] |
| 12_2 | 4 | HPGDS | intronic | prostaglandin D synthase | Yes | | Prostaglandin-D synthase is a sigma class |
| | | | | | | | glutathione-S-transferase family member. The |
| | | | | | | | enzyme catalyzes the conversion of PGH2 to PGD2 |
| | | | | | | | and plays a role in the production of prostanoids in |
| | | | | | | | the immune system and mast cells. The presence of |
| | | | | | | | this enzyme can be used to identify the |
| | | | | | | | differentiation stage of human megakaryocytes. |
| 12_2 | 4 | HPGDS(5′) | | prostaglandin D synthase | Yes | | Prostaglandin-D synthase is a sigma class |
| | | | | | | | glutathione-S-transferase family member. The |
| | | | | | | | enzyme catalyzes the conversion of PGH2 to PGD2 |
| | | | | | | | and plays a role in the production of prostanoids in |
| | | | | | | | the immune system and mast cells. The presence of |
| | | | | | | | this enzyme can be used to identify the |
| | | | | | | | differentiation stage of human megakaryocytes. |
| 12_2 | 4 | RP11-363G15.2 | | spliceosome complex activation | | no | This gene encodes a component of the spliceosome |
| | | | | | | (retinitis pigmentosa) | complex and is one of several retinitis pigmentosa- |
| | | | | | | | causing genes. When the gene product is added to |
| | | | | | | | the spliceosome complex, activation occurs. |
| 12_2 | 4 | SMARCAD1 | 3′-UTR | actin-dependent chromatin regulation | Yes | | This gene encodes a member of the SNF subfamily |
| | | | | | | | of helicase proteins. The encoded protein plays a |
| | | | | | | | critical role in the restoration of heterochromatin |
| | | | | | | | organization and propagation of epigenetic patterns |
| | | | | | | | following DNA replication by mediating histone |
| | | | | | | | H3/H4 deacetylation. Mutations in this gene are |
| | | | | | | | associated with adermatoglyphia. Alternatively |
| | | | | | | | spliced transcript variants encoding multiple |
| | | | | | | | isoforms have been observed for this gene. |
| 12_2 | 4 | SMARCAD1 | intronic | actin-dependent chromatin regulation | Yes | | This gene encodes a member of the SNF subfamily |
| | | | | | | | of helicase proteins. The encoded protein plays a |
| | | | | | | | critical role in the restoration of heterochromatin |
| | | | | | | | organization and propagation of epigenetic patterns |
| | | | | | | | following DNA replication by mediating histone |
| | | | | | | | H3/H4 deacetylation. Mutations in this gene are |
| | | | | | | | associated with adermatoglyphia. Alternatively |
| | | | | | | | spliced transcript variants encoding multiple |
| | | | | | | | isoforms have been observed for this gene. |
| 12_2 | 4 | SMARCAD1 | missense | actin-dependent chromatin regulation | Yes | | This gene encodes a member of the SNF subfamily |
| | | | | | | | of helicase proteins. The encoded protein plays a |
| | | | | | | | critical role in the restoration of heterochromatin |
| | | | | | | | organization and propagation of epigenetic patterns |
| | | | | | | | following DNA replication by mediating histone |
| | | | | | | | H3/H4 deacetylation. Mutations in this gene are |
| | | | | | | | associated with adermatoglyphia. Alternatively |
| | | | | | | | spliced transcript variants encoding multiple |
| | | | | | | | isoforms have been observed for this gene. |
| 12_2 | 4 | SMARCAD1 | synonymous | actin-dependent chromatin regulation | Yes | | This gene encodes a member of the SNF subfamily |
| | | | | | | | of helicase proteins. The encoded protein plays a |
| | | | | | | | critical role in the restoration of heterochromatin |
| | | | | | | | organization and propagation of epigenetic patterns |
| | | | | | | | following DNA replication by mediating histone |
| | | | | | | | H3/H4 deacetylation. Mutations in this gene are |
| | | | | | | | associated with adermatoglyphia. Alternatively |
| | | | | | | | spliced transcript variants encoding multiple |
| | | | | | | | isoforms have been observed for this gene. |
| 13_12 | 14 | EML5 | intronic | WD40 domain protein expressed in brain | | no | echinoderm microtubule associated protein like 5 |
| 13_12 | 14 | SPATA7 | missense | isolated in testis and retina | | no | This gene, originally isolated from testis, is also |
| | | | | | | (retinitis pigmentosa and | expressed in retina. Mutations in this gene are |
| | | | | | | Lieber amaurosis) | associated with Leber congenital amaurosis and |
| | | | | | | | juvenile retinitis pigmentosa. Alternatively spliced |
| | | | | | | | transcript variants encoding different isoforms have |
| | | | | | | | been found for this gene. |
| 13_12 | 14 | U4.15(3′) | | RNA, U4 small nuclear 92, pseudogene? | | | RNA, U4 small nuclear 1 |
| 13_12 | 14 | U4.15(5′) | | RNA, U4 small nuclear 92, pseudogene? | | | RNA, U4 small nuclear 2 |
| 13_12 | 14 | ZC3H14 * | intronic | mRNA stability, nuclear export, and | | yes | ZC3H14 belongs to a family of poly(A)-binding |
| | | | | translation | | (regulation of tau pathology) | proteins that influence gene expression by |
| | | | | | | | regulating mRNA stability, nuclear export, and |
| | | | | | | | translation |
| 14_6 | 15 | NTRK3 | intronic | neurotrophic tyrosine kinase, receptor, | Yes | | This gene encodes a member of the neurotrophic |
| | | | | type 3 | | | tyrosine receptor kinase (NTRK) family. This |
| | | | | | | | kinase is a membrane-bound receptor that, upon |
| | | | | | | | neurotrophin binding, phosphorylates itself and |
| | | | | | | | members of the MAPK pathway. Signalling through |
| | | | | | | | this kinase leads to cell differentiation and may play |
| | | | | | | | a role in the development of proprioceptive neurons |
| | | | | | | | that sense body position. Mutations in this gene |
| | | | | | | | have been associated with medulloblastomas, |
| | | | | | | | secretory breast carcinomas and other cancers. |
| | | | | | | | Several transcript variants encoding different |
| | | | | | | | isoforms have been found for this gene |
| 16_10 | 4 | HPGDS | 3′-UTR | prostaglandin D synthase | Yes | | Prostaglandin-D synthase is a sigma class |
| | | | | | | | glutathione-S-transferase family member. The |
| | | | | | | | enzyme catalyzes the conversion of PGH2 to PGD2 |
| | | | | | | | and plays a role in the production of prostanoids in |
| | | | | | | | the immune system and mast cells. The presence of |
| | | | | | | | this enzyme can be used to identify the |
| | | | | | | | differentiation stage of human megakaryocytes. |
| 16_10 | 4 | HPGDS | intronic | prostaglandin D synthase | Yes | | Prostaglandin-D synthase is a sigma class |
| | | | | | | | glutathione-S-transferase family member. The |
| | | | | | | | enzyme catalyzes the conversion of PGH2 to PGD2 |
| | | | | | | | and plays a role in the production of prostanoids in |
| | | | | | | | the immune system and mast cells. The presence of |
| | | | | | | | this enzyme can be used to identify the |
| | | | | | | | differentiation stage of human megakaryocytes. |
| 16_10 | 4 | HPGDS(5′) | | prostaglandin D synthase | Yes | | Prostaglandin-D synthase is a sigma class |
| | | | | | | | glutathione-S-transferase family member. The |
| | | | | | | | enzyme catalyzes the conversion of PGH2 to PGD2 |
| | | | | | | | and plays a role in the production of prostanoids in |
| | | | | | | | the immune system and mast cells. The presence of |
| | | | | | | | this enzyme can be used to identify the |
| | | | | | | | differentiation stage of human megakaryocytes. |
| 16_10 | 4 | RP11-363G15.2 | | spliceosome complex activation | No | no | This gene encodes a component of the spliceosome |
| | | | | | | (retinitis pigmentosa) | complex and is one of several retinitis pigmentosa- |
| | | | | | | | causing genes. When the gene product is added to |
| | | | | | | | the spliceosome complex, activation occurs. |
| 16_10 | 4 | SMARCAD1 | 3′-UTR | actin-dependent chromatin regulation | Yes | | This gene encodes a member of the SNF subfamily |
| | | | | | | | of helicase proteins. The encoded protein plays a |
| | | | | | | | critical role in the restoration of heterochromatin |
| | | | | | | | organization and propagation of epigenetic patterns |
| | | | | | | | following DNA replication by mediating histone |
| | | | | | | | H3/H4 deacetylation. Mutations in this gene are |
| | | | | | | | associated with adermatoglyphia. Alternatively |
| | | | | | | | spliced transcript variants encoding multiple |
| | | | | | | | isoforms have been observed for this gene. |
| 16_10 | 4 | SMARCAD1 | intronic | actin-dependent chromatin regulation | Yes | | This gene encodes a member of the SNF subfamily |
| | | | | | | | of helicase proteins. The encoded protein plays a |
| | | | | | | | critical role in the restoration of heterochromatin |
| | | | | | | | organization and propagation of epigenetic patterns |
| | | | | | | | following DNA replication by mediating histone |
| | | | | | | | H3/H4 deacetylation. Mutations in this gene are |
| | | | | | | | associated with adermatoglyphia. Alternatively |
| | | | | | | | spliced transcript variants encoding multiple |
| | | | | | | | isoforms have been observed for this gene. |
| 16_10 | 4 | SMARCAD1 | missense | actin-dependent chromatin regulation | Yes | | This gene encodes a member of the SNF subfamily |
| | | | | | | | of helicase proteins. The encoded protein plays a |
| | | | | | | | critical role in the restoration of heterochromatin |
| | | | | | | | organization and propagation of epigenetic patterns |
| | | | | | | | following DNA replication by mediating histone |
| | | | | | | | H3/H4 deacetylation. Mutations in this gene are |
| | | | | | | | associated with adermatoglyphia. Alternatively |
| | | | | | | | spliced transcript variants encoding multiple |
| | | | | | | | isoforms have been observed for this gene. |
| 16_10 | 4 | SMARCAD1 | synonymous | actin-dependent chromatin regulation | Yes | | This gene encodes a member of the SNF subfamily |
| | | | | | | | of helicase proteins. The encoded protein plays a |
| | | | | | | | critical role in the restoration of heterochromatin |
| | | | | | | | organization and propagation of epigenetic patterns |
| | | | | | | | following DNA replication by mediating histone |
| | | | | | | | H3/H4 deacetylation. Mutations in this gene are |
| | | | | | | | associated with adermatoglyphia. Alternatively |
| | | | | | | | spliced transcript variants encoding multiple |
| | | | | | | | isoforms have been observed for this gene. |
| 19_2 | 9 | ARPC5L | | actin-binding protein | | no | actin related protein 2/3 complex, subunit 5-like |
| 19_2 | 9 | ARPC5L | intronic | actin-binding protein | | no | actin related protein 2/3 complex, subunit 5-like |
| 19_2 | 9 | GOLGA1 | | golgi associated protein | | no | The Golgi apparatus, which participates in |
| | | | | | | | glycosylation and transport of proteins and lipids in |
| | | | | | | | the secretory pathway, consists of a series of |
| | | | | | | | stacked cisternae (flattened membrane sacs). |
| | | | | | | | Interactions between the Golgi and microtubules are |
| | | | | | | | thought to be important for the reorganization of the |
| | | | | | | | Golgi after it fragments during mitosis. This gene |
| | | | | | | | encodes one of the golgins, a family of proteins |
| | | | | | | | localized to the Golgi. This encoded protein is |
| | | | | | | | associated with Sjogren's syndrome. |
| 19_2 | 9 | GOLGA1 | 3′-UTR | golgi associated protein | | no | The Golgi apparatus, which participates in |
| | | | | | | | glycosylation and transport of proteins and lipids in |
| | | | | | | | the secretory pathway, consists of a series of |
| | | | | | | | stacked cisternae (flattened membrane sacs). |
| | | | | | | | Interactions between the Golgi and microtubules are |
| | | | | | | | thought to be important for the reorganization of the |
| | | | | | | | Golgi after it fragments during mitosis. This gene |
| | | | | | | | encodes one of the golgins, a family of proteins |
| | | | | | | | localized to the Golgi. This encoded protein is |
| | | | | | | | associated with Sjogren's syndrome. |
| 19_2 | 9 | GOLGA1 | intronic | golgi associated protein | | no | The Golgi apparatus, which participates in |
| | | | | | | | glycosylation and transport of proteins and lipids in |
| | | | | | | | the secretory pathway, consists of a series of |
| | | | | | | | stacked cisternae (flattened membrane sacs). |
| | | | | | | | Interactions between the Golgi and microtubules are |
| | | | | | | | thought to be important for the reorganization of the |
| | | | | | | | Golgi after it fragments during mitosis. This gene |
| | | | | | | | encodes one of the golgins, a family of proteins |
| | | | | | | | localized to the Golgi. This encoded protein is |
| | | | | | | | associated with Sjogren's syndrome. |
| 19_2 | 9 | GOLGA1 | missense | golgi associated protein | | no | The Golgi apparatus, which participates in |
| | | | | | | | glycosylation and transport of proteins and lipids in |
| | | | | | | | the secretory pathway, consists of a series of |
| | | | | | | | stacked cisternae (flattened membrane sacs). |
| | | | | | | | Interactions between the Golgi and microtubules are |
| | | | | | | | thought to be important for the reorganization of the |
| | | | | | | | Golgi after it fragments during mitosis. This gene |
| | | | | | | | encodes one of the golgins, a family of proteins |
| | | | | | | | localized to the Golgi. This encoded protein is |
| | | | | | | | associated with Sjogren's syndrome. |
| 19_2 | 9 | GOLGA1 | synonymous | golgi associated protein | | no | The Golgi apparatus, which participates in |
| | | | | | | | glycosylation and transport of proteins and lipids in |
| | | | | | | | the secretory pathway, consists of a series of |
| | | | | | | | stacked cisternae (flattened membrane sacs). |
| | | | | | | | Interactions between the Golgi and microtubules are |
| | | | | | | | thought to be important for the reorganization of the |
| | | | | | | | Golgi after it fragments during mitosis. This gene |
| | | | | | | | encodes one of the golgins, a family of proteins |
| | | | | | | | localized to the Golgi. This encoded protein is |
| | | | | | | | associated with Sjogren's syndrome. |
| 19_2 | 9 | RPL35 | intronic | ribosomal protein | | no | Ribosomes, the organelles that catalyze protein |
| | | | | | | | synthesis, consist of a small 40S subunit and a large |
| | | | | | | | 60S subunit. Together these subunits are composed |
| | | | | | | | of 4 RNA species and approximately 80 structurally |
| | | | | | | | distinct proteins. This gene encodes a ribosomal |
| | | | | | | | protein that is a component of the 60S subunit. The |
| | | | | | | | protein belongs to the L29P family of ribosomal |
| | | | | | | | proteins. It is located in the cytoplasm. As is typical |
| | | | | | | | for genes encoding ribosomal proteins, there are |
| | | | | | | | multiple processed pseudogenes of this gene |
| | | | | | | | dispersed through the genome. |
| 19_2 | 9 | SCAI | | regulator of Ras pathway of cell | | no | his gene encodes a regulator of cell migration. The |
| | | | | migration | | | encoded protein appears to function in the RhoA |
| | | | | | | | (ras homolog gene family, member A)-Dia1 |
| | | | | | | | (diaphanous homolog 1) signal transduction |
| | | | | | | | pathway. Alternatively spliced transcript variants |
| | | | | | | | have been described. |
| 19_2 | 9 | SCAI | intronic | regulator of Ras pathway of cell | | no | his gene encodes a regulator of cell migration. The |
| | | | | migration | | | encoded protein appears to function in the RhoA |
| | | | | | | | (ras homolog gene family, member A)-Dia1 |
| | | | | | | | (diaphanous homolog 1) signal transduction |
| | | | | | | | pathway. Alternatively spliced transcript variants |
| | | | | | | | have been described. |
| 19_2 | 9 | WDR38 | intronic | WD38 domain protein | | no | WD repeat domain 38 |
| 21_8 | 2 | AC068490.2 | | transcript without known gene product | | | |
| 22_11 | 2 | AC068490.2 | | transcript without known gene product | | | |
| 25_10 | X | AL158819.7 (3′) * | | transfer RNA tanscript | | | PAGE5. This gene is a member of the GAGE |
| | | | | | | | family, which is expressed in a variety of tumors |
| | | | | | | | and in some fetal and reproductive tissues. The |
| | | | | | | | protein encoded by this gene shares a sequence |
| | | | | | | | similarity with other GAGE/PAGE proteins. It may |
| | | | | | | | also belong to a family of CT (cancer-testis) |
| | | | | | | | antigens. Multiple alternatively spliced transcript |
| | | | | | | | variants encoding distinct isoforms have been found |
| | | | | | | | for this gene, but the biological validity of some |
| | | | | | | | variants have not been determined |
| 25_10 | X | FOXR2 * | missense | carcinogenic transcription factor | | no | forkhead box R2 |
| 25_10 | X | FOXR2(3′) * | | carcinogenic transcription factor | | no | forkhead box R3 |
| 25_10 | X | MAGEH1(5′) * | | apoptosis mediator | | no | This gene is thought to be involved in apoptosis. |
| | | | | | | | Multiple polyadenylation sites have been found for |
| | | | | | | | this gene. |
| 25_10 | X | PAGE3 * | | none (prostate associated gene) | | no | P antigen family, member 3 (prostate associated) |
| 25_10 | X | PAGE3 * | missense | none (prostate associated gene) | | no | P antigen family, member 3 (prostate associated) |
| 25_10 | X | PAGE3(3′) * | | none (prostate associated gene) | | no | P antigen family, member 3 (prostate associated) |
| 25_10 | X | PAGE5(3′) * | | inhibition of apoptosis | | no | P antigen family, member 3 (prostate associated) |
| 25_10 | X | PAGE5(5′) * | | inhibition of apoptosis | | no | This gene is a member of the GAGE family, which |
| | | | | | | | is expressed in a variety of tumors and in some fetal |
| | | | | | | | and reproductive tissues. The protein encoded by |
| | | | | | | | this gene shares a sequence similarity with other |
| | | | | | | | GAGE/PAGE proteins. It may also belong to a |
| | | | | | | | family of CT (cancer-testis) antigens. Multiple |
| | | | | | | | alternatively spliced transcript variants encoding |
| | | | | | | | distinct isoforms have been found for this gene, but |
| | | | | | | | the biological validity of some variants have not |
| | | | | | | | been determined. |
| 25_10 | X | RP11-382F24.2 * | | transcript without known gene product | | no | |
| 25_10 | X | RP11-382F24.2(3′) * | | transcript without known gene product | | no | |
| 25_10 | X | RP11-382F24.2(5′) * | | transcript without known gene product | | no | |
| 25_10 | X | RP13-188A5.1 * | | transcript without known gene product | | no | |
| 25_10 | X | RRAGB | intronic | Ras related GTP binding | | no | Ras-homologous GTPases constitute a large family |
| | | | | | | | of signal transducers that alternate between an |
| | | | | | | | activated, GTP-binding state and an inactivated, |
| | | | | | | | GDP-binding state. These proteins represent |
| | | | | | | | cellular switches that are operated by GTP- |
| | | | | | | | exchange factors and factors that stimulate their |
| | | | | | | | intrinsic GTPase activity. All GTPases of the Ras |
| | | | | | | | superfamily have in common the presence of six |
| | | | | | | | conserved motifs involved in GTP/GDP binding, |
| | | | | | | | three of which are phosphate-/magnesium-binding |
| | | | | | | | sites (PM1-PM3) and three of which are guanine |
| | | | | | | | nucleotide-binding sites (G1-G3). Transcript |
| | | | | | | | variants encoding distinct isoforms have been |
| | | | | | | | identified. |
| 25_10 | X | RRAGB(3′) | | Ras related GTP binding | | no | Ras-homologous GTPases constitute a large family |
| | | | | | | | of signal transducers that alternate between an |
| | | | | | | | activated, GTP-binding state and an inactivated, |
| | | | | | | | GDP-binding state. These proteins represent |
| | | | | | | | cellular switches that are operated by GTP- |
| | | | | | | | exchange factors and factors that stimulate their |
| | | | | | | | intrinsic GTPase activity. All GTPases of the Ras |
| | | | | | | | superfamily have in common the presence of six |
| | | | | | | | conserved motifs involved in GTP/GDP binding, |
| | | | | | | | three of which are phosphate-/magnesium-binding |
| | | | | | | | sites (PM1-PM3) and three of which are guanine |
| | | | | | | | nucleotide-binding sites (G1-G3). Transcript |
| | | | | | | | variants encoding distinct isoforms have been |
| | | | | | | | identified. |
| 25_10 | X | RRAGB(5′) | | Ras related GTP binding | | no | Ras-homologous GTPases constitute a large family |
| | | | | | | | of signal transducers that alternate between an |
| | | | | | | | activated, GTP-binding state and an inactivated, |
| | | | | | | | GDP-binding state. These proteins represent |
| | | | | | | | cellular switches that are operated by GTP- |
| | | | | | | | exchange factors and factors that stimulate their |
| | | | | | | | intrinsic GTPase activity. All GTPases of the Ras |
| | | | | | | | superfamily have in common the presence of six |
| | | | | | | | conserved motifs involved in GTP/GDP binding, |
| | | | | | | | three of which are phosphate-/magnesium-binding |
| | | | | | | | sites (PM1-PM3) and three of which are guanine |
| | | | | | | | nucleotide-binding sites (G1-G3). Transcript |
| | | | | | | | variants encoding distinct isoforms have been |
| | | | | | | | identified. |
| 25_10 | X | SNORD112.49(3′) * | | small nucleolar RNA with ribosomal | | no | small nucleolar RNA, C/D box 112 |
| | | | | function | | | |
| 31_22 | 6 | C6orf138 | 3′-UTR | unkown function | | yes | patched domain 5 |
| | | | | | | (smoking cessation) | |
| 31_22 | 6 | C6orf138 | intronic | unkown function | | yes | patched domain 5 |
| | | | | | | (smoking cessation) | |
| 31_22 | 6 | C6orf138 | synonymous | unkown function | | yes | patched domain 5 |
| | | | | | | (smoking cessation) | |
| 31_22 | 6 | C6orf138(3′) | | unkown function | | yes | patched domain 6 |
| | | | | | | (smoking cessation) | |
| 31_22 | 6 | OPN5(3′) * | | neuropsin | | yes | Opsins are members of the guanine nucleotide- |
| | | | | (G protein associated receptor) | | (bipolar disorder) | binding protein (G protein)-coupled receptor |
| | | | | | | | superfamily. This opsin gene is expressed in the |
| | | | | | | | eye, brain, testes, and spinal cord. This gene |
| | | | | | | | belongs to the seven-exon subfamily of mammalian |
| | | | | | | | opsin genes that includes peropsin (RRH) and |
| | | | | | | | retinal G protein coupled receptor (RGR). Like |
| | | | | | | | these other seven-exon opsin genes, this family |
| | | | | | | | member may encode a protein with photoisomerase |
| | | | | | | | activity. Alternative splicing results in multiple |
| | | | | | | | transcript variants. |
| 41_12 | X | GPR119(3′) | | rhodopsin | | no | This gene encodes a member of the rhodopsin |
| | | | | (G protein associated receptor) | | | subfamily of G-protein-coupled receptors that is |
| | | | | | | | expressed in the pancreas and gastrointestinal tract. |
| | | | | | | | The encoded protein is activated by lipid amides |
| | | | | | | | including lysophosphatidylcholine and |
| | | | | | | | oleoylethanolamide and may be involved in glucose |
| | | | | | | | homeostasis. This protein is a potential drug target |
| | | | | | | | in the treatment of type 2 diabetes |
| 41_12 | X | SLC25A14 | intronic | mitochondrial uncoupling in neurons | | but two other UCP genes | Mitochondrial uncoupling proteins (UCP) are |
| | | | | | | are associated to SZ | members of the larger family of mitochondrial |
| | | | | | | | anion carrier proteins (MACP). UCPs separate |
| | | | | | | | oxidative phosphorylation from ATP synthesis with |
| | | | | | | | energy dissipated as heat, also referred to as the |
| | | | | | | | mitochondrial proton leak. UCPs facilitate the |
| | | | | | | | transfer of anions from the inner to the outer |
| | | | | | | | mitochondrial membrane and the return transfer of |
| | | | | | | | protons from the outer to the inner mitochondrial |
| | | | | | | | membrane. They also reduce the mitochondrial |
| | | | | | | | membrane potential in mammalian cells. Tissue |
| | | | | | | | specificity occurs for the different UCPs and the |
| | | | | | | | exact methods of how UCPs transfer H+/OH− are |
| | | | | | | | not known. UCPs contain the three homologous |
| | | | | | | | protein domains of MACPs. This gene is widely |
| | | | | | | | expressed in many tissues with the greatest |
| | | | | | | | abundance in brain and testis |
| 41_12 | X | SLC25A14(3′) | | mitochondrial uncoupling in neurons | | but two other UCP genes are | Mitochondrial uncoupling proteins (UCP) are |
| | | | | | | associated to SZ | members of the larger family of mitochondrial |
| | | | | | | | anion carrier proteins (MACP). UCPs separate |
| | | | | | | | oxidative phosphorylation from ATP synthesis with |
| | | | | | | | energy dissipated as heat, also referred to as the |
| | | | | | | | mitochondrial proton leak. UCPs facilitate the |
| | | | | | | | transfer of anions from the inner to the outer |
| | | | | | | | mitochondrial membrane and the return transfer of |
| | | | | | | | protons from the outer to the inner mitochondrial |
| | | | | | | | membrane. They also reduce the mitochondrial |
| | | | | | | | membrane potential in mammalian cells. Tissue |
| | | | | | | | specificity occurs for the different UCPs and the |
| | | | | | | | exact methods of how UCPs transfer H+/OH− are |
| | | | | | | | not known. UCPs contain the three homologous |
| | | | | | | | protein domains of MACPs. This gene is widely |
| | | | | | | | expressed in many tissues with the greatest |
| | | | | | | | abundance in brain and testis |
| 42_37 | 11 | NCAM1 | | neuronal adhesion | | expression is abnormal in SCH. | This gene encodes a cell adhesion protein which is a |
| | | | | | | | member of the immunoglobulin superfamily. The |
| | | | | | | | encoded protein is involved in cell-to-cell |
| | | | | | | | interactions as well as cell-matrix interactions |
| | | | | | | | during development and differentiation. The |
| | | | | | | | encoded protein has been shown to be involved in |
| | | | | | | | development of the nervous system, and for cells |
| | | | | | | | involved in the expansion of T cells and dendritic |
| | | | | | | | cells which play an important role in immune |
| | | | | | | | surveillance. Alternative splicing results in multiple |
| | | | | | | | transcript variants. |
| 42_37 | 11 | NCAM1 | intronic | neuronal adhesion | | expression is abnormal in SCH. | This gene encodes a cell adhesion protein which is a |
| | | | | | | | member of the immunoglobulin superfamily. The |
| | | | | | | | encoded protein is involved in cell-to-cell |
| | | | | | | | interactions as well as cell-matrix interactions |
| | | | | | | | during development and differentiation. The |
| | | | | | | | encoded protein has been shown to be involved in |
| | | | | | | | development of the nervous system, and for cells |
| | | | | | | | involved in the expansion of T cells and dendritic |
| | | | | | | | cells which play an important role in immune |
| | | | | | | | surveillance. Alternative splicing results in multiple |
| | | | | | | | transcript variants. |
| 42_37 | 11 | RP11-629G13.1 | | novel transcript, antisense to NCAM1 | | expression is abnormal in SCH. | |
| 42_37 | 11 | RP11-629G13.1 | intronic | novel transcript, antisense to NCAM1 | | expression is abnormal in SCH. | |
| 42_37 | 11 | RP11-629G13.1(3′) | | novel transcript, antisense to NCAM1 | | expression is abnormal in SCH. | |
| 42_37 | 2 | AC064837.1 * | intronic | Novel miRNA | | | REAL GeneNAME IPP5: Protein phosphatase-1 |
| | | | | | | | (PP1) is a major serine/threonine phosphatase that |
| | | | | | | | regulates a variety of cellular functions. PP1 |
| | | | | | | | consists of a catalytic subunit (see PPP1CA; MIM |
| | | | | | | | 176875) and regulatory subunits that determine the |
| | | | | | | | subcellular localization of PP1 or regulate its |
| | | | | | | | function. PPP1R1C belongs to a group of PP1 |
| | | | | | | | inhibitory subunits that are themselves regulated by |
| | | | | | | | phosphorylation |
| 42_37 | 2 | PPP1R1C | intronic | protein phosphatase 1, regulatory | | regulates TNF induced apoptosis | REAL GeneNAME IPP5: Protein phosphatase-1 |
| | | | | (inhibitor) subunit | | (p53 mediated) | (PP1) is a major serine/threonine phosphatase that |
| | | | | | | | regulates a variety of cellular functions. PP1 |
| | | | | | | | consists of a catalytic subunit (see PPP1CA; MIM |
| | | | | | | | 176875) and regulatory subunits that determine the |
| | | | | | | | subcellular localization of PP1 or regulate its |
| | | | | | | | function. PPP1R1C belongs to a group of PP1 |
| | | | | | | | inhibitory subunits that are themselves regulated by |
| | | | | | | | phosphorylation |
| 51_28 | X | IGSF1 | | a member of the immunoglobulin- | | central hypothyroidism and | This gene encodes a member of the |
| | | | | like domain-containing superfamily | | testicular enlargement. | immunoglobulin-like domain-containing |
| | | | | | | | superfamily. Proteins in this superfamily contain |
| | | | | | | | varying numbers of immunoglobulin-like domains |
| | | | | | | | and are thought to participate in the regulation of |
| | | | | | | | interactions between cells. Multiple transcript |
| | | | | | | | variants encoding different isoforms have been |
| | | | | | | | found for this gene. |
| 52_42 | 11 | NCAM1 | | neuronal adhesion | | expression is abnormal in SCH. | This gene encodes a cell adhesion protein which is a |
| | | | | | | | member of the immunoglobulin superfamily. The |
| | | | | | | | encoded protein is involved in cell-to-cell |
| | | | | | | | interactions as well as cell-matrix interactions |
| | | | | | | | during development and differentiation. The |
| | | | | | | | encoded protein has been shown to be involved in |
| | | | | | | | development of the nervous system, and for cells |
| | | | | | | | involved in the expansion of T cells and dendritic |
| | | | | | | | cells which play an important role in immune |
| | | | | | | | surveillance. Alternative splicing results in multiple |
| | | | | | | | transcript variants. |
| 52_42 | 11 | NCAM1 | intronic | neuronal adhesion | | expression is abnormal in SCH. | This gene encodes a cell adhesion protein which is a |
| | | | | | | | member of the immunoglobulin superfamily. The |
| | | | | | | | encoded protein is involved in cell-to-cell |
| | | | | | | | interactions as well as cell-matrix interactions |
| | | | | | | | during development and differentiation. The |
| | | | | | | | encoded protein has been shown to be involved in |
| | | | | | | | development of the nervous system, and for cells |
| | | | | | | | involved in the expansion of T cells and dendritic |
| | | | | | | | cells which play an important role in immune |
| | | | | | | | surveillance. Alternative splicing results in multiple |
| | | | | | | | transcript variants. |
| 52_42 | 11 | RP11-629G13.1 | | novel transcript, antisense to NCAM1 | | expression is abnormal in SCH. | |
| 52_42 | 11 | RP11-629G13.1 | intronic | novel transcript, antisense to NCAM1 | | expression is abnormal in SCH. | |
| 52_42 | 11 | RP11-629G13.1(3′) | | novel transcript, antisense to NCAM1 | | expression is abnormal in SCH. | |
| 54_51 | 8 | CSMD1 | intronic | potential tumor suppressor | Yes | deletion related to head and neck | CUB and Sushi multiple domains 1 |
| | | | | | | carcinomas | |
| 56_19 | 11 | SNX19(5′) * | | sorting nexin 19 | Yes | | sorting nexin 19 |
| 56_30 | 1 | 7SK.207(3′) * | | non coding RNA novel transcript | | | snRNA |
| 56_30 | 1 | 7SK.207(5′) * | | non coding RNA novel transcript | | | snRNA |
| 56_30 | 1 | PTBP2 | intronic | controls the assembly of other | Yes | | The protein encoded by this gene binds to the |
| | | | | splicing-regulatory proteins | | | intronic cluster of RNA regulatory elements, |
| | | | | | | | downstream control sequence (DCS). It is |
| | | | | | | | implicated in controlling the assembly of other |
| | | | | | | | splicing-regulatory proteins. This protein is very |
| | | | | | | | similar to the polypyrimidine tract binding protein |
| | | | | | | | but it is expressed primarily in the brain. |
| 56_30 | 1 | PTBP2 | synonymous | controls the assembly of other | Yes | | The protein encoded by this gene binds to the |
| | | | | splicing-regulatory proteins | | | intronic cluster of RNA regulatory elements, |
| | | | | | | | downstream control sequence (DCS). It is |
| | | | | | | | implicated in controlling the assembly of other |
| | | | | | | | splicing-regulatory proteins. This protein is very |
| | | | | | | | similar to the polypyrimidine tract binding protein |
| | | | | | | | but it is expressed primarily in the brain. |
| 56_30 | 1 | PTBP2(5′) | | controls the assembly of other | Yes | | The protein encoded by this gene binds to the |
| | | | | splicing-regulatory proteins | | | intronic cluster of RNA regulatory elements, |
| | | | | | | | downstream control sequence (DCS). It is |
| | | | | | | | implicated in controlling the assembly of other |
| | | | | | | | splicing-regulatory proteins. This protein is very |
| | | | | | | | similar to the polypyrimidine tract binding protein |
| | | | | | | | but it is expressed primarily in the brain. |
| 56_30 | 1 | RP4-726F1.1(3′) * | | non coding RNA novel transcript | | | Rodopsine: Retinitis pigmentosa is an inherited |
| | | | | | | | progressive disease which is a major cause of |
| | | | | | | | blindness in western communities. It can be |
| | | | | | | | inherited as an autosomal dominant, autosomal |
| | | | | | | | recessive, or X-linked recessive disorder. In the |
| | | | | | | | autosomal dominant form, which comprises about |
| | | | | | | | 25% of total cases, approximately 30% of families |
| | | | | | | | have mutations in the gene encoding the rod |
| | | | | | | | photoreceptor-specific protein rhodopsin. This is |
| | | | | | | | the transmembrane protein which, when |
| | | | | | | | photoexcited, initiates the visual transduction |
| | | | | | | | cascade. Defects in this gene are also one of the |
| | | | | | | | causes of congenital stationary night blindness. |
| 56_30 | 16 | GP2 * | intronic | glycoprotein 2 | Yes | | glycoprotein 2 (zymogen granule membrane) |
| 56_30 | 16 | GP2 * | synonymous | glycoprotein 2 | Yes | | glycoprotein 2 (zymogen granule membrane) |
| 56_30 | 16 | GP2(3′) * | | glycoprotein 2 | Yes | | glycoprotein 2 (zymogen granule membrane) |
| 58_29 | 8 | CTD-3025N20.2(3′) * | | Novel long non coding RNA | | | Genomic clone: CTD Coats disease |
| 58_29 | 8 | RP11-1D12.2(5′) * | | Novel long non coding RNA | | | |
| 59_48 | 20 | RP11-128M1.1 | | Novel long non coding RNA | | | |
| 59_48 | 20 | RP11-128M1.1(3′) | | Novel long non coding RNA | | | |
| 59_48 | 8 | TRPS1(3′) | | transcription factor that represses | | | This gene encodes a transcription factor that |
| | | | | GATA-regulated genes and binds | | | represses GATA-regulated genes and binds to a |
| | | | | to a dynein light chain protein | | | dynein light chain protein. Binding of the encoded |
| | | | | | | | protein to the dynein light chain protein affects |
| | | | | | | | binding to GATA consensus sequences and |
| | | | | | | | suppresses its transcriptional activity. Defects in |
| | | | | | | | this gene are a cause of tricho-rhino-phalangeal |
| | | | | | | | syndrome (TRPS) types I-III |
| 61_39 | X | IGSF1 | | a member of the immunoglobulin- | | central hypothyroidism and | This gene encodes a member of the |
| | | | | like domain-containing superfamily | | testicular enlargement. | immunoglobulin-like domain-containing |
| | | | | | | | superfamily. Proteins in this superfamily contain |
| | | | | | | | varying numbers of immunoglobulin-like domains |
| | | | | | | | and are thought to participate in the regulation of |
| | | | | | | | interactions between cells. Multiple transcript |
| | | | | | | | variants encoding different isoforms have been |
| | | | | | | | found for this gene. |
| 65_25 | 20 | C20orf78(5′) * | | exon, codes protein of unknown function | | | chromosome 20 open reading frame 79 |
| 71_55 | 15 | NTRK3(3′) * | | neurotrophic tyrosine receptor kinase | Yes | alcoholism | This gene encodes a member of the neurotrophic |
| | | | | (NTRK) | | | tyrosine receptor kinase (NTRK) family. This |
| | | | | | | | kinase is a membrane-bound receptor that, upon |
| | | | | | | | neurotrophin binding, phosphorylates itself and |
| | | | | | | | members of the MAPK pathway. Signalling through |
| | | | | | | | this kinase leads to cell differentiation and may play |
| | | | | | | | a role in the development of proprioceptive neurons |
| | | | | | | | that sense body position. Mutations in this gene |
| | | | | | | | have been associated with medulloblastomas, |
| | | | | | | | secretory breast carcinomas and other cancers. |
| | | | | | | | Several transcript variants encoding different |
| | | | | | | | isoforms have been found for this gene |
| 75_31 | 1 | AC093577.1 (3′) | | Novel non-coding miRNA | | | genomic clone RELATED to FAM69 family of |
| | | | | | | | cysteine-rich type II transmembrane proteins. These |
| | | | | | | | proteins localize to the endoplasmic reticulum but |
| | | | | | | | their specific functions are unknown. Alternatively |
| | | | | | | | spliced transcript variants encoding multiple |
| | | | | | | | isoforms have been observed for this gene. |
| 75_31 | 1 | AC093577.1 (5′) | | Novel non-coding miRNA | | | genomic clone RELATED to FAM69 family of |
| | | | | | | | cysteine-rich type II transmembrane proteins. These |
| | | | | | | | proteins localize to the endoplasmic reticulum but |
| | | | | | | | their specific functions are unknown. Alternatively |
| | | | | | | | spliced transcript variants encoding multiple |
| | | | | | | | isoforms have been observed for this gene. |
| 75_31 | 1 | U6.1077(5′) | | U6 spliceosomal RNA | | | RNA, U6 small nuclear |
| 75_31 | 11 | SNX19(5′) * | | sorting nexin 19 | Yes | | sorting nexin 19 |
| 75_67 | 1 | SNORA42.4 (5′) * | | small nucleolar RNA, H/ACA box 42; | | | small nucleolar RNA, H/ACA box 42 |
| | | | | regulation of gene expression | | | |
| 75_67 | 1 | VANGL1(5′) * | | tretraspanin family member; NfKB | | | This gene encodes a member of the tretraspanin |
| | | | | regulating microRNA | | | family. The encoded protein may be involved in |
| | | | | | | | mediating intestinal trefoil factor induced wound |
| | | | | | | | healing in the intestinal mucosa. Mutations in this |
| | | | | | | | gene are associated with neural tube defects. |
| | | | | | | | Alternate splicing results in multiple transcript |
| | | | | | | | variants. |
| 75_67 | 10 | RP11-298H24.1(3′) * | | Novel long non coding RNA | | | |
| 75_67 | 12 | STYK1 | intronic | Receptor protein tyrosine kinases | | NOK/STYK1 interacts with GSK-3? | Receptor protein tyrosine kinases, like STYK1, play |
| | | | | | | and mediates Ser9 phosphorylation | important roles in diverse cellular and |
| | | | | | | through activated Akt. | developmental processes, such as cell proliferation, |
| | | | | | | | differentiation, and survival |
| 75_67 | 14 | AL161669.1 (3′) * | | MicroRNA? | | | |
| 75_67 | 14 | AL161669.1 (5′) * | | MicroRNA? | | | |
| 75_67 | 14 | AL161669.2 * | | MicroRNA | | | |
| 75_67 | 14 | AL161669.2 (3′) * | | MicroRNA | | | |
| 75_67 | 15 | 5S_rRNA.496(3′) * | | 5S ribosomal RNA | | | 5S ribosomal RNA |
| 75_67 | 15 | NTRK3(3′) * | | neurotrophic tyrosine receptor kinase | Yes | alcoholism | This gene encodes a member of the neurotrophic |
| | | | | (NTRK) | | | tyrosine receptor kinase (NTRK) family. This |
| | | | | | | | kinase is a membrane-bound receptor that, upon |
| | | | | | | | neurotrophin binding, phosphorylates itself and |
| | | | | | | | members of the MAPK pathway. Signalling through |
| | | | | | | | this kinase leads to cell differentiation and may play |
| | | | | | | | a role in the development of proprioceptive neurons |
| | | | | | | | that sense body position. Mutations in this gene |
| | | | | | | | have been associated with medulloblastomas, |
| | | | | | | | secretory breast carcinomas and other cancers. |
| | | | | | | | Several transcript variants encoding different |
| | | | | | | | isoforms have been found for this gene |
| 75_67 | 16 | 7SK.236(5′) * | | non coding RNA novel transcript | | | snRNA |
| 75_67 | 16 | GP2 * | intronic | glycoprotein 2 | Yes | | glycoprotein 2 (zymogen granule membrane) |
| 75_67 | 16 | GP2 * | synonymous | glycoprotein 2 | Yes | | glycoprotein 2 (zymogen granule membrane) |
| 75_67 | 16 | GP2(3′) * | | glycoprotein 2 | Yes | | glycoprotein 2 (zymogen granule membrane) |
| 75_67 | 22 | CTA-714B7.5 | | Novel transcript, genomic, unknown protein. | | | PCYT1A phosphate cytidylyltransferase 1, choline, |
| | | | | | | | alpha |
| 75_67 | 3 | RP11-436A20.3 | | Novel long non coding RNA | | | Homo sapiens 3 BAC RP11-436A20 (Roswell Park |
| | | | | | | | Cancer Institute Human BAC Library) complete |
| | | | | | | | sequence. |
| 75_67 | 4 | C4orf37 | | sperm-tail PG-rich repeat containing 2 | | | sperm-tail PG-rich repeat |
| 75_67 | 4 | C4orf37(3′) | | sperm-tail PG-rich repeat containing 3 | | | sperm-tail PG-rich repeat |
| 75_67 | 4 | RP11-431J17.1(3′) | | Novel long non coding RNA | | | Homo sapiens BAC clone RP11-431J17 from 4, |
| | | | | | | | complete sequence |
| 75_67 | 8 | 7SK.7(3′) * | | | | | snRNA |
| 75_67 | 8 | DKK4(5′) * | | a Wnt/beta catenin signaling pathway | Yes | gene expression is altered | This gene encodes a protein that is a member of the |
| | | | | member of the dickkopf family | | in schizophrenia | dickkopf family. The secreted protein contains two |
| | | | | involved in embryonic development | | | cysteine rich regions and is involved in embryonic |
| | | | | | | | development through its interactions with the Wnt |
| | | | | | | | signaling pathway. Activity of this protein is |
| | | | | | | | modulated by binding to the Wnt co-receptor and |
| | | | | | | | the co-factor kremen 2. |
| 75_67 | 8 | DUSP4(5′) * | | dual specificity phosphatase 4; | Yes | | The protein encoded by this gene is a member of |
| | | | | gene product inactivates | | | the dual specificity protein phosphatase subfamily. |
| | | | | ERK1, ERK2 and JNK | | | These phosphatases inactivate their target kinases |
| | | | | | | | by dephosphorylating both the |
| | | | | | | | phosphoserine/threonine and phosphotyrosine |
| | | | | | | | residues. They negatively regulate members of the |
| | | | | | | | mitogen-activated protein (MAP) kinase |
| | | | | | | | superfamily (MAPK/ERK, SAPK/JNK, p38), |
| | | | | | | | which are associated with cellular proliferation and |
| | | | | | | | differentiation. Different members of the family of |
| | | | | | | | dual specificity phosphatases show distinct |
| | | | | | | | substrate specificities for various MAP kinases, |
| | | | | | | | different tissue distribution and subcellular |
| | | | | | | | localization, and different modes of inducibility of |
| | | | | | | | their expression by extracellular stimuli. This gene |
| | | | | | | | product inactivates ERK1, ERK2 and JNK, is |
| | | | | | | | expressed in a variety of tissues, and is localized in |
| | | | | | | | the nucleus. Two alternatively spliced transcript |
| | | | | | | | variants, encoding distinct isoforms, have been |
| | | | | | | | observed for this gene. In addition, multiple |
| | | | | | | | polyadenylation sites have been reported. |
| 75_67 | 8 | GSR | intronic | glutathione reductase | | Cerebrovascular disease, | This gene encodes a member of the class-I pyridine |
| | | | | | | metabolic syndrome | nucleotide-disulfide oxidoreductase family. This |
| | | | | | | | enzyme is a homodimeric flavoprotein. It is a |
| | | | | | | | central enzyme of cellular antioxidant defense, and |
| | | | | | | | reduces oxidized glutathione disulfide (GSSG) to |
| | | | | | | | the sulfhydryl form GSH, which is an important |
| | | | | | | | cellular antioxidant. Rare mutations in this gene |
| | | | | | | | result in hereditary glutathione reductase |
| | | | | | | | deficiency. Multiple alternatively spliced transcript |
| | | | | | | | variants encoding different isoforms have been |
| | | | | | | | found. |
| 75_67 | 8 | RP11-401H2.1(5′) * | | exon transcript. | | | |
| | | | | Codes an unknown protein | | | |
| 75_67 | 8 | RP11-486M23.1(5′) * | | Novel long non coding RNA | | | |
| 75_67 | 8 | RP11-738G5.1(3′) * | | Novel long non coding RNA | | | |
| 75_67 | 8 | RP11-770E5.1 | | Novel antisense gene transcript | | | |
| 75_67 | 8 | SLC20A2 | intronic | Type 3 sodium-dependent phosphate | | Mutations in this gene may play a | This gene encodes a member of the inorganic |
| | | | | symporter; confers susceptibility to | | role in familial idiopathic basal | phosphate transporter family. The encoded protein |
| | | | | viral infection as a gamma-retroviral | | ganglia calcification | is a type 3 sodium-dependent phosphate symporter |
| | | | | receptor. | | | that plays an important role in phosphate |
| | | | | | | | homeostasis by mediating cellular phosphate |
| | | | | | | | uptake. The encoded protein also confers |
| | | | | | | | susceptibility to viral infection as a gamma- |
| | | | | | | | retroviral receptor. Mutations in this gene may play |
| | | | | | | | a role in familial idiopathic basal ganglia |
| | | | | | | | calcification. Alternatively spliced transcript |
| | | | | | | | variants encoding multiple isoforms have been |
| | | | | | | | observed for this gene. |
| 75_67 | 8 | SNTG1 | intronic | Syntrophins; mediates dystrophin binding. | | | The protein encoded by this gene is a member of |
| | | | | Specifically expressed in the brain | | | the syntrophin family. Syntrophins are cytoplasmic |
| | | | | | | | peripheral membrane proteins that typically contain |
| | | | | | | | 2 pleckstrin homology (PH) domains, a PDZ |
| | | | | | | | domain that bisects the first PH domain, and a C- |
| | | | | | | | terminal domain that mediates dystrophin binding. |
| | | | | | | | This gene is specifically expressed in the brain. |
| | | | | | | | Transcript variants for this gene have been |
| | | | | | | | described, but their full-length nature has not been |
| | | | | | | | determined. |
| 75_67 | 8 | SNTG1(3′) | | Syntrophins; mediates dystrophin binding. | | | The protein encoded by this gene is a member of |
| | | | | Specifically expressed in the brain | | | the syntrophin family. Syntrophins are cytoplasmic |
| | | | | | | | peripheral membrane proteins that typically contain |
| | | | | | | | 2 pleckstrin homology (PH) domains, a PDZ |
| | | | | | | | domain that bisects the first PH domain, and a C- |
| | | | | | | | terminal domain that mediates dystrophin binding. |
| | | | | | | | This gene is specifically expressed in the brain. |
| | | | | | | | Transcript variants for this gene have been |
| | | | | | | | described, but their full-length nature has not been |
| | | | | | | | determined. |
| 75_67 | 8 | ST18 | intronic | Suppression of tumorigenicity 18 | | | suppression of tumorigenicity 18 (breast carcinoma) |
| | | | | (zinc finger protein); pro apoptotic | | | (zinc finger protein) |
| 75_67 | 8 | VDAC3 * | intronic | voltage-dependent anion channel (VDAC), | | Cerebrovascular disease, | This gene encodes a voltage-dependent anion |
| | | | | and belongs to the mitochondrial | | metabolic syndrome | channel (VDAC), and belongs to the mitochondrial |
| | | | | porin family. Pro apoptotic | | | porin family. VDACs are small, integral membrane |
| | | | | | | | proteins that traverse the outer mitochondrial |
| | | | | | | | membrane and conduct ATP and other small |
| | | | | | | | metabolites. They are known to bind several kinases |
| | | | | | | | of intermediary metabolism, thought to be involved |
| | | | | | | | in translocation of adenine nucleotides, and are |
| | | | | | | | hypothesized to form part of the mitochondrial |
| | | | | | | | permeability transition pore, which results in the |
| | | | | | | | release of cytochrome c at the onset of apoptotic |
| | | | | | | | cell death. Alternatively transcript variants |
| | | | | | | | encoding different isoforms have been described for |
| | | | | | | | this gene. |
| 76_63 | X | IGSF1 | | a member of the immunoglobulin- | | central hypothyroidism and | This gene encodes a member of the |
| | | | | like domain-containing superfamily | | testicular enlargement. | immunoglobulin-like domain-containing |
| | | | | | | | superfamily. Proteins in this superfamily contain |
| | | | | | | | varying numbers of immunoglobulin-like domains |
| | | | | | | | and are thought to participate in the regulation of |
| | | | | | | | interactions between cells. Multiple transcript |
| | | | | | | | variants encoding different isoforms have been |
| | | | | | | | found for this gene. |
| 76_74 | 14 | AL161669.1 (3′) * | | MicroRNA? | | | |
| 76_74 | 14 | AL161669.1 (5′) * | | MicroRNA? | | | |
| 76_74 | 14 | AL161669.2 * | | MicroRNA | | | |
| 76_74 | 14 | AL161669.2 (3′) * | | MicroRNA | | | |
| 76_74 | 16 | ABCC12(3′) | | ATP-binding cassette (ABC) transporters | | | This gene is a member of the superfamily of ATP- |
| | | | | | | | binding cassette (ABC) transporters and the |
| | | | | | | | encoded protein contains two ATP-binding domains |
| | | | | | | | and 12 transmembrane regions. ABC proteins |
| | | | | | | | transport various molecules across extra- and |
| | | | | | | | intracellular membranes. ABC genes are divided |
| | | | | | | | into seven distinct subfamilies: ABC1, MDR/TAP, |
| | | | | | | | MRP, ALD, OABP, GCN20, and White. This gene |
| | | | | | | | is a member of the MRP subfamily which is |
| | | | | | | | involved in multi-drug resistance. This gene and |
| | | | | | | | another subfamily member are arranged head-to-tail |
| | | | | | | | on chromosome 16q12.1. Increased expression of |
| | | | | | | | this gene is associated with breast cancer. |
| 76_74 | 16 | ITFG1 | intronic | Integrin alpha FG GAP repeat | | | integrin alpha FG-GAP repeat containing 1 |
| | | | | containing protein | | | |
| 76_74 | 16 | NETO2 * | | neuropilin (NRP) and tolloid (TLL)- | | rats encodes a protein that | This gene encodes a predicted transmembrane |
| | | | | like 2 | | modulates glutamate signaling | protein containing two extracellular CUB domains |
| | | | | | | in the brain by regulating | followed by a low-density lipoprotein class A |
| | | | | | | kainate receptor function. | (LDLa) domain. A similar gene in rats encodes a |
| | | | | | | | protein that modulates glutamate signaling in the |
| | | | | | | | brain by regulating kainate receptor function. |
| | | | | | | | Expression of this gene may be a biomarker for |
| | | | | | | | proliferating infantile hemangiomas. A pseudogene |
| | | | | | | | of this gene is located on the long arm of |
| | | | | | | | chromosome 8. Alternatively spliced transcript |
| | | | | | | | variants encoding multiple isoforms have been |
| | | | | | | | observed for this gene. |
| 76_74 | 16 | NETO2 * | intronic | neuropilin (NRP) and tolloid (TLL)- | | rats encodes a protein that | This gene encodes a predicted transmembrane |
| | | | | like 2 | | modulates glutamate signaling | protein containing two extracellular CUB domains |
| | | | | | | in the brain by regulating | followed by a low-density lipoprotein class A |
| | | | | | | kainate receptor function. | (LDLa) domain. A similar gene in rats encodes a |
| | | | | | | | protein that modulates glutamate signaling in the |
| | | | | | | | brain by regulating kainate receptor function. |
| | | | | | | | Expression of this gene may be a biomarker for |
| | | | | | | | proliferating infantile hemangiomas. A pseudogene |
| | | | | | | | of this gene is located on the long arm of |
| | | | | | | | chromosome 8. Alternatively spliced transcript |
| | | | | | | | variants encoding multiple isoforms have been |
| | | | | | | | observed for this gene. |
| 76_74 | 16 | PHKB * | intronic | phosphorylase kinase, beta | | | Phosphorylase kinase is a polymer of 16 subunits, |
| | | | | | | | four each of alpha, beta, gamma and delta. The |
| | | | | | | | alpha subunit includes the skeletal muscle and |
| | | | | | | | hepatic isoforms, encoded by two different genes. |
| | | | | | | | The beta subunit is the same in both the muscle and |
| | | | | | | | hepatic isoforms, encoded by this gene, which is a |
| | | | | | | | member of the phosphorylase b kinase regulatory |
| | | | | | | | subunit family. The gamma subunit also includes |
| | | | | | | | the skeletal muscle and hepatic isoforms, encoded |
| | | | | | | | by two different genes. The delta subunit is a |
| | | | | | | | calmodulin and can be encoded by three different |
| | | | | | | | genes. The gamma subunits contain the active site |
| | | | | | | | of the enzyme, whereas the alpha and beta subunits |
| | | | | | | | have regulatory functions controlled by |
| | | | | | | | phosphorylation. The delta subunit mediates the |
| | | | | | | | dependence of the enzyme on calcium |
| | | | | | | | concentration. Mutations in this gene cause |
| | | | | | | | glycogen storage disease type 9B, also known as |
| | | | | | | | phosphorylase kinase deficiency of liver and |
| | | | | | | | muscle. Alternatively spliced transcript variants |
| | | | | | | | encoding different isoforms have been identified in |
| | | | | | | | this gene. Two pseudogenes have been found on |
| | | | | | | | chromosomes 14 and 20, respectively |
| 76_74 | 16 | PHKB * | missense | phosphorylase kinase, beta | | | Phosphorylase kinase is a polymer of 16 subunits, |
| | | | | | | | four each of alpha, beta, gamma and delta. The |
| | | | | | | | alpha subunit includes the skeletal muscle and |
| | | | | | | | hepatic isoforms, encoded by two different genes. |
| | | | | | | | The beta subunit is the same in both the muscle and |
| | | | | | | | hepatic isoforms, encoded by this gene, which is a |
| | | | | | | | member of the phosphorylase b kinase regulatory |
| | | | | | | | subunit family. The gamma subunit also includes |
| | | | | | | | the skeletal muscle and hepatic isoforms, encoded |
| | | | | | | | by two different genes. The delta subunit is a |
| | | | | | | | calmodulin and can be encoded by three different |
| | | | | | | | genes. The gamma subunits contain the active site |
| | | | | | | | of the enzyme, whereas the alpha and beta subunits |
| | | | | | | | have regulatory functions controlled by |
| | | | | | | | phosphorylation. The delta subunit mediates the |
| | | | | | | | dependence of the enzyme on calcium |
| | | | | | | | concentration. Mutations in this gene cause |
| | | | | | | | glycogen storage disease type 9B, also known as |
| | | | | | | | phosphorylase kinase deficiency of liver and |
| | | | | | | | muscle. Alternatively spliced transcript variants |
| | | | | | | | encoding different isoforms have been identified in |
| | | | | | | | this gene. Two pseudogenes have been found on |
| | | | | | | | chromosomes 14 and 20, respectively |
| 76_74 | 16 | PHKB(3′) * | | phosphorylase kinase, beta | | | Phosphorylase kinase is a polymer of 16 subunits, |
| | | | | | | | four each of alpha, beta, gamma and delta. The |
| | | | | | | | alpha subunit includes the skeletal muscle and |
| | | | | | | | hepatic isoforms, encoded by two different genes. |
| | | | | | | | The beta subunit is the same in both the muscle and |
| | | | | | | | hepatic isoforms, encoded by this gene, which is a |
| | | | | | | | member of the phosphorylase b kinase regulatory |
| | | | | | | | subunit family. The gamma subunit also includes |
| | | | | | | | the skeletal muscle and hepatic isoforms, encoded |
| | | | | | | | by two different genes. The delta subunit is a |
| | | | | | | | calmodulin and can be encoded by three different |
| | | | | | | | genes. The gamma subunits contain the active site |
| | | | | | | | of the enzyme, whereas the alpha and beta subunits |
| | | | | | | | have regulatory functions controlled by |
| | | | | | | | phosphorylation. The delta subunit mediates the |
| | | | | | | | dependence of the enzyme on calcium |
| | | | | | | | concentration. Mutations in this gene cause |
| | | | | | | | glycogen storage disease type 9B, also known as |
| | | | | | | | phosphorylase kinase deficiency of liver and |
| | | | | | | | muscle. Alternatively spliced transcript variants |
| | | | | | | | encoding different isoforms have been identified in |
| | | | | | | | this gene. Two pseudogenes have been found on |
| | | | | | | | chromosomes 14 and 20, respectively |
| 76_74 | 4 | C4orf37 | | sperm-tail PG-rich repeat containing 2 | | | sperm-tail PG-rich repeat |
| 76_74 | 4 | C4orf37(3′) | | sperm-tail PG-rich repeat containing 2 | | | sperm-tail PG-rich repeat |
| 76_74 | 4 | RP11-431J17.1(3′) | | Novel long non coding RNA | | | Homo sapiens BAC clone RP11-431J17 from 4, |
| | | | | | | | complete sequence |
| 76_74 | 4 | SOD3(5′) * | | superoxide dismutase (SOD) protein | | | This gene encodes a member of the superoxide |
| | | | | | | | dismutase (SOD) protein family. SODs are |
| | | | | | | | antioxidant enzymes that catalyze the dismutation |
| | | | | | | | of two superoxide radicals into hydrogen peroxide |
| | | | | | | | and oxygen. The product of this gene is thought to |
| | | | | | | | protect the brain, lungs, and other tissues from |
| | | | | | | | oxidative stress. The protein is secreted into the |
| | | | | | | | extracellular space and forms a glycosylated |
| | | | | | | | homotetramer that is anchored to the extracellular |
| | | | | | | | matrix (ECM) and cell surfaces through an |
| | | | | | | | interaction with heparan sulfate proteoglycan and |
| | | | | | | | collagen. A fraction of the protein is cleaved near |
| | | | | | | | the C-terminus before secretion to generate |
| | | | | | | | circulating tetramers that do not interact with the |
| | | | | | | | ECM. [provided by RefSeq, July 2008] |
| 76_74 | 5 | CTD-2292M14.1(3′) * | | non coding long RNA novel transcript | | | Genomic clone: CTD Coats disease |
| 76_74 | 8 | RP11-1D12.2(5′) * | | Novel long non coding RNA | | | |
| 76_74 | 8 | RP11-770E5.1 | | Novel antisense gene transcript | | | |
| 77_5 | 8 | CSMD1 | intronic | potential tumor suppressor | Yes | deletion related to head | CUB and Sushi multiple domains 1 |
| | | | | | | and neck carcinomas | |
| 81_13 | 16 | GP2 * | intronic | glycoprotein 2 | Yes | | glycoprotein 2 (zymogen granule membrane) |
| 81_13 | 16 | GP2 * | synonymous | glycoprotein 2 | Yes | | glycoprotein 2 (zymogen granule membrane) |
| 81_13 | 16 | GP2(3′) * | | glycoprotein 2 | Yes | | glycoprotein 2 (zymogen granule membrane) |
| 81_13 | 8 | RP11-401H2.1(5′) * | | exon transcript. | | | |
| | | | | Codes an unknown protein | | | |
| 81_13 | 8 | SNTG1 | intronic | Syntrophins; mediates dystrophin binding. | | | The protein encoded by this gene is a member of |
| | | | | Specifically expressed in the brain | | | the syntrophin family. Syntrophins are cytoplasmic |
| | | | | | | | peripheral membrane proteins that typically contain |
| | | | | | | | 2 pleckstrin homology (PH) domains, a PDZ |
| | | | | | | | domain that bisects the first PH domain, and a C- |
| | | | | | | | terminal domain that mediates dystrophin binding. |
| | | | | | | | This gene is specifically expressed in the brain. |
| | | | | | | | Transcript variants for this gene have been |
| | | | | | | | described, but their full-length nature has not been |
| | | | | | | | determined. [provided by RefSeq, July 2008] |
| 81_13 | 8 | SNTG1(3′) | | Syntrophins; mediates dystrophin binding. | | | The protein encoded by this gene is a member of |
| | | | | Specifically expressed in the brain | | | the syntrophin family. Syntrophins are cytoplasmic |
| | | | | | | | peripheral membrane proteins that typically contain |
| | | | | | | | 2 pleckstrin homology (PH) domains, a PDZ |
| | | | | | | | domain that bisects the first PH domain, and a C- |
| | | | | | | | terminal domain that mediates dystrophin binding. |
| | | | | | | | This gene is specifically expressed in the brain. |
| | | | | | | | Transcript variants for this gene have been |
| | | | | | | | described, but their full-length nature has not been |
| | | | | | | | determined. [provided by RefSeq, July 2008] |
| 81_3 | 2 | AC068490.2 | | transcript without known gene product | | | |
| 81_73 | 11 | TMEM135 | intronic | transmembrane protein | | Cerebrovascular disease, | transmembrane protein 135 |
| | | | | | | metabolic syndrome | |
| 81_73 | 11 | TMEM135(3′) | | transmembrane protein | | Cerebrovascular disease, | transmembrane protein 136 |
| | | | | | | metabolic syndrome | |
| 81_73 | 15 | RYR3 | intronic | ryanodine receptor, | | Cerebrovascular disease, | The protein encoded by this gene is a ryanodine |
| | | | | | | metabolic syndrome | receptor, which functions to release calcium from |
| | | | | | | | intracellular storage for use in many cellular |
| | | | | | | | processes. For example, the encoded protein is |
| | | | | | | | involved in skeletal muscle contraction by releasing |
| | | | | | | | calcium from the sarcoplasmic reticulum followed |
| | | | | | | | by depolarization of T-tubules. Two transcript |
| | | | | | | | variants encoding different isoforms have been |
| | | | | | | | found for this gene |
| 81_73 | 18 | CHST9 | intronic | carbohydrate (N-acetylgalactosamine | | cell-cell interaction, signal | The protein encoded by this gene belongs to the |
| | | | | 4-0) sulfotransferase 9 | | transduction, and embryonic | sulfotransferase 2 family. It is localized to the golgi |
| | | | | | | development, expressed in | membrane, and catalyzes the transfer of sulfate to |
| | | | | | | pituitary | position 4 of non-reducing N-acetylgalactosamine |
| | | | | | | | (GalNAc) residues in both N-glycans and O- |
| | | | | | | | glycans. Sulfate groups on carbohydrates confer |
| | | | | | | | highly specific functions to glycoproteins, |
| | | | | | | | glycolipids, and proteoglycans, and are critical for |
| | | | | | | | cell-cell interaction, signal transduction, and |
| | | | | | | | embryonic development. Alternatively spliced |
| | | | | | | | transcript variants have been described for this |
| | | | | | | | gene. |
| 83_41 | 13 | ATP8A2 | intronic | ATPase, aminophospholipid transporter | Yes | | ATPase, aminophospholipid transporter, class I, |
| | | | | | | | type 8A, member 2 |
| 85_23 | 18 | CHST9 | intronic | carbohydrate (N-acetylgalactosamine | | cell-cell interaction, signal | The protein encoded by this gene belongs to the |
| | | | | 4-0) sulfotransferase 9 | | transduction, and embryonic | sulfotransferase 2 family. It is localized to the golgi |
| | | | | | | development, expressed in | membrane, and catalyzes the transfer of sulfate to |
| | | | | | | pituitary | position 4 of non-reducing N-acetylgalactosamine |
| | | | | | | | (GalNAc) residues in both N-glycans and O- |
| | | | | | | | glycans. Sulfate groups on carbohydrates confer |
| | | | | | | | highly specific functions to glycoproteins, |
| | | | | | | | glycolipids, and proteoglycans, and are critical for |
| | | | | | | | cell-cell interaction, signal transduction, and |
| | | | | | | | embryonic development. Alternatively spliced |
| | | | | | | | transcript variants have been described for this |
| | | | | | | | gene. |
| 85_84 | 3 | RP11-735B13.1 | | processed transcript | | | Homo sapiens 3 BAC RP11-735B13 (Roswell Park |
| | | | | | | | Cancer Institute Human BAC Library) complete |
| | | | | | | | sequence. |
| 85_84 | 3 | RP11-735B13.1(5′) | | processed transcript | | | Homo sapiens 3 BAC RP11-735B13 (Roswell Park |
| | | | | | | | Cancer Institute Human BAC Library) complete |
| | | | | | | | sequence. |
| 85_84 | 3 | RP11-735B13.2(3′) | | processed transcript | | | |
| 87_26 | 13 | NALCN | intronic | NALCN forms a voltage-independent, | Yes | | NALCN forms a voltage-independent, nonselective, |
| | | | | nonselective, noninactivating cation | | | noninactivating cation channel permeable to Na+, |
| | | | | channel permeable to Na+, K+, | | | K+, and Ca(2+). It is responsible for the neuronal |
| | | | | and Ca(2+). It is responsible for | | | background sodium leak conductance |
| | | | | the neuronal background sodium leak | | | |
| | | | | conductance | | | |
| 87_26 | 13 | RP11-430M15.1 | | novel transcript, antisense to NALCN | Yes | | |
| 87_26 | 13 | RP11-430M15.1 | intronic | novel transcript, antisense to NALCN | Yes | | |
| 87_76 | 8 | TRPS1(3′) | | transcription factor that represses | | | This gene encodes a transcription factor that |
| | | | | GATA-regulated genes and binds to | | | represses GATA-regulated genes and binds to a |
| | | | | a dynein light chain protein | | | dynein light chain protein. Binding of the encoded |
| | | | | | | | protein to the dynein light chain protein affects |
| | | | | | | | binding to GATA consensus sequences and |
| | | | | | | | suppresses its transcriptional activity. Defects in |
| | | | | | | | this gene are a cause of tricho-rhino-phalangeal |
| | | | | | | | syndrome (TRPS) types I-III. [provided by RefSeq, |
| | | | | | | | July 2008 |
| 87_84 | 1 | AC093577.1 (5′) * | | Novel non-coding miRNA | | | genomic clone RELATED to FAM69 family of |
| | | | | | | | cysteine-rich type II transmembrane proteins. These |
| | | | | | | | proteins localize to the endoplasmic reticulum but |
| | | | | | | | their specific functions are unknown. Alternatively |
| | | | | | | | spliced transcript variants encoding multiple |
| | | | | | | | isoforms have been observed for this gene. |
| | | | | | | | [provided by RefSeq, November 2011] |
| 87_84 | 1 | FAM69A | 3′-UTR | cysteine-rich type II transmembrane | Yes | | This gene encodes a member of the FAM69 family |
| | | | | endoplasmic reticulum protein | | | of cysteine-rich type II transmembrane proteins. |
| | | | | | | | These proteins localize to the endoplasmic |
| | | | | | | | reticulum but their specific functions are unknown. |
| | | | | | | | Alternatively spliced transcript variants encoding |
| | | | | | | | multiple isoforms have been observed for this gene. |
| | | | | | | | [provided by RefSeq, November 2011] |
| 87_84 | 1 | FAM69A | intronic | cysteine-rich type II transmembrane | Yes | | This gene encodes a member of the FAM69 family |
| | | | | endoplasmic reticulum protein | | | of cysteine-rich type II transmembrane proteins. |
| | | | | | | | These proteins localize to the endoplasmic |
| | | | | | | | reticulum but their specific functions are unknown. |
| | | | | | | | Alternatively spliced transcript variants encoding |
| | | | | | | | multiple isoforms have been observed for this gene. |
| | | | | | | | [provided by RefSeq, November 2011] |
| 87_84 | 1 | FAM69A(5′) | | cysteine-rich type II transmembrane | Yes | | This gene encodes a member of the FAM69 family |
| | | | | endoplasmic reticulum protein | | | of cysteine-rich type II transmembrane proteins. |
| | | | | | | | These proteins localize to the endoplasmic |
| | | | | | | | reticulum but their specific functions are unknown. |
| | | | | | | | Alternatively spliced transcript variants encoding |
| | | | | | | | multiple isoforms have been observed for this gene. |
| | | | | | | | [provided by RefSeq, November 2011] |
| 87_84 | 1 | RPL5 | intronic | ribosomal protein, protein interacts | Yes | | Ribosomes, the organelles that catalyze protein |
| | | | | specifically with the beta subunit | | | synthesis, consist of a small 40S subunit and a large |
| | | | | of casein kinase II | | | 60S subunit. Together these subunits are composed |
| | | | | | | | of 4 RNA species and approximately 80 structurally |
| | | | | | | | distinct proteins. This gene encodes a ribosomal |
| | | | | | | | protein that is a component of the 60S subunit. The |
| | | | | | | | protein belongs to the L18P family of ribosomal |
| | | | | | | | proteins. It is located in the cytoplasm. The protein |
| | | | | | | | binds 5S rRNA to form a stable complex called the |
| | | | | | | | 5S ribonucleoprotein particle (RNP), which is |
| | | | | | | | necessary for the transport of nonribosome- |
| | | | | | | | associated cytoplasmic 5S rRNA to the nucleolus |
| | | | | | | | for assembly into ribosomes. The protein interacts |
| | | | | | | | specifically with the beta subunit of casein kinase |
| | | | | | | | II. Variable expression of this gene in colorectal |
| | | | | | | | cancers compared to adjacent normal tissues has |
| | | | | | | | been observed, although no correlation between the |
| | | | | | | | level of expression and the severity of the disease |
| | | | | | | | has been found. This gene is co-transcribed with the |
| | | | | | | | small nucleolar RNA gene U21, which is located in |
| | | | | | | | its fifth intron. As is typical for genes encoding |
| | | | | | | | ribosomal proteins, there are multiple processed |
| | | | | | | | pseudogenes of this gene dispersed through the |
| | | | | | | | genome. [provided by RefSeq, July 2008] |
| 87_84 | 1 | RPL5(5′) | | ribosomal protein, protein interacts | Yes | | Ribosomes, the organelles that catalyze protein |
| | | | | specifically with the beta subunit | | | synthesis, consist of a small 40S subunit and a large |
| | | | | of casein kinase II | | | 60S subunit. Together these subunits are composed |
| | | | | | | | of 4 RNA species and approximately 80 structurally |
| | | | | | | | distinct proteins. This gene encodes a ribosomal |
| | | | | | | | protein that is a component of the 60S subunit. The |
| | | | | | | | protein belongs to the L18P family of ribosomal |
| | | | | | | | proteins. It is located in the cytoplasm. The protein |
| | | | | | | | binds 5S rRNA to form a stable complex called the |
| | | | | | | | 5S ribonucleoprotein particle (RNP), which is |
| | | | | | | | necessary for the transport of nonribosome- |
| | | | | | | | associated cytoplasmic 5S rRNA to the nucleolus |
| | | | | | | | for assembly into ribosomes. The protein interacts |
| | | | | | | | specifically with the beta subunit of casein kinase |
| | | | | | | | II. Variable expression of this gene in colorectal |
| | | | | | | | cancers compared to adjacent normal tissues has |
| | | | | | | | been observed, although no correlation between the |
| | | | | | | | level of expression and the severity of the disease |
| | | | | | | | has been found. This gene is co-transcribed with the |
| | | | | | | | small nucleolar RNA gene U21, which is located in |
| | | | | | | | its fifth intron. As is typical for genes encoding |
| | | | | | | | ribosomal proteins, there are multiple processed |
| | | | | | | | pseudogenes of this gene dispersed through the |
| | | | | | | | genome. [provided by RefSeq, July 2008] |
| 87_84 | 1 | SNORA66.1 | intronic | small nucleolar RNA, H/ACA box 66; | | | This gene encodes a non-coding RNA that functions |
| | | | | regulation of gene expression | | | in the biogenesis of other small nuclear RNAs. This |
| | | | | | | | RNA is found in the nucleolus, where it may be |
| | | | | | | | involved in the pseudouridylation of 18S ribosomal |
| | | | | | | | RNA. This RNA is found associated with the |
| | | | | | | | GAR1 protein. [provided by RefSeq, April 2009] |
| 87_84 | 1 | U6.1236(5′) * | | U6 spliceosomal RNA | | | RNA, U6 small nuclear |
| 88_43 | 10 | RP11-428G2.1(5′) * | | Novel long non coding RNA | | | |
| 88_64 | 16 | GP2 * | intronic | glycoprotein 2 | Yes | | glycoprotein 2 (zymogen granule membrane) |
| 88_64 | 16 | GP2 * | synonymous | glycoprotein 2 | Yes | | glycoprotein 2 (zymogen granule membrane) |
| 88_64 | 16 | GP2(3′) * | | glycoprotein 2 | Yes | | glycoprotein 2 (zymogen granule membrane) |
| 88_8 | 1 | AC093577.1 (3′) | | Novel non-coding miRNA | | | genomic clone RELATED to FAM69 family of |
| | | | | | | | cysteine-rich type II transmembrane proteins. These |
| | | | | | | | proteins localize to the endoplasmic reticulum but |
| | | | | | | | their specific functions are unknown. Alternatively |
| | | | | | | | spliced transcript variants encoding multiple |
| | | | | | | | isoforms have been observed for this gene. |
| | | | | | | | [provided by RefSeq, November 2011] |
| 88_8 | 1 | AC093577.1 (5′) | | Novel non-coding miRNA | | | genomic clone RELATED to FAM69 family of |
| | | | | | | | cysteine-rich type II transmembrane proteins. These |
| | | | | | | | proteins localize to the endoplasmic reticulum but |
| | | | | | | | their specific functions are unknown. Alternatively |
| | | | | | | | spliced transcript variants encoding multiple |
| | | | | | | | isoforms have been observed for this gene. |
| | | | | | | | [provided by RefSeq, November 2011] |
| 88_8 | 1 | EVI5 | intronic | ecotropic viral integration site 5 | | Cerebrovascular disease, | ecotropic viral integration site 5 |
| | | | | | | metabolic syndrome | |
| 88_8 | 1 | U6.1077(5′) | | U6 spliceosomal RNA | | | RNA, U6 small nuclear |
| 88_8 | 6 | HACE1(3′) * | | ubiquitin protein ligase 1 | Yes | | HECT domain and ankyrin repeat containing E3 |
| | | | | | | | ubiquitin protein ligase 1 |
| 90_78 | 1 | AC093577.1 (3′) | | Novel non-coding miRNA | | | genomic clone RELATED to FAM69 family of |
| | | | | | | | cysteine-rich type II transmembrane proteins. These |
| | | | | | | | proteins localize to the endoplasmic reticulum but |
| | | | | | | | their specific functions are unknown. Alternatively |
| | | | | | | | spliced transcript variants encoding multiple |
| | | | | | | | isoforms have been observed for this gene. |
| | | | | | | | [provided by RefSeq, November 2011] |
| 90_78 | 1 | AC093577.1 (5′) | | Novel non-coding miRNA | | | genomic clone RELATED to FAM69 family of |
| | | | | | | | cysteine-rich type II transmembrane proteins. These |
| | | | | | | | proteins localize to the endoplasmic reticulum but |
| | | | | | | | their specific functions are unknown. Alternatively |
| | | | | | | | spliced transcript variants encoding multiple |
| | | | | | | | isoforms have been observed for this gene. |
| | | | | | | | [provided by RefSeq, November 2011] |
| 90_78 | 1 | EVI5 | intronic | ecotropic viral integration site 5 | | Cerebrovascular disease, | ecotropic viral integration site 5 |
| | | | | | | metabolic syndrome | |
| 90_78 | 1 | U6.1077(5′) | | U6 spliceosomal RNA | | | RNA, U6 small nuclear |

For example, as disclosed in Table 2, where a SNP set 9_9 is disclosed, specifically contemplated herein is that SNP sets detects polymorphisms in NTRK3 and SEMA3A; where a SNP set 10_4 is disclosed, specifically contemplated herein is that SNP sets detects polymorphisms in C14orf102, C14orf102(5′), PSMC1, PSMC1(3′), and PSMC1(5′); where a SNP set 12_11 is disclosed, specifically contemplated herein is that SNP sets detects polymorphisms in C14orf102, C14orf102(5′), PSMC1, PSMC1(3′), and PSMC1(5′); a SNP set 12_2 is disclosed, specifically contemplated herein is that SNP sets detects polymorphisms in an intronic region and 3′ UTR of HPGDS, HPGDS(5′), an intronic region, missense, and 3′ UTR of SMARCAD1 and RP11-363G15.2; where a SNP set 13_12 is disclosed, specifically contemplated herein is that SNP sets detects polymorphisms in EML5, SPATA7, U4.15(3′), U4.15(5′), and ZC3H14; where a SNP set 14_6 is disclosed, specifically contemplated herein is that SNP sets detects polymorphisms in NTRK3; a SNP set 16_10 is disclosed, specifically contemplated herein is that SNP sets detects polymorphisms in, intronic region and 3′ UTR of HPGDS, HPGDS(5′), RP11-363G15.2 and an intronic region, missense, and 3′ UTR of SMARCAD1; a SNP set 19_2 is disclosed, specifically contemplated herein is that SNP sets detects polymorphisms in ARPC5L, an intronic region, missense, and 3′ UTR of GOLGA1, RPL35, WDR38, and SCA1; where a SNP set 21_8 is disclosed, specifically contemplated herein is that SNP sets detects polymorphisms in AC068490.2; where a SNP set 22_11 is disclosed, specifically contemplated herein is that SNP sets detects polymorphisms in AC068490.2; where a SNP set 25_10 is disclosed, specifically contemplated herein is that SNP sets detects polymorphisms in AL158819.7(3′), FOXR2, FOXR2(3′), MAGEH1(5′), PAGE3, PAGE3(3′), PAGE3(5′), RP11-382F24.2, RP11-382F24.2(3′), RP11-382F24.2(5′), RP13-188A5.1, RRAGB, RRAGB(3′), RRAGB(5′), and SNORD112.49(3′); a SNP set 31_2 is disclosed, specifically contemplated herein is that SNP sets detects polymorphisms in intronic region, and 3′ UTR C6orf138, C6orf138(3′), and OPN5(3′); where a SNP set 41_12 is disclosed, specifically contemplated herein is that SNP sets detects polymorphisms in GPR119(3′), SLC25A14 and SLC25A14(3′); where a SNP set 42_37 is disclosed, specifically contemplated herein is that SNP sets detects polymorphisms in NCAM1, RP11-629G13.1, RP11-629G13.1(3′), AC064837.1, and PPP1R1C; where a SNP set 51_28 is disclosed, specifically contemplated herein is that SNP sets detects polymorphisms in IGSF1; a SNP set 52_42 is disclosed, specifically contemplated herein is that SNP sets detects polymorphisms in NCAM1, RP11-629G13.1, and RP11-629G13.1(3′); where a SNP set 54_51 is disclosed, specifically contemplated herein is that SNP sets detects polymorphisms in CSMD1; where a SNP set 56_19 is disclosed, specifically contemplated herein is that SNP sets detects polymorphisms in SNX19(5′); where a SNP set 56_30 is disclosed, specifically contemplated herein is that SNP sets detects polymorphisms in 7SK.207(3′), 7SK.207(5′), PTBP2, PTBP2(5′), RP4-726F1.1(3′), GP2, GP2(3′); where a SNP set 58_29 is disclosed, specifically contemplated herein is that SNP sets detects polymorphisms in CTD-3025N20.2(3) and RP11-1D12.2(5′); where a SNP set 59_48 is disclosed, specifically contemplated herein is that SNP sets detects polymorphisms in RP11-128M1.1, RP11-128M1.1(3′) and TRPS1(3′); where a SNP set 61_39 is disclosed, specifically contemplated herein is that SNP sets detects polymorphisms in IGSF1; where a SNP set 65_25 is disclosed, specifically contemplated herein is that SNP sets detects polymorphisms in C20orf78(5′); where a SNP set 71_55 is disclosed, specifically contemplated herein is that SNP sets detects polymorphisms in NTRK3(3′); where a SNP set 75_31 is disclosed, specifically contemplated herein is that SNP sets detects polymorphisms in AC093577.1(3′), AC093577.1(5′), U6.1077(5′), and SNX19(5′); where a SNP set 75_67 is disclosed, specifically contemplated herein is that SNP sets detects polymorphisms in SNORA42.4(5′), VANGL1(5′), RP11-298H24.1(3′), STYK1, AL 161669.1(3′), AL161669.1(5′), AL161669.2, AL161669.2(3′), 5S_rRNA.496(3′), NTRK3(3′), 7SK.236(5′), GP2, GP2(3′), CTA-71487.5, RP11-436A20.3, C4orf37, C4orf37(3′), RP11-431J17.1(3′), 7SK.7(3′), DKK4(5′), DUSP4(5′), GSR, RP11-401H2.1 (5′), RP11-486M23.1(5′), RP11-738G5.1(3′), RP11-770E5.1, SLC20A2, SNTG1, SNTGT1(3′), ST18, and VDAC3; where a SNP set 76_63 is disclosed, specifically contemplated herein is that SNP sets detects polymorphisms in IGSF1; where a SNP set 76_74 is disclosed, specifically contemplated herein is that SNP sets detects polymorphisms in AL161669.1(3′), AL161669.1(5′), AL161669.2, AL161669.2(3′), ABCC12(3′), ITFG1, NETO2, PHKB, PHKB(3′), C4orf37, C4orf37(3′), RP11-431J17.1(3′), SOD3(5′), CTD-2292M14.1(3′), RP11-1D12.2(5′), and RP11-770E5.1; where a SNP set 77_5 is disclosed, specifically contemplated herein is that SNP sets detects polymorphisms in CSMD1; a SNP set 81_13 is disclosed, specifically contemplated herein is that SNP sets detects polymorphisms in GP2, GP2(3′), RP11-401H2.1(5′), SNTG1, and SNTG1(3′); where a SNP set 81_3 is disclosed, specifically contemplated herein is that SNP sets detects polymorphisms in AC068490.2; where a SNP set 81_73 is disclosed, specifically contemplated herein is that SNP sets detects polymorphisms in TMEM135, TMEM135(3′), RYR3, and CHST9; where a SNP set 83_41 is disclosed, specifically contemplated herein is that SNP sets detects polymorphisms in ATP8A2; where a SNP set 85_84 is disclosed, specifically contemplated herein is that SNP sets detects polymorphisms in RP11-735B13.1, RP11-735B13.1(5′), and RP11-735B13.2(3′); where a SNP set 85_23 is disclosed, specifically contemplated herein is that SNP sets detects polymorphisms in CHST9; a SNP set 87_26 is disclosed, specifically contemplated herein is that SNP sets detects polymorphisms in NALCN and RP11-430M15.1; where a SNP set 87_76 is disclosed, specifically contemplated herein is that SNP sets detects polymorphisms in TRPS1(3′); where a SNP set 87_84 is disclosed, specifically contemplated herein is that SNP sets detects polymorphisms in AC093577.1(5′), FAM69A, FAM69A(5′), RPL5, RPL5(5′), SNORA66.1, and U6.1236(5′); where a SNP set 88_43 is disclosed, specifically contemplated herein is that SNP sets detects polymorphisms in RP11-428G2.1(5′); where a SNP set 88_64 is disclosed, specifically contemplated herein is that SNP sets detects polymorphisms in GP2 and GP2(3′); where a SNP set 88_8 is disclosed, specifically contemplated herein is that SNP sets detects polymorphisms in AC093577.1(3′), AC093577.1(5′), EVI5, U6.1077(5′), and HACE1(3′); and where a SNP set 90_78 is disclosed, specifically contemplated herein is that SNP sets detects polymorphisms in AC093577.1(3′), AC093577.1(5′), EVI5, and U6.1077(5′).

It is contemplated herein that the disclosed expression panel can comprise a single expression set (such as, for example, the SNP sets disclosed herein 19_2, 88_64, 81_13, 87_76, 58_29, 83_41, 9_9, 10_4, 14_6, 56_30, 42_37, 65_25, 71_55, 12_11, 90_78, 77_5, 88_8, 51_28, 59_48, 41_12, 22_11, 13_12, 31_22, 85_84, 87_84, 16_10, 56_19, 75_31, 81_73, 85_23, 21_8, 76_74, 61_39, 75_67, 76_63, 81_3, 87_26, 88_43, 25_10, 12_2, 52_42, or 54_51). It is further contemplated herein that the disclosed expression panels can comprise any combination of 2, 3, 4, 5, 6, 7, 8, 910, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 or more of the disclosed SNP sets. For example, the expression panel can comprise one or more SNP sets are selected from the group comprising 88_8, 90_78, 65_25, 42_37, 71_55, 56_30, 77_5, 12_11, 51_28, 59_48, 10_4, 83_41, 58_29, 9_9, 14_6, 87_76, 88_64, or 81_13. Also, the expression panel can comprise one or more SNP sets are selected from the group comprising 10_4, 83_41, 58_29, 9_9, 14_6, 87_76, 88_64, or 81_13. Also, the expression panel can comprise one or more SNP sets are selected from the group comprising 87_76, 88_64, or 81_13.

As disclosed herein, through analysis of the complex genotypic and phenotypic relationships certain groupings of SNP sets and clinical/phenotypic features were elucidated. The composition of these designated sets is presented in Table 7. These SNP sets are associated with specific subtypes of the schizophrenias, which are characterized here simultaneously by both their genetic features (snp sets) and their clinical features (phenotypic sets) and are grouped into 8 subtypes (see, Table 7).

TABLE 7
Subset of Genotypic-Phenotypic AND/OR Relationships (Hypergeometric statistics)

| | | | |
|---|---|---|---|
| | Phenotypic | SNP | |
| Schizophrenia Class, Symptoms^{b}, and DSM Ratings | sets | sets | p-value |

Severe process, with positive and negative symptom schizophrenia (I)

| | | | |
|---|---|---|---|
| Positive symptoms; moderate severity of impairment; unable to function since onset | 15_13 | 56_30 | 2.55E−05 |
| Auditory hallucinations (2 or more voices; running commentaries) | 12_11 | | 1.79E−04 |
| Auditory hallucinations (2 or more voices; running commentaries); thought echoing; | 21_1 | | 3.66E−04 |
| withdrawal; insertion and broadcasting; delusions of mind reading | | | |
| Hallucinations (any); auditory hallucinations (ever; 2 or more voices); grossly disorganized | 50_46 | | 5.70E−04 |
| behavior | | | |
| Hallucinations (mood incongruent); auditory hallucinations; somatic hallucinations | 9_6 | | 4.45E−03 |
| (olfactory; gustatory; tactile); religious delusions; delusions of mind reading; | | | |
| delusions of control; thought echoing; withdrawal; insertion and broadcasting | | | |
| Hallucinations (mood incongruent); persecutory delusions; delusions of reference; jealousy | 46_23 | | 4.15E−03 |
| delusions; bizarre delusions; disorganized odd behavior; disorganized odd speech; | | | |
| delusions, fragmented (unrelated themes); delusions, widespread (intrude into most | | | |
| aspects of life); thought insertion; flat affect; avolition and apathy | | | |
| Continuously positive symptoms; severe impairment; continuous course; no affective | 15_13 | 75_67 | 2.31E−13 |
| symptoms | | | |
| Grossly disorganized behavior; severe impairment; continuous course | 54_11 | | 4.90E−06 |
| Delusions of persecution and reference; disorganized speech; severe impairment; unable to | 30_17 | | 2.56E−04 |
| function since onset | | | |
| Auditory hallucinations (ever; 2 or more voices; running commentaries); jealousy delusions | 18_13 | | 3.50E−04 |
| Thought insertion and withdrawal | 27_6 | | 3.62E−03 |
| Hallucinations (any); auditory hallucinations (2 or more voices); grossly disorganized | 50_46 | | 3.61E−03 |
| behavior | | | |
| Delusions, persecutory and reference; delusions widespread (intrude into most aspects of | 61_18 | | 4.28E−03 |
| life); | | | |
| Disorganized; odd speech | 64_11 | | 1.45E−03 |
| Delusions widespread (intrude into most aspects of patient's life); continuous course | 65_64 | | 1.21E−03 |
| Continuously positive symptoms; severe impairment; unable to function since onset; no | 15_13 | 76_74 | 1.07E−07 |
| affective symptoms | | | |
| Delusions widespread (intrude into most aspects of life) | 65_64 | | 1.47E−03 |

Positive and negative schizophrenia (II)

| | | | |
|---|---|---|---|
| Auditory hallucinations; delusions (any); bizarre delusions; disorganized speech and | 12_4 | 59_48 | 1.88E−04 |
| behavior; flat affect; alogia; avolition | | | |
| Auditory hallucinations (2 or more voices; running commentaries); | 42_9 | 71_55 | 1.98E−03 |

Negative schizophrenia (III)

| | | | |
|---|---|---|---|
| Thought insertion and withdrawal | 52_28 | 58_29 | 1.44E−04 |
| Disorganized speech; odd speech | 7_3 | 9_9 | 1.97E−04 |
| Flat affect; persecutory delusions | 48_41 | | 2.23E−03 |
| Delusions of mind reading; guilt delusions; sin delusions; jealousy delusions | 26_8 | | 4.20E−03 |
| Flat affect; apathy; avolition | 69_41 | 22_11 | 5.52E−05 |
| Flat affect; apathy; avolition; alogia; Continuous mixture of positive and negative | 10_5 | | 4.62E−04 |
| symptoms | | | |
| Disorganized and odd speech | 17_2 | | 1.01E−04 |

Positive schizophrenia (IV)

| | | | |
|---|---|---|---|
| Hallucinations (any); auditory hallucinations (ever; 2 or more voices); no affective | 63_24 | 88_64 | 3.45E−04 |
| symptoms | | | |
| Delusions of jealousy; auditory hallucinations (running commentaries) | 69_66 | | 4.49E−03 |

Severe process, positive schizophrenia (V)

| | | | |
|---|---|---|---|
| Continuously positive symptoms; severe impairment; unable to function since onset; | 22_13 | 77_5 | 5.66E−05 |
| no affective symptoms | | | |
| Auditory hallucinations (2+ voices; running commentaries) | 8_13 | | 3.25E−03 |
| Hallucinations (any); auditory hallucinations (2 or more voices; running | 53_6 | | 4.76E−03 |
| commentaries); continuous course | | | |
| Auditory hallucinations (ever; voices; noises; music) | 59_41 | | 1.22E−03 |
| Continuously positive symptoms; severe impairment; unable to function since onset; | 20_19 | 81_13 | 2.83E−04 |
| no affective symptoms | | | |
| Hallucinations (any); auditory hallucinations (ever; 2+ voices); bizarre delusions; | 55_7 | | 8.57E−04 |
| delusions fragmented (unrelated themes); delusions widespread (intrude into | | | |
| most aspects of life) | | | |
| Delusions of reference; Delusions of persecution | 34_17 | | 2.40E−03 |
| Auditory hallucinations (running commentaries); jealousy delusions | 69_66 | | 1.30E−03 |
| Severe impairment; unable to function since onset; no affective symptoms | 27_7 | 25_10 | 4.76E−06 |
| Auditory hallucinations (2 or more voices; running commentaries) | 18_13 | | 9.50E−05 |
| Auditory hallucinations (ever; voices; noises; music); auditory hallucinations (2+ | 4_1 | | 2.49E−03 |
| voices; running commentaries); Thought echoing | | | |
| Delusions of reference; delusions of persecution | 66_54 | | 2.10E−03 |
| Bizarre delusions; delusions of mind reading; delusions widespread (intrude into most | 8_4 | | 1.93E−03 |
| aspects of life) | | | |

Moderate process, disorganized negative (VI)

| | | | |
|---|---|---|---|
| Grossly disorganized or catatonic behavior; disorganized speech | 51_38 | 19_2 | 4.03E−04 |
| Moderate deterioration; unable to function since onset; no affective symptoms | 42_7 | 14_6 | 4.96E−04 |
| Grossly disorganized and inappropriate behavior | 18_3 | | 2.55E−03 |
| Auditory hallucinations (running commentaries); thought echoing | 46_29 | | 3.78E−03 |

Moderate process, positive and negative schizophrenia (VII)

| | | | |
|---|---|---|---|
| Hallucinations (any); auditory hallucinations (ever; voices; noises; music); continuous | 5_2 | 42_37 | 1.32E−04 |
| mixture positive and negative symptoms; continuous course; moderate | | | |
| impairment; unable to function since onset; no affective symptoms | | | |
| Bizarre delusions; delusions of reference | 57_39 | | 4.70E−03 |
| Continuous mixture positive and negative symptoms; continuous course; moderate | 11_5 | 88_43 | 6.88E−04 |
| impairment; unable to function since onset; no affective symptoms | | | |
| Auditory hallucinations (ever); bizarre delusions; delusions fragmented (unrelated to | 24_4 | 51_28 | 9.58E−04 |
| theme) | | | |

Moderate process, continuous positive schizophrenia (VIII)

| | | | |
|---|---|---|---|
| No affective symptoms | 48_7 | 16_10 | 1.44E−03 |
| Continuously positive symptoms; severe impairment; unable to function since onset; no | 28_23 | 83_41 | 3.48E−03 |
| affective symptoms | | | |
| Continuously positive symptoms; no affective symptoms | 25_20 | 87_26 | 4.22E−03 |

^{b}Symptoms were assessed with Diagnostic Interview for Genetic Studies.

Because of these associations it is possible to create panels to assess the risk of a subject to have a particular classification of schizophrenia. These classification specific expression panels can be used individually in the diagnostic system disclosed herein or as one of several classification specific panels in a diagnostic system. For example, in one aspect, disclosed herein are diagnostic systems, wherein the system selects for severe process, with positive and negative symptom schizophrenia (I), and wherein the one or more SNP sets comprise 56_30, 75_67, or 76_74. Also disclosed are diagnostic systems, wherein the system selects for positive and negative Schizophrenia (II), and wherein the one or more SNP sets comprise 59_48, 71_55, 21_8, 54_51, 31_22, 65_25, or 87_84. Also disclosed are diagnostic systems, wherein the system selects for negative Schizophrenia (III), and wherein the one or more SNP sets comprise 58_29, 9_9, 22_11, 81_3, 13_12, 61_39, 10_4, 81_73, 75_31, 56_19, 88_8, or 12_2. Also disclosed are diagnostic systems, wherein the system selects for Positive Schizophrenia (IV), and wherein the one or more SNP sets comprise 88_64, 85_84, or 41_12. Also disclosed are diagnostic systems, wherein the system selects for severe process, positive schizophrenia (V), and wherein the one or more SNP sets comprise 77_5, 81_13, or 25_10. Also disclosed are diagnostic systems, wherein the system selects for moderate process, disorganized negative schizophrenia (VI), and wherein the one or more SNP sets comprise 19_2, 52_42, 90_78, 12_11, 87_76, and 14_6. Also disclosed are diagnostic systems, wherein the system selects for moderate process, positive and negative schizophrenia (VII), and wherein the one or more SNP sets comprise 42_37, 88_43, or 51_28. Also disclosed are diagnostic systems, wherein the system selects for moderate process, continuous positive schizophrenia (VIII), and wherein the one or more SNP sets comprise 16_10, 83_41, or 87_26.

As noted above, the disclosed classification specific expression panels can be used alone or in combination of 2 or more with any other classification specific expression panel. In a non-limiting example, the diagnostic system can comprise classification specific expression panels I; II; III; IV; V; VI; VII; VIII; I and II; I and III; I and IV; I and V; I and VI; I and VII; I and VIII; II and III; II and IV; II and V; II and VI; II and VII; II and VIII; III and IV; III and V; III and VI; III and VII; III and VIII; IV and V; IV and VI; IV and VII; IV and VIII; V and VI; V and VII, V and VIII; VI and VII; VI and VIII; VII and VIII; I, II, and III; III and IV; I, II, and V; I, II, and VI; I, II, and VII, I, II, and VIII; I, III, and IV; I, III, and V; I, III, and VI; I, III, and VII; I, III, and VIII; I, IV, and V; I, IV, and VI; I, IV, and VII; I, IV, and VIII; I, V, and VI; I, V, and VII, I, V, and VIII; I, VI, and VII, I, VI, and VIII; I, VII and VIII; I, II, III, and IV; I, II, III, and V; I, II, III, and VI, I, II, III, and VII; I, II, III, and VIII; I, II, IV, and V; I, II, IV, and VI; I, II, IV; and VI; I, II, IV, and VII; I, II, IV, and VIII; I, II, V, and VI; I, II, V, and VII; I, II, V, and VIII; I, II, VI, and VII; I, II, VI, and VIII; I, II, VII, and VIII; I, III, IV, and V; I, III, IV, and VI; I, III, IV, and VII; I, III, IV, and VIII; I, III, V, and VI; I, III, V, and VII; I, III, V, and VIII; I, IV, V, and VI; I, IV, V, and VII; I, IV, V, and VIII; I, V, VI, and VII; I, V, VI, and VIII; I, VI, VII, and VIII; I, II, III, IV, and V; I, II, III, IV, and VI; I, II, III, IV, and VII; I, II, III, IV, and VIII; I, III, IV, V, and VI; I, III, IV, V, and VII; I, III, IV, V, and VIII; I, II, IV, V, and VI; I, II, IV, V, and VII; I, II, IV, V, and VIII; I, II, III, V, and VI; I, II, III, V, and VII; I, II, III, V, and VIII; I, II, III, VI, and VII; I, II, III, VI, and VIII; I, II, III, VII, and VIII; I, II, III, IV, V, and VI; I, II, III, IV, V, and VII; I, I, II, III, IV, V, and VIII; I, I, II, III, IV, VI, and VII; I, II, III, IV, VI, and VIII; I, II, III, IV, VII, and VIII; I, II, III, IV, V, VI, and VII; I, II, III, IV, V, VI, and VIII; I, II, III, IV, V, VI, VII, and VIII; II, III, and IV; II, III, and V; II, III, and VI; II, III, and VII, II, III, and VIII; II, IV, and V; II, IV, and VI; II, IV, and VII; II, IV, and VIII; II, V, and VI; II, V, and VII; II, V, and VIII; II, VI, and VII, II, VI, and VIII; II, VII and VIII; II, III, IV, and V; II, III, IV, and VI; I, II, III, IV; and VI; II, III, IV, and VII; II, III, IV, and VIII; II, IV, V, and VI; II, IV, V, and VII; II, IV, V, and VIII; II, IV, VI, and VII; II, IV, VI, and VIII; II, IV, VII, and VIII; II, III, V, and V; I, II, III, V, and VI; II, III, V, and VII; and II, III, V, and VIII.

In one aspect, it is understood and herein contemplated that expression panels can be complemented in the claimed diagnostic system with phenotypic panels which provide the results of clinical assessment, hereditary surveys, environmental surveys (which look at oxidative stress during development or delivery (such as maternal pre-eclampsia or delivery with low Apgar score), urban versus rural living conditions—urban life increases risk, use of recreational drugs like marijuana or PCP during adolescence, social isolation, childhood abuse or neglect, and reduction in sensory input such as hearing or visual loss), online surveys, and interviews creating phenotypic sets Accordingly, in one aspect, disclosed herein are diagnostic systems for diagnosing schizophrenia further comprising one or more phenotype panels, wherein each phenotype panel comprises one or more phenotypic sets such as those listed in Table 8. Thus, in one aspect, disclosed herein are diagnostic systems for diagnosing schizophrenia further comprising one or more phenotype panels, wherein each phenotype panel comprises one or more phenotypic sets selected from the group comprising 15_13, 12_11, 21_1, 50_46, 9_6, 46_23, 54_11, 30_17, 18_13, 27_6, 61_18, 64_11, 65_64, 12_4, 42_9, 52_28, 7_3, 48_41, 26_8, 69_41, 10_5, 17_2, 63_24, 69_66, 22_13, 53_6, 59_41, 20_19, 55_7, 34_17, 4_1, 66_54, 8_4, 51_38, 42_7, 18_3, 46_29, 5_2, 57_39, 11_5, 24_4, 48_7, 28_23, and/or 25_20. It is understood and herein contemplated that the disclosed phenotypic panels can comprise any of the phenotypic sets individually or in any combination of 2, 3, 4, 5, 6, 7, 8, 910, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 or more of the disclosed phenotype sets.

As noted in Table 7, the phenotypic sets disclosed herein have been associated with one or more symptoms of one or more schizophrenia classes. Thus, contemplated herein are classification specific phenotype panels that can be used individually in the diagnostic system disclosed herein or as one of several classification specific panels in a diagnostic system. For example, in one aspect, disclosed herein are diagnostic systems, with positive and negative symptom schizophrenia (I), and wherein the one or more phenotypic sets comprise 15_13, 12_11, 21_1, 50_46, 9_6, 46_23, 54_11, 30_17, 18_13, 27_6, 61_18, 64_11, or 65_64. Also disclosed are diagnostic systems, wherein the system selects for positive and negative schizophrenia (II), and wherein the one or more phenotypic sets comprise 12_4 or 42_9. Also disclosed are diagnostic systems, wherein the system selects for negative schizophrenia (III), and wherein the one or more phenotypic sets comprise 52_28, 7_3, 48_41, 26_8, 69_41, 10_5, or 17_2. Also disclosed are diagnostic systems, wherein the system selects for positive schizophrenia (IV), and wherein the one or more phenotypic sets comprise 63_24 and 69_66. Also disclosed are diagnostic systems, wherein the system selects for severe process, positive schizophrenia (V), and wherein the one or more phenotypic sets comprise 22_13, 18_13, 53_6, 59_41, 20_19, 55_7, 34_17, 69_66, 277, 18_13, 4_1, 66_54, or 8_4. Also disclosed are diagnostic systems, wherein the system selects for moderate process, disorganized negative schizophrenia (VI), and wherein the one or more phenotypic sets comprise 51_38, 427, 18_3, or 46_29. Also disclosed are diagnostic systems, wherein the system selects for moderate process, positive and negative schizophrenia (VII), and wherein the one or more phenotypic sets comprise 5_2, 57_39, 11_5, or 24_4. Also disclosed are diagnostic systems, wherein the system selects for moderate process, continuous positive schizophrenia (VIII), and wherein the one or more phenotypic sets comprise 48_7, 28_23, or 25_20. As noted above, the disclosed classification specific phenotype panels can be used alone or in combination of 2 or more with any other classification specific phenotype panel in the disclosed diagnostic system.

As noted above, the disclosed classification specific phenotypic panels can be used alone or in combination of 2 or more with any other classification specific phenotype panel. In a non-limiting example, the diagnostic system can comprise classification specific phenotype panels I; II; III; IV; V; VI; VII; VIII; I and II; I and III; I and IV; I and V; I and VI; I and VII; I and VIII; II and III; II and IV; II and V; II and VI; II and VII; II and VIII; III and IV; III and V; III and VI; III and VII; III and VIII; IV and V; IV and VI; IV and VII; IV and VIII; V and VI; V and VII, V and VIII; VI and VII; VI and VIII; VII and VIII; I, II, and III; III and IV; I, II, and V; I, II, and VI; I, II, and VII, I, II, and VIII; I, III, and IV; I, III, and V; I, III, and VI; I, III, and VII; I, III, and VIII; I, IV, and V; I, IV, and VI; I, IV, and VII; I, IV, and VIII; I, V, and VI; I, V, and VII, I, V, and VIII; I, VI, and VII, I, VI, and VIII; I, VII and VIII; I, I, II, III, and IV; I, II, III, and V; I, II, III, and VI, I, II, III, and VII; I, II, III, and VIII; I, II, IV, and V; I, II, IV, and VI; I, II, IV; and VI; I, II, IV, and VII; I, II, IV, and VIII; I, II, V, and VI; I, II, V, and VII; I, II, V, and VIII; I, II, VI, and VII; I, II, VI, and VIII; I, II, VII, and VIII; I, III, IV, and V; I, III, IV, and VI; I, III, IV, and VII; I, III, IV, and VIII; I, III, V, and VI; I, III, V, and VII; I, III, V, and VIII; I, IV, V, and VI; I, IV, V, and VII; I, IV, V, and VIII; I, V, VI, and VII; I, V, VI, and VIII; I, VI, VII, and VIII; I, II, III, IV, and V; I, II, III, IV, and VI; I, I, II, III, IV, and VII; I, II, III, IV, and VIII; I, III, IV, V, and VI; I, III, IV, V, and VII; I, III, IV, V, and VIII; I, II, IV, V, and VI; I, II, IV, V, and VII; I, II, IV, V, and VIII; I, II, III, V, and VI; I, II, III, V, and VII; I, II, III, V, and VIII; I, II, III, VI, and VII; I, II, III, VI, and VIII; I, II, III, VII, and VIII; I, I, II, III, IV, V, and VI; I, II, III, IV, V, and VII; I, II, III, IV, V, and VIII; I, I, II, III, IV, VI, and VII; I, II, III, IV, VI, and VIII; I, II, III, IV, VII, and VIII; I, II, III, IV, V, VI, and VII; I, II, III, IV, V, VI, and VIII; I, II, III, IV, V, VI, VII, and VIII; II, III, and IV; II, III, and V; II, III, and VI; II, III, and VII, H, III, and VIII; II, IV, and V; II, IV, and VI; II, IV, and VII; II, IV, and VIII; II, V, and VI; II, V, and VII; II, V, and VIII; II, VI, and VII, II, VI, and VIII; II, VII and VIII; II, III, IV, and V; II, III, IV, and VI; I II, III, IV; and VI; II, III, IV, and VII; II, III, IV, and VIII; II, IV, V, and VI; II, IV, V, and VII; II, IV, V, and VIII; II, IV, VI, and VII; II, IV, VI, and VIII; II, IV, VII, and VIII; II, III, V, and V; II, III, V, and VI; II, III, V, and VII; and II, III, V, and VIII.

It is further understood that a diagnostic system can comprise any one or combination two or more phenotype panel in combination with any one or combination of two or more expression panels.

In one aspect, it is disclosed that the diagnostic system can comprise a purpose built analysis and diagnostic system to read the expression panel, analyze the expression panel data, input phenotypic sets, and display data and risk profiles associated with having schizophrenia or any particular class of schizophrenia disclosed herein. Thus, in one aspect, disclosed herein are diagnostic systems of any preceding aspect further comprising a means for reading the one or more expression panels, a computer operationally linked to the means for reading the one or more expression panels, and a display for visualizing the diagnostic risk; wherein the computer identifies the expression profile of an expression panel, compares the expression profile to a control, and catalogs that data, wherein the computer provides an input source for inputting phenotypic into a phenomic database; wherein the computer compares the expression and phenomic data and calculates relationships between the genomic and phenotypic data; wherein the computer compares the genomic and phenotypic relationship data to a reference standard; and wherein the computer outputs the relationship data and the standard on the display.

As noted above, the disclosed expression panel can be analyzed or read by any means known in the art including Northern analysis, RNAse protection assay, PCR, QPCR, genome microarray, DNA microarray, MMCHipslow density PCR array, oligo array, protein array, peptide array, phenotype microarray, SAGE, and/or high throughput sequencing. The readers can comprise any of those known in the art including, but not limited to array readers marked by Affymetrix, Agilent, Applied Microarrays, Arrayit, and Illumina.

As disclosed herein protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, N.J.) and specialised chip designs, such as engineered microchannels in a plate (e.g., The Living Chip™, Biotrove, Woburn, Mass.) and tiny 3D posts on a silicon surface (Zyomyx, Hayward Calif.). Particles in suspension can also be used as the basis of arrays, providing they are coded for identification; systems include colour coding for microbeads (Luminex, Austin, Tex.; Bio-Rad Laboratories) and semiconductor nanocrystals (e.g., QDots™, Quantum Dot, Hayward, Calif.), and barcoding for beads (UltraPlex™, SmartBead Technologies Ltd, Babraham, Cambridge, UK) and multimetal microrods (e.g., Nanobarcodes™ particles, Nanoplex Technologies, Mountain View, Calif.). Beads can also be assembled into planar arrays on semiconductor chips (LEAPS technology, BioArray Solutions, Warren, N.J.).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, Wash.) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, Mass.), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilised on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, Ariz.), rolling circle DNA amplification (Molecular Staging, New Haven Conn.), mass spectrometry (Intrinsic Bioprobes; Ciphergen, Fremont, Calif.), resonance light scattering (Genicon Sciences, San Diego, Calif.) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, Calif.).

Another methodology which can be used diagnostically and in expression profiling is the ProteinChip® array (Ciphergen, Fremont, Calif.), in which solid phase chromatographic surfaces bind proteins with similar characteristics of charge or hydrophobicity from mixtures such as plasma or tumour extracts, and SELDI-TOF mass spectrometry is used to detection the retained proteins.

Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilised on a microarray. Large-scale ‘proteome chips’ promise to be very useful in identification of functional interactions, drug screening, etc. (Proteometrix, Branford, Conn.).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

### C. METHODS

It is understood that use of the disclosed diagnostic system and/or expression and phenotypic panels can provide the capability to diagnose a subject with schizophrenia, assess the risk of having or developing schizophrenia, classifying a schizophrenia, and targeting a treatment of a schizophrenia. Accordingly, in one aspect, disclosed herein are methods of diagnosing a subject with schizophrenia comprising obtaining a biological sample from the subject, obtaining clinical data from the subject, and applying the biological sample and clinical data to the diagnostic system disclosed herein.

In one aspect, disclosed herein are methods of diagnosing a subject with schizophrenia and/or determining the schizophrenia class comprising: obtaining a biological sample from the subject; obtaining clinical data from the subject; applying the biological sample and clinical data to a diagnostic system for diagnosing schizophrenia, wherein the diagnostic system comprises one or more expression panels and one or more phenotypic panels; and comparing the genomic and phenotypic panels results to a reference standard, for example; wherein the presence of one or more SNP sets and one or more phenotypic sets in the subjects sample indicates the presence of schizophrenia, and wherein the genomic and phenotypic profile of the reference standard (such as, for example Table 7) most closely correlating with the subjects genomic and phenotypic profile indicates schizophrenia class of the subject.

It is understood that any one or combination of the SNP sets disclosed herein can be used in the disclosed methods. Thus, disclosed herein are methods of diagnosing a subject with schizophrenia and/or determining the schizophrenia class, wherein the one or more expression panels each comprise one or more of the single nucleotide polymorphism (SNP) sets selected from the group consisting of 19_2, 88_64, 81_13, 87_76, 58_29, 83_41, 9_9, 10_4, 14_6, 56_30, 42_37, 65_25, 71_55, 12_11, 90_78, 77_5, 88_8, 51_28, 59_48, 41_12, 22_11, 13_12, 31_22, 85_84, 87_84, 16_10, 56_19, 75_31, 81_73, 85_23, 21_8, 76_74, 61_39, 75_67, 76_63, 81_3, 87_26, 88_43, 25_10, 12_2, 52_42, and 54_51.

Because of these associations noted above in Table 7, it is possible to create panels to assess the risk of a subject to have a particular classification of schizophrenia. These classification specific expression panels can be used individually in the diagnostic method disclosed herein or as one of several classification specific panels in a diagnostic method. For example, in one aspect, disclosed herein are diagnostic methods, wherein the system selects for severe process, with positive and negative symptom schizophrenia (I), and wherein the one or more SNP sets comprise 56_30, 75_67, or 76_74. Also disclosed are diagnostic methods, wherein the system selects for positive and negative Schizophrenia (II), and wherein the one or more SNP sets comprise 59_48, 71_55, 21_8, 54_51, 31_22, 65_25, or 87_84. Also disclosed are diagnostic methods, wherein the system selects for negative Schizophrenia (III), and wherein the one or more SNP sets comprise 58_29, 9_9, 22_11, 81_3, 13_12, 61_39, 10_4, 81_73, 75_31, 56_19, 88_8, or 12_2. Also disclosed are diagnostic methods, wherein the system selects for Positive Schizophrenia (IV), and wherein the one or more SNP sets comprise 88_64, 85_84, or 41_12. Also disclosed are diagnostic methods, wherein the system selects for severe process, positive schizophrenia (V), and wherein the one or more SNP sets comprise 77_5, 81_13, or 25_10. Also disclosed are diagnostic methods, wherein the system selects for moderate process, disorganized negative schizophrenia (VI), and wherein the one or more SNP sets comprise 19_2, 52_42, 90_78, 12_11, 87_76, and 14_6. Also disclosed are diagnostic methods, wherein the system selects for moderate process, positive and negative schizophrenia (VII), and wherein the one or more SNP sets comprise 42_37, 88_43, or 51_28. Also disclosed are diagnostic methods, wherein the system selects for moderate process, continuous positive schizophrenia (VIII), and wherein the one or more SNP sets comprise 16_10, 83_41, or 87_26. As with the diagnostic systems any combination 2, 3, 4, 5, 6, 7, 8, or more of the disclosed expression panels can be used in the diagnostic methods.

It is understood that any one or combination of the phenotype panels disclosed herein can be used in the disclosed methods. Thus, disclosed herein are methods of diagnosing a subject with schizophrenia and/or determining the schizophrenia class, wherein the one or more phenotype panels each comprise one or more phenotypic sets selected from the group consisting of 15_13, 12_11, 21_1, 50_46, 9_6, 46_23, 54_11, 30_17, 18_13, 27_6, 61_18, 64_11, 65_64, 12_4, 42_9, 52_28, 7_3, 48_41, 26_8, 69_41, 10_5, 17_2, 63_24, 69_66, 22_13, 53_6, 59_41, 20_19, 55_7, 34_17, 27_7, 4_1, 66_54, 8_4, 51_38, 42_7, 18_3, 46_29, 5_2, 57_39, 11_5, 24_4, 48_7, 28_23, and 25_20.

As noted in Table 7, the phenotypic sets disclosed herein have been associated with one or more symptoms of one or more schizophrenia classes. Thus, contemplated herein are classification specific phenotype panels can be used individually in the diagnostic methods disclosed herein or as one of several classification specific panels in a diagnostic method. For example, in one aspect, disclosed herein are diagnostic methods, with positive and negative symptom schizophrenia (I), and wherein the one or more phenotypic sets comprise 15_13, 12_11, 21_1, 50_46, 9_6, 46_23, 54_11, 30_17, 18_13, 27_6, 61_18, 64_11, or 65_64. Also disclosed are diagnostic methods, wherein the system selects for positive and negative schizophrenia (II), and wherein the one or more phenotypic sets comprise 12_4 or 42_9. Also disclosed are diagnostic methods, wherein the system selects for negative schizophrenia (III), and wherein the one or more phenotypic sets comprise 52_28, 7_3, 48_41, 26_8, 69_41, 10_5, or 17_2. Also disclosed are diagnostic methods, wherein the system selects for positive schizophrenia (IV), and wherein the one or more phenotypic sets comprise 63_24 and 69_66. Also disclosed are diagnostic methods, wherein the system selects for severe process, positive schizophrenia (V), and wherein the one or more phenotypic sets comprise 22_13, 18_13, 53_6, 59_41, 20_19, 55_7, 34_17, 69_66, 27_7, 18_13, 4_1, 66_54, or 8_4. Also disclosed are diagnostic methods, wherein the system selects for moderate process, disorganized negative schizophrenia (VI), and wherein the one or more phenotypic sets comprise 51_38, 42_7, 18_3, or 46_29. Also disclosed are diagnostic methods, wherein the system selects for moderate process, positive and negative schizophrenia (VII), and wherein the one or more phenotypic sets comprise 5_2, 57_39, 11_5, or 24_4. Also disclosed are diagnostic methods, wherein the system selects for moderate process, continuous positive schizophrenia (VIII), and wherein the one or more phenotypic sets comprise 48_7, 28_23, or 25_20. As noted above, the disclosed classification specific phenotype panels can be used alone or in combination of 2 or more with any other classification specific phenotype panel in the disclosed diagnostic methods.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in .degree. C. or is at ambient temperature, and pressure is at or near atmospheric.

### 1. Example 1

### Uncovering the Hidden Risk Architecture of the Schizophrenias

a) Identifying Many SNP Sets as Candidates for Schizophrenia Risk

We first investigated the genotypic architecture of schizophrenia in the MGS study to identify SNP sets without knowledge of the subject's clinical status (i.e., case or control). Our exhaustive search uncovered 723 nonidentical and possibly overlapping SNP sets in the MGS samples. The SNP sets varied in terms of numbers of both subjects and SNPs. For example, one group contains 70 subjects and 24 SNPs, as expected because few subjects can share a large number of SNPs. Conversely, another group contains 258 subjects and three SNPs, as expected because a large number of subjects are likely to share only a few SNPs. Initially, we retained a large number of SNP sets merely to identify the genotypic clusters in all subjects whether they had schizophrenia or not.

b) SNP Sets Vary Greatly in Risk for Schizophrenia

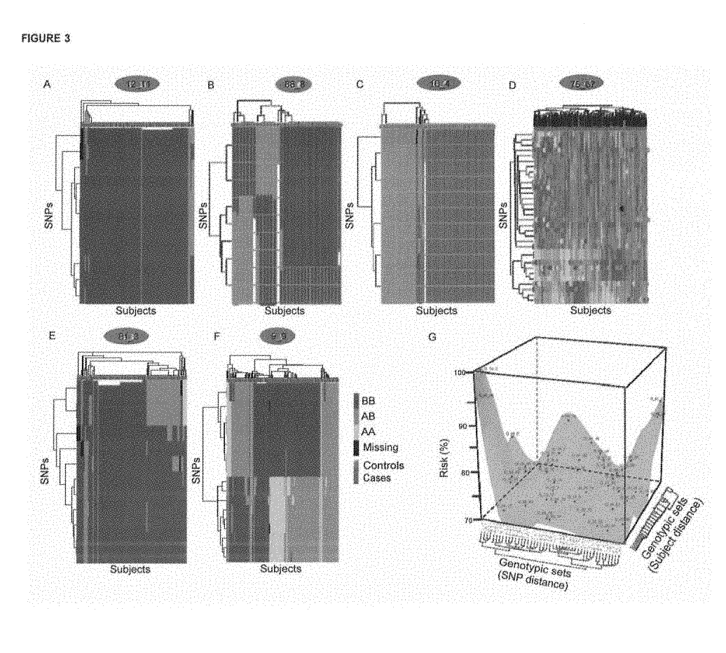

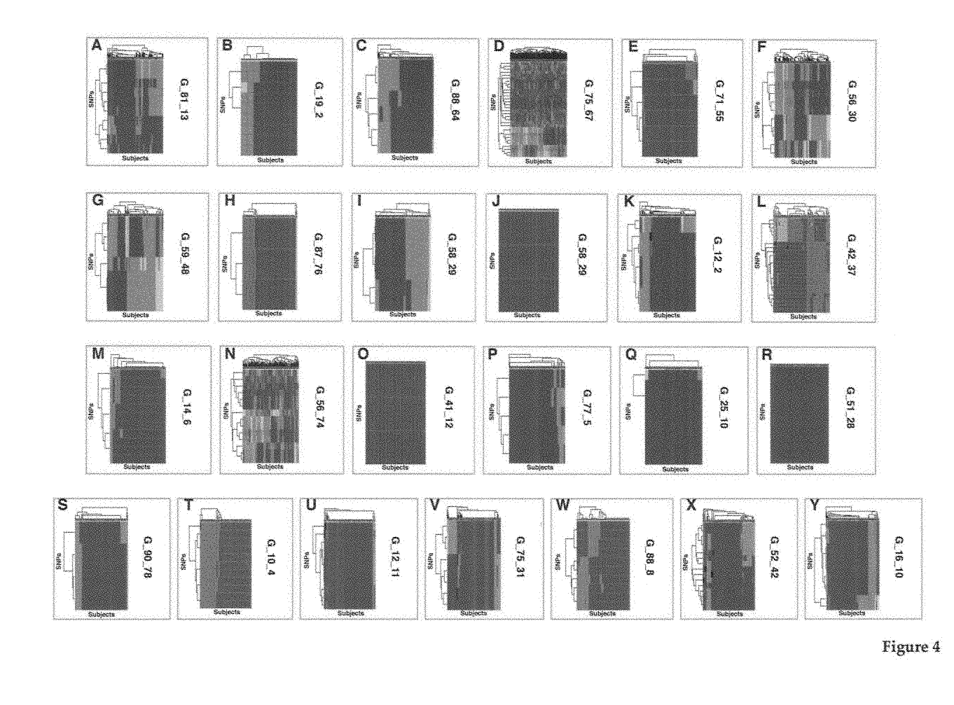

Second, we computed the risk for schizophrenia in carriers of each SNP set (FIG. 3A-F; see also FIG. 4). The risk of schizophrenia was normally distributed, as expected when capturing the full range of variability. Ninety-eight of the 723 SNP sets had a risk of schizophrenia greater than 66% and accounted for 90% of schizophrenia cases in the MGS study. Forty-two SNP sets had a risk of schizophrenia≥70% (Table 1). For example, SNP set 192 had a risk of 100%, meaning that all carriers were schizophrenia cases. The ability of SNP sets to predict schizophrenia risk is illustrated in FIG. 3G. SKAT showed that the association of schizophrenia with particular SNP sets was stronger than with the average effects of their constituent SNPs (Table 1). For example, the SNP set 81_13 has a p value of 1.46E-10, whereas the best and average SNPs within this set have p values of 2.15E-10 and 5.44E-03, respectively. SKAT and PLINK methods estimated similar p values for the individual SNPs (R²=0.99; p values for F statistics, <3.83. times.10⁻⁴⁶), showing that SKAT does not inflate results.

The global variance in liability to schizophrenia explained by the average effects of all SNPs simultaneously in our sample was 24%. While individual SNPs were mostly low penetrant, many high-risk SNP sets were highly penetrant (e.g., 100% to 70%; see Table 1) and much more informative in predicting schizophrenia risk.

c) Relations Among SNP Sets to One Another and to Gene Products

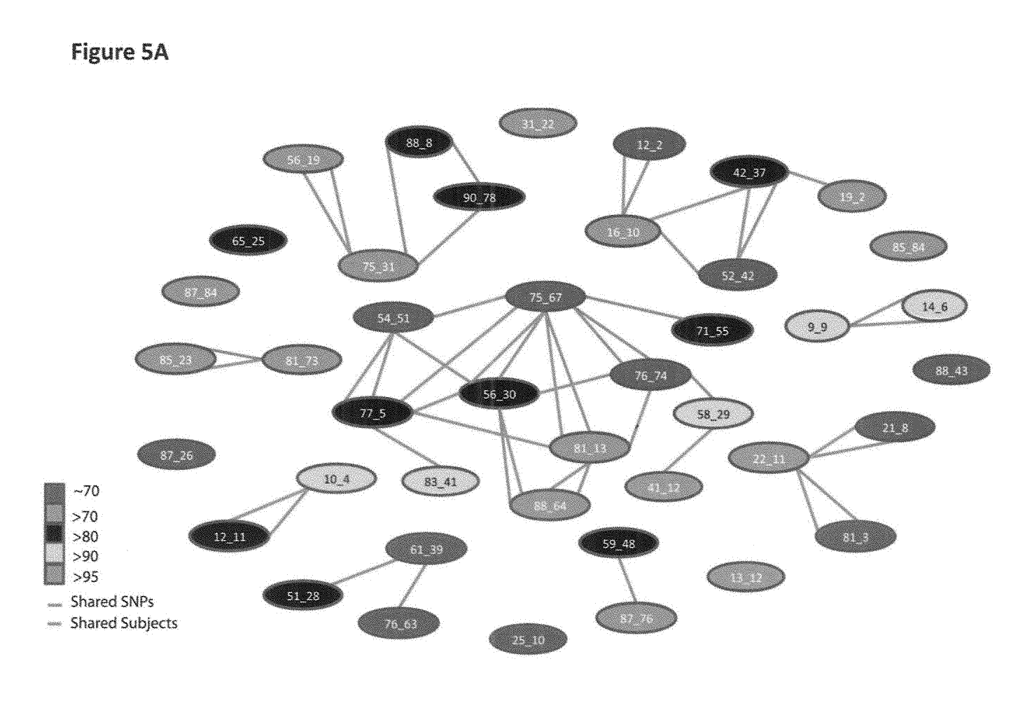

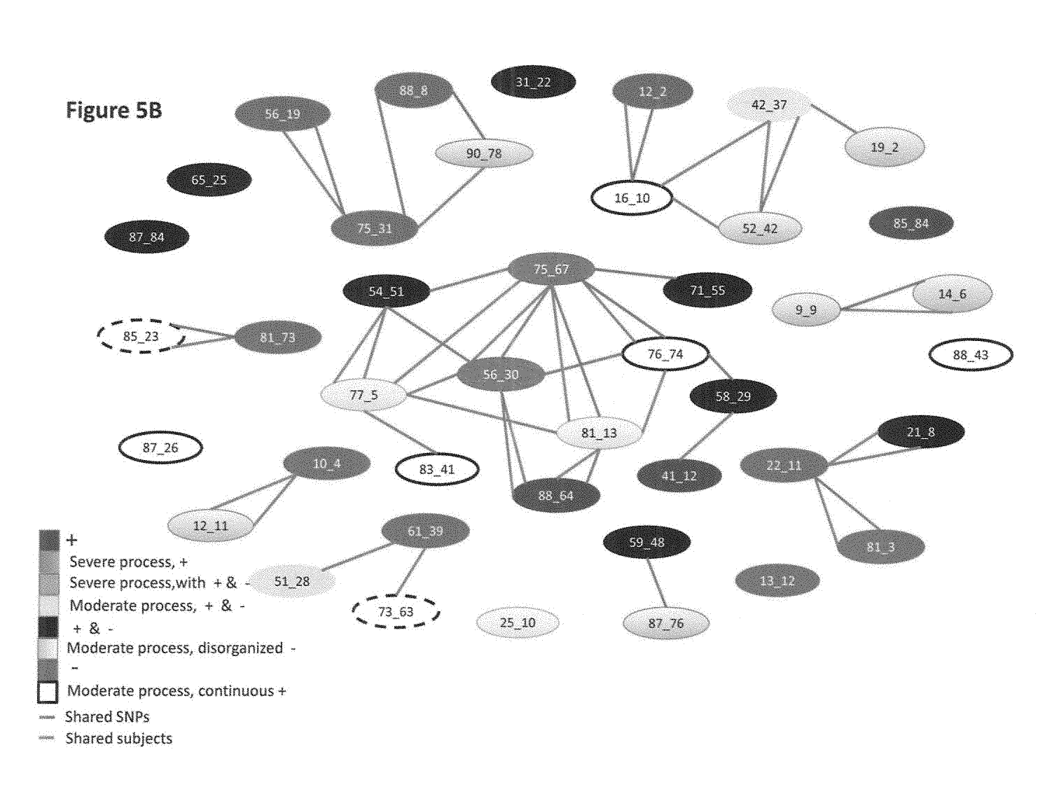

We show herein that schizophrenia may be an etiologically heterogeneous group of illnesses in which some genotypic networks are disjoint, that is, share neither SNPs nor subjects. To test this, we first checked for overlap in constituent SNPs and/or subjects among all the SNP sets at high risk for schizophrenia (see FIG. 8). We found that 17 genotypic networks were disjoint, sharing neither SNPs nor subjects (FIG. 5A), suggesting that these have distinct antecedents of schizophrenia. These networks vary in size and complexity: one highly connected network associates 11 SNP sets, whereas eight networks are composed of only a single isolated SNP set.

We also determined that some SNP sets share SNPs but not subjects (e.g., 59_48 and 87_76; FIG. 5A), as expected because they involve the same SNPs but with different allele values (both alleles of a SNP can act as risk alleles in different genetic contexts). In contrast, we found that the 58_29 and 41_12 SNP sets do not share SNPs, but independently specify almost the same individuals (FIG. 5A), as expected when, for example, distinct subsets of genotypic features influence a common developmental pathway. Finally, some SNP sets overlap in both SNPs and subjects, suggesting that one is a subset within the other (e.g., 88_64 and 81_13; see FIG. 4A, 4C). Therefore, the genotypic networks display distinct topologies differing in the way constituent SNPs and subjects are related.

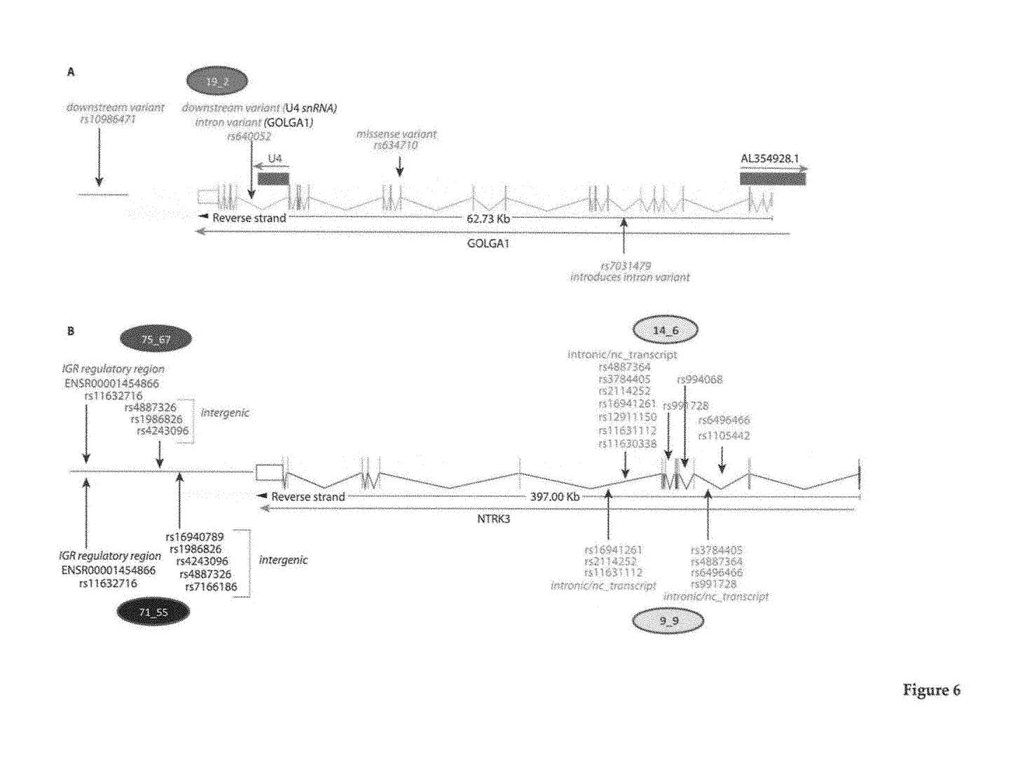

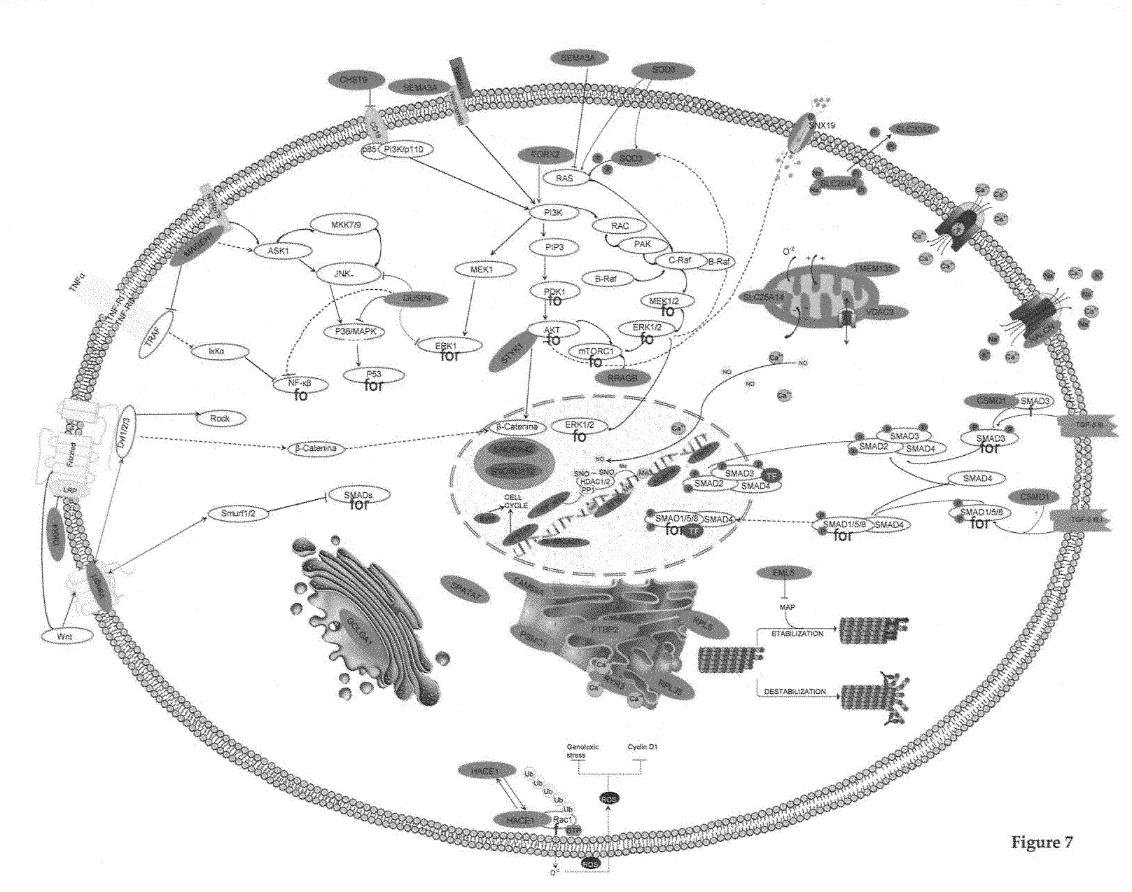

When evaluating whether different genotypic networks operate through distinct mechanisms, we found that high-risk SNP sets mapped to various classes of genes (e.g., protein coding, ncRNA genes, and pseudogenes) related to known functions and causing different effects on their products (FIG. 4A; see also Tables 2-4 and FIG. 6). We identified distinct pathways as exemplified in Table 5. Notably, all of these pathways are interconnected by the overlapping gene products that include genes previously associated with schizophrenia by GWAS, as well as genes known to be abnormally expressed in the brains of schizophrenia patients, and other genes not previously identified in prior work (see Table 6, FIG. 7, and the Pathways section). The emerging picture is suggestive of a possible pathophysiology in which abnormal brain development interacts with environmental events triggering abnormal or exaggerated immune and oxidative processes that increase risk of schizophrenia.

TABLE 5
Examples of products of genes uncovered by the SNP sets included in interconnected
signaling pathways^{a}

| Signaling Pathways/ | | | |
|---|---|---|---|
| Function | Genes | SNP sets | Symptoms |
| Neural development | DKK4 | 75_67 | Severe process, + & − |
| | STKY1 | | |
| | VANGL1 | | |
| | NCAM1 | 42_37 | Moderate process, + & − |
| | | 52_42 | Moderate process, − |
| | CHST9 | 81_73 | − |
| | EML5 | 13_12 | − |
| | SEM3A | 9_9 | Moderate process, − |
| Neurotrophin function | NTRK3 | 75_67 | Severe process, + & − |
| | upstream | 71_55 | + & − |
| | region | | |
| | SNTG1 | 81_13 | Severe process, + |
| | MAGEH1 | 25_10 | Severe process, + |
| Neurotransmission | NETO2, | 76_74, 75_67 | Severe process, with + & − |
| | OPN5 | 31_22, | + |
| | NALCN | 87_26 | Moderate process, continuous + |
| Neuronal function and | SPATA7, | 13_12 | − |
| neurodegenerative disorders | ZC3H14 | | |
| | SLC20A2 | 41_12 | + |

^{a}The 42 SNP sets at high risk for schizophrenia involved at least 96 gene loci, including 54 protein-coding loci and 42 polymorphisms at regulatory sites, as well as 112 polymorphisms in either intergenic or unannotated regions (see full Tables 2 and 6 and FIG. 7)

TABLE 6
Molecular Pathway and Ontologies Identified in the Genotypic-Phenotypic
Architecture of SZ (bold, abnormally expressed in the brains of SZ patients)

| Gene Name | Pathway and | Ontology |
|---|---|---|
| GSR | reactive oxygen species | antioxidant/oxidative stress |
| SOD3 | reactive oxygen species | antioxidant/oxidative stress |
| TMEM135 | reactive oxygen species/FoxO/DAF-16 | antioxidant |
| SLC25A14 | reactive oxygen species | antioxidant/ |
| | | mitochondria/oxidative stress |
| VDAC3 | mitochondria | apoptosis/mitochondria/oxidative |
| | | stress |
| PPP1R1C | TNFa; p21/p53/Bcl-2-antagonist/killer, | apoptosis/regulation of |
| | inhibition of Bcl-2/Bcl-XL | intracellular signaling |
| PAGE5 | wnt/DKK1 | apoptosis |
| WDR38 | | apoptosis |
| RRAGB | mTORC1 | apoptosis/cell growth/regulation |
| | | of intracellular signaling |
| TRPS1 | DNA binding/RNF4/dynein | apoptosis/gene expression |
| ST18 | TNFa; interleukin-1alpha/IL-6. | apoptosis/gene expression/ |
| | | neuroimmune regulation |
| EVI5 | GTPase activating protein/Rab11 | development, cell migration/ |
| | | regulation of intracellular |
| | | signaling |
| HACE1 | Rac1 | development, cell migration |
| SCAI | integrins; RhoA/Dia1 | development, cell migration/ |
| | | transcriptional regulation |
| STYK1 | wnt; Akt/GSK-3β | development, cell proliferation/cell |
| | | differentiation |
| CHST9 | Golgi sulfatation of proteins | development, cell/cell interactions |
| ATP8A2 | CDC50A related ATPase | neurodevelopment |
| PTCHD4 | hedgehog receptor | neurodevelopment |
| NCAM1 | integrins | neurodevelopment |
| IGSF1 | integrins | neurodevelopment |
| SEMA3A | integrins; neuropilin 1/Plexin A1 | neurodevelopment |
| EML5 | MAP | neurodevelopment |
| DKK4 | wnt/bcatenin | neurodevelopment |
| GOLGA1 | wnt/bcatenin; E-cadherin/Rab11a/b/Arl1 | neurodevelopment/protein |
| | GTPase | synthesis and trafficking |
| FOXR2 | wnt/bcatenin; RAS GTPase/MAPK/ERK | neurodevelopment/regulation of |
| | | intracellular signaling |
| VANGL1 | wnt; disheveled 1, 2, 3 | neurodevelopment |
| DUSP4 | ERK1/2/MAPK; a target of NFkB inhibition | neurodevelopment/apoptosis/ |
| | | regulation of intracellular |
| | | signaling |
| CSMD1 | Smad3/TGFa/AKT/p53 | neurodevelopment/apoptosis/ |
| | | neuroimmune regulation |
| ARPC5L | Calmodulin/clathrin | neurodevelopment/synaptogenesis |
| NTRK3 | MAPK | neurotrophins |
| MAGEH1 | p75/NFkB/cJun/ERK | neurotrophins |
| SNTG1 | PI2 binding/dystrophin/dystobrevin/factor | neurotrophins |
| | gamma enolase; effector of cathepsin X; | |
| | effector of TAPP1 | |
| NALCN | non-voltage dependent ion channel | neuronal excitability |
| RYR3 | Calcium/calmodulin | neuronal function/plasticity/ |
| | | regulation of intracellular |
| | | signaling |
| GPR119 | G protein receptor | neurotransmission, cannabioid |
| | | transmission/neuronal function |
| OPN5 | NRG1/Erb4 | neurotransmission, GABAergic |
| | | transmission/neuronal function |
| NETO2 | GluK2 | neurotransmission, glutamatergic |
| | | transmission/neuronal function |
| SPATA7 | consensus sites for PKC/CK-II | neurodegenerative disorder/, |
| | | retinal degeneration |
| ITFG1 | PP2A/rad3 | DNA replication/DNA repair |
| PTBP2 | mRNA binding | mRNA splicing |
| PRPF31 | mRNA binding | mRNA splicing |
| RNU4-1 | mRNA binding | mRNA splicing |
| PSMC1 | Ubiquitin | protein degradation |
| RPL35 | ribosome | protein synthesis |
| RPL5 | ribosome/casein kinase II | protein synthesis/inhibition of cell |
| | | proliferation/protein synthesis and |
| | | trafficking |
| SNX19 | PI2 binding | cell trafficking |
| SMARCAD1 | histone H3/H4 deacetylation | epigenetic gene expression |
| SNORA42 | ribosome | gene expression/protein synthesis |
| | | and trafficking |
| SNORD112 | ribosome | gene expression/protein synthesis |
| | | and trafficking |
| NRDE2 | siRNA | gene expression |
| ABCC12 | ATP transport | immunity |
| FAM69A | | immunity in CNS/neuroimmune |
| | | regulation |
| HPGDS | Prostaglandin D receptors G protein/NFkB | immunity, inflammation, sleep, |
| | | smooth muscle/neuroimmune |
| | | regulation |
| SLC20A2 | Sodium/phosphate symporter | neurodegenerative disorders/ |
| | | phosphate metabolism/viral |
| | | transport |
| PAGE3 | | |
| STPG2 | | |
| GP2 | | |
| PHKB | Calcium/calmodulin | glycogenolysis/regulation of |
| | | intracellular signaling |

d) Complex Genotypic-Phenotypic Relationships in Schizophrenia

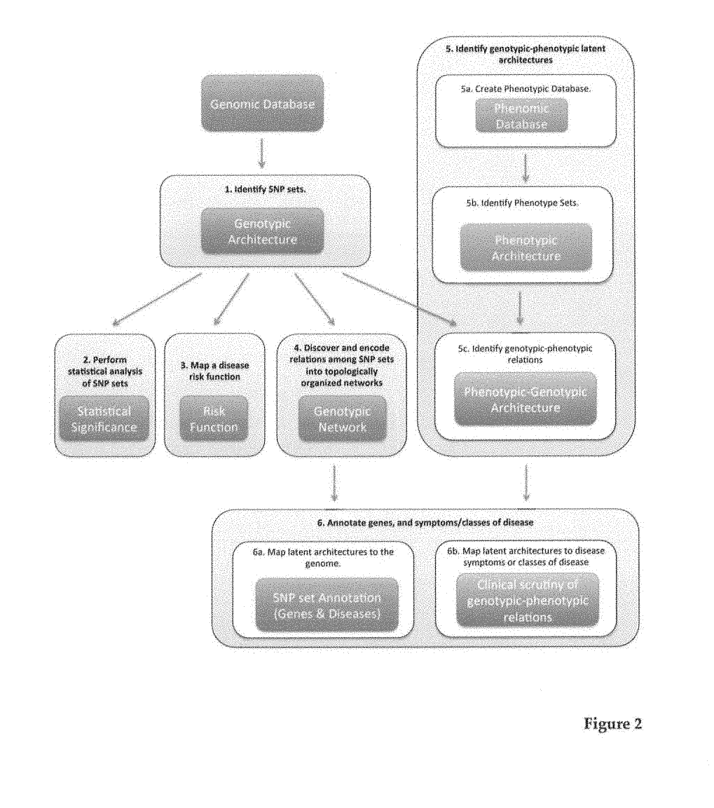

Next we examined whether the complex genetic architecture of schizophrenia leads to phenotypic heterogeneity. Using data from the Diagnostic Interview for Genetic Studies, as well as from the Best Estimate Diagnosis Code Sheet submitted by GAIN/non-GAIN to dbGaP (see FIG. 2), we originally identified 342 nonidentical and possibly overlapping phenotypic sets of distinct clinical features that cluster in particular cases with schizophrenia (i.e., phenotypic sets or clinical syndromes) without regard for their genetic background. Different SNP sets were significantly associated with particular clinical syndromes (hypergeometric statistics, p values from 2E-13 to 1E-03). However, the genotypic-phenotypic relations were complex (i.e., manyto-many): the same genotypic network could be associated with multiple clinical outcomes (i.e., multifinality or pleiotropy) and different genotypic networks could lead to the same clinical outcome (i.e., equifinality or heterogeneity; Table 7; see also Table 8). The genotypic-phenotypic relations were highly significant by a permutation test (empirical p value, 4.7E-13; Table 7; see also Table 8).

TABLE 8
Genotypic-Phenotypic AND/OR Relationships..

| | | Hyper- | |
|---|---|---|---|
| SNP | Phenotype | Geometric | |
| Sets | Sets | p-value | Phenotype features |
| 22_11 | 69_41 | 5.52E−05 | Avolition_Apathy[I13240] & No_Emotions[I13310] |
| | 10_5 | 4.62E−04 | No_Emotions[I13310] & |
| | | | Pattern_Sx[I14350] = ContinuousMixtureOfPositiveAndNegative |
| | | | Symptoms & DSM4_Negative_Sx[A60g] & |
| | | | Avolition_Apathy[I13240] & Alogia[I21400] |
| | 17_2 | 1.01E−04 | Disorganized_Speech[I12990] & Odd_Speech[I13060] & |
| | | | DSM4_Disorganized_Speech[A60e] |
| 25_10 | 27_7 | 4.76E−06 | Severity_Pattern[I14360] = SevereDeterioration & |
| | | | Unable_To_Function_Most_Time_Since_Onset[I21500] & |
| | | | Psychosis_without_Dep_Mania |
| | 18_13 | 9.50E−05 | DSM4_2 + Voices_Commented[A60d] & cs_A2a & |
| | | | Aud_2+_Voices[I12170] & Running_Comment[I12100] |
| | 4_1 | 2.49E−03 | AH(Voices_Noises_Music)[I12030] & |
| | | | DSM4_2 + Voices_Commented[A60d] & |
| | | | Running_Comment[I12100] & Aud_2+_Voices[I12170] & |
| | | | Thought_Echo[I12240] & |
| | | | Auditory_Halns_Ever[I10920] = Present |
| | 66_54 | 2.10E−03 | Del_of_Ref[I11460] & Persecutory_Delusions[I11030] |
| | 8_4 | 1.93E−03 | DSM4_Definite_Bizarre_Del[A60b] & |
| | | | Delusion_Bizarre[I12020] = Definite & |
| | | | Delusion_Widespread[I12010] = Somewhat & |
| | | | Del_Mind_Reading[I11600] |
| 42_37 | 5_2 | 1.32E−04 | Classification_Longitud_SZ[I21560] = Continuous & |
| | | | Unable_To_Function_Most_Time_Since_Onset[I21500] & |
| | | | DSM4_Hallucinations[A60c] & |
| | | | Psychosis_without_Dep_Mania & |
| | | | Auditory_Halns_Ever[I10920] = Present & |
| | | | Severity_Pattern[I14360] = ModerateDeterioration & |
| | | | AH(Voices_Noises_Music)[I12030] & |
| | | | Pattern_Sx[I14350] = ContinuousMixtureOfPositiveAndNegative |
| | | | Symptoms |
| | 57_39 | 4.70E−03 | cs_A1a & Del_of_Ref[I11460] |
| 51_28 | 24_4 | 9.58E−04 | Delusion_Fragment[I12000] & Delusion_Bizarre[I12020] & |
| | | | Auditory_Halns_Ever[I10920] = Suspected |
| | 9_7 | 1.19E−04 | No_Emotions[I13310] & |
| | | | Pattern_Sx[I14350] = ContinuousMixtureOfPositiveAndNegative |
| | | | Symptoms & Psychosis_without_Dep_Mania & |
| | | | Unable_To_Function_Most_Time_Since_Onset[I21500] & |
| | | | Avolition_Apathy[I13240] & DSM4_Negative_Sx[A60g] & |
| | | | Alogia[I21400] |
| | 52_24 | 1.68E−03 | Classification_Longitud_SZ[I21560] = Continuous & |
| | | | Aud_2+_Voices[I12170] & |
| | | | Delusion_Widespread[I12010] = Somewhat |
| | 3_2 | 2.48E−03 | cs_A3 & cs_A1 & cs_A5 & cs_A4 & cs_A2 & |
| | | | Unable_To_Function_Most_Time_Since_Onset[I21500] & |
| | | | cs_A1a & DSM4_Negative_Sx[A60g] |
| 52_42 | 5_2 | 1.12E−04 | Classification_Longitud_SZ[I21560] = Continuous & |
| | | | Unable_To_Function_Most_Time_Since_Onset[I21500] & |
| | | | DSM4_Hallucinations[A60c] & |
| | | | Psychosis_without_Dep_Mania & |
| | | | Severity_Pattern[I14360] = ModerateDeterioration& |
| | | | AH(Voices_Noises_Music)[I12030] & |
| | | | Pattern_Sx[I14350] = ContinuousMixtureOfPositiveAndNegative |
| | | | Symptoms |
| | 67_24 | 1.59E−03 | No_Emotions[I13310] & DSM4_Negative_Sx[A60g] |
| 54_51 | 49_36 | 4.49E−04 | DSM4_2 + Voices_Commented[A60d] & |
| | | | DSM4_Hallucinations[A60c] & |
| | | | Delusion_Fragment[I12000] = Definite & |
| | | | Auditory_Halns_Ever[I10920] = Present & |
| | | | Running_Comment[I12100] |
| | 50_46 | 1.42E−03 | DSM4_Gross_Disorganization[A60f] & |
| | | | DSM4_2 + Voices_Commented[A60d] & |
| | | | DSM4_Hallucinations[A60c] |
| | 47_40 | 4.24E−03 | Thought_Broadcasting[I11670] & Del_of_Ref[I11460] |
| 56_30 | 15_13 | 2.55E−05 | Pattern_Sx[I14350] = ContinuouslyPositive & |
| | | | Unable_To_Function_Most_Time_Since_Onset[I21500] & |
| | | | Severity_Pattern[I14360] = SevereDeterioration |
| | 12_11 | 1.79E−04 | DSM4_2 + Voices_Commented[A60d] & |
| | | | Running_Comment[I12100] & Aud_2+_Voices[I12170] & |
| | | | cs_A2a & AH(Voices_Noises_Music)[I12030] |
| | 21_1 | 3.66E−04 | Thought_Echo[I12240] & Thought_Insert[I11740] & |
| | | | Thought_Withdraw[I11810] & Del_Mind_Reading[I11600] & |
| | | | Thought_Broadcasting[I11670] & |
| | | | Running_Comment[I12100] & Aud_2+_Voices[I12170] |
| | 50_46 | 5.70E−04 | DSM4_Hallucinations[A60c] & |
| | | | DSM4_Gross_Disorganization[A60f] & |
| | | | DSM4_2 + Voices_Commented[A60d] & |
| | | | Auditory_Halns_Ever[I10920] = Present |
| | 9_6 | 4.45E−03 | Thought_Echo[I12240] & Thought_Insert[I11740] & |
| | | | Thought_Withdraw[I11810] & Del_Mind_Reading[I11600] & |
| | | | Thought_Broadcasting[I11670] & |
| | | | Mood_Incongruent_Hal[I17706] & Being_Controlled[I11530] |
| | | | & AH(Voices_Noises_Music)[I12030] & |
| | | | Somatic_Tactile[I12520] & Gustatory_Hal[I12730] & |
| | | | Olfactory_Hal[I12590] & Religious_Delusions[I11320] & |
| | | | Being_Controlled[I11530] |
| | 46_23 | 4.15E−03 | Persecutory_Delusions[I11030] & Odd_Speech[I13060] & |
| | | | Mood_Incongruent_Hal[I17706] & |
| | | | Delusion_Bizarre[I12020] = Somewhat & |
| | | | Odd_Behavior[I12920] & |
| | | | Delusion_Fragment[I12000] = Somewhat & |
| | | | Del_of_Ref[I11460] & Thought_Insert[I11740] & |
| | | | Delusion_Widespread[I12010] = Somewhat & |
| | | | Jealousy_Delusions[I11110] & Disorganized_Speech[I12990] |
| | | | & No_Emotions[I13310] & Avolition_Apathy[I13240] |
| 59_48 | 12_4 | 1.88E−04 | cs_A3 & cs_A4 & cs_A1 & cs_A2 & cs_A5 & cs_A1a |
| 75_67 | 15_13 | 2.31E−13 | Pattern_Sx[I14350] = ContinuouslyPositive & |
| | | | Severity_Pattern[I14360] = SevereDeterioration & |
| | | | Unable_To_Function_Most_Time_Since_Onset[I21500] & |
| | | | Psychosis_without_Dep_Mania |
| | 54_11 | 4.90E−06 | Severity_Pattern[I14360] = SevereDeterioration & |
| | | | Classification_Longitud_SZ[I21560] = Continuous & cs_A4 |
| | 30_17 | 2.56E−04 | Persecutory_Delusions[I11030] & |
| | | | Unable_To_Function_Most_Time_Since_Onset[I21500] & |
| | | | Severity_Pattern[I14360] = SevereDeterioration & |
| | | | Odd_Speech[I13060] & Del_of_Ref[I11460] |
| | 18_13 | 3.50E−04 | DSM4_2 + Voices_Commented[A60d] & |
| | | | Running_Comment[I12100] & cs_A2a & |
| | | | Aud_2+_Voices[I12170] & |
| | | | AH(Voices_Noises_Music)[I12030] & |
| | | | Auditory_Halns_Ever[I10920] = Present & |
| | | | Jealousy_Delusions[I11110] |
| | 27_6 | 3.62E−03 | Thought_Insert[I11740] & Thought_Withdraw[I11810] |
| | 50_46 | 3.61E−03 | DSM4_Gross_Disorganization[A60f] & |
| | | | DSM4_2 + Voices_Commented[A60d] & |
| | | | DSM4_Hallucinations[A60c] |
| | 61_18 | 4.28E−03 | Persecutory_Delusions[I11030] & |
| | | | Delusion_Widespread[I12010] = Somewhat & |
| | | | Del_of_Ref[I11460] |
| | 64_11 | 1.45E−03 | cs_A3 & Odd_Speech[I13060] |
| | 65_64 | 1.21E−03 | Delusion_Widespread[I12010] = Somewhat & |
| | | | Classification_Longitud_SZ[I21560] = Continuous |
| 76_74 | 15_13 | 1.07E−07 | Severity_Pattern[I14360] = SevereDeterioration & |
| | | | Pattern_Sx[I14350] = ContinuouslyPositive & |
| | | | Unable_To_Function_Most_Time_Since_Onset[I21500] & |
| | | | Psychosis_without_Dep_Mania |
| | 65_64 | 1.47E−03 | Delusion_Widespread[I12010] = Somewhat & |
| | | | Classification_Longitud_SZ[I21560] = Continuous & cs_A4 |
| 77_5 | 22_13 | 5.66E−05 | Severity_Pattern[I14360] = SevereDeterioration & |
| | | | Psychosis_without_Dep_Mania & |
| | | | Unable_To_Function_Most_Time_Since_Onset[I21500] & |
| | | | Pattern_Sx[I14350] = ContinuouslyPositive |
| | 18_13 | 3.25E−03 | DSM4_2 + Voices_Commented[A60d] & cs_A2a & |
| | | | Aud_2+_Voices[I12170] & Running_Comment[I12100] |
| | 53_6 | 4.76E−03 | Classification_Longitud_SZ[I21560] = Continuous & |
| | | | DSM4_Hallucinations[A60c] & |
| | | | DSM4_2 + Voices_Commented[A60d] & cs_A2a & |
| | 59_41 | 1.22E−03 | AH(Voices_Noises_Music)[I12030] & |
| | | | Auditory_Halns_Ever[I10920] = Present |
| 81_13 | 20_19 | 2.83E−04 | Pattern_Sx[I14350] = ContinuouslyPositive & |
| | | | Severity_Pattern[I14360] = SevereDeterioration & |
| | | | Unable_To_Function_Most_Time_Since_Onset[I21500] & |
| | | | Psychosis_without_Dep_Mania |
| | 55_7 | 8.57E−04 | DSM4_2 + Voices_Commented[A60d] & |
| | | | DSM4_Hallucinations[A60c] & |
| | | | Delusion_Fragment[I12000] = Somewhat & |
| | | | Delusion_Widespread[I12010] = Somewhat & |
| | | | Delusion_Bizarre[I12020] = Somewhat & |
| | | | Delusion_Fragment[I12000] = Definite & |
| | | | Auditory_Halns_Ever[I10920] = Present |
| | 34_17 | 2.40E−03 | Del_of_Ref[I11460] & Persecutory_Delusions[I11030] |
| | 69_66 | 1.30E−03 | Jealousy_Delusions[I11110] & cs_A2a |
| 90_78 | 22_7 | 7.29E−04 | Pattern_Sx[I14350] = ContinuousMixtureOfPositiveAndNegative |
| | | | Symptoms & No_Emotions[I13310] & |
| | | | Unable_To_Function_Most_Time_Since_Onset[I21500] |
| | 65_55 | 4.51E−04 | Guilt_Sin_Delusions[I11180] & |
| | | | Persecutory_Delusions[I11030] & cs_A4 & |
| | | | Del_of_Ref[I11460] |
| | 70_43 | 4.37E−03 | DSM4_Gross_Disorganization[A60f] & |
| | | | Odd_Behavior[I12920] & Avolition_Apathy[I13240] |
| 10_4 | 66_50 | 2.45E−04 | Unable_To_Function_Most_Time_Since_Onset[I21500] & |
| | | | Classification_Longitud_SZ[I21560] = Continuous |
| | 43_20 | 3.14E−04 | Thought_Insert[I11740] & Thought_Withdraw[I11810] |
| | 64_37 | 3.32E−03 | cs_A3 & cs_A4 |
| 12_11 | 29_13 | 4.30E−04 | Severity_Pattern[I14360] = SevereDeterioration & |
| | | | Pattern_Sx[I14350] = ContinuousMixtureOfPositiveAndNegative |
| | | | Symptoms & Delusion_Widespread[I12010] = Definite & |
| | | | Psychosis_without_Dep_Mania |
| | 33_13 | 1.92E−03 | Guilt_Sin_Delusions[I11180]] & Delusion_Bizarre[I12020] |
| 12_2 | 67_24 | 4.83E−03 | DSM4_Negative_Sx[A60g] & No_Emotions[I13310] |
| | 30_29 | 4.36E−03 | Del_of_Ref[I11460] & Somatic_Tactile[I12520] |
| 13_12 | 27_20 | 6.26E−04 | Psychosis_without_Dep_Mania[A620] & |
| | | | Disorganized_Speech[I12990] & |
| | | | DSM4_Disorganized_Speech[A60e] |
| | 27_22 | 1.38E−03 | Thought_Broadcasting[I11670] & |
| | | | Del_Mind_Reading[I11600] & cs_A1a |
| | 58_16 | 1.56E−03 | DSM4_Negative_Sx[A60g] & |
| | | | Persecutory_Delusions[I11030] & Avolition_Apathy[I13240] |
| 14_6 | 42_7 | 4.96E−04 | Unable_To_Function_Most_Time_Since_Onset[I21500] & |
| | | | Severity_Pattern[I14360] = ModerateDeterioration & |
| | | | Severity_Pattern[I14360] = ModerateDeterioration & |
| | | | Psychosis_without_Dep_Mania |
| | 18_3 | 2.55E−03 | Disorg/Inapp_Behav[I21050] & |
| | | | DSM4_Gross_Disorganization[A60f] |
| | 46_29 | 3.78E−03 | Thought_Echo[I12240] & cs_A2a |
| 16_10 | 48_7 | 1.44E−03 | Psychosis_without_Dep_Mania |
| 21_8 | 13_11 | 1.56E−04 | DSM4_2 + Voices_Commented[A60d] & |
| | | | Aud_2+_Voices[I12170] & Running_Comment[I12100] & |
| | | | cs_A2a & AH(Voices_Noises_Music)[I12030] |
| | 64_46 | 4.19E−04 | Alogia[I21400] & No_Emotions[I13310] & |
| | | | Avolition_Apathy[I13240] |
| | 62_35 | 2.89E−03 | Del_of_Ref[I11460] & Being_Controlled[I11530] |
| 31_22 | 24_8 | 2.93E−03 | Delusion_Fragment[I12000] = Definite & |
| | | | DSM4_Definite_Bizarre_Del[A60b] & |
| | | | Delusion_Bizarre[I12020] = Definite & |
| | | | Delusion_Widespread[I12010] = Somewhat |
| | 62_26 | 1.88E−03 | Thought_Insert[I11740] & Aud_2+_Voices[I12170] & |
| | | | Running_Comment[I12100] |
| 41_12 | 58_28 | 6.04E−04 | Return_Normal_for_2Months[I13600] & |
| | | | Severity_Pattern[I14360] = MildDeterioration |
| | 23_16 | 2.50E−03 | Severity_Pattern[I14360] = MildDeterioration & |
| | | | Classification_Longitud_SZ[I21560] = EpisodicWithInterepisode |
| | | | ResidualSymptoms & |
| | | | Delusion_Widespread[I12010] = Definite & |
| | | | Auditory_Halns_Ever[I10920] & |
| | | | Classification_Longitud_SZ[I21560] = SingleEpisodeInPartial |
| | | | Remission & |
| | | | Pattern_Sx[I14350] = PredominantlyPositiveConvertingToPre |
| | | | dominantlyNegative & |
| | | | Return_Normal_for_2Months[I13600] |
| 56_19 | 33_13 | 4.30E−04 | Guilt_Sin_Delusions[I11180] & |
| | | | Psychosis_without_Dep_Mania |
| 58_29 | 52_28 | 1.44E−04 | Thought_Insert[I11740] & Thought_Withdraw[I11810] |
| 61_39 | 64_48 | 5.11E−05 | Delusion_Widespread[I12010] = Somewhat & |
| | | | Classification_Longitud_SZ[I21560] = Continuous |
| | 32_9 | 2.79E−03 | Thought_Insert[I11740] & Thought_Withdraw[I11810] |
| 65_25 | 36_14 | 5.53E−04 | Thought_Broadcasting[I11670] & |
| | | | Del_Mind_Reading[I11600] & cs_A1a |
| | 31_29 | 3.76E−04 | cs_A3 & cs_A4 & cs_A5 & cs_A2 & cs_A1 & cs_A1a |
| | 61_21 | 5.55E−03 | Del_Mind_Reading[I11600] & |
| | | | Thought_Broadcasting[I11670] & Thought_Insert[I11740] & |
| | | | Psychosis_without_Dep_Mania[A620] |
| 75_31 | 44_3 | 6.37E−04 | cs_A4 & |
| | | | Unable_To_Function_Most_Time_Since_Onset[I21500] & |
| | | | cs_A3 |
| | 64_6 | 1.55E−03 | DSM4_Disorganized_Speech[A60e] & |
| | | | Disorganized_Speech[I12990] & |
| | | | Pattern_Sx[I14350] = ContinuousMixtureOfPositiveAndNegative |
| | | | Symptoms |
| 81_3 | 34_33 | 1.96E−03 | Psychosis_without_Dep_Mania & |
| | | | Delusion_Fragment[I12000] = Somewhat |
| | 46_25 | 4.51E−03 | Avolition_Apathy[I13240] & No_Emotions[I13310] & |
| | | | DSM4_2 + Voices_Commented[A60d] |
| 81_73 | 19_12 | 2.46E−04 | Disorg/Inapp_Behav[I21050] & |
| | | | DSM4_Gross_Disorganization[A60f] |
| | 59_12 | 2.20E−04 | Odd_Behavior[I12920] & Disorg/Inapp_Behav[I21050] |
| 85_84 | 38_2 | 6.10E−04 | Delusion_Bizarre[I12020] = Definite & |
| | | | DSM4_Definite_Bizarre_Del[A60b] & |
| | | | Delusion_Fragment[I12000] = Definite |
| | 49_36 | 3.28E−03 | DSM4_2 + Voices_Commented[A60d] & |
| | | | DSM4_Hallucinations[A60c] & |
| | | | Delusion_Fragment[I12000] = Definite & |
| | | | Auditory_Halns_Ever[I10920] = Present |
| | 58_4 | 4.81E−03 | Auditory_Halns_Ever[I10920] = Present & |
| | | | DSM4_Hallucinations[A60c] & cs_A2 |
| 87_26 | 25_20 | 4.22E−03 | Pattern_Sx[I14350] = ContinuouslyPositive & |
| | | | Psychosis_without_Dep_Mania |
| 87_76 | 14_10 | 5.12E−04 | Pattern_Sx[I14350] = ContinuousMixtureOfPositiveAndNegative |
| | | | Symptoms & |
| | | | Unable_To_Function_Most_Time_Since_Onset[I21500] |
| | 64_6 | 2.19E−04 | DSM4_Disorganized_Speech[A60e] & |
| | | | Disorganized_Speech[I12990] & cs_A4 |
| | 62_60 | 1.83E−03 | Avolition_Apathy[I13240] & |
| | | | Classification_Longitud_SZ[I21560] = Continuous |
| | 59_13 | 4.12E−03 | No_Emotions[I13310] & |
| | | | Classification_Longitud_SZ[I21560] = Continuous & |
| | | | Pattern_Sx[I14350] = ContinuousMixtureOfPositiveAndNegative |
| | | | Symptoms & DSM4_Negative_Sx[A60g] |
| 88_43 | 11_5 | 6.88E−04 | Pattern_Sx[I14350] = ContinuousMixtureOfPositiveAndNegative |
| | | | Symptoms & |
| | | | Unable_To_Function_Most_Time_Since_Onset[I21500] & |
| | | | Psychosis_without_Dep_Mania & |
| | | | Severity_Pattern[I14360] = ModerateDeterioration |
| | 16_1 | 7.77E−04 | Delusion_Fragment[I12000] & Delusion_Bizarre[I12020] |
| | 52_8 | 1.68E−03 | Disorg/Inapp_Behav[I21050] & cs_A4 & |
| | | | DSM4_Gross_Disorganization[A60f] |
| | 18_17 | 2.90E−03 | Del_Mind_Reading[I11600] & |
| | | | Thought_Broadcasting[I11670] & Thought_Insert[I11740] |
| | 66_12 | 2.25E−03 | AH(Voices_Noises_Music)[I12030] & |
| | | | Auditory_Halns_Ever[I10920] = Present & |
| | | | DSM4_Hallucinations[A60c] |
| 88_64 | 63_24 | 3.45E−04 | DSM4_2 + Voices_Commented[A60d] & |
| | | | DSM4_Hallucinations[A60c] & |
| | | | Auditory_Halnss_Ever[I10920] = Present & |
| | | | Psychosis_without_Dep_Mania[A620] |
| | 69_66 | 4.49E−03 | Jealousy_Delusions[I11110] & cs_A2a |
| 88_8 | 13_4 | 4.49E−03 | DSM4_Disorganized_Speech[A60e] & |
| | | | Disorganized_Speech[I12990] & Odd_Speech[I13060] |
| 9_9 | 7_3 | 1.97E−04 | DSM4_Disorganized_Speech[A60e] & Odd_Speech[I13060] |
| | | | & Disorganized_Speech[I12990] |
| | 48_41 | 2.23E−03 | No_Emotions[I13310] & Persecutory_Delusions[I11030] |
| | 26_8 | 4.20E−03 | Jealousy_Delusions[I11110] & Guilt_Sin_Delusions[I11180] |
| | | | & Del_Mind_Reading[I11600] |
| 19_2 | 51_38 | 4.03E−04 | cs_A4 & cs_A3 |
| 71_55 | 42_9 | 1.98E−03 | Running_Comment[I12100] & |
| | | | DSM4_2 + Voices_Commented[A60d] |
| 83_41 | 28_23 | 3.48E−03 | Pattern_Sx[I14350] = ContinuouslyPositive & |
| | | | Severity_Pattern[I14360] = SevereDeterioration & |
| | | | Unable_To_Function_Most_Time_Since_Onset[I21500] & |
| | | | Psychosis_without_Dep_Mania |
| 87_84 | 68_19 | 8.19E−04 | cs_A1a & Del_of_Ref[I11460] |

Specifically, we identified a phenotypic set indicating a general process of severe deterioration (i.e., continuous positive symptoms with marked and progressive impairment) that was associated with many SNP sets (e.g., SNP sets 75_67 and 56_30, with p values, 2.3E-13 and 2.55E-05, respectively; Table 7, FIG. 5A). Other SNP sets were associated with a general process of moderate deterioration (moderate or fluctuating impairment despite a continuous mixture of symptoms), as in SNP sets 14_6, and 42_37 (p values, 5F-04; Table 7, FIG. 5A). We identified specific clinical syndromes that were unambiguously associated with particular genotypic networks. For example, specific phenotypic sets differentiate among SNP sets even within the same network, which illustrate similar but not identical forms of multifinality in schizophrenia (e.g., 76_74 and 58_29; Table 7, FIG. 5A, blue lines). Particular phenotype sets can also distinguish SNP sets connected only by shared subjects (FIG. 5A, red lines). For example, SNP set 76_74 shares subjects with 56_30 and with 81_13; however, the latter SNP sets are associated with a specific phenotypic set not present in 76_74 (Table 7).

e) Positive and Negative Symptoms Differentiate Classes of Schizophrenia

Genotypic and phenotypic relationships could be grouped into eight classes of schizophrenia, as shown in FIG. 3B and Table 3. First, we identified SNP sets involving subjects with predominantly positive symptoms (e.g., 41_12 and 88_64) and few residual symptoms. Second, we identified SNP sets represented by predominantly negative and disorganized symptoms (e.g., 10_4 and 61_39), decreased psychosocial function, and continuous residual symptoms. Bizarre delusions and symptoms of cognitive and behavioral disorganization, such as thought insertion and disorganized speech among others, were accepted as fuzzy indicators of either positive or negative classes of schizophrenia but were considered to be more common in negative and disorganized classes (e.g., in Table 7, thought echo and commenting hallucinations in “negative schizophrenia” with phenotypic set 46_29 associated with SNP set 14_6). Third, several SNP sets harbor mixed positive and negative symptoms (e.g., 59_48 and 54_51). These three classes were enriched by considering the general severe and moderate patterns, which were frequent in several networks (FIG. 5B), as described above. Because the latter patterns appear in combination with a set of only positive symptoms (e.g., 81_13), both positive and negative symptoms (e.g., 75_67), and only negative symptoms (e.g., 19_2), we were able to classify schizophrenia into eight classes (FIG. 5B).

f) Replication of Results in Two Independent Samples

We tested the replicability of our findings in the MGS study by carrying out the same analyses of the genotypic and phenotypic architecture of schizophrenia in the CATIE and Portuguese Island samples. A total of 1,303 SNPs were shared between the selected SNPs in the MGS and CATIE samples, and 1,234 SNPs between the MGS and Portuguese Island samples. Imputed variants were not considered, to avoid possible biases.

Together, both samples reproduced at least 81% of the SNP sets at risk (see Table 9). In addition, most of the SNP sets replicated in the two PGC samples achieved risk values as high as those of the MGS sample (>70%: 70% of those identified exhibit >70% risk, and 90% show >60% risk. Some SNP sets exhibited slightly higher risk values than those in the MGS sample. The genotypic-phenotypic relations in CATIE and the Portuguese Island studies closely matched those observed in the MGS study (hypergeometric statistics, p values 2E-13 to 1E-03). The eight schizophrenia classes exhibited high reproducibility. For example, except for one relation (“−” in the MGS study and “+ and −” in CATIE; see Table 9), all relations exhibited similar positive and negative symptoms in the MGS study and CATIE. Three relations showed less specific symptoms in CATIE than in the MGS study, as expected because CATIE did not use the Diagnostic Interview for Genetic Studies.

TABLE 9
Summary of the Reproducibility of the Molecular Genetics of Schizophrenia
Dataset in the CATIE and the Portuguese Islands Studies

| Gain/nonGain | | | CATIE | | | | Portuguese | | |
|---|---|---|---|---|---|---|---|---|---|
| SNP | | | SNP | | | Symptom | SNP | | Symptom |
| sets | Risk | Symptoms | sets | | Risk | Variation* | sets | Risk | Variation* |
| 9_9 | 0.92 | − | 9_9 | 5_1 | 0.97 | | 40_40 | 0.67 | |
| 19_2 | 1.00 | moderate − | 19_2 | 25_7 | 1.00 | | 26_3 | 0.88 | |
| 21_8 | 0.71 | +− | 21_8 | 25_19 | 0.61 | general +− | 10_2 | 0.88 | |
| 81_13 | 0.95 | severe + | 81_13 | 12_3 | 0.60 | | | | |
| 22_11 | 0.75 | − | 22_11 | 16_10 | 0.71 | general − | 15_9 | 0.71 | |
| 25_10 | 0.70 | severe + | 25_10 | 33_28 | 0.70 | general +− | | | |
| 10_4 | 0.91 | − | 10_4 | 13_2 | 0.64 | | 35_11 | 0.86 | |
| 59_48 | 0.80 | +− | | | | | 36_18 | 0.68 | severe +− |
| 12_11 | 0.84 | moderate − | 12_11 | 14_9 | 0.70 | | 35_11 | 0.86 | |
| 56_30 | 0.88 | severe +− | 56_30 | 32_10 | 0.60 | | 35_31 | 0.83 | severe/moderate +− |
| 12_2 | 0.70 | − | 12_2 | 37_11 | 0.84 | | 14_5 | 0.88 | |
| 13_12 | 0.75 | − | 13_12 | 11_8 | 0.80 | | 29_13 | 0.70 | |
| 14_6 | 0.90 | moderate − | 14_6 | 12_12 | 0.60 | | 40_40 | 0.67 | |
| 16_10 | 0.73 | general − | 16_10 | 14_3 | 1.00 | | 14_5 | 0.88 | |
| 31_22 | 0.74 | +− | 31_22 | 25_16 | 0.71 | | 19_5 | 0.76 | |
| 41_12 | 0.76 | + | | | | | | | |
| 42_37 | 0.86 | moderate +− | 42_37 | 19_14 | 0.92 | | 25_21 | 0.74 | |
| 51_28 | 0.81 | moderate +− | | | | | | | |
| 76_74 | 0.71 | severe +− | 76_74 | 33_11 | 1.00 | | 40_37 | 0.78 | moderate |
| 52_42 | 0.70 | moderate − | 52_42 | 40_18 | 0.60 | − | 25_21 | 0.74 | +− |
| 54_51 | 0.70 | +− | | | | | 36_1 | 0.55 | no match |
| 56_19 | 0.73 | − | | | | | | | |
| 58_29 | 0.94 | − | 58_29 | 31_6 | 1.00 | | 32_6 | 0.65 | +− |
| 61_39 | 0.71 | − | | | | | | | |
| 65_25 | 0.86 | +− | | | | | | | |
| 90_78 | 0.83 | moderate − | 90_78 | 4_2 | 0.93 | | 3_1 | 0.62 | |
| 71_55 | 0.86 | +− | 71_55 | 35_11 | 0.65 | | 27_22 | 0.73 | |
| 75_31 | 0.73 | − | 75_31 | 39_30 | 1.00 | | 3_1 | 0.62 | |
| 75_67 | 0.71 | severe +− | 75_67 | 8_3 | 0.70 | | 23_5 | 0.76 | |
| 76_63 | 0.71 | general/mild | | | | | | | |
| 88_64 | 0.96 | + | 88_64 | 35_2 | 0.61 | | | | |
| 77_5 | 0.82 | severe + | | | | | 36_1 | 0.55 | no match |
| 81_3 | 0.71 | − | 81_3 | 16_10 | 0.71 | | 10_2 | 0.88 | −+ |
| 81_73 | 0.73 | − | 81_73 | 36_12 | 0.74 | | 27_23 | 0.73 | general − |
| 83_41 | 0.93 | general/mild | 83_41 | 39_3 | 0.60 | | | | |
| 85_23 | 0.73 | general/mild | | | | | | | |
| 85_84 | 0.74 | + | | | | | | | |
| 87_26 | 0.71 | general/mild | 87_26 | 38_30 | 0.50 | | 38_7 | 0.75 | general +− |
| 87_76 | 0.95 | moderate − | 87_76 | 3_3 | 0.50 | | 34_22 | 0.68 | |
| 87_84 | 0.74 | +− | 87_84 | 9_4 | 0.50 | | 40_9 | 1.00 | |
| 88_43 | 0.71 | moderate +− | 88_43 | 30_21 | 0.50 | | 15_11 | 0.74 | |
| 88_8 | 0.82 | − | 88_8 | 39_30 | 1.00 | | 39_31 | 0.56 | +− |

(*empty values indicates similar results to those corresponding to Gain/nonGain)

We found few differences when comparing the MGS and Portuguese Island studies (see Table 9), except differences in severity that preserved the sign of the symptoms. Three relations with negative symptoms in the MGS study exhibited negative and positive symptoms in the Portuguese Island sample (see Table 9). Only two SNP sets in the Portuguese Island sample had no significant crossmatch with the phenotypic features expected from the MGS study.

### 2. Example 2

We first identified sets of interacting single-nucleotide polymorphisms (SNPs) that cluster within subgroups of individuals (SNP sets) regardless of clinical status in the MGS Consortium study, employing our generalized factorization method combined with non-negative matrix factorization to identify candidates for functional clusters (see FIG. 2). This approach performs an unsupervised co-clustering of subjects together with distinguishing genotypic/phenotypic features based on the empirical data alone. We combined the Genetic Association Information Network (GAIN) and non-GAIN samples of the MGS study, which constitute one GWAS. The 4,196 cases and 3,827 controls in the MGS study were combined to identify SNP sets. We had data of good quality on 696,788 SNPs on these cases and controls, and from these we preselected 2,891 SNPs that had at least a loose association (p values<1.0.times.10⁻²) with a global phenotype of schizophrenia. SNP sets were labeled by a pair of numbers based on the order in which they were chosen by the algorithm. Each SNP set was composed of a particular group of subjects described by a particular set of homozygotic and/or heterozygotic alleles; subjects and/or SNPs may be present in more than one set. The SNP sets identified by our generalized factorization method are optimal clusters of SNPs in particular subjects that encode AND/OR interactions between SNPs and subjects (FIG. 3A-F, Table 1; see also FIG. 4). These SNP sets and their relations with one another characterize the genetic architecture of schizophrenia-associated SNPs in all subjects, including cases and controls (FIG. 1A).

Second, we examined the risk of schizophrenia for each SNP set and identified those with high risk. The statistical significance of the association of SNP sets with schizophrenia was calculated using the SNP-Set Kernel Association Test (SKAT) program, which properly accounts for multiple comparisons.

Third, we checked for significant overlap among SNP sets in terms of subjects and/or SNPs using hypergeometric statistics (see FIG. 2). This allowed us to characterize the relations among SNP sets and to identify SNP sets that were connected to each other by having certain SNPs or subjects in common, thereby composing genotypic networks. Disjoint networks shared neither SNPs nor subjects, as expected if schizophrenia is a heterogeneous group of diseases.

Fourth, we identified sets of distinct clinical features that cluster in particular cases with schizophrenia (i.e., phenotypic sets or clinical syndromes) without regard for their genetic background, again using non-negative matrix factorization. Ninety-three clinical features of schizophrenia from interviews based on the Diagnostic Interview for Genetic Studies, as well as the Best Estimate Diagnosis Code Sheet submitted by GAIN/non-GAIN to dbGaP, were initially considered with the MGS sample. The Diagnostic Interview for Genetic Studies was utilized for the Portuguese Island samples. Corresponding features were extracted in CATIE kern the Positive and Negative Syndrome Scale, the Quality of Life Questionnaire, and the Structured Clinical Interview for DSM-IV. These phenotypic sets and their relations with one another characterize the phenotypic architecture of schizophrenia (FIG. 1B).

Fifth, we tested whether SNP sets were associated with distinct phenotypic sets in the MGS sample, and we tested the replicability of these relations in the two other independent studies. Replication was evaluated in terms of replication of the SNP sets and their corresponding risk, as well as the relationships between SNP sets and phenotypic sets. In the samples that used the Diagnostic Interview for Genetic Studies (the MGS and Portuguese Island samples), the specific phenotypic features can be compared. Since the CATIE study did not use the Diagnostic Interview for Genetic Studies, we estimated the corresponding symptoms from available phenotypic data (based on the Positive and Negative Syndrome Scale, the Quality of Life Questionnaire, and the Structured Clinical Interview for DSM-IV). Genotypic and phenotypic data were available for 738 cases in CATIE and 346 cases in the Portuguese Island study. The significance of cohesive relations among SNP sets and clinical syndromes was tested using hypergeometric statistics. The relations between the genotypic and phenotypic clusters characterize the genotypic-phenotypic architecture (FIG. 1C).

a) Genomics Dataset: Gain and NonGain Studies

We first investigated the architecture of schizophrenia (SZ) using the Gain and NonGain genome wide association studies (GWAS) as our main targets, which are coherent case-control studies performed in a single lab under similar conditions. This study contains data from 8023 subjects, 4196 patients and 3827 controls, combining data from Euro-American ancestry (EA) and African-American ancestry (AA). Genotyping was carried using the Affymetrix 6.0 array, which assays 906,600 SNPs.

This study was originally performed in part at Washington University. Study population, ascertainment, phenomics and genomic datasets, as well as other information relative to this study can be accessed in the dbGaP by their identifiers: phs000021.v3.p2 and phs000167.vl.p1 for GAIN and NonGAIN projects, respectively.

The genotype data was codified in a matrix [SNPs.times.subjects], where the columns and rows correspond to subjects and SNPs, respectively. In each cell of the matrix, the value for the corresponding SNP and subject is assigned as 1, 2, and 3 for the SNP allele values AA, AB, and BB, respectively. Missing values were initialized by 0.

b) Data Cleaning

The quality control (QC) of the genotypic data was performed following the steps removing consequently all the SNPs satisfying the next criteria:

1) SNP call rate <95% in either GAIN or NonGAIN or combined datasets.

2) Hardy-Weinberg (HWE) p-value <10E-06 based on control samples in either GAIN or NonGAIN or combined, (using only females for chr X SNPs).

3) Minor Allele Frequency (MAF) <1% in combined dataset.

4) Failed plate effect test in GAIN, NonGAIN or combined dataset.

5) MENDEL errors>2 in either GAIN or NonGAIN.

6) >1 disconcordant genotypes in either GAIN 29 duplicates or NonGAIN 32 duplicates.

7) >2 disconcordant genotypes for 93 (=3.times.31 trios) samples genotyped in both GAIN and NonGAIN.

A total of 209,321 SNPs were excluded due to the restrictions described above from the total 906,109 SNPs genotyped. Therefore, 696,788 SNPs passed the QC filters. Then, 2891 SNPs were pre-selected to reduce the large search space using the logistic association function included in the PLINK software suite, taking sex and ancestry as co-variates, and establishing a generous threshold (p-value <0.01). This threshold was established as 0.01 because this is approximately the value used in the supplementary tables reported in previously for AA, EA and AA-EA analyses.

c) Methodology: A Divide & Conquer Strategy to Dissect a GWAS into the Genotypic-Phenotypic Architecture of a Disease

To uncover the architecture of SZ we applied a “Divide & Conquer” strategy (see FIG. 2) that is commonly used in computer science to solve complex problems such as those of proteomics and transcriptomics and cancer identification. Here we applied this strategy to dissect a single GWAS into multiple genotypic and/or phenotypic networks, as an attempt to extract the maximum information even from one dataset.

The “divide” step deconstructs genotypic and phenotypic data independently, and explores multiple local patterns (i.e., SNP sets and phenotypic sets). We used non-negative matrix factorization methods that have been applied to characterize complex genomic and social profiles, and generalized them to approach GWA data in a purely data-driven and unbiased fashion.

Thus, our systematic grouping strategy is not directed by previous knowledge of polygenic involvement in SZ, does not limit subjects to only one SNP set, and does not predefine the number of SNP sets, avoiding possible biases and 4 assumptions that relationships are linear, regular, or random. Unlike other approaches, we do not constrain SNP sets to a particular genome feature or to be in linkage disequilibrium (LD), and the phenotypic status of the subjects is not considered in SNP set formation (i.e., it is unsupervised).

After incorporating phenotypic status a posteriori within each set (e.g., cases and controls), we establish their statistical significance with powerful and well-founded test methods that perform the appropriate corrections for the use of SNP sets, as well as provide an unbiased risk surface of disease to test predictions.

The “conquer” step consists of three stages. First, assembling the uncovered local components of the genotypic architecture into genotypic networks of SNP sets, where two SNP sets are connected if they (i) comprise different sets of subjects described by similar sets of SNPs, (ii) and/or if they have similar sets of subjects but characterized by distinct sets of SNPs, (iii) and/or if one of the two SNP sets contains a subset of subjects and SNPs of the other SNP set. Second, optimally combining the local components of the phenotypic architecture (i.e., phenotypic sets) with the genotypic sets to expose the joint genotypic-phenotypic architecture of the disease. Third, evaluating complexity in the pathway from SNP sets to phenotypic sets; some connected SNP-set networks may be candidates to converge to equifinality, whereas other disjoint networks can lead to multifinality (i.e., recognizing a collection of diseases).

Finally, we carried out independent analyses to test for possible confirmations of the heterogeneous architecture of SZ. We performed bioinformatics analysis of genes related to each uncovered relationship and their molecular consequences. Then, we computationally and clinically evaluated the genotypic-phenotypic relations to determine sub-classes of the disease based on whether the groups of SZ patients varied on a range of positive and/or negative symptoms.

d) Method

Given a genotype database from a GWAS represented as a matrix [SNPs.times.subjects], the method for dissecting the architecture of a disease is composed of 6 steps (FIG. 2), where a SNP set is a sub-matrix harboring subjects described by a set of SNPs sharing similar allele values:

(1) Identify SNP Sets

Use a Generalized Factorization Method (GFM) to dissect a GWAS into SNP sets (see below for a mathematical description of NMF). The GFM applies recurrently a basic factorization method to generate multiple matrix partitions using various initializations with different maximum numbers of sub-matrices k (e.g., 2.Itoreq.k.Itoreq. n), where n is the number of subjects, and thus, avoids any pre-assumption about the ideal number of sub-matrices (see below for a rationale about the use of unconstrained number of sub-matrices or clusters). Particularly, we developed a new version of the basic bioNMF method termed Fuzzy Nonnegative Matrix Factorization method (FNMF), and used it as a default basic factorization method. FNMF allows overlapping among sub-matrices, and detection of outliers. For each run of the basic factorization method (2.Itoreq.k.Itoreq. n)), all sub-matrices are selected to compose a family of genotypic SNP sets G_k={G_k_i}, where 1.Itoreq.Itoreq.k Each G_k family, as well as all families together G={G_k} for all k, may include overlapped, partially redundant and different-size sub-matrices.

(2) Perform a Statistical Analysis of SNP Sets

Use the R-project package SKAT to evaluate the significance of each SNP set. We used the identity-by-state (IBS) as a kernel because the analyzed variants are not rare but common, and therefore, using the “weighted IBS” kernel would not be adequate. Since the SNP sets can overlap, we run each one separately. The sex and ancestry of the subjects were used as covariates, and the default remaining parameters were utilized.

(3) Map a Disease Risk Function

3.1) Estimate the risk of a SNP set. Incorporate a posteriori the status of the subjects in a weighted average of epidemiological risks function of all subjects in a particular SNP set:

$$Risk ( G\_k \_i ) = \frac{Σιϵ ST \vert {ST}_{i} \vert {Q}_{i}}{Σιϵ ST \vert {ST}_{i} \vert} \qquad ( 1 )$$

with ST being the status of the instances (i.e., cases and controls) and Q the weights given by epidemiologic risk of SZ in each SNP set (e.g., 0 and 1 for controls and cases; 0.01, 0.1 and 1 for cases, relatives and controls, respectively).

3.2) Plot the genotype risk surface of the disease. Encode each SNP set into a 3-tuple (X, Y, Z), where SNP sets are placed along the x- and y-axis using a dendrogram based on their distances in the SNP (see step 4.1, M_{SNPs}) and subject (see step 4.2, W_{subjects}) domains, respectively, and Z is the risk variable calculated in (eqn. 1). Interpolate and plot the surface by using the tgp and latticeExtra packages in R-project, respectively.

(4) Discover and Encode Relations Among SNP Sets into Topologically Organized Networks

4.1) Identify optimal and non-redundant relations between SNP sets based on their shared SNPs and, separately, based on their shared subjects. Overlap of SNP sets refers to overlap of SNP loci, which, in most of our cases leads also to sharing allele values. The sharing of alleles is fully true when there is overlap of both loci and subjects.

4.1.1) Co-cluster all G_k_i SNP sets within G by calculating the pairwise probability of intersection among them using the Hypergeometric statistics (PI_{hyp}) on intersected SNPs: PI_{hyp} (G_e_q, G_r_w) (eqn. 2, see below), where q and w are SNP sets generated in runs with a maximum of e and r number of sub-matrices, respectively, and p in (eqn. 2) is the intersection of SNPs. Then, encode all PI_{hyp}-values, which encompass—in some extent—the distance between SNP sets, in a square [SNP set.times.SNP set] matrix M_{SNPs}.

4.1.2) Repeat the Former Procedure Based on Intersected Subjects and Determine the M_{subjects} Matrix.

4.1.3) Eliminate highly overlapped/redundant SNP sets, which may occur due to the repetitive application of the factorization methods, by deleting all except one SNP set where Max(M_{SNPs}[i,j], M_{subjects}[i, j])≤δ, for all i, j indices in the matrices. Here, we used δ10E-15.

4.2) Organize SNP Sets Sharing SNPs and/or Subjects into Subnetworks.

4.2.1) For each row i and column j in M_{SNPs}, M_{SNPs}[i, j]≤ϕ, connect the corresponding SNP sets with a blue line, indicating that they share SNPs. In our case, we established ϕ≤3E-09. This value results from adjusting typical p-value of 0.01 by the total number of pairwise comparisons between all possible generated SNP sets [4094.times.4094, by using the Hypergeometric-based test (eqn. 2)], likewise a Bonferroni correction.

4.2.2) For Each Row i and Column j in M_{SNPs}, M_{subjects}[i_{i, j}]≤ϕ, Connect the Corresponding SNP Sets with a Red Line, Indicating that they Share Subjects.

(5) Identify Genotype-Phenotype Latent Architectures

5.1) Create a phenotype database. Dissect the questionnaire based on DIGS and the Best Estimate Diagnosis into individual variables. The variables can be numerical or categorical. For efficiency, in our case, each categorical variable was re-coded into different variables with binary values. The phenotype data was codified in a [phenotype features.times.subjects] matrix, where the columns and rows correspond to subjects and phenotypic features, respectively. In our case, because the phenotypic features from cases are different from those from the controls, we only considered the cases.

5.2) Identify phenotype sets (Implemented in the PGMRA web server). Use step 1) with the phenotype database from 5.1) instead of genotype database to identify phenotypic sets, where a phenotypic set is a sub-matrix harboring subjects described by a set of phenotypic features sharing similar values (i.e., P_h_j, where j is a phenotypic set generated in a run with a maximum of h number of sub-matrices).

5.3) Identify genotypic-phenotypic relations. Co-cluster SNP sets with phenotype sets into relations using the Hypergeometric statistics on intersected subjects, where R_{i,j}=PI_{hyp} (G_k_i, P_h_j) (see below, eqn, 2), G_k_i, P_h_j are SNP and phenotypic sets, respectively, and p in (see below, eqn. 2) is the intersection of subjects. Relations R_{i,j}<T constitute the genotypic-phenotypic architecture of a disease. The significance of the relations (T) was established by the p-value (PI_{hyp}) provided by the Hypergeometric-based test (see below, eqn. 2).

(6) Annotate Genes, and Symptoms/Classes of Disease

6.1) Map latent architectures to the genome. For each SNP set, we analyze all genes being affected by each of the SNPs in a SNP set. This analysis includes the SNP location with respect to a gene, the type and number of genes being affected by one SNP (e.g., protein coding, ncRNA genes, and pseudogenes), the possible transcripts being affected and the position where they are affected (e.g. coding region, distance to stop codon, splicing site, intron, UTR, etc.), and finally promoter and intergenic regions' features are inspected for annotation if the SNP does not overlap with a gene then regulatory. Moreover the possible molecular consequences of each SNP over function is provided, as well as, the corresponding allele values. Annotation information was obtained from the Haploreg DB and from the Ensembl and NCBI web services (see below).

Once we obtain the information described above, we generate a list of relevant genes that it is used to query the Nextbio web site in order to find diseases related to each gene. NextBio uses proprietary algorithms to calculate and rank the diseases and drugs most significantly correlated with a queried gene, where rank values are established relative to the top-scored result (score set to 100). Therefore, although a low-scoring result might have less statistical significance compared to the top-ranked result, it could still have real biological relevance. In our case, out of all possible diseases, only the categories “Mental Disorders” and “Brain and Nervous System Disorders” were considered from the “Disease Atlas”.

6.2) Map Latent Architectures to Disease Symptoms or Classes of Disease.

6.2.1) Characterize each phenotypic feature by the type of symptoms that they represent. First, explore the distribution of the phenotypic dataset by calculating the principal components (PCA, Statistic Toolbox, Matlab R2011a) of the Phenotypic sample, where the columns are subjects and the rows are the phenotypic variables. Here we used as many PCs as needed to account for the 75% of the sample (5 PCs). In the sample with the phenotypic features as rows and the PCs as columns, cluster the rows by using Hierarchical Clustering (Correlation and Maximum as inter and intra-clustering measurements, Statistic Toolbox, Matlab R2011a). This clustering process generates natural groups of features constitution natural partition hypotheses about the phenotypic features. Second, evaluate each phenotypic feature included in the phenotype database using curated information from experts and the literature and individually classify each item based on the symptoms as purely positive (1), purely negative (4), primarily positive (2) or primarily negative symptoms (3).

6.2.2) For each phenotypic set P_h_j related to a SNP set G_k_i in R_{i,j} re-code each phenotypic feature by their positive and/or negative symptoms in a [R_{i,j} X phenotypic feature] matrix M_{symptons}.

6.2.3) Cluster the encoded features by factorizing M.sub.symptoms into sub matrices using a basic factorization method with a maximum number of sub-matrices defined by the Cophenetic index.

6.2.4) Label the latent classes of the diseases. (The current results provided 8 classes, see FIG. 5B.)

e) Mathematical Description of NMF

We consider a GWA data set consisting of a collection of NM subject samples (e.g., cases and controls), which we use to characterize a domain of genotypic (SNPs) states of interest. The data are represented as an nM.times.NM matrix M, whose rows contain the allele values of the nM SNPs in the NM subject samples. Using the FNMF, we find a manageable number of SNP sets k, positive local and linear combinations of the NM subjects and the nM SNPs, which can be used to distinguish the genetic profiles of the subtypes contained in the data set. Mathematically, this corresponds to finding an approximate factoring, M.about.WM.times.HM, where both factors have only positive entries and hence are biologically meaningful. WM is an nM.times.k matrix that defines the SNP set decomposition model whose columns specify how much each of the subjects contributes to each of the k SNP set. HM is a k.times.NM matrix whose entries represent the SNP allele values of the k SNP sets for each of the NM subject samples. In our implementation either a subject or SNP can belong to more than one SNP set.

f) Rationale for the Use of Unconstrained Number of Clusters

Although there are many indices that estimate the appropriate number of clusters for a given partition, we previously demonstrated that they are often constrained by the type of cluster, and metrics utilized. Therefore, it is hard to obtain a consensus from all of them, and they very often provide contradictory results. Moreover, given that the target of the method is to obtain good relations among clusters from different domains of knowledge, it is not known which cluster in one domain will match another cluster in a different domain, and thus, the more varied the clusters, the better the chance of identifying posterior inter-domain relations. To do so, we repeatedly applied a basic clustering method in one domain of knowledge to generate multiple clustering results using various numbers of clusters initializations (from 2 to where n is the number of observations/subjects).

g) Coincident Test Index: Co-Clustering and Establishing Relations Between Sets

The degree of overlapping between two SNP or phenotypic sets was assessed by calculating the pairwise probability of intersection among them based on the Hypergeometric distribution (PI_{hyp}):

$$Risk ( G\_k \_i ) = \frac{Σιϵ ST \vert {ST}_{i} \vert {Q}_{i}}{Σιϵ ST \vert {ST}_{i} \vert} \qquad ( 1 )$$

where p observations belong to a set of size h, and also belong to a set of size n; and g is the total number of observations. Therefore, the lower the PI_{hyp}, the higher the overlapping. The (p-value of) hypergeometric “test” is used here as a measure of association strength. The real test (p-value) of genotypic-phenotypic relationship was provided through the permutation procedure.

h) Permutation Test for Genotypic-Phenotypic Relations

Statistical significance reported values were obtained by 4000 independent permutations due to the comparisons between all possible generated SNP sets (i.e., 4094, from 2 to √n), and possible overlapped SNP sets here identified were generated as following: a) assign random subjects to a phenotypic cluster of random size; b) assign random subjects to a genotype cluster (set) of random size; c) calculate the Hypergeometric statistic (PI_{hyp}, eqn 2) between the two clusters and accumulate the value. These values form an empirical null distribution of PI_{hyp} used to calculate the empirical p-value of an identified relation. All optimal relations had empirical p-value≤value<4.7E-03.

i) Resampling Statistics of the NMF Sets

To guarantee the submatrices converge to the same solution and, given the non-deterministic nature of NMF and its dependence on the initialization of the W and H vectors, we run it 40 times for any k maximum number of allowed submatrices with different random initializations of the vectors to select those that that best approximates the input matrix. Besides, to estimate the precision of sample statistics of the SNP sets (variance of the W and H vectors) we use a leave-one-out technique (jackknifing) 1000 times on the SNP domain and obtained a 94% support for all identified sets with an average variance of c.a.±0.5% of their corresponding W and H vectors. Finally, we already modified this sampling technique to ensure the occurrence of the remaining sets after a leave-one-set-out and applied to our current sample with >90% of support.

j) Data Reduction

Data reduction was not applied because many Principal Components (PCs) were required in this study, consistent with the demonstration that clustering with the PCs instead of the original variables does not necessarily improve, and often degrades, cluster quality and interpretability. Moreover, likewise in phenomics, partially correlated variables reinforce the association and clarify the symptom identification process. Therefore, we used initially 93 phenotypic features listed in Appendix I, catalog of phenotypic features.

Briefly, phenotypic features used in the search process included all available data from the interviews. That is, replies to DIGS as well as to the Best Estimate Diagnosis code sheet submitted by GAIN/NONGAIN to dbGaP. Unbiased compilation of all of the data resulted in an initial set of 93 features. To capture items specific for positive and negative schizophrenia and avoid symptoms with affective elements, symptoms reported by acutely psychotic patients, and redundant items the original set of was pruned based on authors clinical experience, and computational feature validation (above in Method, step 6.2.1).

### 3. Bioinformatics Analysis: Genotypic Organization of the SZ Architecture Accounts for Multiple Genetic Sources of the Disease

Given that genotypic SZ architecture is composed of multiple networks, we matched each SNP set composing these networks with the corresponding genomic location of their SNPs, and in turn, with the mapped genes (FIG. 5A, Table 2) to investigate what these SNP sets represent in terms of genomic information. We uncovered a list of genes with many different functions and distinct roles in different molecular networks (Tables 2-4).

### 4. A Single SNP Set can Map Different Classes of Genes, Located in Different Chromosomes, and Distinct Types of Genetic Variants

The uncovered SNP sets contain SNPs that map gene, promoter and intergenic regions (IGRs) located anywhere in the genome, without being constrained by genomic features such as a specific gene or haplotype (28). For example, SNP set 81_13 contains SNPs in chromosomes 8 and 16, whereas SNP set 42_37 has SNPs located in chromosomes 2 and 11 (FIG. 5A, Table 2). SNP set 75_67 has SNPs in chromosomes 4, 8, 15, and 16, among others, and maps >30 genes, as expected by its generality (FIG. 5A, Table 2). The latter SNP set is in the same network as SNP sets 56_30, 76_74 and 81_13, and thus shares some genes with them. Despite being in the same network, the last three SNP sets map to particular genes specific to each of them (FIG. 5A, Table 2).

In addition to mapping genes in different locations, SNP variants within the SNP sets affect distinct classes of genes including protein-coding, non-coding (ncRNA) genes, and pseudogenes, with different molecular consequences depending on the altered region (coding, UTRs, introns, Table 4). For example, only 25% of SNPs in SNP set 75_67 affect protein-coding genes, which are the targets most often considered in genetic studies of diseases, whereas another 25% of SNPs affect ncRNAs (lincRNAs, antisense RNAs, miRNAs). One of these lincRNAs is SOX2-OT, which is associated with >15 possible transcripts (Table 4); it is contained inside the SOX2 transcription factor that is predominantly expressed in the human brain where SOX2-OT is also highly enriched.

TABLE 4
Molecular Consequences of SNP Variants.

| | | | | | Regulatory element | Ensembl gene | | EntrezGene |
|---|---|---|---|---|---|---|---|---|
| Variation | Group | Location | Allele | Gene | (Ensembl) | name | UniProt ID | ID |
| rs10488268 | 9_9 | 7: 83733446 | T | ENSG00000075213 | | SEMA3A | SEMA3A | 10371 |
| rs11631112 | 9_9 | 15: 88659906 | T | ENSG00000140538 | | NTRK3 | NTRK3 | 4916 |
| rs13228082 | 9_9 | 7: 83726968 | G | ENSG00000075213 | | SEMA3A | SEMA3A | 10371 |
| rs16941261 | 9_9 | 15: 88655520 | C | ENSG00000140538 | | NTRK3 | NTRK3 | 4916 |
| rs17298417 | 9_9 | 7: 83730162 | C | ENSG00000075213 | | SEMA3A | SEMA3A | 10371 |
| rs3784405 | 9_9 | 15: 88688010 | C | ENSG00000140538 | | NTRK3 | NTRK3 | 4916 |
| rs3784405 | 9_9 | 15: 88688010 | C | ENSG00000259183 | | RP11-356B18.1 | | |
| rs3801629 | 9_9 | 7: 83734593 | G | ENSG00000075213 | | SEMA3A | SEMA3A | 10371 |
| rs6496466 | 9_9 | 15: 88717708 | C | ENSG00000140538 | | NTRK3 | NTRK3 | 4916 |
| rs7806871 | 9_9 | 7: 83727983 | G | ENSG00000075213 | | SEMA3A | SEMA3A | 10371 |
| rs994068 | 9_9 | 15: 88666646 | C | ENSG00000140538 | | NTRK3 | NTRK3 | 4916 |
| rs995866 | 9_9 | 7: 83745039 | C | ENSG00000075213 | | SEMA3A | SEMA3A | 10371 |
| rs11630338 | 9_9 | 15: 88661632 | C | ENSG00000140538 | | NTRK3 | NTRK3 | 4916 |
| rs2114252 | 9_9 | 15: 88664676 | A | ENSG00000140538 | | NTRK3 | NTRK3 | 4916 |
| rs3801616 | 9_9 | 7: 83721051 | A | ENSG00000075213 | | SEMA3A | SEMA3A | 10371 |
| rs4887364 | 9_9 | 15: 88660115 | C | ENSG00000140538 | | NTRK3 | NTRK3 | 4916 |
| rs727650 | 9_9 | 7: 83735838 | G | ENSG00000075213 | | SEMA3A | SEMA3A | 10371 |
| rs727651 | 9_9 | 7: 83735893 | G | ENSG00000075213 | | SEMA3A | SEMA3A | 10371 |
| rs764116 | 9_9 | 7: 83738481 | A | ENSG00000075213 | | SEMA3A | SEMA3A | 10371 |
| rs991728 | 9_9 | 15: 88662946 | G | ENSG00000140538 | | NTRK3 | NTRK3 | 4916 |
| rs11159957 | 10_4 | 14: 90715972 | A | | | | | |
| rs11621045 | 10_4 | 14: 90714003 | A | | ENSR00001459588 | | | |
| rs11621045 | 10_4 | 14: 90714003 | A | | | | | |
| rs11623741 | 10_4 | 14: 90804474 | G | | | | | |
| rs11628812 | 10_4 | 14: 90713720 | C | | | | | |
| rs7150093 | 10_4 | 14: 90724661 | G | ENSG00000100764 | | PSMC1 | PSMC1 | 5700 |
| rs7154695 | 10_4 | 14: 90795705 | G | ENSG00000119720 | | C14orf102 | C14ORF102 | 55051 |
| rs11159957 | 12_11 | 14: 90715972 | A | | | | | |
| rs11621045 | 12_11 | 14: 90714003 | A | | ENSR00001459588 | | | |
| rs11621045 | 12_11 | 14: 90714003 | A | | | | | |
| rs11623741 | 12_11 | 14: 90804474 | G | | | | | |
| rs11626869 | 12_11 | 14: 90788985 | G | ENSG00000119720 | | C14orf102 | C14ORF102 | 55051 |
| rs11628812 | 12_11 | 14: 90713720 | C | | | | | |
| rs7150093 | 12_11 | 14: 90724661 | G | ENSG00000100764 | | PSMC1 | PSMC1 | 5700 |
| rs7154695 | 12_11 | 14: 90795705 | G | ENSG00000119720 | | C14orf102 | C14ORF102 | 55051 |
| rs11159956 | 12_11 | 14: 90715890 | C | | | | | |
| rs17188598 | 12_11 | 14: 90722473 | T | ENSG00000100764 | | PSMC1 | PSMC1 | 5700 |
| rs3783838 | 12_11 | 14: 90733012 | G | ENSG00000100764 | | PSMC1 | PSMC1 | 5700 |
| rs7146640 | 12_11 | 14: 90720114 | A | ENSG00000100764 | | PSMC1 | PSMC1 | 5700 |
| rs10030713 | 12_2 | 4: 95238536 | C | ENSG00000163106 | | HPGDS | PGDS | 27306 |
| rs12646184 | 12_2 | 4: 95183216 | T | ENSG00000163104 | | SMARCAD1 | SMARCAD1 | 56916 |
| rs17021364 | 12_2 | 4: 95047893 | C | | ENSR00001433195 | | | |
| rs17021364 | 12_2 | 4: 95047893 | C | ENSG00000246541 | | RP11-363G15.2 | | |
| rs2059606 | 12_2 | 4: 95255278 | A | ENSG00000163106 | | HPGDS | PGDS | 27306 |
| rs2664871 | 12_2 | 4: 95146281 | T | ENSG00000163104 | | SMARCAD1 | SMARCAD1 | 56916 |
| rs6532482 | 12_2 | 4: 95277414 | G | | | | | |
| rs6839224 | 12_2 | 4: 95279214 | G | | | | | |
| rs11097407 | 12_2 | 4: 95146135 | C | ENSG00000163104 | | SMARCAD1 | SMARCAD1 | 56916 |
| rs1991316 | 12_2 | 4: 95268272 | T | ENSG00000163106 | | HPGDS | PGDS | 27306 |
| rs2059605 | 12_2 | 4: 95255212 | C | ENSG00000163106 | | HPGDS | PGDS | 27306 |
| rs2087170 | 12_2 | 4: 95162960 | G | ENSG00000163104 | | SMARCAD1 | SMARCAD1 | 56916 |
| rs2632401 | 12_2 | 4: 95147055 | G | ENSG00000163104 | | SMARCAD1 | SMARCAD1 | 56916 |
| rs1144918 | 13_12 | 14: 89102558 | C | ENSG00000165521 | | EML5 | EML5 | 161436 |
| rs11845781 | 13_12 | 14: 89276431 | T | | | | | |
| rs1287660 | 13_12 | 14: 89286845 | G | ENSG00000165533 | | TTC8 | TTC8 | 123016 |
| rs1287660 | 13_12 | 14: 89286845 | G | ENSG00000200653 | | U4 | | |
| rs12880096 | 13_12 | 14: 89218815 | C | ENSG00000165521 | | EML5 | EML5 | 161436 |
| rs1956411 | 13_12 | 14: 89134360 | T | | ENSR00001459464 | | | |
| rs1956411 | 13_12 | 14: 89134360 | T | ENSG00000165521 | | EML5 | EML5 | 161436 |
| rs4904448 | 13_12 | 14: 88852166 | A | | ENSR00000099273 | | | |
| rs4904448 | 13_12 | 14: 88852166 | A | ENSG00000042317 | | SPATA7 | SPATA7 | 55812 |
| rs7147796 | 13_12 | 14: 89228569 | G | ENSG00000165521 | | EML5 | EML5 | 161436 |
| rs10132509 | 13_12 | 14: 89203781 | G | ENSG00000165521 | | EML5 | EML5 | 161436 |
| rs10140896 | 13_12 | 14: 89218538 | G | ENSG00000165521 | | EML5 | EML5 | 161436 |
| rs1287825 | 13_12 | 14: 89105536 | G | ENSG00000165521 | | EML5 | EML5 | 161436 |
| rs3784405 | 14_6 | 15: 88688010 | C | ENSG00000140538 | | NTRK3 | NTRK3 | 4916 |
| rs3784405 | 14_6 | 15: 88688010 | C | ENSG00000259183 | | RP11-356B18.1 | | |
| rs994068 | 14_6 | 15: 88666646 | C | ENSG00000140538 | | NTRK3 | NTRK3 | 4916 |
| rs1105442 | 14_6 | 15: 88724647 | T | ENSG00000140538 | | NTRK3 | NTRK3 | 4916 |
| rs11630338 | 14_6 | 15: 88661632 | C | ENSG00000140538 | | NTRK3 | NTRK3 | 4916 |
| rs11631112 | 14_6 | 15: 88659906 | T | ENSG00000140538 | | NTRK3 | NTRK3 | 4916 |
| rs12911150 | 14_6 | 15: 88668691 | G | ENSG00000140538 | | NTRK3 | NTRK3 | 4916 |
| rs16941261 | 14_6 | 15: 88655520 | C | ENSG00000140538 | | NTRK3 | NTRK3 | 4916 |
| rs2114252 | 14_6 | 15: 88664676 | A | ENSG00000140538 | | NTRK3 | NTRK3 | 4916 |
| rs4887364 | 14_6 | 15: 88660115 | C | ENSG00000140538 | | NTRK3 | NTRK3 | 4916 |
| rs6496466 | 14_6 | 15: 88717708 | C | ENSG00000140538 | | NTRK3 | NTRK3 | 4916 |
| rs991728 | 14_6 | 15: 88662946 | G | ENSG00000140538 | | NTRK3 | NTRK3 | 4916 |
| rs10030713 | 16_10 | 4: 95238536 | C | ENSG00000163106 | | HPGDS | PGDS | 27306 |
| rs12646184 | 16_10 | 4: 95183216 | T | ENSG00000163104 | | SMARCAD1 | SMARCAD1 | 56916 |
| rs17021364 | 16_10 | 4: 95047893 | C | | ENSR00001433195 | | | |
| rs17021364 | 16_10 | 4: 95047893 | C | ENSG00000246541 | | RP11-363G15.2 | | |
| rs2059606 | 16_10 | 4: 95255278 | A | ENSG00000163106 | | HPGDS | PGDS | 27306 |
| rs2664871 | 16_10 | 4: 95146281 | T | ENSG00000163104 | | SMARCAD1 | SMARCAD1 | 56916 |
| rs6532482 | 16_10 | 4: 95277414 | G | | | | | |
| rs6839224 | 16_10 | 4: 95279214 | G | | | | | |
| rs11097407 | 16_10 | 4: 95146135 | C | ENSG00000163104 | | SMARCAD1 | SMARCAD1 | 56916 |
| rs1991316 | 16_10 | 4: 95268272 | T | ENSG00000163106 | | HPGDS | PGDS | 27306 |
| rs2059605 | 16_10 | 4: 95255212 | C | ENSG00000163106 | | HPGDS | PGDS | 27306 |
| rs2059606 | 16_10 | 4: 95255278 | A | ENSG00000163106 | | HPGDS | PGDS | 27306 |
| rs2087170 | 16_10 | 4: 95162960 | G | ENSG00000163104 | | SMARCAD1 | SMARCAD1 | 56916 |
| rs2632401 | 16_10 | 4: 95147055 | G | ENSG00000163104 | | SMARCAD1 | SMARCAD1 | 56916 |
| rs10819000 | 19_2 | 9: 127619553 | G | ENSG00000136918 | | WDR38 | WDR38 | 401551 |
| rs10819000 | 19_2 | 9: 127619553 | G | ENSG00000136942 | | RPL35 | RPL35 | 11224 |
| rs10819000 | 19_2 | 9: 127619553 | G | ENSG00000136950 | | ARPC5L | ARPC5L | 81873 |
| rs10819019 | 19_2 | 9: 127750409 | G | ENSG00000173611 | | SCAI | SCAI | 286205 |
| rs10986471 | 19_2 | 9: 127635713 | G | ENSG00000136935 | | GOLGA1 | GOLGA1 | 2800 |
| rs10986471 | 19_2 | 9: 127635713 | G | ENSG00000136950 | | ARPC5L | ARPC5L | 81873 |
| rs388704 | 19_2 | 9: 127801357 | T | ENSG00000173611 | | SCAI | SCAI | 286205 |
| rs634710 | 19_2 | 9: 127661645 | A | ENSG00000136935 | | GOLGA1 | GOLGA1 | 2800 |
| rs634710 | 19_2 | 9: 127661645 | A | ENSG00000264641 | | AL354928.1 | | |
| rs640052 | 19_2 | 9: 127647800 | A | ENSG00000136935 | | GOLGA1 | GOLGA1 | 2800 |
| rs640052 | 19_2 | 9: 127647800 | A | ENSG00000199313 | | U4 | | |
| rs687434 | 19_2 | 9: 127643456 | C | ENSG00000136935 | | GOLGA1 | GOLGA1 | 2800 |
| rs687434 | 19_2 | 9: 127643456 | C | ENSG00000136950 | | ARPC5L | ARPC5L | 81873 |
| rs7031479 | 19_2 | 9: 127686126 | T | ENSG00000136935 | | GOLGA1 | GOLGA1 | 2800 |
| rs7022663 | 19_2 | 9: 127673385 | C | ENSG00000136935 | | GOLGA1 | GOLGA1 | 2800 |
| rs13413863 | 21_8 | 2: 22615313 | G | ENSG00000234207 | | AC096570.2 | | |
| rs13424767 | 21_8 | 2: 22612275 | C | ENSG00000231200 | | AC068490.2 | | |
| rs13424767 | 21_8 | 2: 22612275 | C | ENSG00000234207 | | AC096570.2 | | |
| rs1396725 | 21_8 | 2: 22612638 | A | ENSG00000231200 | | AC068490.2 | | |
| rs1396725 | 21_8 | 2: 22612638 | A | ENSG00000234207 | | AC096570.2 | | |
| rs1509355 | 21_8 | 2: 22613819 | T | ENSG00000231200 | | AC068490.2 | | |
| rs1509355 | 21_8 | 2: 22613819 | T | ENSG00000234207 | | AC096570.2 | | |
| rs1509360 | 21_8 | 2: 22616777 | A | ENSG00000231200 | | AC068490.2 | | |
| rs1509360 | 21_8 | 2: 22616777 | A | ENSG00000234207 | | AC096570.2 | | |
| rs1949038 | 21_8 | 2: 22616534 | C | ENSG00000231200 | | AC068490.2 | | |
| rs1949038 | 21_8 | 2: 22616534 | C | ENSG00000234207 | | AC096570.2 | | |
| rs6741194 | 21_8 | 2: 22616209 | T | ENSG00000231200 | | AC068490.2 | | |
| rs6741194 | 21_8 | 2: 22616209 | T | ENSG00000234207 | | AC096570.2 | | |
| rs6749647 | 21_8 | 2: 22618537 | T | ENSG00000231200 | | AC068490.2 | | |
| rs6749647 | 21_8 | 2: 22618537 | T | ENSG00000234207 | | AC096570.2 | | |
| rs9308959 | 21_8 | 2: 22553001 | T | ENSG00000231200 | | AC068490.2 | | |
| rs6743484 | 21_8 | 2: 22553712 | T | ENSG00000231200 | | AC068490.2 | | |
| rs7569716 | 21_8 | 2: 22568713 | T | ENSG00000231200 | | AC068490.2 | | |
| rs13413863 | 22_11 | 2: 22615313 | G | ENSG00000234207 | | AC096570.2 | | |
| rs13424767 | 22_11 | 2: 22612275 | C | ENSG00000231200 | | AC068490.2 | | |
| rs13424767 | 22_11 | 2: 22612275 | C | ENSG00000234207 | | AC096570.2 | | |
| rs1396725 | 22_11 | 2: 22612638 | A | ENSG00000231200 | | AC068490.2 | | |
| rs1396725 | 22_11 | 2: 22612638 | A | ENSG00000234207 | | AC096570.2 | | |
| rs1509355 | 22_11 | 2: 22613819 | T | ENSG00000231200 | | AC068490.2 | | |
| rs1509355 | 22_11 | 2: 22613819 | T | ENSG00000234207 | | AC096570.2 | | |
| rs1509360 | 22_11 | 2: 22616777 | A | ENSG00000231200 | | AC068490.2 | | |
| rs1509360 | 22_11 | 2: 22616777 | A | ENSG00000234207 | | AC096570.2 | | |
| rs1949038 | 22_11 | 2: 22616534 | C | ENSG00000231200 | | AC068490.2 | | |
| rs1949038 | 22_11 | 2: 22616534 | C | ENSG00000234207 | | AC096570.2 | | |
| rs6741194 | 22_11 | 2: 22616209 | T | ENSG00000231200 | | AC068490.2 | | |
| rs6741194 | 22_11 | 2: 22616209 | T | ENSG00000234207 | | AC096570.2 | | |
| rs6749647 | 22_11 | 2: 22618537 | T | ENSG00000231200 | | AC068490.2 | | |
| rs6749647 | 22_11 | 2: 22618537 | T | ENSG00000234207 | | AC096570.2 | | |
| rs9308959 | 22_11 | 2: 22553001 | T | ENSG00000231200 | | AC068490.2 | | |
| rs1605834 | 22_11 | 2: 22576100 | G | ENSG00000231200 | | AC068490.2 | | |
| rs7569716 | 22_11 | 2: 22568713 | T | ENSG00000231200 | | AC068490.2 | | |
| rs6743484 | 22_11 | 2: 22553712 | T | ENSG00000231200 | | AC068490.2 | | |
| rs1325566 | 25_10 | X: 55791497 | T | | | | | |
| rs1325567 | 25_10 | X: 55791441 | C | | | | | |
| rs1325572 | 25_10 | X: 55828681 | T | | | | | |
| rs1473761 | 25_10 | X: 55748820 | G | ENSG00000083750 | | RRAGB | RRAGB | 10325 |
| rs2104429 | 25_10 | X: 55827933 | A | | | | | |
| rs5914459 | 25_10 | X: 55823342 | C | | | | | |
| rs5914490 | 25_10 | X: 55873522 | C | | | | | |
| rs942846 | 25_10 | X: 55841702 | C | | | | | |
| rs1075145 | 25_10 | X: 55823685 | T | | | | | |
| rs2396841 | 31_22 | 6: 47862920 | T | ENSG00000244694 | | PTCHD4 | PTCHD4 | 442213 |
| rs473606 | 31_22 | 6: 47808177 | T | | | | | |
| rs9395325 | 31_22 | 6: 47854343 | T | ENSG00000244694 | | PTCHD4 | PTCHD4 | 442213 |
| rs1328974 | 31_22 | 6: 47833487 | C | | | | | |
| rs2022333 | 31_22 | 6: 47864831 | A | ENSG00000244694 | | PTCHD4 | PTCHD4 | 442213 |
| rs6912591 | 31_22 | 6: 47853375 | G | ENSG00000244694 | | PTCHD4 | PTCHD4 | 442213 |
| rs7756106 | 31_22 | 6: 47852752 | C | ENSG00000244694 | | PTCHD4 | PTCHD4 | 442213 |
| rs5932754 | 41_12 | X: 129515071 | T | ENSG00000147262 | | GPR119 | GPR119 | 139760 |
| rs5977248 | 41_12 | X: 129501487 | T | ENSG00000102078 | | SLC25A14 | SLC25A14 | 9016 |
| rs4830188 | 41_12 | X: 129514423 | T | ENSG00000147262 | | GPR119 | GPR119 | 139760 |
| rs10502161 | 42_37 | 11: 112843425 | G | ENSG00000149294 | | NCAM1 | NCAM1 | 4684 |
| rs10502161 | 42_37 | 11: 112843425 | G | ENSG00000238998 | | U7 | | |
| rs10502170 | 42_37 | 11: 113040118 | G | ENSG00000149294 | | NCAM1 | NCAM1 | 4684 |
| rs11214533 | 42_37 | 11: 113048466 | C | | ENSR00001573647 | | | |
| rs11214533 | 42_37 | 11: 113048466 | C | ENSG00000149294 | | NCAM1 | NCAM1 | 4684 |
| rs1196185 | 42_37 | 2: 182884959 | A | ENSG00000150722 | | PPP1R1C | LOC151242 | 151242 |
| rs2011507 | 42_37 | 11: 112988280 | C | ENSG00000149294 | | NCAM1 | NCAM1 | 4684 |
| rs2212450 | 42_37 | 11: 112826867 | C | ENSG00000247416 | | RP11-629G13.1 | | |
| rs2701664 | 42_37 | 2: 182908664 | A | ENSG00000150722 | | PPP1R1C | LOC151242 | 151242 |
| rs2701664 | 42_37 | 2: 182908664 | A | ENSG00000222418 | | RNA5SP113 | | |
| rs6589360 | 42_37 | 11: 113050292 | T | ENSG00000149294 | | NCAM1 | NCAM1 | 4684 |
| rs6732434 | 42_37 | 2: 182901257 | G | ENSG00000150722 | | PPP1R1C | LOC151242 | 151242 |
| rs7110628 | 42_37 | 11: 112842988 | G | ENSG00000149294 | | NCAM1 | NCAM1 | 4684 |
| rs12575544 | 42_37 | 11: 112918985 | A | ENSG00000149294 | | NCAM1 | NCAM1 | 4684 |
| rs1273044 | 42_37 | 11: 112993848 | C | ENSG00000149294 | | NCAM1 | NCAM1 | 4684 |
| rs1245133 | 42_37 | 11: 113011721 | G | ENSG00000149294 | | NCAM1 | NCAM1 | 4684 |
| rs17114705 | 42_37 | 11: 112899832 | A | ENSG00000149294 | | NCAM1 | NCAM1 | 4684 |
| rs17114685 | 42_37 | 11: 112889330 | T | ENSG00000149294 | | NCAM1 | NCAM1 | 4684 |
| rs12272966 | 42_37 | 11: 113034787 | T | ENSG00000149294 | | NCAM1 | NCAM1 | 4684 |
| rs17114687 | 42_37 | 11: 112889357 | G | ENSG00000149294 | | NCAM1 | NCAM1 | 4684 |
| rs17114757 | 42_37 | 11: 112951637 | T | ENSG00000149294 | | NCAM1 | NCAM1 | 4684 |
| rs17582738 | 42_37 | 11: 112840745 | T | ENSG00000149294 | | NCAM1 | NCAM1 | 4684 |
| rs17114689 | 42_37 | 11: 112894450 | G | ENSG00000149294 | | NCAM1 | NCAM1 | 4684 |
| rs1436109 | 42_37 | 11: 112991618 | T | ENSG00000149294 | | NCAM1 | NCAM1 | 4684 |
| rs1196160 | 42_37 | 2: 182928012 | A | ENSG00000150722 | | PPP1R1C | LOC151242 | 151242 |
| rs1196155 | 42_37 | 2: 182921272 | C | ENSG00000150722 | | PPP1R1C | LOC151242 | 151242 |
| rs1196183 | 42_37 | 2: 182888983 | T | ENSG00000150722 | | PPP1R1C | LOC151242 | 151242 |
| rs5932896 | 51_28 | X: 130470292 | T | ENSG00000147255 | | IGSF1 | IGSF1 | 3547 |
| rs4462056 | 51_28 | X: 130438580 | A | ENSG00000147255 | | IGSF1 | IGSF1 | 3547 |
| rs4415478 | 51_28 | X: 130438656 | A | ENSG00000147255 | | IGSF1 | IGSF1 | 3547 |
| rs10502161 | 52_42 | 11: 112843425 | G | ENSG00000149294 | | NCAM1 | NCAM1 | 4684 |
| rs10502161 | 52_42 | 11: 112843425 | G | ENSG00000238998 | | U7 | | |
| rs10502170 | 52_42 | 11: 113040118 | G | ENSG00000149294 | | NCAM1 | NCAM1 | 4684 |
| rs11214533 | 52_42 | 11: 113048466 | C | | ENSR00001573647 | | | |
| rs17582738 | 52_42 | 11: 112840745 | T | ENSG00000149294 | | NCAM1 | NCAM1 | 4684 |
| rs2212450 | 52_42 | 11: 112826867 | C | ENSG00000247416 | | RP11-629G13.1 | | |
| rs7110628 | 52_42 | 11: 112842988 | G | ENSG00000149294 | | NCAM1 | NCAM1 | 4684 |
| rs12575544 | 52_42 | 11: 112918985 | A | ENSG00000149294 | | NCAM1 | NCAM1 | 4684 |
| rs1273044 | 52_42 | 11: 112993848 | C | ENSG00000149294 | | NCAM1 | NCAM1 | 4684 |
| rs17114705 | 52_42 | 11: 112899832 | A | ENSG00000149294 | | NCAM1 | NCAM1 | 4684 |
| rs1245133 | 52_42 | 11: 113011721 | G | ENSG00000149294 | | NCAM1 | NCAM1 | 4684 |
| rs12272966 | 52_42 | 11: 113034787 | T | ENSG00000149294 | | NCAM1 | NCAM1 | 4684 |
| rs17114685 | 52_42 | 11: 112889330 | T | ENSG00000149294 | | NCAM1 | NCAM1 | 4684 |
| rs17114687 | 52_42 | 11: 112889357 | G | ENSG00000149294 | | NCAM1 | NCAM1 | 4684 |
| rs17114757 | 52_42 | 11: 112951637 | T | ENSG00000149294 | | NCAM1 | NCAM1 | 4684 |
| rs6589360 | 52_42 | 11: 113050292 | T | ENSG00000149294 | | NCAM1 | NCAM1 | 4684 |
| rs17114689 | 52_42 | 11: 112894450 | G | ENSG00000149294 | | NCAM1 | NCAM1 | 4684 |
| rs2725046 | 54_51 | 8: 4467853 | G | ENSG00000183117 | | CSMD1 | CSMD1 | 64478 |
| rs1382250 | 54_51 | 8: 4465300 | T | ENSG00000183117 | | CSMD1 | CSMD1 | 64478 |
| rs2617104 | 54_51 | 8: 4467788 | C | ENSG00000183117 | | CSMD1 | CSMD1 | 64478 |
| rs2725037 | 54_51 | 8: 4471486 | G | ENSG00000183117 | | CSMD1 | CSMD1 | 64478 |
| rs2725045 | 54_51 | 8: 4467334 | T | ENSG00000183117 | | CSMD1 | CSMD1 | 64478 |
| rs10791112 | 56_19 | 11: 130870215 | T | | ENSR00000571552 | | | |
| rs10791112 | 56_19 | 11: 130870215 | T | ENSG00000242673 | | Metazoa_SRP | | |
| rs10894294 | 56_19 | 11: 130830748 | A | | | | | |
| rs1433976 | 56_19 | 11: 130875123 | G | ENSG00000242673 | | Metazoa_SRP | | |
| rs1991899 | 56_19 | 11: 130801649 | G | | | | | |
| rs10874067 | 56_30 | 1: 80207766 | T | | | | | |
| rs1524183 | 56_30 | 1: 80179889 | C | | | | | |
| rs1591865 | 56_30 | 1: 97177244 | G | | | | | |
| rs1591866 | 56_30 | 1: 97177209 | G | | | | | |
| rs4402575 | 56_30 | 16: 20297138 | A | | | | | |
| rs6497455 | 56_30 | 16: 20283920 | C | | | | | |
| rs6497465 | 56_30 | 16: 20288797 | A | | | | | |
| rs6699242 | 56_30 | 1: 97258468 | A | ENSG00000117569 | | PTBP2 | PTBP2 | 58155 |
| rs7191525 | 56_30 | 16: 20276957 | G | | | | | |
| rs8050244 | 56_30 | 16: 20277579 | T | | | | | |
| rs8054898 | 56_30 | 16: 20290454 | C | | | | | |
| rs4581094 | 58_29 | 8: 66065387 | A | ENSG00000239261 | | RPL31P41 | | |
| rs4599855 | 58_29 | 8: 66088232 | C | | | | | |
| rs4737704 | 58_29 | 8: 66072703 | T | ENSG00000239261 | | RPL31P41 | | |
| rs6982800 | 58_29 | 8: 66074511 | A | | | | | |
| rs6998613 | 58_29 | 8: 66074310 | C | | | | | |
| rs12544654 | 58_29 | 8: 66102770 | C | | | | | |
| rs231150 | 59_48 | 8: 116420327 | T | ENSG00000104447 | | TRPS1 | TRPS1 | 7227 |
| rs6047529 | 59_48 | 20: 2215286 | C | | | | | |
| rs6137352 | 59_48 | 20: 2198288 | A | ENSG00000226644 | | RP11-128M1.1 | | 388780 |
| rs2049863 | 59_49 | 8: 116409435 | T | | | | | |
| rs231146 | 59_50 | 8: 116416989 | G | ENSG00000104447 | | TRPS1 | TRPS1 | 7227 |
| rs6082408 | 59_51 | 20: 2192516 | C | ENSG00000226644 | | RP11-128M1.1 | | 388780 |
| rs6082421 | 59_52 | 20: 2197908 | A | ENSG00000226644 | | RP11-128M1.1 | | 388780 |
| rs5932896 | 61_39 | X: 130470292 | T | ENSG00000147255 | | IGSF1 | IGSF1 | 3547 |
| rs4462056 | 61_39 | X: 130438580 | A | ENSG00000147255 | | IGSF1 | IGSF1 | 3547 |
| rs4415478 | 61_39 | X: 130438656 | A | ENSG00000147255 | | IGSF1 | IGSF1 | 3547 |
| rs2208760 | 65_25 | 20: 18910490 | T | | | | | |
| rs4814813 | 65_25 | 20: 18930034 | G | | | | | |
| rs6045692 | 65_25 | 20: 18901412 | T | | | | | |
| rs6045706 | 65_25 | 20: 18929348 | T | | | | | |
| rs1555510 | 65_25 | 20: 18942562 | C | | | | | |
| rs11632716 | 71_55 | 15: 88360283 | C | | ENSR00001454866 | | | |
| rs16940789 | 71_55 | 15: 88322461 | A | | | | | |
| rs1986826 | 71_55 | 15: 88327131 | C | | | | | |
| rs4243096 | 71_55 | 15: 88366975 | C | | | | | |
| rs4887326 | 71_55 | 15: 88341400 | G | | | | | |
| rs7166186 | 71_55 | 15: 88345483 | T | | | | | |
| rs10791112 | 75_31 | 11: 130870215 | T | | ENSR00000571552 | | | |
| rs10791112 | 75_31 | 11: 130870215 | T | ENSG00000242673 | | Metazoa_SRP | | |
| rs10894294 | 75_31 | 11: 130830748 | A | | | | | |
| rs1433976 | 75_31 | 11: 130875123 | G | ENSG00000242673 | | Metazoa_SRP | | |
| rs1991899 | 75_31 | 11: 130801649 | G | | | | | |
| rs514235 | 75_31 | 1: 93438456 | C | ENSG00000239710 | | Metazoa_SRP | | |
| rs514235 | 75_31 | 1: 93438456 | C | ENSG00000252121 | | U6 | | |
| rs521428 | 75_31 | 1: 93445497 | A | ENSG00000238787 | | AC093577.1 | | |
| rs521428 | 75_31 | 1: 93445497 | A | ENSG00000239710 | | Metazoa_SRP | | |
| rs660870 | 75_31 | 1: 93445417 | A | ENSG00000238787 | | AC093577.1 | | |
| rs660870 | 75_31 | 1: 93445417 | A | ENSG00000239710 | | Metazoa_SRP | | |
| rs10791109 | 75_31 | 11: 130850377 | G | | | | | |
| rs11632716 | 75_67 | 15: 88360283 | C | | | | | |
| rs11785991 | 75_67 | 8: 51750040 | A | | | | | |
| rs11945291 | 75_67 | 4: 98184296 | G | ENSG00000163116 | | STPG2 | C4ORF37 | 285555 |
| rs12908584 | 75_67 | 15: 86643080 | G | ENSG00000260477 | | RP11-553E24.2 | | |
| rs134432 | 75_67 | 22: 35588844 | G | ENSG00000233080 | | CTA-714B7.5 | | |
| rs134432 | 75_67 | 22: 35588844 | G | ENSG00000243453 | | COX7BP1 | | |
| rs1805610 | 75_67 | 3: 180772241 | T | ENSG00000242808 | | SOX2-OT | | 347689 |
| rs1805610 | 75_67 | 3: 180772241 | T | ENSG00000243341 | | RP11-436A20.3 | | |
| rs1979268 | 75_67 | 12: 10776513 | G | ENSG00000060140 | | STYK1 | STYK1 | 55359 |
| rs1986826 | 75_67 | 15: 88327131 | C | | | | | |
| rs2161850 | 75_67 | 8: 30577906 | C | | ENSR00001440140 | | | |
| rs2161850 | 75_67 | 8: 30577906 | C | ENSG00000104687 | | GSR | GSR | 2936 |
| rs2317837 | 75_67 | 16: 82324743 | T | | | | | |
| rs2763529 | 75_67 | 14: 103654939 | T | ENSG00000251533 | | LINC00605 | | 100131366 |
| rs2763529 | 75_67 | 14: 103654939 | T | ENSG00000259525 | | GCSHP2 | | |
| rs3888124 | 75_67 | 8: 42285336 | C | ENSG00000168575 | | SLC20A2 | SLC20A2 | 6575 |
| rs4243096 | 75_67 | 15: 88366975 | C | | | | | |
| rs4402575 | 75_67 | 16: 20297138 | A | | | | | |
| rs4603135 | 75_67 | 1: 116171383 | T | | | | | |
| rs4699310 | 75_67 | 4: 98147844 | T | ENSG00000163116 | | STPG2 | C4ORF37 | 285555 |
| rs4732942 | 75_67 | 8: 29297518 | C | | | | | |
| rs4887326 | 75_67 | 15: 88341400 | G | | | | | |
| rs6497455 | 75_67 | 16: 20283920 | C | | | | | |
| rs6497465 | 75_67 | 16: 20288797 | A | | | | | |
| rs6984059 | 75_67 | 8: 52148019 | C | | | | | |
| rs7006725 | 75_67 | 8: 53055353 | A | ENSG00000147488 | | ST18 | ST18 | 9705 |
| rs717509 | 75_67 | 8: 51566749 | G | ENSG00000147481 | | SNTG1 | SNTG1 | 54212 |
| rs7191525 | 75_67 | 16: 20276957 | G | | | | | |
| rs7819847 | 75_67 | 8: 50367785 | C | | | | | |
| rs7832529 | 75_67 | 8: 42306813 | C | ENSG00000168575 | | SLC20A2 | SLC20A2 | 6575 |
| rs8050244 | 75_67 | 16: 20277579 | T | | | | | |
| rs8054898 | 75_67 | 16: 20290454 | C | | | | | |
| rs900237 | 75_67 | 8: 49596141 | C | ENSG00000233858 | | AC026904.1 | | |
| rs900237 | 75_67 | 8: 49596141 | C | ENSG00000253608 | | RP11-770E5.1 | | |
| rs962392 | 75_67 | 10: 108014282 | T | | | | | |
| rs9917982 | 75_67 | 4: 98107638 | T | ENSG00000163116 | | STPG2 | C4ORF37 | 285555 |
| rs7009058 | 75_67 | 8: 51493707 | C | ENSG00000147481 | | SNTG1 | SNTG1 | 54212 |
| rs5932896 | 76_63 | X: 130470292 | T | ENSG00000147255 | | IGSF1 | IGSF1 | 3547 |
| rs4462056 | | X: 130438580 | A | ENSG00000147255 | | IGSF1 | IGSF1 | 3547 |
| rs4415478 | | X: 130470292 | T | ENSG00000147255 | | IGSF1 | IGSF1 | 3547 |
| rs11945291 | 76_74 | 4: 98184296 | G | ENSG00000163116 | | STPG2 | C4ORF37 | 285555 |
| rs2763529 | 76_74 | 14: 103654939 | T | ENSG00000251533 | | LINC00605 | | 100131366 |
| rs2763529 | 76_74 | 14: 103654939 | T | ENSG00000259525 | | GCSHP2 | | |
| rs2875373 | 76_74 | 4: 24700151 | T | | | | | |
| rs4581094 | 76_74 | 8: 66065387 | A | ENSG00000239261 | | RPL31P41 | | |
| rs4697472 | 76_74 | 4: 24698303 | C | | | | | |
| rs4699310 | 76_74 | 4: 98147844 | T | ENSG00000163116 | | STPG2 | C4ORF37 | 285555 |
| rs4737704 | 76_74 | 8: 66072703 | T | ENSG00000239261 | | RPL31P41 | | |
| rs6812181 | 76_74 | 4: 24711351 | T | | | | | |
| rs6888272 | 76_74 | 5: 73355560 | T | | | | | |
| rs6982800 | 76_74 | 8: 66074511 | A | | | | | |
| rs6998613 | 76_74 | 8: 66074310 | C | | | | | |
| rs900237 | 76_74 | 8: 49596141 | C | ENSG00000233858 | | AC026904.1 | | |
| rs900237 | 76_74 | 8: 49596141 | C | ENSG00000253608 | | RP11-770E5.1 | | |
| rs9917982 | 76_74 | 4: 98107638 | T | ENSG00000163116 | | STPG2 | C4ORF37 | 285555 |
| rs9938516 | 76_74 | 16: 47926261 | C | ENSG00000261231 | | RP11-523L20.2 | | |
| rs2725046 | 77_5 | 8: 4467853 | G | ENSG00000183117 | | CSMD1 | CSMD1 | 64478 |
| rs1382250 | 77_5 | 8: 4465300 | T | ENSG00000183117 | | CSMD1 | CSMD1 | 64478 |
| rs2617104 | 77_5 | 8: 4467788 | C | ENSG00000183117 | | CSMD1 | CSMD1 | 64478 |
| rs2725037 | 77_5 | 8: 4471486 | G | ENSG00000183117 | | CSMD1 | CSMD1 | 64478 |
| rs2725045 | 77_5 | 8: 4467334 | T | ENSG00000183117 | | CSMD1 | CSMD1 | 64478 |
| rs4402575 | 81_13 | 16: 20297138 | A | | | | | |
| rs6497455 | 81_13 | 16: 20283920 | C | | | | | |
| rs6497465 | 81_13 | 16: 20288797 | A | | | | | |
| rs6984059 | 81_13 | 8: 52148019 | C | | | | | |
| rs717509 | 81_13 | 8: 51566749 | G | ENSG00000147481 | | SNTG1 | SNTG1 | 54212 |
| rs7191525 | 81_13 | 16: 20276957 | G | | | | | |
| rs8050244 | 81_13 | 16: 20277579 | T | | | | | |
| rs8054898 | 81_13 | 16: 20290454 | C | | | | | |
| rs11785991 | 81_13 | 8: 51750040 | A | | | | | |
| rs7009058 | 81_13 | 8: 51493707 | C | ENSG00000147481 | | SNTG1 | SNTG1 | 54212 |
| rs13413863 | 81_3 | 2: 22615313 | G | ENSG00000234207 | | AC096570.2 | | |
| rs13424767 | 81_3 | 2: 22612275 | C | ENSG00000231200 | | AC068490.2 | | |
| rs13424767 | 81_3 | 2: 22612275 | C | ENSG00000234207 | | AC096570.2 | | |
| rs1396725 | 81_3 | 2: 22612638 | A | ENSG00000231200 | | AC068490.2 | | |
| rs1396725 | 81_3 | 2: 22612638 | A | ENSG00000234207 | | AC096570.2 | | |
| rs1509355 | 81_3 | 2: 22613819 | T | ENSG00000231200 | | AC068490.2 | | |
| rs1509355 | 81_3 | 2: 22613819 | T | ENSG00000234207 | | AC096570.2 | | |
| rs1509360 | 81_3 | 2: 22616777 | A | ENSG00000231200 | | AC068490.2 | | |
| rs1509360 | 81_3 | 2: 22616777 | A | ENSG00000234207 | | AC096570.2 | | |
| rs1949038 | 81_3 | 2: 22616534 | C | ENSG00000231200 | | AC068490.2 | | |
| rs1949038 | 81_3 | 2: 22616534 | C | ENSG00000234207 | | AC096570.2 | | |
| rs6741194 | 81_3 | 2: 22616209 | T | ENSG00000231200 | | AC068490.2 | | |
| rs6741194 | 81_3 | 2: 22616209 | T | ENSG00000234207 | | AC096570.2 | | |
| rs6749647 | 81_3 | 2: 22618537 | T | ENSG00000231200 | | AC068490.2 | | |
| rs6749647 | 81_3 | 2: 22618537 | T | ENSG00000234207 | | AC096570.2 | | |
| rs9308959 | 81_3 | 2: 22553001 | T | ENSG00000231200 | | AC068490.2 | | |
| rs1605834 | 81_3 | 2: 22576100 | G | ENSG00000231200 | | AC068490.2 | | |
| rs6743484 | 81_3 | 2: 22553712 | T | ENSG00000231200 | | AC068490.2 | | |
| rs7569716 | 81_3 | 2: 22568713 | T | ENSG00000231200 | | AC068490.2 | | |
| rs12956646 | 81_73 | 18: 24685369 | C | ENSG00000154080 | | CHST9 | CHST9 | 83539 |
| rs12956646 | 81_73 | 18: 24685369 | C | ENSG00000260372 | | CHST9-AS1 | | 147429 |
| rs12956990 | 81_73 | 18: 24713270 | C | ENSG00000154080 | | CHST9 | CHST9 | 83539 |
| rs12956990 | 81_73 | 18: 24713270 | C | ENSG00000260372 | | CHST9-AS1 | | 147429 |
| rs2030234 | 81_73 | 11: 86965391 | G | ENSG00000166575 | | TMEM135 | TMEM135 | 65084 |
| rs2030234 | 81_73 | 11: 86965391 | G | ENSG00000213287 | | RP11-680L20.1 | | |
| rs2572189 | 81_73 | 15: 33763472 | G | ENSG00000198838 | | RYR3 | RYR3 | 6263 |
| rs61552 | 81_73 | 11: 86920178 | G | ENSG00000166575 | | TMEM135 | TMEM135 | 65084 |
| rs7240658 | 81_73 | 18: 24687347 | A | ENSG00000154080 | | CHST9 | CHST9 | 83539 |
| rs7240658 | 81_73 | 18: 24687347 | A | ENSG00000260372 | | CHST9-AS1 | | 147429 |
| rs919140 | 81_73 | 18: 24689706 | C | ENSG00000154080 | | CHST9 | CHST9 | 83539 |
| rs11235109 | 81_73 | 11: 87059742 | G | | | | | |
| rs186198 | 81_73 | 11: 86911919 | C | ENSG00000166575 | | RYR3 | RYR3 | 6263 |
| rs2572175 | 81_73 | 15: 33777705 | C | ENSG00000198838 | | RYR3 | RYR3 | 6263 |
| rs4770836 | 83_41 | 13: 26037909 | C | | ENSR00000513160 | | | |
| rs668001 | 83_41 | 13: 26005056 | C | ENSG00000132932 | | ATP8A2 | ATP8A2 | 51761 |
| rs668001 | 83_41 | 13: 26005056 | C | ENSG00000132932 | | ATP8A2 | ATP8A2 | 51761 |
| rs640894 | 83_41 | 13: 26006474 | G | ENSG00000132932 | | ATP8A2 | ATP8A2 | 51761 |
| rs12956646 | 85_23 | 18: 24685369 | C | ENSG00000154080 | | CHST9 | CHST9 | 83539 |
| rs12956646 | 85_23 | 18: 24685369 | C | ENSG00000260372 | | CHST9-AS1 | | 147429 |
| rs12956990 | 85_23 | 18: 24713270 | C | ENSG00000154080 | | CHST9 | CHST9 | 83539 |
| rs12956990 | 85_23 | 18: 24713270 | C | ENSG00000260372 | | CHST9-AS1 | | 147429 |
| rs7240658 | 85_23 | 18: 24687347 | A | ENSG00000154080 | | CHST9 | CHST9 | 83539 |
| rs7240658 | 85_23 | 18: 24687347 | A | ENSG00000260372 | | CHST9-AS1 | | 147429 |
| rs919140 | 85_23 | 18: 24689706 | C | ENSG00000154080 | | CHST9 | CHST9 | 83539 |
| rs919140 | 85_23 | 18: 24689706 | C | ENSG00000260372 | | CHST9-AS1 | | 147429 |
| rs1146745 | 85_84 | 3: 84904026 | T | ENSG00000242641 | | RP11-735B13.1 | | 440970 |
| rs1248821 | 85_84 | 3: 84930747 | C | ENSG00000242339 | | RP11-735B13.2 | | |
| rs385115 | 85_84 | 3: 84892835 | A | ENSG00000242641 | | RP11-735B13.1 | | 440970 |
| rs1248845 | 85_84 | 3: 84871763 | A | ENSG00000242641 | | RP11-735B13.1 | | 440970 |
| rs12430088 | 87_26 | 13: 101704076 | T | ENSG00000233009 | | NALCN-AS1 | | 100885778 |
| rs3751403 | 87_26 | 13: 101701747 | T | | ENSR00001511846 | | | |
| rs3751403 | 87_26 | 13: 101701747 | T | ENSG00000102452 | | NALCN | NALCN | 259232 |
| rs3751403 | 87_26 | 13: 101701747 | T | ENSG00000233009 | | NALCN-AS1 | | 100885778 |
| rs638732 | 87_26 | 13: 101709598 | G | ENSG00000102452 | | NALCN | NALCN | 259232 |
| rs638732 | 87_26 | 13: 101709598 | G | ENSG00000233009 | | NALCN-AS1 | | 100885778 |
| rs9554752 | 87_26 | 13: 101726313 | T | ENSG00000102452 | | NALCN | NALCN | 259232 |
| rs7986657 | 87_26 | 13: 101736999 | G | ENSG00000102452 | | NALCN | NALCN | 259232 |
| rs10782945 | 87_84 | 1: 93304272 | T | ENSG00000122406 | | RPL5 | RPL5 | 6083 |
| rs10782945 | 87_84 | 1: 93304272 | T | ENSG00000154511 | | FAM69A | FAM69A | 388650 |
| rs10782945 | 87_84 | 1: 93304272 | T | ENSG00000206680 | | SNORD21 | | 6083 |
| rs10782945 | 87_84 | 1: 93304272 | T | ENSG00000207523 | | SNORA66 | | 26782 |
| rs10782945 | 87_84 | 1: 93304272 | T | ENSG00000251795 | | SNORA66 | | |
| rs11164835 | 87_84 | 1: 93379093 | A | ENSG00000154511 | | FAM69A | FAM69A | 388650 |
| rs12066638 | 87_84 | 1: 93375391 | G | | ENSR00001522451 | | | |
| rs12745968 | 87_84 | 1: 93401837 | G | ENSG00000154511 | | FAM69A | FAM69A | 388650 |
| rs12745968 | 87_84 | 1: 93401837 | G | ENSG00000229052 | | RP11-386123.1 | | |
| rs35183060 | 87_84 | 1: 93346928 | T | ENSG00000154511 | | FAM69A | FAM69A | 388650 |
| rs6604026 | 87_84 | 1: 93303603 | C | | ENSR00000540793 | | | |
| rs6604026 | 87_84 | 1: 93303603 | C | ENSG00000122406 | | RPL5 | RPL5 | 6083 |
| rs6604026 | 87_84 | 1: 93303603 | C | ENSG00000154511 | | FAM69A | FAM69A | 388650 |
| rs6604026 | 87_84 | 1: 93303603 | C | ENSG00000206680 | | SNORD21 | | 6083 |
| rs6604026 | 87_84 | 1: 93303603 | C | ENSG00000207523 | | SNORA66 | | 26782 |
| rs6604026 | 87_84 | 1: 93303603 | C | ENSG00000251795 | | SNORA66 | | |
| rs9651257 | 87_84 | 1: 93385136 | C | ENSG00000154511 | | FAM69A | FAM69A | 388650 |
| rs10874753 | 87_84 | 1: 93429087 | A | ENSG00000154511 | | FAM69A | FAM69A | 388650 |
| rs2255723 | 87_84 | 1: 93368309 | T | ENSG00000154511 | | FAM69A | FAM69A | 388650 |
| rs2811593 | 87_84 | 1: 93343891 | C | ENSG00000154511 | | FAM69A | FAM69A | 388650 |
| rs2811600 | 87_84 | 1: 93334138 | T | ENSG00000154511 | | FAM69A | FAM69A | 388650 |
| rs7514280 | 87_84 | 1: 93320869 | T | ENSG00000154511 | | FAM69A | FAM69A | 388650 |
| rs7536563 | 87_84 | 1: 93349046 | G | ENSG00000154511 | | FAM69A | FAM69A | 388650 |
| rs12411340 | 88_43 | 10: 67037492 | T | | | | | |
| rs12411779 | 88_43 | 10: 67038698 | T | | | | | |
| rs12414755 | 88_43 | 10: 67014534 | G | | | | | |
| rs17792002 | 88_43 | 10: 66963409 | C | | | | | |
| rs7097087 | 88_43 | 10: 67031903 | G | | | | | |
| rs7912511 | 88_43 | 10: 66977696 | G | | | | | |
| rs10509215 | 88_43 | 10: 66988617 | A | | | | | |
| rs6497455 | 88_64 | 16: 20283920 | C | | | | | |
| rs6497465 | 88_64 | 16: 20288797 | A | | | | | |
| rs7191525 | 88_64 | 16: 20276957 | G | | | | | |
| rs8050244 | 88_64 | 16: 20277579 | T | | | | | |
| rs8054898 | 88_64 | 16: 20290454 | C | | | | | |
| rs4402575 | 88_64 | 16: 20297138 | A | | | | | |
| rs11164798 | 88_8 | 1: 93172782 | A | ENSG00000067208 | | EVI5 | EVI5 | 7813 |
| rs1341118 | 88_8 | 6: 104754646 | T | | | | | |
| rs1341118 | 88_8 | 6: 104754646 | G | | | | | |
| rs169282 | 88_8 | 6: 104765744 | G | | | | | |
| rs270666 | 88_8 | 6: 104753237 | C | | | | | |
| rs514235 | 88_8 | 1: 93438456 | C | ENSG00000239710 | | Metazoa_SRP | | |
| rs514235 | 88_8 | 1: 93438456 | C | ENSG00000252121 | | U6 | | |
| rs521428 | 88_8 | 1: 93445497 | A | ENSG00000238787 | | AC093577.1 | | |
| rs521428 | 88_8 | 1: 93445497 | A | ENSG00000239710 | | Metazoa_SRP | | |
| rs6571178 | 88_8 | 6: 104766876 | C | | | | | |
| rs660870 | 88_8 | 1: 93445417 | A | ENSG00000238787 | | AC093577.1 | | |
| rs660870 | 88_8 | 1: 93445417 | A | ENSG00000239710 | | Metazoa_SRP | | |
| rs7764670 | 88_8 | 6: 104774231 | G | | ENSR00001223173 | | | |
| rs7764670 | 88_8 | 6: 104774231 | G | | | | | |
| rs9391181 | 88_8 | 6: 104759143 | T | | | | | |

Likewise, SNPs from SNP set 22_11 are located within a large intergenic region corresponding to two overlapping and newly characterized long ncRNAs AC068490.2 and AC096570.2 (Table 4). Moreover, two SNP variants of SNP set G19_2 affect miRNA AL354928.1 and small nuclear RNA U4, as well as protein-coding GOLGA1 gene (FIG. 6A, Table 4). Finally, the SNP sets can map to large genomic regions. That is the case with all SNPs in SNP set 22_11 (with risk of 73%), and a few in SNP set 81_13 (with risk of 95%), which correspond to two different structural CNVs already annotated. These results point to accumulation of possible regulatory alterations of gene expression pattern in these groups (Table 4), which suggests an underlying complex and dynamic architecture of molecular processes that influence vulnerability to distinct forms of SZ.

### 5. Bioinformatics Analysis of the SNP Set-Related Genes Reveals Disparate Molecular Consequences

A detailed analysis of SNPs and mapped genes revealed at least three complex scenarios affecting multiple genes in different fashions (activation, repression, antisense modulation) and producing different molecular consequences (Table 4). First, we determined that even a single SNP within a SNP set could produce different consequences in affected transcripts (Table 4). For example, one SNP from SNP set 81_13 was located in a protein-coding region of the SNTG1 gene, which can produce either a change in an intron or in a transcript affecting nonsense-mediated protein decay that would be eliminated by a surveillance pathway containing a premature stop codon (Table 4). Second, we found that multiple SNPs within a SNP set can affect multiple genes in different ways. This heterogeneity is exemplified by SNPs from SNP set 19_2 intersecting with both ncRNAs and the GOLGA1 gene (FIG. 4a). Third, we uncovered that multiple SNPs within different SNP sets can distinctively affect single genes. For example, SNP sets 71_55 and 146 are located in different networks since they have neither SNPs nor subjects in common (FIG. 5). Yet, all SNPs within both SNP sets are located in the same NTRK3 gene, which influences hippocampal function, but at different locations (FIG. 6B), which thereby may modify risk for SZ differentially. Consequently it is not surprising that each SNP set is observed in different individuals with distinct phenotypic consequences. Overall, since a single SNP can affect multiple gene transcripts, or multiple SNP sets may influence a single gene transcript, we must consider the specific transcription pathway in order to understand antecedent mechanisms that result in equifinality and multifinality.

### 6. Genes Mapped by SNP Sets at Risk Correlate with Different Aspects of Neurodevelopment

Most genes mapped by the SNP sets are involved in neurodevelopment (Table 3). For example, the SNP set 81_13 (FIG. 5A) maps to SNTG1, PXDNL, and GP2 genes (Table 2). SNTG1 is a syntrophin that mediates dystrophin binding in brain specifically. It is down-regulated in neurodevelopmental disorders, sleep disorders, and dementia (Table 3). PXDNL encodes a peroxidasin-like protein, which affects risk of SZ and dementia (Table 3). GP2 encodes glycoprotein 2 (zymogen granule membrane) and is down-regulated in neuropathy and basal ganglia disorders, but up-regulated in Alzheimer's disease (Table 3). Cumulatively, characterization of all genes in terms of related diseases supports the biological impact of these SNP sets.

TABLE 3
Mapping Genes Targeted by SNP Sets to Mental and
Brain and Nervous System Disorder Categories.
(Information obtained fron Nextbio database)

| | | | Up/Down |
|---|---|---|---|
| Gene | Disease | Score | regulated |
| 7SK | Autistic disorder | 39 | up-regulated |
| 7SK | Encephalomyelopathy | 32 | up-regulated |
| 7SK | Mood disorder | 51 | down-regulated |
| 7SK | Multiple sclerosis | 27 | up-regulated |
| ABCC12 | Alzheimer's disease | 55 | down-regulated |
| ABCC12 | Dementia | 55 | down-regulated |
| ABCC12 | Disorder of basal ganglia | 2 | up-regulated |
| ABCC12 | Hypoxia of brain | 8 | up-regulated |
| ABCC12 | Meningitis | 14 | up-regulated |
| ABCC12 | Movement disorder | 1 | up-regulated |
| ABCC12 | Multiple sclerosis | 37 | down-regulated |
| ABCC12 | Nerve Injury | 25 | down-regulated |
| ABCC12 | Neuropathy | 14 | down-regulated |
| ABCC12 | Parkinson's disease | 10 | up-regulated |
| ABCC12 | Psychotic disorder | 47 | up-regulated |
| ABCC12 | Schizophrenia | 47 | up-regulated |
| ARPC5L | Alzheimer's disease | 26 | down-regulated |
| ARPC5L | Amyotrophic lateral sclerosis | 14 | down-regulated |
| ARPC5L | Anxiety disorder | 73 | up-regulated |
| ARPC5L | Autistic disorder | 45 | down-regulated |
| ARPC5L | Cerebrovascular disease | 45 | up-regulated |
| ARPC5L | Chronic fatigue syndrome | 100 | down-regulated |
| ARPC5L | Dementia | 26 | down-regulated |
| ARPC5L | Developmental mental | 41 | up-regulated |
| | disorder | | |
| ARPC5L | Disorder of basal ganglia | 74 | down-regulated |
| ARPC5L | Disorder of brain | 38 | up-regulated |
| ARPC5L | Huntington's disease | 85 | down-regulated |
| ARPC5L | Meningitis | 69 | down-regulated |
| ARPC5L | Mental retardation | 38 | up-regulated |
| ARPC5L | Motor neuron disease | 28 | up-regulated |
| ARPC5L | Movement disorder | 71 | down-regulated |
| ARPC5L | Nerve Injury | 1 | down-regulated |
| ARPC5L | Parkinson's disease | 50 | down-regulated |
| ARPC5L | Prion disease | 26 | down-regulated |
| ARPC5L | Psychotic disorder | 36 | down-regulated |
| ARPC5L | Schizophrenia | 36 | down-regulated |
| ATP8A2 | Alzheimer's disease | 44 | down-regulated |
| ATP8A2 | Autistic disorder | 23 | up-regulated |
| ATP8A2 | Cerebrovascular disease | 29 | down-regulated |
| ATP8A2 | Dementia | 43 | down-regulated |
| ATP8A2 | Disorder of basal ganglia | 84 | down-regulated |
| ATP8A2 | Encephalitis | 46 | down-regulated |
| ATP8A2 | Encephalomyelopathy | 37 | up-regulated |
| ATP8A2 | Huntington's disease | 80 | down-regulated |
| ATP8A2 | Hypoxia of brain | 32 | down-regulated |
| ATP8A2 | Meningitis | 55 | up-regulated |
| ATP8A2 | Movement disorder | 81 | down-regulated |
| ATP8A2 | Nerve Injury | 31 | up-regulated |
| ATP8A2 | Neuropathy | 33 | down-regulated |
| ATP8A2 | Parkinson's disease | 84 | down-regulated |
| ATP8A2 | Prion disease | 40 | down-regulated |
| ATP8A2 | Psychotic disorder | 30 | 0.0001 p-value |
| ATP8A2 | Schizophrenia | 30 | 0.0001 p-value |
| ATP8A2 | Sleep disorder | 34 | down-regulated |
| C14orf102 | Alzheimer's disease | 48 | up-regulated |
| C14orf102 | Anxiety disorder | 17 | up-regulated |
| C14orf102 | Autistic disorder | 27 | up-regulated |
| C14orf102 | Cerebrovascular disease | 20 | down-regulated |
| C14orf102 | Dementia | 48 | up-regulated |
| C14orf102 | Disorder of basal ganglia | 18 | up-regulated |
| C14orf102 | Huntington's disease | 24 | down-regulated |
| C14orf102 | Hypoxia of brain | 22 | down-regulated |
| C14orf102 | Meningitis | 51 | up-regulated |
| C14orf102 | Movement disorder | 15 | up-regulated |
| C14orf102 | Neural tube defect | 42 | down-regulated |
| C14orf102 | Neuropathy | 14 | down-regulated |
| C14orf102 | Parkinson's disease | 8 | up-regulated |
| C14orf102 | Psychotic disorder | 20 | 0.0002 p-value |
| C14orf102 | Schizophrenia | 21 | 0.0002 p-value |
| C14orf102 | Sleep disorder | 42 | down-regulated |
| C20orf78 | Anxiety disorder | 32 | down-regulated |
| C20orf78 | Disorder of basal ganglia | 42 | down-regulated |
| C20orf78 | Huntington's disease | 55 | down-regulated |
| C20orf78 | Movement disorder | 39 | down-regulated |
| C20orf78 | Psychotic disorder | 35 | up-regulated |
| C20orf78 | Schizophrenia | 35 | up-regulated |
| C4orf37 | Autistic disorder | 3 | up-regulated |
| C4orf37 | Meningitis | 10 | up-regulated |
| C4orf37 | Multiple sclerosis | 14 | up-regulated |
| C4orf37 | Psychotic disorder | 1 | down-regulated |
| C4orf37 | Schizophrenia | 1 | down-regulated |
| C4orf37 | Sleep disorder | 16 | up-regulated |
| C6orf138 | Amnestic disorder | 88 | up-regulated |
| C6orf138 | Cerebrovascular disease | 48 | down-regulated |
| C6orf138 | Disorder of basal ganglia | 62 | down-regulated |
| C6orf138 | Huntington's disease | 54 | down-regulated |
| C6orf138 | Hypoxia of brain | 51 | down-regulated |
| C6orf138 | Meningitis | 75 | down-regulated |
| C6orf138 | Movement disorder | 59 | down-regulated |
| C6orf138 | Multiple sclerosis | 71 | down-regulated |
| C6orf138 | Nerve injury | 46 | down-regulated |
| C6orf138 | Neuropathy | 83 | down-regulated |
| C6orf138 | Parkinson's disease | 63 | down-regulated |
| CHST9 | Alzheimer's disease | 21 | up-regulated |
| CHST9 | Amnestic disorder | 79 | down-regulated |
| CHST9 | Amyotrophic lateral sclerosis | 37 | down-regulated |
| CHST9 | Dementia | 21 | up-regulated |
| CHST9 | Disorder of basal ganglia | 33 | up-regulated |
| CHST9 | Huntington's disease | 47 | up-regulated |
| CHST9 | Meningitis | 31 | up-regulated |
| CHST9 | Motor neuron disease | 46 | down-regulated |
| CHST9 | Movement disorder | 30 | up-regulated |
| CHST9 | Multiple sclerosis | 56 | up-regulated |
| CHST9 | Nerve injury | 24 | down-regulated |
| CHST9 | Neuropathy | 11 | down-regulated |
| CHST9 | Psychotic disorder | 69 | down-regulated |
| CHST9 | Schizophrenia | 69 | down-regulated |
| CSMD1 | Alzheimer's disease | 38 | 8.7E−6 p-value |
| CSMD1 | Attention deficit hyperactivity | 35 | |
| | disorder | | |
| CSMD1 | Autistic disorder | 38 | down-regulated |
| CSMD1 | Cerebrovascular disease | 10 | 5.4E−5 p-value |
| CSMD1 | Dementia | 37 | 8.7E−6 p-value |
| CSMD1 | Disorder of basal ganglia | 49 | down-regulated |
| CSMD1 | Huntington's disease | 33 | down-regulated |
| CSMD1 | Hypoxia of brain | 13 | 5.4E−5 p-value |
| CSMD1 | Meningitis | 28 | up-regulated |
| CSMD1 | Mood disorder | 38 | 3.6E−6 p-value |
| CSMD1 | Movement disorder | 46 | down-regulated |
| CSMD1 | Multiple sclerosis | 45 | up-regulated |
| CSMD1 | Nerve injury | 23 | down-regulated |
| CSMD1 | Neuropathy | 29 | down-regulated |
| CSMD1 | Parkinson's disease | 49 | down-regulated |
| CSMD1 | Psychotic disorder | 71 | down-regulated |
| CSMD1 | Schizophrenia | 71 | down-regulated |
| DKK4 | Autistic disorder | 33 | up-regulated |
| DKK4 | Disorder of basal ganglia | 1 | up-regulated |
| DKK4 | Encephalomyelopathy | 3 | up-regulated |
| DKK4 | Meningitis | 28 | down-regulated |
| DKK4 | Mood disorder | 43 | down-regulated |
| DKK4 | Movement disorder | 1 | up-regulated |
| DKK4 | Multiple sclerosis | 4 | up-regulated |
| DUSP4 | Alzheimer's disease | 1 | down-regulated |
| DUSP4 | Anxiety disorder | 38 | up-regulated |
| DUSP4 | Cerebrovascular disease | 6 | up-regulated |
| DUSP4 | Disorder of basal ganglia | 38 | down-regulated |
| DUSP4 | Disorder of brain | 46 | down-regulated |
| DUSP4 | Encephalitis | 29 | up-regulated |
| DUSP4 | Encephalomyelopathy | 31 | down-regulated |
| DUSP4 | Huntington's disease | 46 | down-regulated |
| DUSP4 | Hypoxia of brain | 16 | up-regulated |
| DUSP4 | Meningitis | 53 | up-regulated |
| DUSP4 | Mood disorder | 23 | down-regulated |
| DUSP4 | Movement disorder | 35 | down-regulated |
| DUSP4 | Multiple sclerosis | 11 | down-regulated |
| DUSP4 | Nerve injury | 20 | up-regulated |
| DUSP4 | Neural tube defect | 29 | down-regulated |
| DUSP4 | Neuropathy | 17 | down-regulated |
| DUSP4 | Paralytic syndrome | 24 | up-regulated |
| DUSP4 | Parkinson's disease | 12 | down-regulated |
| DUSP4 | Psychotic disorder | 22 | down-regulated |
| DUSP4 | Schizophrenia | 22 | down-regulated |
| DUSP4 | Sleep disorder | 91 | up-regulated |
| DUSP4 | Spinocerebellar ataxia | 51 | down-regulated |
| EML5 | Alzheimer's disease | 11 | down-regulated |
| EML5 | Amnestic disorder | 45 | up-regulated |
| EML5 | Dementia | 11 | down-regulated |
| EML5 | Disorder of basal ganglia | 66 | up-regulated |
| EML5 | Huntington's disease | 78 | up-regulated |
| EML5 | Meningitis | 73 | down-regulated |
| EML5 | Movement disorder | 63 | up-regulated |
| EML5 | Nerve injury | 77 | down-regulated |
| EML5 | Neuropathy | 73 | down-regulated |
| EML5 | Parkinson's disease | 30 | up-regulated |
| EML5 | Psychotic disorder | 79 | 9.5E−7 p-value |
| EML5 | Schizophrenia | 79 | 9.5E−7 p-value |
| EML5 | Sleep disorder | 76 | down-regulated |
| EVI5 | Amnestic disorder | 65 | up-regulated |
| EVI5 | Anxiety disorder | 14 | up-regulated |
| EVI5 | Autistic disorder | 29 | up-regulated |
| EVI5 | Cerebral palsy | 17 | up-regulated |
| EVI5 | Disorder of basal ganglia | 34 | up-regulated |
| EVI5 | Huntington's disease | 39 | up-regulated |
| EVI5 | Meningitis | 49 | up-regulated |
| EVI5 | Mood disorder | 25 | down-regulated |
| EVI5 | Motor neuron disease | 3 | down-regulated |
| EVI5 | Movement disorder | 31 | up-regulated |
| EVI5 | Multiple sclerosis | 100 | 6.5E−12 p-value |
| EVI5 | Nerve injury | 72 | up-regulated |
| EVI5 | Neural tube defect | 25 | up-regulated |
| EVI5 | Neuropathy | 4 | up-regulated |
| EVI5 | Parkinson's disease | 23 | down-regulated |
| EVI5 | Psychotic disorder | 61 | up-regulated |
| EVI5 | Schizophrenia | 62 | up-regulated |
| EVI5 | Sleep disorder | 42 | up-regulated |
| FAM69A | Alzheimer's disease | 1 | down-regulated |
| FAM69A | Autistic disorder | 1 | down-regulated |
| FAM69A | Cerebral palsy | 32 | down-regulated |
| FAM69A | Dementia | 1 | down-regulated |
| FAM69A | Disorder of basal ganglia | 1 | up-regulated |
| FAM69A | Disorder of brain | 29 | up-regulated |
| FAM69A | Encephalitis | 44 | down-regulated |
| FAM69A | Encephalomyelitis | 29 | down-regulated |
| FAM69A | Encephalomyelopathy | 9 | down-regulated |
| FAM69A | Meningitis | 7 | down-regulated |
| FAM69A | Mood disorder | 1 | down-regulated |
| FAM69A | Motor neuron disease | 1 | up-regulated |
| FAM69A | Movement disorder | 1 | up-regulated |
| FAM69A | Multiple sclerosis | 90 | 0.8E−7 p-value |
| FAM69A | Myoneural disorder | 40 | up-regulated |
| FAM69A | Nerve injury | 17 | down-regulated |
| FAM69A | Neuropathy | 11 | up-regulated |
| FAM69A | Paralytic syndrome | 20 | down-regulated |
| FAM69A | Parkinson's disease | 5 | up-regulated |
| FAM69A | Prion disease | 6 | down-regulated |
| FAM69A | Psychotic disorder | 51 | 0.0E−6 p-value |
| FAM69A | Schizophrenia | 51 | 0.0E−6 p-value |
| FAM69A | Sleep disorder | 39 | down-regulated |
| FOXR2 | Nerve injury | 83 | up-regulated |
| FOXR2 | Neuropathy | 86 | up-regulated |
| GOLGA1 | Alzheimer's disease | 24 | 0.0007 p-value |
| GOLGA1 | Autistic disorder | 44 | down-regulated |
| GOLGA1 | Dementia | 24 | 0.0007 p-value |
| GOLGA1 | Disorder of basal ganglia | 55 | up-regulated |
| GOLGA1 | Disorder of brain | 50 | down-regulated |
| GOLGA1 | Encephalomyelopathy | 51 | down-regulated |
| GOLGA1 | Huntington's disease | 52 | up-regulated |
| GOLGA1 | Meningitis | 51 | down-regulated |
| GOLGA1 | Movement disorder | 52 | up-regulated |
| GOLGA1 | Multiple sclerosis | 33 | down-regulated |
| GOLGA1 | Nerve injury | 66 | down-regulated |
| GOLGA1 | Neuropathy | 35 | down-regulated |
| GOLGA1 | Paralytic syndrome | 61 | up-regulated |
| GOLGA1 | Parkinson's disease | 55 | up-regulated |
| GOLGA1 | Psychotic disorder | 50 | 0.0002 p-value |
| GOLGA1 | Schizophrenia | 51 | 0.0002 p-value |
| GOLGA1 | Sleep disorder | 91 | down-regulated |
| GP2 | Alzheimer's disease | 1 | up-regulated |
| GP2 | Amnestic disorder | 20 | up-regulated |
| GP2 | Anxiety disorder | 1 | down-regulated |
| GP2 | Dementia | 1 | up-regulated |
| GP2 | Disorder of basal ganglia | 1 | down-regulated |
| GP2 | Huntington's disease | 1 | down-regulated |
| GP2 | Meningitis | 9 | down-regulated |
| GP2 | Movement disorder | 1 | down-regulated |
| GP2 | Nerve injury | 35 | down-regulated |
| GP2 | Neuropathy | 38 | down-regulated |
| GP2 | Psychotic disorder | 12 | up-regulated |
| GP2 | Schizophrenia | 12 | up-regulated |
| GPR119 | Alzheimer's disease | 59 | 7.8E−5 p-value |
| GPR119 | Anxiety disorder | 48 | down-regulated |
| GPR119 | Dementia | 58 | 7.8E−5 p-value |
| GPR119 | Nerve injury | 27 | up-regulated |
| GPR119 | Neuropathy | 29 | up-regulated |
| HACE1 | Alzheimer's disease | 1 | down-regulated |
| HACE1 | Autistic disorder | 1 | up-regulated |
| HACE1 | Cerebrovascular disease | 1 | up-regulated |
| HACE1 | Dementia | 1 | down-regulated |
| HACE1 | Disorder of basal ganglia | 11 | down-regulated |
| HACE1 | Encephalitis | 1 | down-regulated |
| HACE1 | Huntington's disease | 16 | down-regulated |
| HACE1 | Meningitis | 3 | up-regulated |
| HACE1 | Mood disorder | 1 | 0.0003 p-value |
| HACE1 | Movement disorder | 8 | down-regulated |
| HACE1 | Multiple sclerosis | 1 | up-regulated |
| HACE1 | Nerve injury | 6 | up-regulated |
| HACE1 | Neuropathy | 1 | down-regulated |
| HACE1 | Parkinson's disease | 1 | down-regulated |
| HACE1 | Psychotic disorder | 7 | 0.5E−6 p-value |
| HACE1 | Schizophrenia | 7 | 0.5E−6 p-value |
| HACE1 | Sleep disorder | 8 | up-regulated |
| HPGDS | Alzheimer's disease | 37 | 4.0E−5 p-value |
| HPGDS | Amnestic disorder | 49 | up-regulated |
| HPGDS | Anxiety disorder | 27 | up-regulated |
| HPGDS | Cerebral palsy | 54 | up-regulated |
| HPGDS | Childhood disorder of conduct | 59 | down-regulated |
| | and emotion | | |
| HPGDS | Dementia | 37 | 4.0E−5 p-value |
| HPGDS | Disorder of basal ganglia | 37 | down-regulated |
| HPGDS | Disorder of brain | 44 | down-regulated |
| HPGDS | Huntington's disease | 42 | down-regulated |
| HPGDS | Meningitis | 23 | down-regulated |
| HPGDS | Movement disorder | 34 | down-regulated |
| HPGDS | Multiple sclerosis | 13 | up-regulated |
| HPGDS | Nerve injury | 78 | up-regulated |
| HPGDS | Neuropathy | 43 | down-regulated |
| HPGDS | Parkinson's disease | 29 | down-regulated |
| HPGDS | Prion disease | 75 | up-regulated |
| HPGDS | Psychotic disorder | 16 | 0.0003 p-value |
| HPGDS | Schizophrenia | 16 | 0.0003 p-value |
| HPGDS | Sleep disorder | 45 | down-regulated |
| IGSF1 | Amnestic disorder | 39 | up-regulated |
| IGSF1 | Autistic disorder | 20 | up-regulated |
| IGSF1 | Disorder of basal ganglia | 60 | up-regulated |
| IGSF1 | Disorder of brain | 16 | up-regulated |
| IGSF1 | Encephalitis | 47 | down-regulated |
| IGSF1 | Encephalomyelopathy | 20 | up-regulated |
| IGSF1 | Epilepsy | 14 | up-regulated |
| IGSF1 | Huntington's disease | 70 | up-regulated |
| IGSF1 | Meningitis | 31 | up-regulated |
| IGSF1 | Mood disorder | 6 | up-regulated |
| IGSF1 | Motor neuron disease | 21 | up-regulated |
| IGSF1 | Movement disorder | 57 | up-regulated |
| IGSF1 | Multiple sclerosis | 1 | up-regulated |
| IGSF1 | Nerve injury | 48 | down-regulated |
| IGSF1 | Neuropathy | 32 | down-regulated |
| IGSF1 | Parkinson's disease | 29 | down-regulated |
| IGSF1 | Psychotic disorder | 17 | up-regulated |
| IGSF1 | Schizophrenia | 18 | up-regulated |
| IGSF1 | Sleep disorder | 84 | down-regulated |
| ITFG1 | Alzheimer's disease | 44 | down-regulated |
| ITFG1 | Autistic disorder | 12 | down-regulated |
| ITFG1 | Cerebral palsy | 27 | up-regulated |
| ITFG1 | Cerebrovascular disease | 9 | down-regulated |
| ITFG1 | Chronic fatigue syndrome | 78 | up-regulated |
| ITFG1 | Dementia | 43 | down-regulated |
| ITFG1 | Disorder of basal ganglia | 78 | down-regulated |
| ITFG1 | Disorder of brain | 20 | up-regulated |
| ITFG1 | Encephalomyelopathy | 21 | down-regulated |
| ITFG1 | Epilepsy | 8 | down-regulated |
| ITFG1 | Huntington's disease | 86 | down-regulated |
| ITFG1 | Hypoxia of brain | 2 | down-regulated |
| ITFG1 | Meningitis | 44 | up-regulated |
| ITFG1 | Mood disorder | 37 | down-regulated |
| ITFG1 | Movement disorder | 75 | down-regulated |
| ITFG1 | Multiple sclerosis | 24 | down-regulated |
| ITFG1 | Nerve injury | 28 | down-regulated |
| ITFG1 | Neuropathy | 10 | down-regulated |
| ITFG1 | Paralytic syndrome | 42 | down-regulated |
| ITFG1 | Parkinson's disease | 62 | down-regulated |
| ITFG1 | Prion disease | 20 | down-regulated |
| ITFG1 | Psychotic disorder | 22 | down-regulated |
| ITFG1 | Schizophrenia | 23 | down-regulated |
| ITFG1 | Sleep disorder | 1 | down-regulated |
| ITFG1 | Spinocerebellar ataxia | 16 | up-regulated |
| MAGEH1 | Anxiety disorder | 46 | up-regulated |
| MAGEH1 | Autistic disorder | 22 | down-regulated |
| MAGEH1 | Disorder of basal ganglia | 44 | up-regulated |
| MAGEH1 | Encephalomyelopathy | 33 | down-regulated |
| MAGEH1 | Huntington's disease | 48 | up-regulated |
| MAGEH1 | Meningitis | 41 | up-regulated |
| MAGEH1 | Mood disorder | 8 | down-regulated |
| MAGEH1 | Movement disorder | 41 | up-regulated |
| MAGEH1 | Myoneural disorder | 54 | up-regulated |
| MAGEH1 | Nerve injury | 57 | down-regulated |
| MAGEH1 | Neuropathy | 41 | up-regulated |
| MAGEH1 | Paralytic syndrome | 40 | up-regulated |
| MAGEH1 | Parkinson's disease | 36 | down-regulated |
| MAGEH1 | Prion disease | 30 | down-regulated |
| MAGEH1 | Psychotic disorder | 22 | down-regulated |
| MAGEH1 | Schizophrenia | 23 | down-regulated |
| MAGEH1 | Spinocerebellar ataxia | 43 | down-regulated |
| NALCN | Alzheimer's disease | 68 | down-regulated |
| NALCN | Amnestic disorder | 54 | down-regulated |
| NALCN | Anxiety disorder | 56 | up-regulated |
| NALCN | Cerebrovascular disease | 23 | down-regulated |
| NALCN | Dementia | 67 | down-regulated |
| NALCN | Disorder of basal ganglia | 44 | up-regulated |
| NALCN | Epilepsy | 76 | 3.6E−6 p-value |
| NALCN | Huntington's disease | 47 | up-regulated |
| NALCN | Hypoxia of brain | 25 | down-regulated |
| NALCN | Meningitis | 48 | down-regulated |
| NALCN | Mood disorder | 45 | 3.3E−5 p-value |
| NALCN | Movement disorder | 41 | up-regulated |
| NALCN | Multiple sclerosis | 8 | down-regulated |
| NALCN | Myoneural disorder | 39 | down-regulated |
| NALCN | Nerve injury | 55 | down-regulated |
| NALCN | Neuropathy | 40 | down-regulated |
| NALCN | Parkinson's disease | 39 | up-regulated |
| NALCN | Prion disease | 30 | down-regulated |
| NALCN | Psychotic disorder | 51 | up-regulated |
| NALCN | Schizophrenia | 52 | up-regulated |
| NCAM1 | Amnestic disorder | 1 | down-regulated |
| NCAM1 | Autistic disorder | 1 | down-regulated |
| NCAM1 | Dementia | 1 | up-regulated |
| NCAM1 | Disorder of basal ganglia | 32 | down-regulated |
| NCAM1 | Huntington's disease | 36 | up-regulated |
| NCAM1 | Meningitis | 33 | up-regulated |
| NCAM1 | Movement disorder | 29 | down-regulated |
| NCAM1 | Parkinson's disease | 23 | up-regulated |
| NCAM1 | Psychotic disorder | 16 | down-regulated |
| NCAM1 | Schizophrenia | 17 | down-regulated |
| NCAM1 | Sleep disorder | 11 | down-regulated |
| NETO2 | Amnestic disorder | 41 | down-regulated |
| NETO2 | Anxiety disorder | 36 | up-regulated |
| NETO2 | Dementia | 43 | down-regulated |
| NETO2 | Disorder of basal ganglia | 79 | down-regulated |
| NETO2 | Huntington's disease | 90 | down-regulated |
| NETO2 | Mood disorder | 21 | down-regulated |
| NETO2 | Movement disorder | 76 | down-regulated |
| NETO2 | Nerve injury | 54 | down-regulated |
| NETO2 | Parkinson's disease | 48 | down-regulated |
| NETO2 | Psychotic disorder | 32 | up-regulated |
| NETO2 | Schizophrenia | 32 | up-regulated |
| NETO2 | Sleep disorder | 52 | up-regulated |
| NTRK3 | Alzheimer's disease | 26 | up-regulated |
| NTRK3 | Amnestic disorder | 59 | up-regulated |
| NTRK3 | Autistic disorder | 48 | down-regulated |
| NTRK3 | Cerebral palsy | 65 | down-regulated |
| NTRK3 | Cerebrovascular disease | 33 | down-regulated |
| NTRK3 | Chronic fatigue syndrome | 85 | down-regulated |
| NTRK3 | Dementia | 26 | up-regulated |
| NTRK3 | Developmental mental | 50 | down-regulated |
| | disorder | | |
| NTRK3 | Disorder of basal ganglia | 69 | down-regulated |
| NTRK3 | Encephalitis | 68 | down-regulated |
| NTRK3 | Huntington's disease | 76 | down-regulated |
| NTRK3 | Hypoxia of brain | 36 | down-regulated |
| NTRK3 | Meningitis | 80 | down-regulated |
| NTRK3 | Mental retardation | 48 | down-regulated |
| NTRK3 | Movement disorder | 66 | down-regulated |
| NTRK3 | Multiple sclerosis | 56 | up-regulated |
| NTRK3 | Nerve injury | 91 | down-regulated |
| NTRK3 | Neural tube defect | 53 | up-regulated |
| NTRK3 | Neuropathy | 68 | down-regulated |
| NTRK3 | Parkinson's disease | 53 | down-regulated |
| NTRK3 | Prion disease | 63 | up-regulated |
| NTRK3 | Psychotic disorder | 94 | up-regulated |
| NTRK3 | Schizophrenia | 94 | up-regulated |
| NTRK3 | Sleep disorder | 64 | down-regulated |
| OPN5 | Disorder of basal ganglia | 27 | down-regulated |
| OPN5 | Meningitis | 70 | up-regulated |
| OPN5 | Movement disorder | 24 | down-regulated |
| OPN5 | Neuropathy | 29 | down-regulated |
| OPN5 | Parkinson's disease | 35 | down-regulated |
| OPN5 | Psychotic disorder | 68 | up-regulated |
| OPN5 | Schizophrenia | 68 | up-regulated |
| PAGE3 | Disorder of basal ganglia | 77 | down-regulated |
| PAGE3 | Movement disorder | 74 | down-regulated |
| PAGE3 | Parkinson's disease | 85 | down-regulated |
| PAGE5 | Disorder of basal ganglia | 52 | down-regulated |
| PAGE5 | Huntington's disease | 36 | down-regulated |
| PAGE5 | Meningitis | 47 | down-regulated |
| PAGE5 | Movement disorder | 49 | down-regulated |
| PAGE5 | Multiple sclerosis | 36 | up-regulated |
| PAGE5 | Parkinson's disease | 56 | down-regulated |
| PAGE5 | Psychotic disorder | 86 | up-regulated |
| PAGE5 | Schizophrenia | 87 | up-regulated |
| PHKB | Alzheimer's disease | 2 | down-regulated |
| PHKB | Anxiety disorder | 12 | up-regulated |
| PHKB | Autistic disorder | 7 | up-regulated |
| PHKB | Cerebral palsy | 36 | down-regulated |
| PHKB | Childhood disorder of conduct | 16 | up-regulated |
| | and emotion | | |
| PHKB | Chronic fatigue syndrome | 67 | up-regulated |
| PHKB | Dementia | 2 | down-regulated |
| PHKB | Disorder of basal ganglia | 35 | down-regulated |
| PHKB | Disorder of brain | 2 | up-regulated |
| PHKB | Encephalomyelopathy | 26 | down-regulated |
| PHKB | Epilepsy | 1 | down-regulated |
| PHKB | Huntington's disease | 29 | up-regulated |
| PHKB | Meningitis | 35 | down-regulated |
| PHKB | Movement disorder | 32 | down-regulated |
| PHKB | Multiple sclerosis | 1 | down-regulated |
| PHKB | Nerve injury | 25 | down-regulated |
| PHKB | Neuropathy | 23 | down-regulated |
| PHKB | Paralytic syndrome | 46 | down-regulated |
| PHKB | Parkinson's disease | 36 | down-regulated |
| PHKB | Prion disease | 15 | up-regulated |
| PHKB | Sleep disorder | 1 | up-regulated |
| PHKB | Spinocerebellar ataxia | 9 | up-regulated |
| PPP1R1C | Attention deficit hyperactivity | 1 | 0.0003 p-value |
| | disorder | | |
| PPP1R1C | Developmental mental | 11 | down-regulated |
| | disorder | | |
| PPP1R1C | Disorder of basal ganglia | 1 | up-regulated |
| PPP1R1C | Meningitis | 8 | up-regulated |
| PPP1R1C | Mental retardation | 9 | down-regulated |
| PPP1R1C | Mood disorder | 1 | 0.0008 p-value |
| PPP1R1C | Movement disorder | 1 | up-regulated |
| PPP1R1C | Multiple sclerosis | 11 | up-regulated |
| PPP1R1C | Myoneural disorder | 20 | down-regulated |
| PPP1R1C | Nerve injury | 26 | up-regulated |
| PPP1R1C | Neural tube defect | 27 | down-regulated |
| PPP1R1C | Neuropathy | 17 | down-regulated |
| PPP1R1C | Parkinson's disease | 1 | up-regulated |
| PPP1R1C | Psychotic disorder | 4 | 7.9E−5 p-value |
| PPP1R1C | Schizophrenia | 4 | 7.9E−5 p-value |
| PSMC1 | Alzheimer's disease | 41 | up-regulated |
| PSMC1 | Anxiety disorder | 40 | up-regulated |
| PSMC1 | Autistic disorder | 23 | down-regulated |
| PSMC1 | Cerebrovascular disease | 54 | down-regulated |
| PSMC1 | Dementia | 41 | up-regulated |
| PSMC1 | Disorder of basal ganglia | 59 | down-regulated |
| PSMC1 | Huntington's disease | 48 | down-regulated |
| PSMC1 | Hypoxia of brain | 40 | up-regulated |
| PSMC1 | Movement disorder | 56 | down-regulated |
| PSMC1 | Nerve injury | 34 | down-regulated |
| PSMC1 | Neuropathy | 67 | down-regulated |
| PSMC1 | Parkinson's disease | 62 | down-regulated |
| PSMC1 | Prion disease | 82 | down-regulated |
| PSMC1 | Psychotic disorder | 39 | down-regulated |
| PSMC1 | Schizophrenia | 40 | down-regulated |
| PSMC1 | Sleep disorder | 27 | down-regulated |
| PTBP2 | Amnestic disorder | 6 | down-regulated |
| PTBP2 | Amyotrophic lateral sclerosis | 10 | down-regulated |
| PTBP2 | Anxiety disorder | 45 | up-regulated |
| PTBP2 | Autistic disorder | 14 | up-regulated |
| PTBP2 | Cerebral palsy | 28 | up-regulated |
| PTBP2 | Disorder of basal ganglia | 51 | down-regulated |
| PTBP2 | Encephalomyelopathy | 11 | down-regulated |
| PTBP2 | Epilepsy | 23 | 0.0002 p-value |
| PTBP2 | Huntington's disease | 31 | up-regulated |
| PTBP2 | Meningitis | 51 | down-regulated |
| PTBP2 | Mood disorder | 56 | down-regulated |
| PTBP2 | Motor neuron disease | 22 | down-regulated |
| PTBP2 | Movement disorder | 48 | down-regulated |
| PTBP2 | Nerve injury | 47 | down-regulated |
| PTBP2 | Neuropathy | 26 | down-regulated |
| PTBP2 | Paralytic syndrome | 32 | up-regulated |
| PTBP2 | Parkinson's disease | 57 | down-regulated |
| PTBP2 | Prion disease | 17 | down-regulated |
| PTBP2 | Psychotic disorder | 42 | up-regulated |
| PTBP2 | Schizophrenia | 42 | up-regulated |
| PTBP2 | Sleep disorder | 1 | down-regulated |
| RP11 | Amnestic disorder | 30 | up-regulated |
| RP11 | Anxiety disorder | 64 | down-regulated |
| RP11 | Autistic disorder | 52 | up-regulated |
| RP11 | Cerebrovascular disease | 27 | down-regulated |
| RP11 | Developmental mental | 68 | up-regulated |
| | disorder | | |
| RP11 | Disorder of basal ganglia | 70 | down-regulated |
| RP11 | Disorder of brain | 49 | down-regulated |
| RP11 | Encephalomyelopathy | 39 | up-regulated |
| RP11 | Huntington's disease | 82 | down-regulated |
| RP11 | Hypoxia of brain | 24 | up-regulated |
| RP11 | Meningitis | 81 | down-regulated |
| RP11 | Mental retardation | 65 | up-regulated |
| RP11 | Mood disorder | 17 | up-regulated |
| RP11 | Movement disorder | 67 | down-regulated |
| RP11 | Nerve injury | 25 | up-regulated |
| RP11 | Neuropathy | 43 | up-regulated |
| RP11 | Paralytic syndrome | 49 | up-regulated |
| RP11 | Parkinson's disease | 34 | down-regulated |
| RP11 | Prion disease | 48 | down-regulated |
| RP11 | Psychotic disorder | 41 | up-regulated |
| RP11 | Schizophrenia | 41 | up-regulated |
| RP11 | Sleep disorder | 59 | down-regulated |
| RP11 | Spinocerebellar ataxia | 44 | up-regulated |
| RP13 | Alzheimer's disease | 51 | down-regulated |
| RP13 | Attention deficit hyperactivity | 79 | |
| | disorder | | |
| RP13 | Autistic disorder | 68 | down-regulated |
| RP13 | Cerebrovascular disease | 19 | down-regulated |
| RP13 | Dementia | 51 | down-regulated |
| RP13 | Developmental mental | 99 | |
| | disorder | | |
| RP13 | Disorder of basal ganglia | 25 | up-regulated |
| RP13 | Encephalitis | 55 | down-regulated |
| RP13 | Encephalomyelopathy | 24 | up-regulated |
| RP13 | Huntington's disease | 27 | up-regulated |
| RP13 | Hypoxia of brain | 33 | down-regulated |
| RP13 | Meningitis | 71 | up-regulated |
| RP13 | Mental retardation | 97 | |
| RP13 | Movement disorder | 23 | up-regulated |
| RP13 | Nerve injury | 24 | down-regulated |
| RP13 | Neuropathy | 16 | up-regulated |
| RP13 | Paralytic syndrome | 44 | up-regulated |
| RP13 | Parkinson's disease | 21 | down-regulated |
| RP13 | Sleep disorder | 29 | down-regulated |
| RP4 | Anxiety disorder | 25 | down-regulated |
| RP4 | Autistic disorder | 25 | down-regulated |
| RP4 | Cerebral palsy | 46 | down-regulated |
| RP4 | Developmental mental | 32 | down-regulated |
| | disorder | | |
| RP4 | Disorder of basal ganglia | 8 | down-regulated |
| RP4 | Encephalitis | 33 | down-regulated |
| RP4 | Encephalomyelopathy | 16 | up-regulated |
| RP4 | Huntington's disease | 9 | down-regulated |
| RP4 | Meningitis | 34 | down-regulated |
| RP4 | Mental retardation | 29 | down-regulated |
| RP4 | Mood disorder | 36 | 3.1E−5 p-value |
| RP4 | Motor neuron disease | 3 | down-regulated |
| RP4 | Movement disorder | 5 | down-regulated |
| RP4 | Nerve injury | 31 | down-regulated |
| RP4 | Neuropathy | 27 | down-regulated |
| RP4 | Parkinson's disease | 4 | up-regulated |
| RPL35 | Alzheimer's disease | 2 | up-regulated |
| RPL35 | Amnestic disorder | 20 | up-regulated |
| RPL35 | Autistic disorder | 30 | up-regulated |
| RPL35 | Cerebrovascular disease | 16 | up-regulated |
| RPL35 | Dementia | 2 | up-regulated |
| RPL35 | Disorder of basal ganglia | 26 | up-regulated |
| RPL35 | Encephalitis | 29 | down-regulated |
| RPL35 | Encephalomyelitis | 40 | down-regulated |
| RPL35 | Encephalomyelopathy | 6 | down-regulated |
| RPL35 | Huntington's disease | 35 | up-regulated |
| RPL35 | Hypoxia of brain | 10 | up-regulated |
| RPL35 | Meningitis | 87 | up-regulated |
| RPL35 | Mood disorder | 4 | down-regulated |
| RPL35 | Motor neuron disease | 23 | up-regulated |
| RPL35 | Movement disorder | 23 | up-regulated |
| RPL35 | Multiple sclerosis | 3 | up-regulated |
| RPL35 | Myoneural disorder | 27 | up-regulated |
| RPL35 | Nerve injury | 26 | up-regulated |
| RPL35 | Neuropathy | 28 | up-regulated |
| RPL35 | Parkinson's disease | 4 | down-regulated |
| RPL35 | Prion disease | 15 | down-regulated |
| RPL35 | Psychotic disorder | 1 | 0.0008 p-value |
| RPL35 | Schizophrenia | 1 | 0.0008 p-value |
| RPL35 | Sleep disorder | 43 | down-regulated |
| RPL5 | Alzheimer's disease | 3 | down-regulated |
| RPL5 | Amyotrophic lateral sclerosis | 29 | down-regulated |
| RPL5 | Autistic disorder | 23 | up-regulated |
| RPL5 | Cerebrovascular disease | 6 | up-regulated |
| RPL5 | Dementia | 3 | down-regulated |
| RPL5 | Disorder of basal ganglia | 33 | up-regulated |
| RPL5 | Disorder of brain | 12 | up-regulated |
| RPL5 | Encephalitis | 58 | down-regulated |
| RPL5 | Encephalomyelitis | 37 | down-regulated |
| RPL5 | Encephalomyelopathy | 2 | down-regulated |
| RPL5 | Huntington's disease | 40 | up-regulated |
| RPL5 | Hypoxia of brain | 1 | up-regulated |
| RPL5 | Meningitis | 52 | down-regulated |
| RPL5 | Motor neuron disease | 38 | down-regulated |
| RPL5 | Movement disorder | 30 | up-regulated |
| RPL5 | Multiple sclerosis | 70 | 2.5E−6 p-value |
| RPL5 | Myoneural disorder | 17 | up-regulated |
| RPL5 | Nerve injury | 22 | down-regulated |
| RPL5 | Neuropathy | 7 | up-regulated |
| RPL5 | Paralytic syndrome | 17 | up-regulated |
| RPL5 | Parkinson's disease | 18 | up-regulated |
| RPL5 | Prion disease | 13 | down-regulated |
| RPL5 | Psychotic disorder | 54 | 2.2E−6 p-value |
| RPL5 | Schizophrenia | 55 | 2.2E−6 p-value |
| RPL5 | Sleep disorder | 24 | down-regulated |
| RRAGB | Alzheimer's disease | 22 | down-regulated |
| RRAGB | Dementia | 21 | down-regulated |
| RRAGB | Disorder of basal ganglia | 36 | down-regulated |
| RRAGB | Disorder of brain | 17 | up-regulated |
| RRAGB | Encephalitis | 27 | down-regulated |
| RRAGB | Encephalomyelopathy | 6 | down-regulated |
| RRAGB | Huntington's disease | 19 | down-regulated |
| RRAGB | Meningitis | 11 | up-regulated |
| RRAGB | Mood disorder | 1 | up-regulated |
| RRAGB | Motor neuron disease | 1 | up-regulated |
| RRAGB | Movement disorder | 33 | down-regulated |
| RRAGB | Multiple sclerosis | 9 | down-regulated |
| RRAGB | Nerve injury | 48 | down-regulated |
| RRAGB | Neuropathy | 6 | down-regulated |
| RRAGB | Parkinson's disease | 41 | down-regulated |
| RRAGB | Psychotic disorder | 13 | down-regulated |
| RRAGB | Schizophrenia | 13 | down-regulated |
| RRAGB | Sleep disorder | 18 | down-regulated |
| RYR3 | Alzheimer's disease | 26 | down-regulated |
| RYR3 | Anxiety disorder | 63 | up-regulated |
| RYR3 | Autistic disorder | 21 | up-regulated |
| RYR3 | Cerebral palsy | 85 | up-regulated |
| RYR3 | Cerebrovascular disease | 65 | 6.5E−6 p-value |
| RYR3 | Dementia | 25 | down-regulated |
| RYR3 | Developmental mental | 36 | down-regulated |
| | disorder | | |
| RYR3 | Disorder of basal ganglia | 56 | up-regulated |
| RYR3 | Disorder of brain | 49 | up-regulated |
| RYR3 | Encephalitis | 50 | up-regulated |
| RYR3 | Encephalomyelitis | 61 | up-regulated |
| RYR3 | Encephalomyelopathy | 34 | up-regulated |
| RYR3 | Epilepsy | 60 | 0.7E−5 p-value |
| RYR3 | Huntington's disease | 68 | up-regulated |
| RYR3 | Meningitis | 57 | up-regulated |
| RYR3 | Mental retardation | 34 | down-regulated |
| RYR3 | Mood disorder | 57 | 8.3E−6 p-value |
| RYR3 | Movement disorder | 53 | up-regulated |
| RYR3 | Multiple sclerosis | 24 | up-regulated |
| RYR3 | Myoneural disorder | 46 | up-regulated |
| RYR3 | Nerve injury | 70 | down-regulated |
| RYR3 | Neuropathy | 44 | down-regulated |
| RYR3 | Parkinson's disease | 10 | up-regulated |
| RYR3 | Prion disease | 47 | down-regulated |
| RYR3 | Psychotic disorder | 57 | up-regulated |
| RYR3 | Schizophrenia | 58 | up-regulated |
| RYR3 | Sleep disorder | 46 | up-regulated |
| SCAI | Alzheimer's disease | 38 | down-regulated |
| SCAI | Amyotrophic lateral sclerosis | 41 | up-regulated |
| SCAI | Autistic disorder | 16 | up-regulated |
| SCAI | Cerebrovascular disease | 14 | down-regulated |
| SCAI | Dementia | 38 | down-regulated |
| SCAI | Disorder of basal ganglia | 77 | down-regulated |
| SCAI | Huntington's disease | 66 | down-regulated |
| SCAI | Hypoxia of brain | 17 | down-regulated |
| SCAI | Meningitis | 54 | down-regulated |
| SCAI | Mood disorder | 26 | down-regulated |
| SCAI | Motor neuron disease | 38 | up-regulated |
| SCAI | Movement disorder | 74 | down-regulated |
| SCAI | Multiple sclerosis | 3 | down-regulated |
| SCAI | Nerve injury | 41 | up-regulated |
| SCAI | Neuropathy | 14 | up-regulated |
| SCAI | Parkinson's disease | 78 | down-regulated |
| SCAI | Prion disease | 43 | up-regulated |
| SCAI | Psychotic disorder | 35 | down-regulated |
| SCAI | Schizophrenia | 35 | down-regulated |
| SCAI | Sleep disorder | 53 | up-regulated |
| SEMA3A | Alzheimer's disease | 1 | 5.9E−5 p-value |
| SEMA3A | Amnestic disorder | 1 | down-regulated |
| SEMA3A | Autistic disorder | 1 | down-regulated |
| SEMA3A | Childhood disorder of conduct | 26 | up-regulated |
| | and emotion | | |
| SEMA3A | Dementia | 1 | 5.9E−5 p-value |
| SEMA3A | Disorder of basal ganglia | 7 | down-regulated |
| SEMA3A | Huntington's disease | 17 | down-regulated |
| SEMA3A | Lissencephaly | 100 | |
| SEMA3A | Mood disorder | 1 | 0.0003 p-value |
| SEMA3A | Motor neuron disease | 1 | up-regulated |
| SEMA3A | Movement disorder | 4 | down-regulated |
| SEMA3A | Multiple sclerosis | 1 | up-regulated |
| SEMA3A | Nerve injury | 8 | up-regulated |
| SEMA3A | Neuropathy | 71 | down-regulated |
| SEMA3A | Parkinson's disease | 1 | up-regulated |
| SEMA3A | Prion disease | 45 | 2.7E−6 p-value |
| SEMA3A | Psychotic disorder | 26 | down-regulated |
| SEMA3A | Schizophrenia | 26 | down-regulated |
| SEMA3A | Sleep disorder | 30 | up-regulated |
| SLC20A2 | Amnestic disorder | 19 | up-regulated |
| SLC20A2 | Autistic disorder | 7 | up-regulated |
| SLC20A2 | Disorder of basal ganglia | 28 | down-regulated |
| SLC20A2 | Disorder of brain | 26 | up-regulated |
| SLC20A2 | Encephalomyelopathy | 14 | down-regulated |
| SLC20A2 | Huntington's disease | 29 | down-regulated |
| SLC20A2 | Meningitis | 8 | up-regulated |
| SLC20A2 | Mood disorder | 19 | 8.5E−5 p-value |
| SLC20A2 | Motor neuron disease | 5 | down-regulated |
| SLC20A2 | Movement disorder | 25 | down-regulated |
| SLC20A2 | Multiple sclerosis | 50 | up-regulated |
| SLC20A2 | Nerve injury | 50 | up-regulated |
| SLC20A2 | Neuropathy | 28 | down-regulated |
| SLC20A2 | Paralytic syndrome | 24 | down-regulated |
| SLC20A2 | Parkinson's disease | 24 | down-regulated |
| SLC20A2 | Prion disease | 40 | up-regulated |
| SLC20A2 | Psychotic disorder | 17 | up-regulated |
| SLC20A2 | Schizophrenia | 17 | up-regulated |
| SLC20A2 | Sleep disorder | 10 | down-regulated |
| SLC25A14 | Alzheimer's disease | 27 | down-regulated |
| SLC25A14 | Autistic disorder | 1 | down-regulated |
| SLC25A14 | Cerebral palsy | 20 | down-regulated |
| SLC25A14 | Dementia | 26 | down-regulated |
| SLC25A14 | Disorder of basal ganglia | 45 | down-regulated |
| SLC25A14 | Encephalitis | 24 | up-regulated |
| SLC25A14 | Encephalomyelopathy | 12 | up-regulated |
| SLC25A14 | Huntington's disease | 47 | down-regulated |
| SLC25A14 | Meningitis | 16 | down-regulated |
| SLC25A14 | Movement disorder | 42 | down-regulated |
| SLC25A14 | Multiple sclerosis | 2 | down-regulated |
| SLC25A14 | Nerve injury | 27 | down-regulated |
| SLC25A14 | Neuropathy | 18 | down-regulated |
| SLC25A14 | Parkinson's disease | 41 | down-regulated |
| SLC25A14 | Prion disease | 29 | down-regulated |
| SLC25A14 | Psychotic disorder | 25 | up-regulated |
| SLC25A14 | Schizophrenia | 25 | up-regulated |
| SLC25A14 | Spinocerebellar ataxia | 14 | up-regulated |
| SMARCAD1 | Alzheimer's disease | 19 | down-regulated |
| SMARCAD1 | Amnestic disorder | 1 | up-regulated |
| SMARCAD1 | Anxiety disorder | 28 | up-regulated |
| SMARCAD1 | Autistic disorder | 1 | down-regulated |
| SMARCAD1 | Cerebrovascular disease | 11 | up-regulated |
| SMARCAD1 | Dementia | 18 | down-regulated |
| SMARCAD1 | Disorder of basal ganglia | 1 | up-regulated |
| SMARCAD1 | Encephalomyelopathy | 1 | down-regulated |
| SMARCAD1 | Huntington's disease | 11 | up-regulated |
| SMARCAD1 | Meningitis | 39 | down-regulated |
| SMARCAD1 | Mood disorder | 13 | up-regulated |
| SMARCAD1 | Movement disorder | 1 | up-regulated |
| SMARCAD1 | Nerve injury | 17 | down-regulated |
| SMARCAD1 | Neuropathy | 14 | down-regulated |
| SMARCAD1 | Paralytic syndrome | 11 | up-regulated |
| SMARCAD1 | Prion disease | 12 | down-regulated |
| SMARCAD1 | Psychotic disorder | 1 | 0.0002 p-value |
| SMARCAD1 | Schizophrenia | 1 | 0.0002 p-value |
| SMARCAD1 | Sleep disorder | 26 | up-regulated |
| SMARCAD1 | Spinocerebellar ataxia | 8 | down-regulated |
| SNORA42 | Attention deficit hyperactivity | 90 | 4.9E−6 p-value |
| | disorder | | |
| SNORA42 | Encephalomyelopathy | 51 | up-regulated |
| SNORA42 | Neuropathy | 52 | up-regulated |
| SNORA66 | Autistic disorder | 33 | down-regulated |
| SNORA66 | Multiple sclerosis | 100 | 2.5E−6 p-value |
| SNORA66 | Psychotic disorder | 83 | 2.2E−6 p-value |
| SNORA66 | Schizophrenia | 83 | 2.2E−6 p-value |
| SNTG1 | Alzheimer's disease | 1 | down-regulated |
| SNTG1 | Cerebrovascular disease | 1 | down-regulated |
| SNTG1 | Dementia | 1 | down-regulated |
| SNTG1 | Developmental mental | 68 | down-regulated |
| | disorder | | |
| SNTG1 | Disorder of basal ganglia | 30 | down-regulated |
| SNTG1 | Huntington's disease | 38 | down-regulated |
| SNTG1 | Hypoxia of brain | 7 | down-regulated |
| SNTG1 | Meningitis | 1 | up-regulated |
| SNTG1 | Mental disorder | 100 | down-regulated |
| SNTG1 | Movement disorder | 27 | down-regulated |
| SNTG1 | Multiple sclerosis | 3 | up-regulated |
| SNTG1 | Neuropathy | 1 | down-regulated |
| SNTG1 | Parkinson's disease | 13 | down-regulated |
| SNTG1 | Sleep disorder | 5 | down-regulated |
| SNX19 | Disorder of basal ganglia | 49 | down-regulated |
| SNX19 | Encephalomyelopathy | 12 | down-regulated |
| SNX19 | Huntington's disease | 55 | down-regulated |
| SNX19 | Meningitis | 67 | up-regulated |
| SNX19 | Mood disorder | 23 | down-regulated |
| SNX19 | Movement disorder | 46 | down-regulated |
| SNX19 | Multiple sclerosis | 12 | down-regulated |
| SNX19 | Myoneural disorder | 44 | down-regulated |
| SNX19 | Nerve injury | 32 | down-regulated |
| SNX19 | Neuropathy | 43 | down-regulated |
| SNX19 | Paralytic syndrome | 33 | down-regulated |
| SNX19 | Parkinson's disease | 38 | down-regulated |
| SNX19 | Prion disease | 36 | up-regulated |
| SNX19 | Psychotic disorder | 82 | down-regulated |
| SNX19 | Schizophrenia | 83 | down-regulated |
| SNX19 | Sleep disorder | 51 | up-regulated |
| SOD3 | Alzheimer's disease | 1 | down-regulated |
| SOD3 | Anxiety disorder | 1 | up-regulated |
| SOD3 | Cerebrovascular disease | 1 | down-regulated |
| SOD3 | Dementia | 18 | up-regulated |
| SOD3 | Disorder of basal ganglia | 1 | up-regulated |
| SOD3 | Disorder of brain | 1 | down-regulated |
| SOD3 | Huntington's disease | 1 | up-regulated |
| SOD3 | Meningitis | 2 | down-regulated |
| SOD3 | Motor neuron disease | 1 | down-regulated |
| SOD3 | Movement disorder | 1 | up-regulated |
| SOD3 | Nerve injury | 20 | up-regulated |
| SOD3 | Neuropathy | 20 | up-regulated |
| SOD3 | Prion disease | 32 | up-regulated |
| SOD3 | Psychotic disorder | 1 | up-regulated |
| SOD3 | Schizophrenia | 1 | up-regulated |
| SOD3 | Sleep disorder | 1 | up-regulated |
| SPATA7 | Alzheimer's disease | 23 | down-regulated |
| SPATA7 | Autistic disorder | 39 | down-regulated |
| SPATA7 | Dementia | 23 | down-regulated |
| SPATA7 | Disorder of basal ganglia | 71 | up-regulated |
| SPATA7 | Disorder of brain | 77 | up-regulated |
| SPATA7 | Encephalomyelopathy | 36 | up-regulated |
| SPATA7 | Huntington's disease | 81 | up-regulated |
| SPATA7 | Meningitis | 54 | up-regulated |
| SPATA7 | Mood disorder | 30 | down-regulated |
| SPATA7 | Movement disorder | 68 | up-regulated |
| SPATA7 | Nerve injury | 76 | down-regulated |
| SPATA7 | Neuropathy | 61 | down-regulated |
| SPATA7 | Parkinson's disease | 50 | down-regulated |
| SPATA7 | Psychotic disorder | 75 | down-regulated |
| SPATA7 | Schizophrenia | 76 | down-regulated |
| SPATA7 | Sleep disorder | 98 | down-regulated |
| ST18 | Alzheimer's disease | 63 | down-regulated |
| ST18 | Amnestic disorder | 37 | up-regulated |
| ST18 | Dementia | 62 | down-regulated |
| ST18 | Disorder of basal ganglia | 68 | up-regulated |
| ST18 | Disorder of brain | 69 | up-regulated |
| ST18 | Epilepsy | 58 | 4.8E−5 p-value |
| ST18 | Huntington's disease | 76 | up-regulated |
| ST18 | Mood disorder | 35 | down-regulated |
| ST18 | Movement disorder | 65 | up-regulated |
| ST18 | Multiple sclerosis | 53 | down-regulated |
| ST18 | Nerve injury | 49 | up-regulated |
| ST18 | Neuropathy | 46 | down-regulated |
| ST18 | Parkinson's disease | 51 | up-regulated |
| ST18 | Prion disease | 49 | down-regulated |
| ST18 | Psychotic disorder | 48 | up-regulated |
| ST18 | Schizophrenia | 48 | up-regulated |
| ST18 | Sleep disorder | 36 | down-regulated |
| STYK1 | Alzheimer's disease | 52 | down-regulated |
| STYK1 | Dementia | 51 | down-regulated |
| STYK1 | Disorder of basal ganglia | 49 | down-regulated |
| STYK1 | Huntington's disease | 55 | down-regulated |
| STYK1 | Hypoxia of brain | 33 | up-regulated |
| STYK1 | Mood disorder | 8 | 0.0003 p-value |
| STYK1 | Movement disorder | 47 | down-regulated |
| STYK1 | Neural tube defect | 100 | down-regulated |
| STYK1 | Neuropathy | 7 | down-regulated |
| STYK1 | Parkinson's disease | 38 | down-regulated |
| STYK1 | Psychotic disorder | 41 | down-regulated |
| STYK1 | Schizophrenia | 41 | down-regulated |
| TMEM135 | Cerebral palsy | 57 | up-regulated |
| TMEM135 | Dementia | 24 | down-regulated |
| TMEM135 | Disorder of basal ganglia | 43 | down-regulated |
| TMEM135 | Disorder of brain | 44 | up-regulated |
| TMEM135 | Mood disorder | 22 | down-regulated |
| TMEM135 | Paralytic syndrome | 62 | up-regulated |
| TMEM135 | Parkinson's disease | 47 | down-regulated |
| TMEM135 | Psychotic disorder | 54 | up-regulated |
| TMEM135 | Schizophrenia | 54 | up-regulated |
| TRPS1 | Alzheimer's disease | 19 | up-regulated |
| TRPS1 | Autistic disorder | 1 | up-regulated |
| TRPS1 | Cerebrovascular disease | 23 | 5.0E−5 p-value |
| TRPS1 | Dementia | 18 | up-regulated |
| TRPS1 | Disorder of basal ganglia | 57 | up-regulated |
| TRPS1 | Encephalomyelopathy | 1 | down-regulated |
| TRPS1 | Huntington's disease | 66 | up-regulated |
| TRPS1 | Hypoxia of brain | 14 | up-regulated |
| TRPS1 | Meningitis | 51 | up-regulated |
| TRPS1 | Mood disorder | 1 | 0.0004 p-value |
| TRPS1 | Motor neuron disease | 13 | down-regulated |
| TRPS1 | Movement disorder | 54 | up-regulated |
| TRPS1 | Multiple sclerosis | 27 | up-regulated |
| TRPS1 | Nerve injury | 27 | up-regulated |
| TRPS1 | Neuropathy | 29 | up-regulated |
| TRPS1 | Parkinson's disease | 36 | up-regulated |
| TRPS1 | Psychotic disorder | 18 | up-regulated |
| TRPS1 | Schizophrenia | 18 | up-regulated |
| TRPS1 | Sleep disorder | 15 | down-regulated |
| TRPS1 | Spinocerebellar ataxia | 12 | down-regulated |
| VANGL1 | Autistic disorder | 1 | down-regulated |
| VANGL1 | Disorder of basal ganglia | 1 | up-regulated |
| VANGL1 | Epilepsy | 11 | down-regulated |
| VANGL1 | Huntington's disease | 1 | up-regulated |
| VANGL1 | Meningitis | 1 | up-regulated |
| VANGL1 | Mood disorder | 1 | down-regulated |
| VANGL1 | Neural tube defect | 100 | |
| VANGL1 | Psychotic disorder | 1 | down-regulated |
| VANGL1 | Schizophrenia | 1 | down-regulated |
| VDAC3 | Anxiety disorder | 27 | up-regulated |
| VDAC3 | Autistic disorder | 18 | up-regulated |
| VDAC3 | Dementia | 20 | down-regulated |
| VDAC3 | Disorder of basal ganglia | 48 | down-regulated |
| VDAC3 | Encephalomyelopathy | 50 | down-regulated |
| VDAC3 | Meningitis | 65 | up-regulated |
| VDAC3 | Myoneural disorder | 56 | up-regulated |
| VDAC3 | Parkinson's disease | 53 | down-regulated |
| WDR38 | Disorder of basal ganglia | 41 | up-regulated |
| WDR38 | Huntington's disease | 54 | up-regulated |
| WDR38 | Meningitis | 38 | up-regulated |
| WDR38 | Movement disorder | 38 | up-regulated |
| WDR38 | Multiple sclerosis | 40 | up-regulated |
| WDR38 | Nerve injury | 75 | up-regulated |
| WDR38 | Neuropathy | 64 | up-regulated |
| WDR38 | Psychotic disorder | 54 | down-regulated |
| WDR38 | Schizophrenia | 54 | down-regulated |
| ZC3H14 | Alzheimer's disease | 9 | up-regulated |
| ZC3H14 | Amyotrophic lateral sclerosis | 33 | down-regulated |
| ZC3H14 | Anxiety disorder | 43 | up-regulated |
| ZC3H14 | Autistic disorder | 16 | up-regulated |
| ZC3H14 | Cerebrovascular disease | 29 | up-regulated |
| ZC3H14 | Dementia | 8 | up-regulated |
| ZC3H14 | Disorder of basal ganglia | 59 | up-regulated |
| ZC3H14 | Disorder of brain | 16 | down-regulated |
| ZC3H14 | Encephalitis | 41 | down-regulated |
| ZC3H14 | Encephalomyelitis | 52 | down-regulated |
| ZC3H14 | Encephalomyelopathy | 18 | down-regulated |
| ZC3H14 | Huntington's disease | 63 | up-regulated |
| ZC3H14 | Meningitis | 51 | down-regulated |
| ZC3H14 | Mood disorder | 25 | down-regulated |
| ZC3H14 | Motor neuron disease | 30 | down-regulated |
| ZC3H14 | Movement disorder | 56 | up-regulated |
| ZC3H14 | Multiple sclerosis | 57 | down-regulated |
| ZC3H14 | Myoneural disorder | 49 | up-regulated |
| ZC3H14 | Nerve injury | 24 | down-regulated |
| ZC3H14 | Neuropathy | 32 | down-regulated |
| ZC3H14 | Paralytic syndrome | 41 | up-regulated |
| ZC3H14 | Parkinson's disease | 53 | up-regulated |
| ZC3H14 | Prion disease | 43 | up-regulated |
| ZC3H14 | Psychotic disorder | 37 | down-regulated |
| ZC3H14 | Schizophrenia | 38 | down-regulated |
| ZC3H14 | Sleep disorder | 68 | down-regulated |

### Pathways

We identified distinct pathways (see Tables 2 and 6, and FIG. 7) including genes that have already been reported as associated with SZ by GWAS, as well as genes known to be abnormally expressed in the brain of SZ patients. Overall, the products of genes uncovered by the SNP sets are included in several well-known, relevant and interconnected signaling pathways. Annotation information was manually curated and obtained from the Haploreg DB and from the Ensembl and NCBI web services.

### PI3K/Akt Signaling.

Akt is a Serine/threonine Kinase, it is activated by tyrosine kinase receptors, integrins, T and B cell receptors, cytokine receptors, G-proteins-coupled receptors and other stimuli that involves the production of PIP3 triphosphate (phosphatidylinositol triphosphate) by PI3K (phosphoinositide 3 kinase). PI3K can be activated by different ways:

FOXR2 (forkhead box R2) is a proto-oncogene when it is mutated, maintained cell growth and proliferation through activation of RAS (GTPase) increase aberrant signaling through pathways PI3K/AKT/mTOR and RAS/MAP/ERK, inhibiting apoptosis.

SOD3 (superoxide dismutase 3) causes increased of phosphorylation of ERK/Ras and PIP3 because PI3K, SOD3 may be Phosphorilated by Erk½.

SEMA3A inhibits the proliferation and cell growth in neurons and prevents axonal growth by inhibiting the PI3K/Akt via inhibition of Ras. Neuropilin and SEMA1 bound active apoptosis via PI3K/Akt.

RAS (GTPase) can be activated by FOXR2 mutated by SOD3 and inhibited by Sema3A. Ras and PI3K can activate mTORC1 by cRaf/MEK/ERK.

SNX19 inhibits Akt phosphorylation resulting in apoptosis.

STYK1 oncogene that binds to Akt to activate the cascade signaling downstream and leading to increased tumor cells and increasing the risk of metastasis.

CHST9 catalyzes the sulfates transfer to N-acetylgalactosamine residues, inhibits Cd19/p85/PI3K-p110 complex.

RRAGB is part of RAG proteins that interact with mTORC1 family and are required for activation of amino acids via mTORC1.

### Signaling Pathways Activating MAPK/p38/p53.

p38 MAPKs (.alpha., .beta, .gamma., and .delta.) are members of the MAPK family that are activated by a variety of environmental stresses and inflammatory cytokines. As with other MAPK cascades, the membrane-proximal component is a MAPKKK, typically a MEKK or a mixed lineage kinase (MLK). The MAPKKK phosphorylates and activates MKK3/6, the p38 MAPK kinases. MKK3/6 can also be activated directly by ASK1, which is stimulated by apoptotic stimuli. p38 MAPK is involved in regulation of HSP27, MAPKAPK-2 (MK2), MAPKAPK-3 (MK3), and several transcription factors including ATF-2, Statl, the Max/Myc complex, MEF-2, Elk-1, and indirectly CREB via activation of MSK1. This pathway may be activated by activation of PI3K way Rac/MEK/ERK.

DUSP4 is a MKP able of inhibiting p38MAPK 12 and 14a, is regulated by TNF-α expression. Decreases ERK ½ and reducing the cellular viability by alteration of the NF-.kappa.B/MAPK pathways.

MAGEH1 expression causes apoptosis of melanoma cells through the interaction with the inner region to the membrane of the p75 neurotrophin receptor (p75NTR) one TNF receptor type, and possibly also through competition with the TNF receptor associated factor-6 (TRAF6) and catalytic neurotrophin receptor (TRK) for the same site of interaction with p75.

### Nucleus

TRPS1 The gene encodes for an atypical member of the GATA family. It can activate Snail 1 to produce inhibition of cadherines inside of nucleus.

ST18 is a promoter of hypermethylation, ST18 loss of expression in tumor cells suggests that this epigenetic mechanism responsible for the specific down-regulation of tumor.

SPATA7 may be involved in the preparation of chromatin in early meiotic prophase in the nuclei for the initiation of meiotic recombination.

ZC3H14 a protein with zinc finger Cys3His evolutionarily conserved that specifically binds to RNA and polyadenosine therefore postulated to modulate post-transcriptional gene expression.

U4, is part of snRNP small nucleolar ribonucleic particles (RNA-protein), each one bind specifically to individual RNA. The function of the human U4 3″SL micro RNA is unclear. It exists to enable the formation of nucleoplasm in Cajal bodies.

PPP1R1C (Protein phosphatase 1, regulatory subunit 1C) is a protein-coding gene and inhibitor of PP 1, and is itself regulated by phosphorylation. It promotes cell growth and may protect against cell death, particularly when induced by pathological stress.

PRPF31 main function is thought to recruit and strap for U4/U6 U5 tri-snRNP.

EVI5 works in G1/S phases, prevents phosphorylation of Emi 1 by Plk1 and therefore inactive APC/C and accumulates cyclin A. In prometaphase, Plk1 phosphorylates to EVI5, producing its inactivation and subsequent activation of APC/C and downstream signaling pathways to complete the mitotic cycle.

SNORA42: The main functions of snoRNAs has long been thought to modify, mature and stabilize rRNAs. These posttranslational modifications-transcriptional are important for production of accurate and efficient ribosome. Moreover, some snoRNAs are processed to produce small RNAs.

SNORD112. SnoRNAs act as small nucleolar ribonucleoproteins (snoRNPs), each of which consists of a C/D box or box H/ACA RNA guide, and four C/D and H/ACA snoRNP associated proteins. In both cases, snoRNAs specifically hybridize to the complementary sequence in the RNA, and protein complexes associated then perform the appropriate modification to the nucleotide that is identified by the snoRNAs.

SMARCAD1 contributes as part of a large complex with HDAC1, HDAC2, and KAP1 G9A to integrate with nucleosome spacing and histone deacetylation. H3K9 methylation is required for heterochromatin restore apparently facilitates histone deacetylation and H3K9mc3. How chromatin remodeling is done by deacetylation is unknown, but it seems to coordinate spacing between nucleosomes with H3K9 acetylation and monomethylation.

### Mitochondria

SLC25A14 uncoupling protein that facilitates the transfer of anions from the inside of the mitochondria to the outer mitochondrial membrane and the return transfer of protons from the outside to the inner mitochondrial membrane. SLC25A14 functional role in cellular energy supply and the production of superoxide after it overexpressed in neuronal cells. In untreated culture conditions, overexpression of MMP and SLC25A14 significantly decreased content of intracellular ATP.

TMEM135, some studies have demonstrated TMEM135 association with mitochondrial's fat metabolism, and a possible role for TMEM135 recently identified in improving fat storage.

VDAC3 selective Anions voltage-dependent channels (VDACs) are proteins that form pores allowing permeability of the mitochondrial outer membrane. A growing body of evidence indicates that VDAC plays a major role in metabolite flow in and out of mitochondria, resulting in regulation of mitochondrial functions.

### Membrane

SLC20A2 the proteins of this group transport stream comprises an initial joining of a Na+ion, followed by a random interaction between Pi (inorganic phosphorus) monovalent and second ion Na+. Reorientation loaded carrier, then leads to the release substrate in the cytosol.

NALCN encoding a voltage-independent, cationic, non-selective, non-inactivating, permeable to sodium, potassium and calcium channel when expressed exogenously in HEK293 cells. Sodium is important for neuronal excitability in vivo, the NALCN channel seems to be the main source of sodium leak in hippocampal neurons and because these two processes are strongly altered in schizophrenia is the hypothesis had to NALCN could show a genetic association with schizophrenia.

HACE1 is a tumor suppressor, catalyses poly-Rac1 ubiquitylation at lysine 147 upon activation by HGF, resulting in its proteasomal degradation. HACE1 controls NADPH oxidase. HACE1 promotes increased binding to Rac1 regulating the NADPH oxidase, decrease the production of oxygen free radicals, and inhibit the expression of cyclin D1 and decrease susceptibility to damage DNA. HACE1 loss leads to overactive NADPH oxidase, increased ROS generation, also the expression of cyclin D1 and DNA damage induced by ROS.

NCAM1 is a constitutive molecule expressed on the surface of various cells, promotes neurite outgrowth, nerve branching, fasciculation and cell migration.

OPN5 apparent gabaergic interaction in Synaptic space.

NETO2 is an auxiliary subunit determines the functional propiedadde KARS proteins (kainate, a subfamily of ionotropic glutamate receptors—iGluRs—) that mediate excitatory synaptic transmission, regulate the release of neurotransmitters and in selective distribution in brain.

VANGL1 This gene encodes a member of the family tretraspanin. Mutations in this gene are associated with neural tube defects. Alternative splicing results in multiple transcript variants.

DKK4 is a DKK to block the expression of LRP and thus union with the complex Frizzled and Wnt/SFRP/WIF blocking the release of b-catenin.

NTRK3 is a member of the family of neurotrophin receptors and is critical for the development of the nervous system. Published studies suggested that NTRK3 is a dependence receptor, which signals both the ligand-bound state (“on”) and the free ligand (“off”) state (see chart). When present the ligand neurotrophin-3 (NT-3), NTRK3 trigger signals within the cell via a tyrosine kinase domain in promoting cell proliferation and survival. In the absence of NT-3, NTRK3 signals for cell death by triggering apoptosis. Therefore, NTRK3 have the potential to be an oncogene or tumor suppressor gene function of the presence of NT-3.

### Reticular Endoplasmic Reticulum

PSMC1 is involved in the destruction of the protein in bulk at a fast or slow rate in a wide variety of biological processes such as cell cycle progression, apoptosis, regulation of metabolism, signal transduction, and antigen processing.

PTBP2 Ptbp1 and Ptbp2 regulate the alternative splicing of various RNA target assemblies, suggesting that the roles of Ptbp½ proteins are different in different cellular contexts. Ptbp2 functions in the brain are not clear.

RyR3s is a type of ion channel that intracellular free Ca2+ when opened from the endoplasmic reticulum (ER). It is very similar to the inositol triphosphate receptor (inositol-1,4,5-triphosphate) IP3R. The main signal to trigger the opening of RyRs are Ca2+ has usually entered through voltage-dependent channels of cell membrane. RyR3 is expressed in several cell types including the brain in small quantities, RyR3 deficient mice have impaired hippocampal synaptic plasticity and impaired learning. ATP also stimulates the activity of the channels RyR3. The therapeutic targets focus on molecules that induce release control, internalization and calcium mobilization.

RPL35 is a protein binding to the signal recognition particle (SPR) and its receptor (SR). They mediate targeting complexes nascent chain-ribosome to the endoplasmic reticulum.

RPL5 is an MDM2 binding protein (MDM2 oncogene, protein E3 ubiquitin ligase) and SRSF 1 (serine/rich splicing factor arginine 1) to stabilize p53 oncogene and to induce cell senescence. RPL can join RPL11 and other ribosomal proteins to silence Hdm2 and p53.

FAM69A calico dependent kinase, extracellular and intracellular, localized in the endoplasmic reticulum.

### Other Organelles

GOLGA1 is part transport proteins of the Golgi apparatus, which participates in glycosylation and transport of proteins and lipids in the secretory pathway.

EMLS blocks EMAP via MAP or stabilization of microtubules.

ARPC5L component can function as Arp⅔ complex which is involved in the regulation of actin polymerization and together with the activation of factor inducing nucleation (NPF) mediates the formation of branched networks of actin. It belongs to the family Arpc5.

CSMD1 in the TGF-.beta. pathway, CSMD1 permits the TGF-.beta. receptor I junction, allowing it to phosphorylate Smad3 and thus allow complex formation: phosphorylated Smad3/phosphorylated Smad2/Smad4; the complex is internalized into the cellular nucleus and bound to a transforming factor leads to apoptosis. In addition, the TGF-.beta. receptor II binds the phosphorylated complex, allowing for subsequent binding Smad1/5/8 with Smad4, and nuclear internalizing inducing apoptosis mediated by binding to a transforming factor.

### E. REFERENCES

Aberg K A, Liu Y, Bukszar J, McClay J L, Khachane A N, Andreassen O A, et al. A comprehensive family-based replication study of schizophrenia genes. JAMA Psychiatry. 2013; 701:573-81.
Alabert C, Groth A. Chromatin replication and epigenome maintenance. Nat Rev Mol Cell Biol. 2012; 131:153-67.
Albert R. Scale-free networks in cell biology. J Cell Sci. 2005; 1181:4947-57.
Alcina A, Fernandez O, Gonzalez J R, Catala-Rabasa A, Fedetz M, Ndagire D, et al. Tag-SNP analysis of the GFI1-EV15-RPL5-FAM69 risk locus for multiple sclerosis. Eur J Hum Genet. 2010; 181:827-31.
Alzhanova D, Hruby D E. A host cell membrane protein, golgin-97, is essential for poxvirus morphogenesis. Virology. 2007; 3621:421-7.
Amaral P P, Neyt C, Wilkins S J, Askarian-Amiri M E, Sunkin S M, Perkins A C, et al. Complex architecture and regulated expression of the Sox2ot locus during vertebrate development. RNA. 2009; 151:2013-27.
American_Psychiatric_Association. Diagnostic and Statistical Manual of Mental Disorders. Washington, D.C.: American Psychiatric Publishing Inc.; 1994.
Anderson C A, Pettersson F H, Clarke G M, Cardon L R, Morris A P, Zondervan K T. Data quality control in genetic case-control association studies. Nat Protoc. 2010; 51:1564-73.
Arnedo J, del Val C, de Erausquin G A, Romero-Zaliz R, Svrakic D, Cloninger C R, Zwir I: PGMRA: a web server for (phenotype.times.genotype) many-to-many relation analysis in GWAS. Nucleic Acids Res 2013; 41:W142-W149
Babcock D F. Development. Smelling the roses? Science. 2003; 2991:1993-4.
Balko J M, Schwarz U, Bhola N E, Kurupi R, Owens P, Miller T W, et al. Activation of MAPK pathways due to DUSP4 loss promotes cancer stem cell-like phenotypes in basal-like breast cancer. Cancer Res. 2013; 731; 6346-58.
Beer M A, Tavazoie S. Predicting gene expression from sequence. Cell. 2004; 1171:185-98.
Beyene J, Tritchler D. Multivariate analysis of complex gene expression and clinical phenotypes with genetic marker data. Genetic Epidemiology. 2007; 311:S103-S9.
Bezdek J C. Pattern Analysis. In: Pedrycz W, Bonissone P P, Ruspini E H, editors. Handbook of Fuzzy Computation. Bristol: Institute of Physics; 1998. p.F6.1.-F6.20.
Biber J, Hernando N, Forster I. Phosphate transporters and their function. Annu Rev Physiol. 2013; 751:535-50.
Blanchard J J, Cohen A S: The structure of negative symptoms within schizophrenia; implications for assessment. Schizophr Bull 2006; 32:238-245
Blayney L M, Zissimopoulos S, Ralph E, Abbot E, Matthews L, Lai F A. Ryanodine receptor oligomeric interaction: identification of a putative binding region. J Biol Chem. 2004; 2791:14639-48.
Bonnard C, Strobl A C, Shboul M, Lee H, Merriman B, Nelson S F, et al. Mutations in IRX5 impair craniofacial development and germ cell migration via SDF1. Nat Genet. 2012; 441:709-13.
Braskie M N, Kohannim O, Jahanshad N, Chiang M C, Barysheva M, Toga A W, et al. Relation between variants in the neurotrophin receptor gene, NTRK3, and white matter integrity in healthy young adults. Neuroimage. 2013; 821:146-53.
Brennand K J, Simone A, Jou J, Gelboin-Burkhart C, Tran N, Sangar S, et al. Modelling schizophrenia using human induced pluripotent stem cells. Nature. 2011; 4731:221-5.
Cagnol S, Rivard N. Oncogenic KRAS and BRAF activation of the MEK/ERK signaling pathway promotes expression of dual-specificity phosphatase 4 (DUSP4/MKP2) resulting in nuclear ERK½ inhibition. Oncogene. 2013; 321:564-76.
Castillo-Lluva S, Tan C T, Daugaard M, Sorensen P H, Malliri A. The tumour suppressor HACE1 controls cell migration by regulating Rac1 degradation. Oncogene. 2013; 321:1735-42.
Chacon M R, Fernandez G, Rico B. Focal adhesion kinase functions downstream of Sema3A signaling during axonal remodeling. Mol Cell Neurosci. 2010; 441:30-42.
Chen G, Zhou X, Florea S, Qian J, Cai W, Zhang Z, et al. Expression of active protein phosphatase 1 inhibitor—1 attenuates chronic beta-agonist-inducedcardiac apoptosis. Basic Res Cardiol. 2010; 1051:573-81.
Chen J, Lee G, Fanous A H, Zhao Z, Jia P, O'Neill A, et al. Two non-synonymous markers in PTPN21, identified by genome-wide association study data-mining and replication, are associated with schizophrenia. Schizophr Res. 2011; 1311:43-51.
Cicchetti D, Rogosch F A: Equifinality and multifinality in developmental psychopathology. Dev Psychopathol 1996; 81:597-600
Ciliberti S, Martin O C, Wagner A. Innovation and robustness in complex regulatory gene networks. Proc Natl Acad Sci USA. 2007; 1041:13591-6.
Cloninger C R, et al. PGMRA: a web server for (phenotype.times.genotype) many-to-many relation analysis in GWAS. Nucleic Acid Research. 2013; 75.
Consortium Cross-Disorder Group of the Psychiatric Genomics Consortium: Genetic relationship between five psychiatric disorders estimated from genome-wide SNPs. Nat Genet 2012; 45:984-994
Consortium I. Rare chromosomal deletions and duplications increase risk of schizophrenia. Nature. 2008; 4551:237-41.
Consortium SPG-WASG. Genome-wide association study identifies five new schizophrenia loci. Nat Genet. 2011; 431:969-76.
Consortium TSPG. Genome-wide association study identifies five new schizophrenia loci. Nat Genet. 2011; 431:969-76.
Consortium TSPG; Schizophrenia Psychiatric Genome-Wide Association Study (GWAS) Consortium: Genome-wide association study identifies five new schizophrenia loci. Nat Genet 2011; 43:969-976
Daugaard M, Nitsch R, Razaghi B, McDonald L, Jarrar A, Torrino S, et al. Hace 1 controls ROS generation of vertebrate Rac1—dependent NADPH oxidase complexes. Nat Commun 2013; 41:2180.
de Vries P J: Genetics and neuropsychiatric disorders: genome-wide, yet narrow. Nat Med 2009; 15:850-851
Deery E C, Vithana E N, Newbold R J, Gallon V A, Bhattacharya S S, Warren M J, et al. Disease mechanism for retinitis pigmentosa (RP11) caused by mutations in the splicing factor gene PRPF31. Hum Mol Genet. 2002; 111:3209-19.
Di Leva G, Briskin D, Croce C M. MicroRNA in cancer: new hopes for antineoplastic chemotherapy. Ups J Med Sci. 2012; 1171:202-16.
Dudkiewicz M, Lenart A, Pawlowski K. A novel predicted calcium-regulated kinase family implicated in neurological disorders. PLoS One. 2013; 81:e66427.
Dunn O J. Multiple Comparisons Among Means. Journal of the American Statistical Association. 1961; 561:52-64.
Durany N, Michel T, Zochling R, Boissl K W, Cruz—Sanchez F F, Riederer P, et al. Brain—derived neurotrophic factor and neurotrophin 3 in schizophrenic psychoses. Schizophr Res. 2001; 521:79-86.
Durany N, Thome J. Neurotrophic factors and the pathophysiology of schizophrenic psychoses. Eur Psychiatry. 2004; 191:326-37.
Eastwood S L, Law A J, Everall I P, Harrison P J. The axonal chemorepellant semaphorin 3A is increased in the cerebellum in schizophrenia and may contribute to its synaptic pathology. Mal Psychiatry. 2003; 81:148-55.
Echtay K S. Mitochondrial uncoupling proteins-what is their physiological role? Free Radic Biol Med. 2007; 431:1351-71.
Eichler E E, Flint J, Gibson G, Kong A, Leal S M, Moore J H, Nadeau J H: Missing heritability and strategies for finding the underlying causes of complex disease. Nat Rev Genet 2010; 11:446-450
Faraone S V, Blehar M, Pepple J, Moldin S O, Norton J, Nurnberger J I, Malaspina D, Kaufmann C A, Reich T, Cloninger C R, DePaulo J R, Berg K, Gershon E S, Kirch D G, Tsuang M T: Diagnostic accuracy and confusability analyses: an application to the Diagnostic Interview for Genetic Studies. Psychol Med 1996; 26:401-410
Fattman C L, Schaefer L M, Oury T D. Extracellular superoxide dismutase in biology and medicine. Free Radic Biol Med. 2003; 351:236-56.
Fisher J L, Mott D D. The auxiliary subunits Neto1 and Neto2 reduce voltage-dependent inhibition of recombinant kainate receptors. J Neurosci. 2012; 3211:12928-33.
Forster I C, Hernando N, Biber J, Murer H. Phosphate transporters of the SLC20 and SLC34 families. Mol Aspects Med. 2013; 341:386-95.
Fregoso O I, Das S, Akerman M, Krainer A R. Splicing-factor oncoprotein SRSF1 stabilizes p53 via RPL5 and induces cellular senescence. Mol Cell. 2013; 501:56-66.
Gottesman I I, Shields J, Meehl P E: Schizophrenia: The Epigenetic Puzzle. New York, Cambridge University Press, 1982
Guardavaccaro D, Pagano M. Stabilizers and destabilizers controlling cell cycle oscillators. Mol Cell. 2006; 221:1-4.
Hallmayer J F, Kalaydjieva L, Badcock J, Dragovic M, Howell S, Michie P T, Rock D, Vile D, Williams R, Corder E H, Hollingsworth K, Jablensky A: Genetic evidence for a distinct subtype of schizophrenia characterized by pervasive cognitive deficit. Am J Hum Genet 2005; 77:468-476
Harari O, Park S Y, Huang H, Groisman E A, Zwir I. Defining the plasticity of transcription factor binding sites by Deconstructing DNA consensus sequences: the PhoP-binding sites among gamma/enterobacteria. PLoS Comput Biol. 2010; 61:e1000862.
Hill W G, Zhang X S: On the pleiotropic structure of the genotype-phenotype map and the evolvability of complex organisms. Genetics 2012; 190:1131-1137
Holliday E G, McLean D E, Nyholt D R, Mowry B J: Susceptibility locus on chromosome 1q23-25 for a schizophrenia subtype resembling deficit schizophrenia identified by latent class analysis. Arch Gen Psychiatry 2009; 66:1058-1067
Houle D, Govindaraju D R, Omholt S: Phenomics: the next challenge. Nat Rev Genet 2010; 11:855-866
Inden M, Iriyama M, Takagi M, Kaneko M, Hozumi I Localization of type-III sodium-dependent phosphate transporter 2 in the mouse brain. Brain Res. 2013; 15311:75-83.
International Schizophrenia Consortium: Rare chromosomal deletions and duplications increase risk of schizophrenia. Nature 2008; 455:237-241
Ivakine E A, Acton B A, Mahadevan V, Ormond J, Tang M, Pressey J C, et al. Neto2 is a KCC2 interacting protein required for neuronal C1-regulation in hippocampal neurons. Proc Natl Acad Sci USA. 2013; 1101:3561-6.
Jeong H, Tombor B, Albert R, Oltvai Z N, Barabasi A L. The large-scale organization of metabolic networks. Nature. 2000; 4071:651-4.
Jezek P. Possible physiological roles of mitochondrial uncoupling proteins-UCPn. Int J Biochem Cell Biol. 2002; 341:1190-206.
Kan A, Ikeda T, Saito T, Yano F, Fukai A, Hojo H, et al. Screening of chondrogenic factors with a real-time fluorescence—monitoring cell line ATDC5-C2ER: identification of sorting nexin 19 as a novel factor. Arthritis Rheum. 2009; 601:3314-23.
Katoh M, Igarashi M, Fukuda H, Nakagama H. Cancer genetics and genomics of human FOX family genes. Cancer Lett. 2013; 3281:198-206.
Katoh M. Human FOX gene family (Review). Int J Oncol. 2004; 251:1495-500.
Katoh M. Identification and characterization of human FOXK1 gene in silico. Int J Mol Med. 2004; 141:127-32.
Katoh M. WNT/PCP signaling pathway and human cancer (review). Oncol Rep. 2005; 141:1583-8.
Katoh Y, Katoh M. Comparative genomics on Vangl1 and Vangl2 genes. Int J Oncol. 2005; 261:1435-40.
Kendler K S, Karkowski-Shuman L, O'Neill F A, Straub R E, MacLean C J, Walsh D: Resemblance of psychotic symptoms and syndromes in affected sibling pairs from the Irish Study of High-Density Schizophrenia Families: evidence for possible etiologic heterogeneity. Am J Psychiatry 1997; 154:191-198 Abstract, Medline
Keppetipola N, Sharma S, Li Q, Black D L. Neuronal regulation of pre-mRNA splicing by polypyrimidine tract binding proteins, PTBP1 and PTBP2. Crit Rev Biochem Mol Biol. 2012; 471360-78.
Kobayashi H, Hino M, Inoue T, Nil E, Ikeda K, Son C, et al. GC79/TRPS1 and tumorigenesis in humans. Am J Med Genet A. 2005; 1341341-3.
Kupershmidt I, Su Q J, Grewal A, Sundaresh S, Halperin I, Flynn J, et al. Ontology-based meta-analysis of global collections of high-throughput public data. PLoS One. 2010; 51:epublish.
Kwok K H, Ho P W, Chu A C, Ho J W, Liu H F, Yiu D C, et al. Mitochondrial UCPS is neuroprotective by preserving mitochondrial membrane potential, ATP levels, and reducing oxidative stress in MPP+ and dopamine toxicity. Free Radic Biol Med. 2010; 491: 1023-35.
Laurila J P, Castellone M D, Curcio A, Laatikainen L E, Haaparanta-Solin M, Gronroos T J, et al. Extracellular superoxide dismutase is a growth regulatory mediator of tissue injury recovery. Mol Ther. 2009; 171:448-54.
Lee D D, Seung H S. Learning the parts of objects by non-negative matrix factorization. Nature. 1999; 4011:788-91.
Lee S H, DeCandia T R, Ripke S, Yang J, Sullivan P F, Goddard M E, et al. Estimating the proportion of variation in susceptibility to schizophrenia captured by common SNPs. Nature Genetics. 2012; 441:247-U35.
Lee S H, DeCandia T R, Ripke S, Yang J, Sullivan P F, Goddard M E, Keller M C, Visscher P M, Wray N R; Schizophrenia Psychiatric Genome-Wide Association Study Consortium (PGC-SCZ); International Schizophrenia Consortium (ISC); Molecular Genetics of Schizophrenia Collaboration (MGS): Estimating the proportion of variation in susceptibility to schizophrenia captured by common SNPs. Nat Genet 2012; 44:247-250
Leung S W, Apponi L H, Cornejo O E, Kitchen C M, Valentini S R, Pavlath G K, et al. Splice variants of the human ZC3H14 gene generate multiple isoforms of a zinc finger polyadenosine RNA binding protein. Gene. 2009; 4391:71-8.
Li J, Wu F, Sheng F, Li Y J, Jin D, Ding X, et al. NOK/STYK1 interacts with GSK-3beta and mediates Ser9 phosphorylation through activated Akt. FEBS Lett. 2012; 5861:3787-92.
Lieberman J A: Comparative effectiveness of antipsychotic drugs: a commentary on: Cost Utility of the Latest Antipsychotic Drugs in Schizophrenia Study (CUtLASS 1) and Clinical Antipsychotic Trials of Intervention Effectiveness (CATIE). Arch Gen Psychiatry 2006; 63:1069-1072
Lin X, Cai T, Wu M C, Zhou Q, Liu G, Christiani D C, et al. Kernel machine SNP-set analysis for censored survival outcomes in genome-wide association studies. Genet Epidemiol. 2011; 351:620-31.
Mailman M D, Feolo M, Jin Y, Kimura M, Tryka K, Bagoutdinov R, Hao L, Kiang A, Paschall J, Phan L, Popova N, Pretel S, Ziyabari L, Lee M, Shao Y, Wang Z Y, Sirotkin K, Ward M, Kholodov M, Zbicz K, Beck J, Kimelman M, Shevelev S, Preuss D, Yaschenko E, Graeff A, Ostell J, Sherry S T: The NCBI dbGaP database of genotypes and phenotypes. Nat Genet 2007; 39:1181-1186
Mannoor K, Liao J, Jiang F. Small nucleolar RNAs in cancer. Biochim Biophys Acta. 2012; 18261:121-8.
Manolio T A: Genomewide association studies and assessment of the risk of disease. N Engl J Med 2010; 363:166-176
Mettouchi A, Lemichez E. Ubiquitylation of active Rac1 by the E3 ubiquitin-ligase HACE1. Small GTPases. 2012; 31:102-6.
Miao L, St Clair D K. Regulation of superoxide dismutase genes: implications in disease. Free Radic Biol Med. 2009; 471:344-56.
Millard T H, Behrendt B, Launay S, Futterer K, Machesky L M. Identification and characterisation of a novel human isoform of Arp⅔ complex subunit p16-ARC/ARPC5. Cell Motil Cytoskeleton. 2003; 541:81-90.
Mudge J, Miller N A, Khrebtukova I, Lindquist I E, May G D, Huntley J J, et al. Genomic convergence analysis of schizophrenia: mRNA sequencing reveals altered synaptic vesicular transport in post-mortem cerebellum. PLoS One. 2008; 31:e3625.
Neufeld G, Kessler O. The semaphorins: versatile regulators of tumour progression and tumour angiogenesis. Nat Rev Cancer. 2008; 81:632-45.
Novotny I, Blazikova M, Stanek D, Herman P, Malinsky J. In vivo kinetics of U4/U6.U5 tri-snRNP formation in Cajal bodies. Mol Biol Cell. 2011; 221:513-23.
Ojima H, Yoshikawa D, Ino Y, Shimizu H, Miyamoto M, Kokubu A, et al. Establishment of six new human biliary tract carcinoma cell lines and identification of MAGEH1 as a candidate biomarker for predicting the efficacy of gemcitabine treatment. Cancer Sci. 2010; 1011:882-8.
Otnaess M K, Djurovic S, Rimol L M, Kulle B, Kahler A K, Jonsson E G, et al. Evidence for a possible association of neurotrophin receptor (NTRK-3) gene polymorphisms with hippocampal function and schizophrenia. Neurobiol Dis. 2009; 341:518-24,
Overexpression of the autoantigen IA-2 puts beta cells into a pre-apoptotic state: autoantigen-induced, but non-autoimmune-mediated, tissue destruction. Clin Exp Immunol. 2007; 1501:49-60.
Ozkan S B, Wu G A, Chodera J D, Dill K A. Protein folding by zipping and assembly. Proc Natl Acad Sci USA. 2007; 1041:11987-92.
Pan Y, Zhang J, Liu W, Shu P, Yin B, Yuan J, et al. Dok5 is involved in the signaling pathway of neurotrophin-3 against TrkC-induced apoptosis. Neurosci Lett. 2013; 5531:46-51.
Pascual-Montano A, Carmona-Saez P, Chagoyen M, Tirado F, Carazo J M, Pascual-Marqui RD. bioNMF: a versatile tool for non-negative matrix factorization in biology. BMC Bioinformatics. 2006; 71:366.
Peppercorn J, Perou C M, Carey L A. Molecular subtypes in breast cancer evaluation and management: divide and conquer. Cancer Invest. 2008; 261:1-10.
Perrault I, Hanein S, Gerard X, Delphin N, Fares—Taie L, Gerber S, et al. Spectrum of SPATA7 mutations in Leber congenital amaurosis and delineation of the associated phenotype. Hum Mutat. 2010; 311:E1241-50.
Purcell S, Neale B, Todd-Brown K, Thomas L, Ferreira M A, Bender D, Mailer J, Sklar P, de Bakker P I, Daly M J, Sham P C: PLINK: a tool set for whole-genome association and population-based linkage analyses. Am J Hum Genet 2007; 81:559-575
Purcell S M, Wray N R, Stone J L, Visscher P M, O'Donovan M C, Sullivan P F, Sklar P; International Schizophrenia Consortium: Common polygenic variation contributes to risk of schizophrenia and bipolar disorder. Nature 2009; 460:748-752
Reina S, Palermo V, Guarnera A, Guarino F, Messina A, Mazzoni C, et al. Swapping of the N-terminus of VDAC1 with VDAC3 restores full activity of the channel and confers anti-aging features to the cell. FEBS Lett. 2010; 5841:2837-44.
Ripke S, et al.; Multicenter Genetic Studies of Schizophrenia Consortium; Psychosis
Endophenotypes International Consortium; Welcome Trust Case Control Consortium 2: Genome-wide association analysis identifies 13 new risk loci for schizophrenia. Nat Genet 2013; 45:1150-1159
Romero-Zaliz R, Del Val C, Cobb J P, Zwir I: Onto-CC: a web server for identifying Gene Ontology conceptual clusters. Nucleic Acids Res 2008; 36:W352-W357
Ruspini E H, Zwir I. Automated generation of qualitative representations of complex objects by hybrid soft-computing methods. In: Pal S K, Pal A, editors. Pattern recognition: from classical to modern approaches. New Jersey: World Scientific; 2002. p. 454-74.
Saini S, Majid S, Dahiya R. The complex roles of Wnt antagonists in RCC. Nat Rev Urol. 2011; 81:690-9.
Schachtner R, Lutter D, Knoilmuller P, Tome A M, Theis F J, Schmitz G, Stetter M, Vilda P G, Lang E W: Knowledge-based gene expression classification via matrix factorization. Bioinformatics 2008; 24:1688-1697
Scheideler M, Elabd C, Zaragosi L E, Chiellini C, Hackl H, Sanchez-Cabo F, et al. Comparative transcriptomics of human multipotent stem cells during adipogenesis and osteoblastogenesis. BMC Genomics. 2008; 91:340.
Sekiguchi T, Hirose E, Nakashima N, Ii M, Nishimoto T. Novel G proteins, Rag C and Rag D, interact with GTP-binding proteins, Rag A and Rag B. J Biol Chem. 2001; 2761:7246-57.
Selimovic D, Sprenger A, Hannig M, Haikel Y, Hassan M. Apoptosis related protein-1 triggers melanoma cell death via interaction with the juxtamembrane region of p75 neurotrophin receptor. J Cell Mol Med. 2012; 161:349-61.
Shi J, Levinson D F, Duan J, Sanders A R, Zheng Y, Pe'er I, Dudbridge F, Holmans P A, Whittemore A S, Mowry B J, Olincy A, Amin F, Cloninger C R, Silverman J M, Buccola N G, Byerley W F, Black D W, Crowe R R, Oksenberg J R, Mirel D B, Kendler K S, Freedman R, Gejman P V: Common variants on chromosome 6p22.1 are associated with schizophrenia. Nature 2009; 460:753-757
Shimamura A, Alter B P. Pathophysiology and management of inherited bone marrow failure syndromes. Blood Rev. 2010; 241:101-22.
Shimizu K, Okamoto N, Miyake N, Taira K, Sato Y, Matsuda K, et al. Delineation of dermatan 4 O-sulfotransferase 1 deficient Ehlers-Danlos syndrome: observation of two additional patients and comprehensive review of 20 reported patients. Am J Med Genet A. 2011; 155A1:1949-58.
Shukla R, Upton K R, Munoz-Lopez M, Gerhardt D J, Fisher M E, Nguyen T, et al. Endogenous retrotransposition activates oncogenic pathways in hepatocellular carcinoma. Cell. 2013; 1531:101-11.
Sklar P, Pato M T, Kirby A, Petryshen T L, Medeiros H, Carvalho C, Macedo A, Dourado A, Coelho I, Valente J, Soares M J, Ferreira C P, Lei M, Verner A, Hudson T J, Morley C P, Kennedy J L, Azevedo M H, Lander E, Daly M J, Pato C N: Genome-wide scan in Portuguese Island families identifies 5q31-5q35 as a susceptibility locus for schizophrenia and psychosis. Mol Psychiatry 2004; 9:213-218
Sokal R, Rohif J. The Comparison of Dendrograms by Objective Methods. Taxon. 1962; 11.
Souza R P, Rosa D V, Romano-Silva M A, Zhen M, Meltzer H Y, Lieberman J A, et al. Lack of association of NALCN genetic variants with schizophrenia. Psychiatry Res. 2011; 1851:450-2.
Spehr M, Gisselmann G, Poplawski A, Riffell J A, Wetzel C H, Zimmer R K, et al. Identification of a testicular odorant receptor mediating human sperm chemotaxis. Science. 2003; 2991:2054-8.
Stroup T S, McEvoy J P, Swartz M S, Byerly M J, Glick I D, Canive J M, McGee M F, Simpson G M, Stevens M C, Lieberman J A: The National Institute of Mental Health Clinical Antipsychotic Trials of Intervention Effectiveness (CATIE) project: schizophrenia trial design and protocol development. Schizophr Bull 2003; 29:15-31
Sullivan P F, Keefe R S, Lange L A, Lange E M, Stroup T S, Lieberman J, et al. NCAM1 and neurocognition in schizophrenia. Biol Psychiatry. 2007; 611:902-10.
Sullivan P F, Lin D, Tzeng J Y, van den Cord E, Perkins D, Stroup T S, Wagner M, Lee S, Wright F A, Zou F, Liu W, Downing A M, Lieberman J, Close S L: Genomewide association for schizophrenia in the CATIE study: results of stage 1. Mol Psychiatry 2008; 13:570-584
Svrakic D M, Zorumski C F, Svrakic N M, Zwir I, Cloninger C R: Risk architecture of schizophrenia: the role of epigenetics. Curr Opin Psychiatry 2013; 26:188-195
Tamagnone L, Giordano S. Semaphorin pathways orchestrate osteogenesis. Nat Cell Biol. 2006; 81:545-7.
Tamayo P, Scanfeld D, Ebert B L, Gillette M A, Roberts C W, Mesirov J P. Metagene projection for cross-platform, cross-species characterization of global transcriptional states. Proc Natl Acad Sci USA. 2007; 1041:5959-64.
Tamura H, Kawata M, Hamaguchi S, Ishikawa Y, Shiosaka S. Processing of neuregulin-1 by neuropsin regulates GABAergic neuron to control neural plasticity of the mouse hippocampus. J Neurosci. 2012; 321:12657-72.
Tanahashi N, Suzuki M, Fujiwara T, Takahashi E, Shimbara N, Chung C H, et al. Chromosomal localization and immunological analysis of a family of human 26S proteasomal ATPases. Biochem Biophys Res Commun 1998; 2431:229-32.
Tang M R, Wang Y X, Guo S, Han S Y, Wang D. CSMD1 exhibits antitumor activity in A375 melanoma cells through activation of the Smad pathway. Apoptosis. 2012; 171:927-37.
Tantelin E E, Bellingham J, Hankins M W, Foster R G, Lucas R J. Neuropsin (Opn5): a novel opsin identified in mammalian neural tissue. FEBS Lett. 2003; 5541:410-6.
Tavazoie S, Hughes J D, Campbell M J, Cho R J, Church G M: Systematic determination of genetic network architecture. Nat Genet 1999; 22:281-285
Tcherpakov M, Bronfman F C, Conticello S G, Vaskovsky A, Levy Z, Niinobe M, et al. The p75 neurotrophin receptor interacts with multiple MAGE proteins. J Biol Chem. 2002; 2771:49101-4.
Thompson J R, Attia J, Minelli C: The meta-analysis of genome-wide association studies. Brief Bioinform 2011; 12:259-269
Torrino S, Visvikis O, Doye A, Boyer L, Stefani C, Munro P, et al. The E3 ubiquitin-ligase HACE1 catalyzes the ubiquitylation of active Racl. Dev Cell. 2011; 211:959-65.
Wagner A. Robustness against mutations in genetic networks of yeast. Nat Genet. 2000; 241:355-61.
Wang H, den Hollander A I, Moayedi Y, Abulimiti A, Li Y, Collin R W, et al. Mutations in SPATA7 cause Leber congenital amaurosis and juvenile retinitis pigmentosa. Am J Hum Genet. 2009; 841:380-7.
Wang X, Liu B, Li N, Li H, Qiu J, Zhang Y, et al. IPP5, a novel protein inhibitor of protein phosphatase 1, promotes G1/S progression in a Thr-dependent manner. J Biol Chem. 2008; 2831:12076-84.
Ward L D, Kellis M. HaploReg: a resource for exploring chromatin states, conservation, and regulatory motif alterations within sets of genetically linked variants. Nucleic Acids Res. 2012; 401:D930-4.
Wilkie S E, Vaclavik V, Wu H, Bujakowska K, Chakarova C F, Bhattacharya S S, et al. Disease mechanism for retinitis pigmentosa (RP11) caused by missense mutations in the splicing factor gene PRPF31. Mol Vis. 2008; 141:683-90.
Williams S M, Haines J L: Correcting away the hidden heritability. Ann Hum Genet 2011; 75:348-350
Wray N R, Visscher P M: Narrowing the boundaries of the genetic architecture of schizophrenia. Schizophr Bull 2010; 36:14-23
Wu M C, Kraft P, Epstein M P, Taylor D M, Chanock S J, Hunter D J, Lin X: Powerful SNP-set analysis for case-control genome-wide association studies. Am J Hum Genet 2010; 86:929-942
Wu M C, Lee S, Cai T, Li Y, Boehnke M, Lin X: Rare-variant association testing for sequencing data with the sequence kernel association test. Am J Hum Genet 2011; 89:82-93
Wu M C, Maity A, Lee S, Simmons E M, Harmon Q E, Lin X, et al. Kernel Machine SNP-Set Testing Under Multiple Candidate Kernels. Genet Epidemiol. 2013; 371:267-75.
Wuchty S. Scale-free behavior in protein domain networks. Mol Biol Evol. 2001; 181:1694-702.
Xu Z, He Z, Huang K, Tang W, Li Z, Tang R, et al. No genetic association between NCAM1 gene polymorphisms and schizophrenia in the Chinese population. Prog Neuropsychopharmacol Biol Psychiatry. 2008; 321:1633-6.
Yang J, Lee S H, Goddard M E, Visscher P M: GCTA: a tool for genome-wide complex trait analysis. Am J Hum Genet 2011; 88:76-82
Yang J, Siqueira M F, Behl Y, Alikhani M, Graves D T. The transcription factor ST18 regulates proapoptotic and proinflammatory gene expression in fibroblasts. FASEB J. 2008; 221:3956-67.
Yeung K Y, Ruzzo W L. Principal component analysis for clustering gene expression data. Bioinformatics. 2001; 171:763-74.
Yngvadottir B, Macarthur D G, Jin H, Tyler-Smith C: The promise and reality of personal genomics. Genome Biol 2009; 10:237
Yu H. Cdc20: a WD40 activator for a cell cycle degradation machine. Mol Cell. 2007; 271:3-16.
Zhang K, Chang S, Cui S, Guo L, Zhang L, Wang J: ICSNPathway: identify candidate causal SNPs and pathways from genome-wide association study by one analytical framework. Nucleic Acids Res 2011; 39:W437-W443
Zhao X, Wu Q, Fu X, Yu B, Shao Y, Yang H, et al. Examination of copy number variations of CHST9 in multiple types of hematologic malignancies. Cancer Genet Cytogenet. 2010; 2031:176-9.
Zhou T, Kuscsik Z, Liu J G, Medo M, Wakeling J R, Zhang Y C. Solving the apparent diversity-accuracy dilemma of recommender systems. Proc Natl Acad Sci USA. 2010; 1071:4511-5.
Zissimopoulos S, Seifan S, Maxwell C, Williams A J, Lai F A. Disparities in the association of the ryanodine receptor and the FK506-binding proteins in mammalian heart. J Cell Sci. 2012; 1251:1759-69.
Zwir I, Huang H, Groisman E A: Analysis of differentially-regulated genes within a regulatory network by GPS genome navigation. Bioinformatics 2005; 21:4073-4083
Zwir I, Shin D, Kato A, Nishino K, Latifi T, Solomon F, Hare J M, Huang H, Groisman E A: Dissecting the PhoP regulatory network of Escherichia coli and Salmonella enterica. Proc Natl Acad Sci USA 2005; 102:2862-2867

What is claimed is:

1. A method of predicting schizophrenia type in a subject having schizophrenia, comprising:
obtaining a biological sample from a subject comprising DNA (e.g., plasma or tissue extracts);
detecting by genome array, low density PCR array or oligo array single nucleotide polymorphisms (SNPs) consisting of 19_2, 88_64, 81_13, 87_76, 58_29, 83_41, 9_9, 10_4, 14_6, 56_30, 42_37, 65_25, 71_55, 12_11, 90_78, 77_5, 88_8, 51_28, 59_48, 41_12, 22_11, 13_12, 31_22, 85_84, 87_84, 16_10, 56_19, 75_31, 81_73, 85_23, 21_8, 76_74, 61_39, 75_67, 76_63, 81_3, 87_26, 88_43, 25_10, 12_2, 52_42, and 54_51; and
assigning the subject to a schizophrenia type selected from (i) severe process, with positive and negative symptom schizophrenia; (ii) positive and negative schizophrenia; (iii) negative schizophrenia; (iv) positive schizophrenia; (v) severe process, positive schizophrenia; (vi) moderate process, disorganized negative schizophrenia; (vii) moderate process, positive and negative schizophrenia; or (viii) moderate process, continuous positive schizophrenia.

2. The method of claim 1, wherein the one or more SNP sets are selected from the group consisting of 88_8, 90_78, 65_25, 42_37, 71_55, 56_30, 77_5, 12_11, 51_28, 59_48, 10_4, 83_41, 58_29, 9_9, 14_6, 87_76, 88_64, and 81_13.

3. The method of claim 1, wherein the one or more SNP sets are selected from the group consisting of 10_4, 83_41, 58_29, 9_9, 14_6, 87_76, 88_64, and 81_13.

4. The method of claim 1, wherein the one or more SNP sets are selected from the group consisting of 87_76, 88_64, and 81_13.

5. The method of claim 1, wherein the system selects for severe process, with positive and negative symptom schizophrenia, and wherein the one or more SNP sets comprise 56_30, 75_67, or 76_74.

6. The method of claim 1, wherein the system selects for positive and negative Schizophrenia, and wherein the one or more SNP sets comprise 59_48, 71_55, 21_8, 54_51, 31_22, 65_25, or 87_84.

7. The method of claim 1, wherein the system selects for negative Schizophrenia, and wherein the one or more SNP sets comprise 58_29, 9_9, 22_11, 81_3, 13_12, 61_39, 10_4, 81_73, 75_31, 56_19, 88_8, or 12_2.

8. The method of claim 1, wherein the system selects for Positive Schizophrenia, and wherein the one or more SNP sets comprise 88_64, 85_84, or 41_12.

9. The diagnostic system of claim 1, wherein the system selects for severe process, positive schizophrenia, and wherein the one or more SNP sets comprise 77_5, 81_13, or 25_10.

10. The method of claim 1, wherein the system selects for moderate process, disorganized negative schizophrenia, and wherein the one or more SNP sets comprise 19_2, 52_42, 90_78, 12_11, 87_76, or 14_6.

11. The method of claim 1, wherein the system selects for moderate process, positive and negative schizophrenia, and wherein the one or more SNP sets comprise 42_37, 88_43, or 51_28.

12. The method of claim 1, wherein the system selects for moderate process, continuous positive schizophrenia, and wherein the one or more SNP sets comprise 16_10, 83_41, or 87_26.

13. The method of claim 1, further comprising one or more phenotype panels, wherein each phenotype panel comprises one or more phenotypic sets selected from the group comprising 15_13, 12_11, 21_1, 50_46, 9_6, 46_23, 54_11, 30_17, 18_13, 27_6, 61_18, 64_11, 65_64, 12_4, 42_9, 52_28, 7_3, 48_41, 26_8, 69_41, 10_5, 17_2, 63_24, 69_66, 22_13, 53_6, 59_41, 20_19, 55_7, 34_17, 27_7, 4_1, 66_54, 8_4, 51_38, 42_7, 18_3, 46_29, 5_2, 57_39, 11_5, 24_4, 48_7, 28_23, or 25_20.

14. The method of claim 13, wherein the system selects for severe process, with positive and negative symptom schizophrenia, and wherein the one or more phenotypic sets comprise 15_13, 12_11, 21_1, 50_46, 9_6, 46_23, 54_11, 30_17, 18_13, 27_6, 61_18, 64_11, or 65_64.

15. The method of claim 13, wherein the system selects for positive and negative schizophrenia, and wherein the one or more phenotypic sets comprise 12_4 or 42_9.

16. The diagnostic system of claim 14, wherein the system selects for negative schizophrenia, and wherein the one or more phenotypic sets comprise 52_28, 7_3, 48_41, 26_8, 69_41, 10_5, or 17_2.

17. The diagnostic system of claim 14, wherein the system selects for positive schizophrenia, and wherein the one or more phenotypic sets comprise 63_24 and 69_66.

18. The diagnostic system of claim 14, wherein the system selects for severe process, positive schizophrenia, and wherein the one or more phenotypic sets comprise 22_13, 18_13, 53_6, 59_41, 20_19, 55_7, 34_17, 69_66, 27_7, 18_13, 4_1, 66_54, or 8_4.

19. The method of claim 13, wherein the system selects for moderate process, disorganized negative schizophrenia, and wherein the one or more phenotypic sets comprise 51_38, 42_7, 18_3, or 46_29.

20. The method of claim 13, wherein the system selects for moderate process, positive and negative schizophrenia, and wherein the one or more phenotypic sets comprise 5_2, 57_39, 11_5, or 24_4.

21. The method of claim 13, wherein the system selects for moderate process, continuous positive schizophrenia, and wherein the one or more phenotypic sets comprise 48_7, 28_23, or 25_20.

22. The method of claim 1, wherein the method further comprises a means for reading the one or more SNP sets, a computer operationally linked to the means for reading the one or more SNP sets, and a display for visualizing the diagnostic risk; wherein the computer identifies the SNP, compares the SNP profile to a control, and catalogs that data, wherein the computer provides an input source for inputting phenotypic data into a phenomic database; wherein the computer compares the SNP and phenotypic data and calculates relationships between the genomic and phenotypic data; wherein the computer compares the genomic and phenotypic relationship data to a reference standard; and wherein the computer outputs the relationship data and the standard on the display.

23. A method of diagnosing a subject with schizophrenia comprising obtaining a biological sample from the subject, obtaining clinical data from the subject, and applying the biological sample and clinical data to the diagnostic system of claim 1.

24. A method of diagnosing a subject with schizophrenia and determining the schizophrenia class comprising: a. obtaining a biological sample from the subject; b. obtaining clinical data from the subject; c. applying the biological sample and clinical data to a diagnostic system for diagnosing schizophrenia, wherein the diagnostic system comprises one or more expression panels and one or more phenotypic panels; d. comparing the genomic and phenotypic panels results to a reference standard; wherein the presence of one or more SNP sets and phenotypic sets in the subjects sample indicates the presence of schizophrenia, and wherein the genomic and phenotypic profile of the reference standard most closely correlating with the subjects genomic and phenotypic profile indicates schizophrenia class of the subject.

25. The method of claim 23, wherein the one or more expression panels each comprise one or more of the single nucleotide polymorphism (SNP) sets selected from the group comprising 19_2, 88_64, 81_13, 87_76, 58_29, 83_41, 9_9, 10_4, 14_6, 56_30, 42_37, 65_25, 71_55, 12_11, 90_78, 77_5, 88_8, 51_28, 59_48, 41_12, 22_11, 13_12, 31_22, 85_84, 87_84, 16_10, 56_19, 75_31, 81_73, 85_23, 21_8, 76_74, 61_39, 75_67, 76_63, 81_3, 87_26, 88_43, 25_10, 12_2, 52_42, or 54_51.

26. The method of claim 23, wherein the one or more phenotype panels each comprise one or more phenotypic sets selected from the group comprising 15_13, 12_11, 21_1, 50_46, 9_6, 46_23, 54_11, 30_17, 18_13, 27_6, 61_18, 64_11, 65_64, 12_4, 42_9, 52_28, 7_3, 48_41, 26_8, 69_41, 10_5, 17_2, 63_24, 69_66, 22_13, 53_6, 59_41, 20_19, 55_7, 34_17, 27_7, 4_1, 66_54, 8_4, 51_38, 42_7, 18_3, 46_29, 5_2, 57_39, 11_5, 24_4, 48_7, 28_23, or 25_20.
